SEQUENCE LISTING (138 sequences)

SEQ ID NO: 1 - protein, 348 aa, Amyelois transitella
MVPNKGSSNILPETPEGQFKKLIAPQAEPRKHKIVYRNLLTFGYWHLSAVYGLYLCFTSAKRATIIFAIFIYIIAKIGITGGAHRLWAHRAYKAKLPLEILLLIMNSIAFQDTVLTWARDHRLHHKYSDTDADPHNPTRGFFFAHMGWVLVRKHPEVLARGKALPIDDLKNNPLLRFQKKYAIPMIGTLCFLMPTFVPVYFWGESISTAWNINLLRYVTNLNVIFLINSWAHLIGNKPYDKNIAATQSIPISIATLGEGFHNYHHTYPWDYKASELGNNKINLTTKFIDFFAWVGWAYDLKSMPSEGVAKRRAKTGDGTDLWGRKVKINENIVKRTPPIVLIGREAKE

SEQ ID NO: 2 - DNA, 1044 bp, Amyelois transitella
  source 1..1044: mol_type = other DNA
gtgcccaacaagggttcttccaacattctgcccgagactcccgagggccagttcaagaagctgattgctccccaggctgagccccgaaagcacaagatcgtgtaccgaaacctgctgaccttcggatactggcacctgtctgccgtctacggtctgtacctgtgtttcacctctgccaagcgagctaccattatcttcgccatcttcatctacatcatcgctaagatcggcattaccggcggagcccaccgactgtgggctcaccgagcctacaaggccaagctgcccctggagatcctgctgctgattatgaactctatcgctttccaggacaccgtgctgacctgggctcgagatcaccgactgcaccacaagtactctgacaccgacgctgaccctcacaaccctacccgaggtttcttcttcgcccacatgggctgggtgctcgtccgaaagcaccccgaggtgctggcccggggaaaggctctgcccatcgacgacctgaagaacaaccccctgctgcgattccagaagaagtacgctatccccatgattggcaccctgtgtttcctgatgcccaccttcgtgcccgtctacttctggggtgagtctatttctaccgcctggaacatcaacctgctgcgatacgtgaccaacctgaacgtgatcttcctgatcaactcctgggctcacctgatcggcaacaagccctacgacaagaacattgccgccactcagtctatccccatctccatcgctaccctcggagagggtttccacaactaccaccacacctacccttgggactacaaggcttctgagctgggcaacaacaagatcaacctgaccaccaagttcattgacttcttcgcctgggtcggatgggcttacgacctgaagtccatgccctctgaaggcgtcgctaagcgacgagctaagaccggcgacggaaccgacctgtggggacgaaaggtgaagatcaacgagaacatcgtgaagcgaacccctccaatcgtgctgatcggccgagaggccaaggaatag

SEQ ID NO: 3 - protein, 338 aa, Spodoptera litura
MAQTIQTTTILEQKEEKTVTLLVPQAGKRKFEIVYFNLVSFAYWHIAGLYGLYLCFTSAKWATILFSFFLFVVAEVGVTAGAHRLWSHKTYKAKLPLQILLMVMNSLAFQNTAIDWVRDHRLHHKYSDTDADPHNASRGFFYSHIGWLFVRKHPDVKKRGKEIDISDIYNNPVLRFQKKYAIPFIGAVCFALPTLIPVYGWGETWTNAWHVAMLRYIMNLNVTFLVNSAAHIYGKRPYDKKILPSQNIAVSIATFGEGFHNYHHVFPWDYRAAELGNNCLNFTTKFIDFFAWIGWAYDLKTVSKEMIKQRSKRTGDGTNLWGLEDVDTPEDLKNTKGE

SEQ ID NO: 4 - DNA, 1017 bp, Spodoptera litura
  source 1..1017: mol_type = other DNA
atggctcagaccatccagaccaccaccatcctcgagcagaaggaggagaagaccgtgaccctgctggtccctcaggctggtaagagaaagttcgagatcgtctatttcaacctggtctcttttgcctactggcatatcgctggcctgtacggtctttacctgtgcttcacctccgccaagtgggccaccattctgttctccttcttcctgttcgtcgtcgccgaggttggagttaccgccggagctcaccgactgtggtcccacaagacttacaaggccaagctcccccttcagatccttctgatggtcatgaactccctggccttccagaataccgccattgactgggtccgagaccaccggcttcaccataagtactccgacaccgatgccgaccctcataacgcctcccgaggcttcttctactcccacatcggctggctgttcgtgcgaaagcaccccgatgtcaagaagcgaggtaaggagattgacatctccgacatctacaataaccccgtcctgagattccagaagaagtacgccatccctttcattggcgctgtctgcttcgccctgcccacccttatccccgtctatggttggggcgagacctggaccaacgcctggcacgtcgccatgctgcgatacatcatgaacctcaatgttaccttcctggtcaactctgccgcccacatctacggaaagcgaccctacgacaagaagatccttccctctcagaacatcgccgtctccattgccacctttggtgagggtttccacaactaccaccacgtgttcccttgggattaccgagctgccgagctgggcaacaactgcctgaacttcaccaccaagttcattgacttcttcgcttggatcggatgggcctacgacctcaagacggtctccaaggagatgatcaagcagcgatccaagcgaactggcgacggaactaacctgtggggcctggaggacgtcgatactcctgaggacctgaagaacaccaagggcgagtaa

SEQ ID NO: 5 - protein, 338 aa, Spodoptera littoralis
MAQCVQTTTILEQKEEKTVTLLVPQAGKRKFEIVYFNIITFAYWHIAGLYGLYLCFTSTKWATVLFSFFLFVVAEVGVTAGSHRLWSHKTYKAKLPLQILLMVMNSLAFQNTVIDWVRDHRLHHKYSDTDADPHNASRGFFYSHVGWLLVRKHPDVKKRGKEIDISDIYNNPVLRFQKKYAIPFIGAVCFVLPTLIPVYGWGETWTNAWHVAMLRYIMNLNVTFLVNSAAHIYGKRPYDKKILPSQNIAVSIATFGEGFHNYHHVFPWDYRAAELGNNSLNFPTKFIDFFAWIGWAYDLKTVSKEMIKQRSKRTGDGTNLWGLEDVDTPEDLKNTKGE

SEQ ID NO: 6 - DNA, 1017 bp, Spodoptera littoralis
  source 1..1017: mol_type = other DNA
atggcccagtgcgtccagaccaccaccatcctcgagcagaaggaagagaaaaccgtgaccctcctggtgccccaggccggaaagcgaaagttcgagatcgtgtacttcaacattattaccttcgcttactggcacattgccggactttacggcctgtacctgtgcttcacctctaccaagtgggccaccgttcttttctctttcttcctgttcgtggtggctgaggttggagttaccgccggctctcaccgactttggtctcataagacctacaaggccaagctccccctgcagattcttcttatggtgatgaactctctggcttttcaaaatactgttatcgactgggtgcgagaccatcggctgcaccataagtactctgacaccgacgctgatccccacaacgcttctcgaggattcttctactctcacgtgggatggcttctggtgcgaaagcaccccgacgtcaagaagcgaggaaaggagatcgacatttctgacatctacaacaatcccgtgcttcgattccagaagaagtacgctatccccttcattggcgccgtgtgtttcgtgctgcccaccctcatccctgtttacggctggggagagacctggaccaacgcctggcatgtggccatgctgcgatacattatgaacctgaacgtgactttcctggtgaactctgccgctcacatttacggcaagcgaccctacgataagaagatccttccctctcagaacatcgccgtctctatcgccaccttcggagagggatttcacaactaccaccacgtgttcccctgggattaccgagctgctgagctgggaaacaactctctgaacttccctaccaagtttattgacttcttcgcctggattggatgggcctacgacctgaagaccgtgtctaaggagatgatcaagcagcgatcgaagcgaaccggcgacggcacaaacctgtggggactggaggacgtcgacactcccgaggaccttaagaacaccaagggcgagtaa

SEQ ID NO: 7 - protein, 326 aa, Amyelois transitella
MVPNKGSSDVLSEHSEPQFTKLIAPQAGPRKYKIVYRNLLTFGYWHLSAVYGLYLCFTCAKWATILFAFFLYVIAEIGITGGAHRLWAHRTYKAKLPLEILLLIMNSIAFQDTAFTWARDHRLHHKYSDTDADPHNATRGFFYSHVGWLLVKKHPEVKARGKYLSLDDLKNNPLLKFQKKYAILVIGTLCFLMPTFVPVYFWGEGISTAWNINLLRYVMNLNMTFLVNSAAHIFGNKPYDKSIASVQNISVSLATFGEGFHNYHHTYPWDYRAAELGNNRLNMTTAFIDFFAWIGWAYDLKSVPQEAIAKRCAKTGDGTDMWGRKR

SEQ ID NO: 8 - DNA, 981 bp, Amyelois transitella
  source 1..981: mol_type = other DNA
atggtgcccaacaagggttcttccgacgtcctgtctgagcactccgagccccagttcaccaagctgattgctccccaggctggcccccgaaagtacaagatcgtgtaccgaaacctgctgaccttcggatactggcacctgtctgccgtctacggtctgtacctgtgtttcacctgcgccaagtgggctaccattctgttcgccttcttcctgtacgtgatcgctgagatcggcattaccggcggagcccaccgactgtgggctcaccgaacctacaaggccaagctgcccctggagatcctgctgctgattatgaactctatcgctttccaggacaccgccttcacctgggctcgagatcaccgactgcaccacaagtactctgacaccgacgctgaccctcacaacgctacccgaggtttcttctactcccacgtgggctggctgctggtcaagaagcaccccgaggtgaaggcccggggaaagtacctgtccctggacgacctgaagaacaaccccctgctgaagttccagaagaagtacgctatcctggtcattggcaccctgtgtttcctgatgcccaccttcgtgcccgtctacttctggggtgagggcatttctaccgcctggaacatcaacctgctgcgatacgtgatgaacctgaacatgaccttcctggtcaactccgccgctcacattttcggcaacaagccctacgacaagtctattgcctccgtgcagaacatctctgtctccctggctaccttcggagagggtttccacaactaccaccacacctacccttgggactaccgagctgctgagctgggcaacaaccgactgaacatgaccaccgccttcattgacttcttcgcctggatcggatgggcttacgacctgaagtccgtcccccaggaagccatcgctaagcgatgcgctaagaccggcgacggaaccgacatgtggggacgaaagcgatag

SEQ ID NO: 9 - protein, 336 aa, Agrotis segetum
MAQGVQTTTILREEEPSLTFVVPQEPRKYQIVYPNLITFGYWHIAGLYGLYLCFTSAKWQTILFSFMLVVLAELGITAGAHRLWAHKTYKAKLPLQIILMILNSIAFQNSAIDWVRDHRLHHKYSDTDADPHNATRGFFYSHVGWLLVRKHPEVKRRGKELDMSDIYNNPVLRFQKKYAIPFIGAMCFGLPTFIPVYFWGETWSNAWHITMLRYILNLNITFLVNSAAHIWGYKPYDIKILPAQNIAVSIVTGGEVSITTTTFFPWDYRAAELGNNYLNLTTKFIDFFAWIGWAYDLKTVSSDVIKSKAERTGDGTNLWGLEDKGEEDFLKIWKDN

SEQ ID NO: 10 - DNA, 1011 bp, Agrotis segetum
  source 1..1011: mol_type = other DNA
atggctcaaggtgttcaaactaccaccattttgagagaagaagaaccatctttgaccttcgttgttccacaagaacctagaaagtaccaaatcgtttacccaaacttgatcactttcggttactggcatattgctggtttgtacggtttgtatttgtgtttcacttctgctaagtggcaaactatcttgttctccttcatgttggttgttttggccgaattgggtattactgctggtgctcatagattgtgggctcataagacttacaaagctaagttgccattgcaaatcatcttgatgatcttgaactccattgccttccaaaactctgctattgattgggttagagatcacagattgcatcacaagtactctgatactgatgctgatccacataatgctactagaggttttttctactctcacgtcggttggttattggttagaaaacatccagaagtcaagagaagaggtaaagaattggatatgtccgacatctacaacaacccagttttgagattccaaaagaagtacgccattccattcattggtgctatgtgttttggtttgccaactttcatcccagtttacttttggggtgaaacttggtctaatgcttggcatattaccatgttgagatatatcttgaatttgaacatcactttcttggtcaactccgctgctcatatttggggttacaaaccatacgacattaagattttgccagcccaaaacattgccgtttctatagttactggtggtgaagtttctattaccaccactacattttttccatgggattacagagctgccgaattaggtaacaattacttgaacttgaccaccaagttcatcgatttctttgcttggattggttgggcctacgatttgaaaactgtttcctctgatgtcatcaagtccaaggctgaaagaactggtgatggtactaatttgtggggtttggaagataagggtgaagaagatttcttgaagatctggaaggacaactaa

SEQ ID NO: 11 - protein, 349 aa, Trichoplusia ni
MAVMAQTVQETATVLEEEARTVTLVAPKTTPRKYKYIYTNFLTFSYAHLAALYGLYLCFTSAKWETLLFSFVLFHMSNIGITAGAHRLWTHKTFKAKLPLEIVLMIFNSLAFQNTAITWAREHRLHHKYSDTDADPHNASRGFFYSHVGWLLVKKHPDVLKYGKTIDMSDVYNNPVLKFQKKYAVPLIGTVCFALPTLIPVYCWGESWNNAWHIALFRYIFNLNVTFLVNSAAHIWGNKPYDKSILPAQNLLVSFLASGEGFHNYHHVFPWDYRTAELGNNFLNLTTLFIDFCAWFGWAYDLKSVSEDIIKQRAKRTGDGSSGVIWGWDDKDMDRDIKSKANIFYAKKE

SEQ ID NO: 12 - DNA, 1050 bp, Trichoplusia ni
  source 1..1050: mol_type = other DNA
atggctgttatggctcaaactgttcaagaaactgctaccgttttggaagaagaagctagaactgttactttggttgctccaaaaactaccccaagaaagtacaagtacatctacaccaatttcttgaccttctcctatgctcatttggctgcattatatggtttgtacttgtgtttcacttccgctaagtgggaaactttgttgttctctttcgttttgttccacatgtccaacattggtattactgctggtgctcatagattgtggactcataagacttttaaggccaagttgccattggaaatcgtcttgatgattttcaactccttggctttccaaaacactgctattacttgggctagagaacatagattgcatcacaagtactctgatactgatgctgatccacataatgcttctagaggtttcttctactctcatgttggttggttgttggttaagaaacacccagatgttttgaagtacggtaagaccattgatatgtccgatgtttacaacaacccagtcttgaagttccaaaagaaatacgccgttccattgattggtactgtttgttttgctttgccaaccttgattccagtttactgttggggtgaatcttggaacaatgcttggcatattgctttgttcagatatatcttcaacttgaacgtcaccttcttggttaattccgctgctcatatttggggtaacaagccatatgataagtccattttgccagcccaaaacttgttggtttcttttttggcttctggtgaaggtttccataactaccatcatgtttttccatgggattacagaactgctgaattgggtaacaacttcttgaacttgaccaccttgttcattgatttctgtgcttggtttggttgggcttacgatttgaaatctgtctccgaagatatcatcaagcaaagagctaaaagaaccggtgatggttcttctggtgttatatggggttgggatgataaggatatggatagagatattaagtccaaggccaacattttctacgctaagaaagaatga

SEQ ID NO: 13 - protein, 455 aa, Helicoverpa armigera
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHRYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKSYE

SEQ ID NO: 14 - DNA, 1368 bp, Helicoverpa armigera
  source 1..1368: mol_type = other DNA
atggtggtcctgacctctaaggagactaagccctccgtggccgagttctacgctggcaagtctgtcttcatcaccggcggaaccggtttcctgggcaaggtcttcattgagaagctgctgtactcctgtcccgacatcggcaacatctacatgctgatccgagagaagaagggactgtctgtgtccgagcgaattaagcacttcctggacgaccccctgttcacccgactgaaggagaagcgacccgccgacctggagaagatcgtgctgattcccggagacatcaccgctcccgacctgggtattacctctgagaacgagaagatgctgatcgagaaggtgtctgtcatcattcactccgccgctaccgtcaagttcaacgagcccctgcccaccgcctggaagatcaacgtggagggaacccgaatgatgctggctctgtctcgacgaatgaagcgaattgaggtcttcatccacatttccaccgcctacaccaacaccaaccgagaggtggtggacgagatcctgtaccctgctcctgctgacattgaccaggtgcaccgatacgtcaaggacggtatctctgaggaagagactgagaagattctgaacggccgacccaacacctacaccttcaccaaggccctgaccgagcacctggtggctgagaaccaggcttacgtgcccaccatcattgtccgaccctccgtggtcgccgctatcaaggacgagcccattaagggatggctgggtaactggtacggagctaccggactgaccgtgttcaccgctaagggtctgaaccgagtcatctacggccactcttccaacatcgtggacctgattcccgtggactacgtcgccaacctggtcattgccgctggcgctaagtcttccaagtccaccgagctgaaggtgtacaactgttgctcttccgcctgcaaccccatcaccattggaaagctgatgtctatgttcgccgaggacgctatcaagcagaagtcctacgctatgcccctgcccggttggtacatcttcaccaagtacaagtggctggtcctgctgctgaccattctgttccaggtcatccccgcctacattaccgacctgtaccgacacctgatcggcaagaacccccgatacattaagctgcagtctctggtcaaccagacccgatcttccattgacttcttcacctctcactcctgggtcatgaaggctgaccgagtccgagagctgttcgcctctctgtcccccgctgacaagtacctgttcccctgtgaccccaccgacatcaactggacccactacattcaggactactgctggggagtgcgacacttcctggagaagaagtcctacgagtag

SEQ ID NO: 15 - protein, 543 aa, Amyelois transitella
MAAKCEIGVSQFFAGKHVFITGATGFMGKVLVERLLSTCADVGRIYLLMRSKKDHSPEQRLQELKASQAFDNLRQSQPSQLEKLRIVAGDIMLPGLGISQEGLEDLQEVSVVFHSAATVKFNEALKIAIEQNLMSVIRLLEICDRLPHMQAFVHVSTAYSNPELSTVEERVYPPPVPLDYLLDLAAEDSPLLDNVESFISPKPNSYTFTKAMAEAAVQRHGCQYYPIAIVRPTIVISSLKHPFPGWLENANGPTGVIFGAGRGLLRVFRCRQSALADLLPVDIGIDTLIAAAWEVASDRPPSPRVYNCSISENPTTWGDLKRAINLHTRSQPQCGAVWYPGLHTAENGFLYKILEVLLQTTPLYIGEILVRILGYSHKRLNLIRVSTQMKNMQNAMKYFALREWRFQSDNVRRLRERMSDVDKRIYNLDPCSIVWDEHYSNYVKGVRLYLLREKELDQPAAKKRLKKLYYLHYGLSVFMPFLLFRAFVRRKHRQNILEVLFAILSIFNAIFRRSTQNILYIYKVSLTFLNDNVFLNKVFNESS

SEQ ID NO: 16 - DNA, 1632 bp, Amyelois transitella
  source 1..1632: mol_type = other DNA
atggccgccaagtgcgagatcggcgtgtctcagttcttcgccggcaagcacgtgttcatcaccggcgccaccggcttcatgggcaaggtgctggtcgagcgactgctgtctacctgcgccgacgtgggccgaatctacctgctgatgcgatctaagaaggaccactctcctgagcagcgactgcaagagctgaaggcctctcaggccttcgacaacctgcgacagtctcagccctctcagctcgagaagctgcgaatcgtggccggcgacatcatgctgcccggcctgggcatctctcaagagggcctcgaggacctgcaagaggtgtctgtggtgttccactctgccgccaccgtgaagttcaacgaggccctgaagatcgccatcgagcagaacctgatgtctgtgatccgactgctcgagatctgcgaccgactgcctcacatgcaggctttcgtgcacgtgtctaccgcctactctaaccccgagctgtccaccgtcgaggaacgagtgtaccctcctcctgtgcctctggactacctgctcgacctggccgccgaggactctcccctgctggacaacgtcgagtctttcatctctcccaagcctaactcttacaccttcaccaaggccatggctgaggccgccgtgcagcgacacggctgccagtactaccccattgccatcgtgcgacccaccatcgtgatctcttcgctgaagcacccctttccaggctggctcgagaacgccaacggacccaccggcgtgattttcggagccggccgaggcctgctgcgagtgttccgatgccgacagtctgccctggccgacctgctgcctgtggacatcggcatcgacaccctgatcgccgctgcctgggaagtcgcctctgaccgacctccttcgcctcgagtgtacaactgctctatctctgagaaccccaccacctggggcgacctgaagcgagccatcaacctgcacacccgatctcagccccagtgtggcgccgtgtggtatcccggactgcataccgccgagaacggcttcctgtacaagatcctcgaggtgctgctgcagaccactcctctgtacatcggcgagatcctggtgcgaatcctgggctactctcacaagcgactgaacctgatccgagtgtctacccagatgaagaacatgcagaacgccatgaagtactttgccctgcgagagtggcgattccagtctgacaacgtgcgacgactgcgagagcgaatgtctgacgtggacaagcgaatctacaacctggatccttgctctatcgtgtgggacgagcactactccaactacgtgaagggcgtgcgactgtacctcctccgagagaaggaactggaccagcctgccgccaagaagcgactcaagaagctctactacctgcactacggcctgtctgtgttcatgccctttctgctgttccgagccttcgtccgacgaaagcaccgacagaacattctcgaggtcctgttcgccatcctgtctatcttcaacgccatcttccgacgatcgacccagaacatcctgtacatctacaaggtgtctctgaccttcctgaacgacaacgtgttcctgaacaaggtgtttaacgagtcctcttag

SEQ ID NO: 17 - DNA, 54 bp, synthetic construct
  misc_feature 1..54: note = Primer sequence PR25094
  misc_feature 1..53: note = PR25094
  source 1..54: mol_type = other DNA
cacgcgatatggtctcacagagaagacttcagacgatttgtcttagaggaacgc

SEQ ID NO: 18 - DNA, 55 bp, synthetic construct
  misc_feature 1..55: note = Primer sequence PR25381
  misc_feature 1..55: note = PR25381
  source 1..55: mol_type = other DNA
cgtgcgatatggtctcagcgggaagactttacgacgcaactaacatgaatgaata

SEQ ID NO: 19 - protein, 453 aa, Heliothis subflexa
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTNHSWVMKADRVRELFASLSPADKYLFPCDPVNINWRQYIQDYCWGVRHFLEKKT

SEQ ID NO: 20 - DNA, 1362 bp, Heliothis subflexa
  source 1..1362: mol_type = other DNA
atggttgtcttgacctccaaagaaactaagccatctgttgctgaattttacgctggtaagtctgttttcattactggtggtactggtttcttgggtaaggttttcattgaaaagttgttgtactcctgcccagatatcggtaatatctacatgttgatcagagaaaagaagggtttgtccgtttccgaaagaatcaagcactttttggatgatcctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcactcttctaacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtacatttttactaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcaccaaccattcttgggttatgaaggctgatagagtcagagaattattcgcttctttgtctccagcagataagtacttgtttccatgtgatccagtcaacatcaattggagacaatatatccaagattactgctggggtgttagacatttcttggaaaaaaagacttaa

SEQ ID NO: 21 - protein, 456 aa, Helicoverpa assulta
MVVLTSKETKPSVAEFYAGKSVFITGGTGFLGKIFIEKLLYSCPDIGNIYMLIREKKGLSVSERIKQFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSYIVDLIPVDYVANLVIAAGAKSSKSTELKVYNCCSSACNPITIGKLMSMFAEDAIKQKSYAMPLPGWYVFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKTTNK

SEQ ID NO: 22 - DNA, 1371 bp, Helicoverpa assulta
  source 1..1371: mol_type = other DNA
atggttgtcttgacctccaaagaaactaagccatctgttgctgaattttacgctggtaagtctgttttcattactggtggtactggtttcttgggtaagatcttcattgaaaagttgttgtactcctgcccagatatcggtaatatctacatgttgatcagagaaaagaagggtttgtccgtttccgaaagaatcaagcaatttttggatgaccctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcattcctcttacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtatgtttttacaaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcacctctcattcttgggttatgaaggctgatagagtcagagaattattcgcttctttgtctccagcagataagtacttgtttccatgtgatccaaccgatattaactggacccattacattcaagattactgctggggtgttagacacttcttggaaaaaaagactaccaacaagtaa

SEQ ID NO: 23 - protein, 547 aa, Drosophila grimshawi
MGDSTLKLPGAAAAPLKPPAVAALSVSSLQLPSSAPQKLTSAASNGNVSGSATGNPRNKCALKPGYSLMSWIRLCNSGADLSGTAGRVVPVSRTELARHNQVNDAWMAIRGKVFNVTRYMDYHPGGVDELMRGVGRDATKLFDEVHAWVNYPQLLGKCYVGPLKDTVASKKATDAELLRLPPPSAPAELKPRFDWIQQRRELTLYFYTRRWANPGVLLKRKTAEELEVRVQLAEIWHLYNFKLHSGIIWPPKALRVSPETGKIELVLSKQEEQPWSSYGSHEASKTSGAPEEELLDFMVQHCSHFNHDSFELCLESVEQELLLHLPVGYHLDIEVPHNGERVQRSYTPVPHNCLLAEAYSSSSSPNSQRFLIKHYENGIVSSQLNQLESGGNLQLTLPRGNFNLAQLEAHRHILLLAAGSGLTPMLSLLRPLLERHTNRIERLQLLYFNKTQADIWWKDKLLALQSSDERFLCQHILSQEVEEQPPTPNQNTRICPELLAPLFEQQQPLGERFSYVAICGPTGFNTAALDSLRRLEFKLTNVHVFQG

SEQ ID NO: 24 - protein, 535 aa, Drosophila melanogaster
MSDPSLKPPASNQPLRPPQLMAPAGSGGGLQLPVTAPQKLNSNGSSASGSATGNPRNKCALKPGYSLMNWIRLCNSGADLSGTGGRLVPVSRTELARHNKIDDAWMAIRGRVFNVTRYMDFHPGGVDELMRGVGRDATKLFDEVHAWVNYPQLLGKCYVGPLKDNETKPAKESPQITNVILQPPEIVPRFDWIQQRSELTLIFYTRSLANPGLVVRRKDLQQLSVRVLVQHNWHSFDFQLTNNVEWPPKLAKIGSETGKIELVFVKEEAEPWPTYGTHVSNRLDSSRLHDETFEYEVVHSKDFNHDSFELCLQSVGQKVLMVLPAGYHVDIEVPLEGRVIQRSYTPVDHTYLRLENIRSSRSECLHFLIKRYPNGPVSSHLQKLETGSRVHWSAPRGSFQLSDLTAHRNILLLAAGSGLTPILSLIQPILKRNTNRIESLQLLYFNKTNEDIWLKEKLHELHTDDERFSCTNYVSQSEDNPQRIALELLAPLFQKNQPERCTYVLICGPSGFNTAALDILSQLDVKANQIHVFRG

SEQ ID NO: 25 - protein, 521 aa, Homo sapiens
MLNVPSQSFPAPRSQQRVASGGRSKVPLKQGRSLMDWIRLTKSGKDLTGLKGRLIEVTEEELKKHNKKDDCWICIRGFVYNVSPYMEYHPGGEDELMRAAGSDGTELFDQVHRWVNYESMLKECLVGRMAIKPAVLKDYREEEKKVLNGMLPKSQVTDTLAKEGPSYPSYDWFQTDSLVTIAIYTKQKDINLDSIIVDHQNDSFRAETIIKDCLYLIHIGLSHEVQEDFSVRVVESVGKIEIVLQKKENTSWDFLGHPLKNHNSLIPRKDTGLYYRKCQLISKEDVTHDTRLFCLMLPPSTHLQVPIGQHVYLKLPITGTEIVKPYTPVSGSLLSEFKEPVLPNNKYIYFLIKIYPTGLFTPELDRLQIGDFVSVSSPEGNFKISKFQELEDLFLLAAGTGFTPMVKILNYALTDIPSLRKVKLMFFNKTEDDIIWRSQLEKLAFKDKRLDVEFVLSAPISEWNGKQGHISPALLSEFLKRNLDKSKVLVCICGPVPFTEQGVRLLHDLNFSKNEIHSFTA

SEQ ID NO: 26 - protein, 671 aa, Spodoptera litura
MADSPEMEQKPTISNTHRVSLPTVLCTPESTDDNSDISIKVNSIISSDSKMSLPSNLGTPTSPLRLSSPLSLPSSPCVKNSPQSTNSLPNPNPKPMTSSTASNKTVVNSGSATGNPRNKCALQPGHSLMDWIRLGNSGKDLTGVGGRMRPVTPAELSTHNTTNDAWLAIRGRVYNITHYLPYHPGGPEELMRGAGMDATQLFDKVHPWVNYDSLLAKCLVGPLRFDLPDAEELFDTSNPSPKSDRLREPSKAQELVRKSMENLANCITPVRKKITKGDDNSKGSPPSKIMQSLVQSADLPMSISRRAASSPVKKTDKSPDSPLPLRHDWIQTSTKLTISVYTGHLANPGGCARITEGFLLIEVATNGWLRTLKIKPEAKLKEPLQLRVFAESGKIEITALKAEPSVWKGCGEVTLGAASRVSSPRTVECRVMEVSRVSHDTSLLSVCPRAGPVVVPLGHHVRVHRRVEGSECVRSYTPVGEGWGPPDGTEIFSALRLAVKRYDSGVLSPHLTALKVGDLITLSGPYGNFQLQKLKTVKSLYLIAAGTGITPMLGLIKFMMSRSNPRCERTHLIFFNKTEADILFLENFEEMAKEDDRLKITHVLSDASSSWSGHKGRIRDELLTEILGQVKCDDKCSHYSCLCGPTEFTYAGLDLLKKHGFKDDCIHAFMG

SEQ ID NO: 27 - protein, 679 aa, Cydia pomonella
MADSPPEDNTPTRNGSLKISLPTVLCTPESSENSDILCKTKTFISSESKLSLPSNLGTPTSPLRLSSPLSLPTSPTPYMKITQPQPEKTVAATQMNSPPTTIVQQTGPKQPLVNTGSATGNPRNKCALQPGHSLMDWIRLGNSGKDLTGVGGRMRPVSPDELAAHNTRNDAWLAIRGRVYNITHYLPYHPGGPEELMRGAGIDATQLFDKVHPWVNYDSLLAKCLVGPLRLDRPDAEELFGPSTPSPKSENRLREPSKAQELVRKSMENLANCITPVRKKMSAKSEENIKGSPPSKIMQSLIQSSDLPVSISRRAAASPVKTSEKSDIAPSPLRFDWIQTSTKLTLSIYTGPLANPGGCARIIEGILFVEVATNGWVRTLKLVPEAKLLESLQLRVFAESGKIEVTAHKAEPGVWKTCGEAMAGAASRVSTPRSVSCRVARMLRVSHDTVLLSLAAPAQALVAPLGHHVRVHRTISDVECVRSYTPVGEGWGPDAERDSAIHLAVKGYDTGALSPYLTALQPGDEVTVSGPYGNFQLHTLKGVKEIYFVAAGTGITPMLGLLKFMLPRSNPRCERIHLLFFNKTEEDILFRENFEEIAREDDRFTVTHVLSDAGPSWSGHKGRVTTNLLTQVIGKSFLKCRDQCTHFACVCGPTEFTHTAIDLLKKLDMKDSCIHAFMG

SEQ ID NO: 28 - DNA, 1017 bp, Helicoverpa zea
  source 1..1017: mol_type = other DNA
atggcccagtcttaccagtctaccaccgtgctgtctgaggaaaaggaactgaccctgcagcacctggtgcctcaggcttcgccccgaaagtaccagatcgtgtaccccaacctgatcaccttcggctactggcacattgccggcctgtacggactgtacctgtgcttcacctctgccaagtgggccaccatcctgttctcttacattctgttcgtgctggccgagatcggcatcaccgctggcgcccaccgactgtgggctcacaagacctacaaggccaagctgcccctcgagatcctgctgatggtgttcaactctatcgccttccagaactctgccatcgactgggtgcgagatcaccgactgcaccacaagtactctgacaccgacgctgaccctcacaacgcctctcgaggcttcttctactctcacgtcggctggctgctggtgcgaaagcaccccgaggtgaagaagcgaggcaaggaactcaacatgtctgacatctacaacaaccccgtgctgcgattccagaagaagtacgctatccccttcatcggcgccgtgtgcttcgctctgcccactatgatccccgtgtacttctggggcgagacttggtctaacgcctggcacatcaccatgctgcggtacatcatgaacctgaacgtgaccttcctggtgaactctgctgcccacatctggggcaacaagccctacgacgccaagattctgcccgctcagaacgtggccgtgtctgtggccaccggcggagagggcttccacaactaccaccacgtgttcccttgggactaccgagccgccgagctgggcaacaactctctgaacctgaccaccaagttcatcgacctgttcgccgccatcggctgggcctacgacctcaagaccgtgtccgaggacatgatcaagcagcgaatcaagcgaaccggcgacggcaccgacctctggggccacgagcagaactgcgacgaggtgtgggacgtgaaggacaagtcctcttaa

SEQ ID NO: 29 - protein, 338 aa, Helicoverpa zea
MAQSYQSTTVLSEEKELTLQHLVPQASPRKYQIVYPNLITFGYWHIAGLYGLYLCFTSAKWATILFSYILFVLAEIGITAGAHRLWAHKTYKAKLPLEILLMVFNSIAFQNSAIDWVRDHRLHHKYSDTDADPHNASRGFFYSHVGWLLVRKHPEVKKRGKELNMSDIYNNPVLRFQKKYAIPFIGAVCFALPTMIPVYFWGETWSNAWHITMLRYIMNLNVTFLVNSAAHIWGNKPYDAKILPAQNVAVSVATGGEGFHNYHHVFPWDYRAAELGNNSLNLTTKFIDLFAAIGWAYDLKTVSEDMIKQRIKRTGDGTDLWGHEQNCDEVWDVKDKSS

SEQ ID NO: 30 - DNA, 1017 bp, Spodoptera exigua
  source 1..1017: mol_type = other DNA
atggctcagaccatccagaccaccaccatcctcgagcagaaggaggagaagaccgtgaccctgctggtcccccaggctggtaagagaaagttcgagttcgtctatcagaacctgattacttttgcctactggcatatcgctggcctgtacggtctttacctgtgcttcacctccgccaagtgggccaccattctgttctccttcatcctgttcgtcatcgccgagattggaatcaccgccggagctcaccgactgtggtcccacaagtcttacaaggtcaagctcccccttgagatccttctgatggtcatgaactccattgccttccagaataccgtcattgactgggtccgagaccaccggcttcaccataagtactccgacaccgatgccgaccctcataacgcctcccgaggcttcttctactcccacatcggctggctgttcgtgcgaaagcaccccgaggtcaagaagcgaggtaaggagctggacatgtccgacatctacaataaccccgtcctgagattccagcggaagtacgccgttcctttcattggcgctgtctgcttcggcctgcccacccttatccccgtctattgctggggcgagtcctggaccaacgcctggcacatcaccatgctgcgatacatcatgaacctcaatgccaccttcctggtcaactctgccgcccacatctacggaaagcgaccctacgacaagaagatccttcccgcccagaacatcggcgtctccattgccacctttggtgagggtttccacaactaccaccacgtgttcccttgggattaccgagctgccgagctgggcaacaacggtctgaacctgaccaccaagttcattgacttcttcgcttggatcggatgggcctacgacctcaagacggtctccaaggagatgatcaagcagcgatccaagcgaactggcgacggaactaacctgtggggcctggaggacaaggatactcctgagaacctgaagaacatcaagggcgagtaa

SEQ ID NO: 31 - protein, 338 aa, Spodoptera exigua
MAQTIQTTTILEQKEEKTVTLLVPQAGKRKFEFVYQNLITFAYWHIAGLYGLYLCFTSAKWATILFSFILFVIAEIGITAGAHRLWSHKSYKVKLPLEILLMVMNSIAFQNTVIDWVRDHRLHHKYSDTDADPHNASRGFFYSHIGWLFVRKHPEVKKRGKELDMSDIYNNPVLRFQRKYAVPFIGAVCFGLPTLIPVYCWGESWTNAWHITMLRYIMNLNATFLVNSAAHIYGKRPYDKKILPAQNIGVSIATFGEGFHNYHHVFPWDYRAAELGNNGLNLTTKFIDFFAWIGWAYDLKTVSKEMIKQRSKRTGDGTNLWGLEDKDTPENLKNIKGE

SEQ ID NO: 32 - DNA, 1050 bp, Plutella xylostella
  source 1..1050: mol_type = other DNA
atgtgccccaagtctcccgagcaggcctctgtggtcgaggaacgagaggaaaagtctgccgagttcgagaagctgatcgctccccaggctggaccccgaaagttcaacatcgtgtacttcaacctgctgaccttcggctactggcacctggctggcgcctacggcctgtacctgtgcttcacctctgccaagttcgccaccgtgctgttcgccatcctgacctacaccgccgctgagatcggcatcaccgctggcgcccaccgactgtggtcccacaaggcctacaaggccaagctgcccctgcagatcatcctgatgaccttcaacaccctggccttccagaactctgccatcgagtgggtgcgagatcaccgactgcaccacaagtactctgacaccgacgctgaccctcacaacgctacccgaggcttcttctactctcacgtcggctggctgctggtgcgaaagcactctgaggtgaagaagcgaggcaagaccatcgacatgtctgatatgtactctaaccccgtgctggctttccaaaagcggtacatcgtgccctgggtgattctggtgaccttcctgctgcctaccatcattcccgtctacctgtggaacgagtctctgtggacctcttggcacgtgaccatgctgcgatacgtggccaacctgaacgccaccttcctggtgaactctgctgcccacctgtggggctacaagccctacgacaagaacatcatgcccgctcagaacatctctgtgtctctggccaccttcggcgagggcttccacaactaccaccacgtgttcccttgggactacaaggctgccgagctgggcaacaaccgatacaacctgactaccaagttcatcgactttttcgcctggatcggatgggcctacgacctgaagtctgtgtctgaggaactggtggtgaaccgaatgcgacgaaccggcgacggctctaacctctggggctggggcgacaaggacatgaccgaagaggaccgaaacggcgccctgctcaccaccaagtccgtggtcgagtag

SEQ ID NO: 33 - protein, 349 aa, Plutella xylostella
MCPKSPEQASVVEEREEKSAEFEKLIAPQAGPRKFNIVYFNLLTFGYWHLAGAYGLYLCFTSAKFATVLFAILTYTAAEIGITAGAHRLWSHKAYKAKLPLQIILMTFNTLAFQNSAIEWVRDHRLHHKYSDTDADPHNATRGFFYSHVGWLLVRKHSEVKKRGKTIDMSDMYSNPVLAFQKRYIVPWVILVTFLLPTIIPVYLWNESLWTSWHVTMLRYVANLNATFLVNSAAHLWGYKPYDKNIMPAQNISVSLATFGEGFHNYHHVFPWDYKAAELGNNRYNLTTKFIDFFAWIGWAYDLKSVSEELVVNRMRRTGDGSNLWGWGDKDMTEEDRNGALLTTKSVVE

SEQ ID NO: 34 - DNA, 1569 bp, Plodia interpunctella
  source 1..1569: mol_type = other DNA
atggtggcccgacgagactctgaggccgtgcctctgattccccagttctacgccggacgatctatcttcatcaccggcgccaccggcttcatgggcaaggtgctgatcgagcgaatcctggccacctgtcctgacgtggaccgactgtacctgctggtgcgacacaagaaggacgtttctcccgagaagcgactgcagcagctgatcgactctcccgtgttcgaccccgtgcgagctactgaccccgctcagctggacaagctgtctatgctggccggcgacatcaccaagcctgacctgggcatgtctcaagagtctctgctgcgactgcacgacgtgtctgtggtgttccactctgccgccactctgaagttcgacgaggccatgaagatggccgtcgagcagaacgtgctgtctgtgatccgactgctcgagatctgcgaccgactgcctaacatgcaggccttcgtgcacgtgtctaccgcctactctaacgccgagctgcgagaggtcgaggaacacgtgtaccctccaaagatctcgctccagcagctcctggccgtgtctgagtcgctgcccgaggacctgatgatggaaatcacccctaagttcatctctcccaagcctaacacctacaccttcaccaaggccatggctgagcacgccgtgcagcagcacggaaacaagggctacgcctgcgccatcttccgacctaccatcgtgatctcttccgtgaagcacccctatcctggctggatcgagaacctgaacggcccctctggcgtgatcgccgctgccggcaagggcctgctgcatgtgttctgctgcaaccccgacgctgccgccgacatgctgcccgtggacattgccatcgacaccctgctggccgtggcctgggagactgccattgacaagtctaaggaagtgcgagtctacaactgctctacccgagagaaccccaccgactggcgaggcttcgagcaggctctgcgacggtatatcgtcgagcaccctctggaccgagcctactggtatccctccggcgcctctgtgggcaacacctacgctcagaaggccatcgagctgctcacccagactgtgcccctgcacctggtggcctacaccattcgactgctgggcatcaagacccagctgaacctgatcaccgtgtctcaccgactgcaggctatgaacaaggtcctgaagttcttcgcccagcgagagtgggtgtttcacaacgagaacgtgcgacgactgcgagcccgaatctcgccccaggacgccaagatctacaacctggatcctaactctttcgactgggacgagcactactgcaacttcatcaagggcacccgaaagtacctgctcaaggaaaaggaacaggacatcgaggaagcccgaaagcacatcaagaagatgttctacgtccactactccctgctgctgttcgtggtggtgctgctcctgcgattcgccctgaacaactccttcgtgcgacagttcgtgtacggcgtggtgcgaaccctgaccgccgtggctggcgccgtgttctctcgaatctctgcctag

SEQ ID NO: 35 - protein, 522 aa, Plodia interpunctella
MVARRDSEAVPLIPQFYAGRSIFITGATGFMGKVLIERILATCPDVDRLYLLVRHKKDVSPEKRLQQLIDSPVFDPVRATDPAQLDKLSMLAGDITKPDLGMSQESLLRLHDVSVVFHSAATLKFDEAMKMAVEQNVLSVIRLLEICDRLPNMQAFVHVSTAYSNAELREVEEHVYPPKISLQQLLAVSESLPEDLMMEITPKFISPKPNTYTFTKAMAEHAVQQHGNKGYACAIFRPTIVISSVKHPYPGWIENLNGPSGVIAAAGKGLLHVFCCNPDAAADMLPVDIAIDTLLAVAWETAIDKSKEVRVYNCSTRENPTDWRGFEQALRRYIVEHPLDRAYWYPSGASVGNTYAQKAIELLTQTVPLHLVAYTIRLLGIKTQLNLITVSHRLQAMNKVLKFFAQREWVFHNENVRRLRARISPQDAKIYNLDPNSFDWDEHYCNFIKGTRKYLLKEKEQDIEEARKHIKKMFYVHYSLLLFVVVLLLRFALNNSFVRQFVYGVVRTLTAVAGAVFSRISA

SEQ ID NO: 36 - DNA, 1365 bp, Spodoptera exigua
  source 1..1365: mol_type = other DNA
atggtggtgctgacctctaaggaaaagtctaacatgtctgtggccgacttctacgccggcaagtctgtgttcattaccggcggcaccggattcctcggcaaggtgttcatcgagaagctgctgtactcttgccccgacatcgacaagatctacatgctgatccgagagaagaagggccagtctatccgagagcgactgaccaagatcgtggacgaccctctgttcaaccgactgaaggaaaagcgacccggcgacctggacaagattgtgctgatccccggcgacgtgaccgtgcctggcctgggcatctctgacgagaacgaggccatcctgatcgacaaggtgtctgtggtgatccactctgccgccaccgtgaagttcaacgagcccctcgagactgcctggaacgtgaacgtcgagggcacccgaatgatcatggccctgtctcgaaagatgaagcgaatcgagatcttcatccacatctctaccgcctacaccaacaccaaccgagccgtggtggacgaggtgctgtaccctcctcctgccgacatcaacgaggtgcaccagtacgtgaagaacggcatcaccgaggaagagactgagaagatcctgaacggacgacccaacacctacaccttcaccaaggctctgaccgagcacctggtggccgagaaccaggcctacatgcccaccatcatcgtgcgaccctctatcgtgggcgccatcaaggacgaccccatccgaggctggctggccaactggtacggcgccaccggcctgtccgtgttcaccgccaagggcctgaaccgagtgatctacggccagtcctctcacgtggtggatctgattcccgtggactacgtggccaacctggtgatcgtggctggcgccaagacctaccgatctaacgaggtgactatctacaactcttgttcttcttcttgcaaccccatcaccatggaacgactggtgggcctgttcattgacgacaccgtcaagcacaactcttacgtgatgcccctgcctggctggtacgtgtactctaactaccgatggctggtgtacctggtgaccatcatcttccagatgatccccgcctacctggccgacatcggccgacgactgctgggcaagaaccctcggtactacaagctgcagtctctggtggctcagacccaagaggccgtgcacttcttcacctctcacacctgggagatcaagtctaagcgaacctctgagctgttcgcctctctgtctcataccgaccagcgaatcttcccatgcgacgccaagaagatcgactggaccgactacatcaccgactactgctctggcgtgcgacagttcctggaaaagaagaagtag

SEQ ID NO: 37 - protein, 454 aa, Spodoptera exigua
MVVLTSKEKSNMSVADFYAGKSVFITGGTGFLGKVFIEKLLYSCPDIDKIYMLIREKKGQSIRERLTKIVDDPLFNRLKEKRPGDLDKIVLIPGDVTVPGLGISDENEAILIDKVSVVIHSAATVKFNEPLETAWNVNVEGTRMIMALSRKMKRIEIFIHISTAYTNTNRAVVDEVLYPPPADINEVHQYVKNGITEEETEKILNGRPNTYTFTKALTEHLVAENQAYMPTIIVRPSIVGAIKDDPIRGWLANWYGATGLSVFTAKGLNRVIYGQSSHVVDLIPVDYVANLVIVAGAKTYRSNEVTIYNSCSSSCNPITMERLVGLFIDDTVKHNSYVMPLPGWYVYSNYRWLVYLVTIIFQMIPAYLADIGRRLLGKNPRYYKLQSLVAQTQEAVHFFTSHTWEIKSKRTSELFASLSHTDQRIFPCDAKKIDWTDYITDYCSGVRQFLEKKK

SEQ ID NO: 38 - DNA, 1365 bp, Spodoptera litura
  source 1..1365: mol_type = other DNA
atggtggtgctgacctctaaggaaaagtctaacatgtctgtggccgacttctacgccggcaagtctgtgttcattaccggcggcaccggattcctcggcaaggtgttcatcgagaagctgctgtactcttgccccgacatcgacaagatctacatgctgatccgagagaagaagggccagtctatccgagagcgactgaccaagatcgtggacgaccctctgttcaaccgactgaaggacaagcgacccggcgacctgggcaagatcattctgatccccggcgacatcaccgtgcctggcctgggcatctctgaggaaaacgagactatcctgatcgagaaggtgtctgtggtgatccactctgccgccaccgtgaagttcaacgagcccctggccaccgcctggaacgtgaacgtcgagggcacccgaatgatcatggccctgtctcgacgaatgaagcgaatcgaggtgtttatccacatctctaccgcctacaccaacaccaaccgagccgtgatcgacgaggtgctgtaccctcctcctgccgacatcaacgacgtgcaccagcacgtgaagaacggcgtgaccgaggaagagactgagaagatcctgaacggacgacccaacacctacaccttcaccaaggctctgaccgagcacctggtggccgagaaccagtcttacatgcccaccatcatcgtgcgaccctctatcgtgggcgccatcaaggacgaccccatccgaggctggctggccaactggtacggcgccaccggcctgtccgtgttcaccgccaagggcctgaaccgagtgatctacggccactctaaccacgtcgtggatctgattcccgtggactacgtggccaacctggtgatcgtggctggcgccaagacctaccactccaacgaggtgaccatctacaactcttgttcttcttcttgcaaccccatcaccatgaagcgactggtgggcctgttcattgactacaccgtcaagcacaagtcttacgtgatgcccctgcctggctggtacgtgtactctaactaccgatggctggtgttcctggtgaccctgatcttccaggtgatccccgcctacctgggcgacatcggccgacgactgctgggaaagaaccctcggtactacaagctgcagaacctggtcgctcagacccaagaggccgtgcacttcttcacctctcacacctgggagattaagtctaagcgaacctctgagctgttctcttctctgtccctgaccgaccagcgaatgttcccctgcgacgccaaccgaatcgactggaccgactacatcaccgactactgctctggcgtgcgacagttcctcgagaagatcaagtag

SEQ ID NO: 39 - protein, 454 aa, Spodoptera litura
MVVLTSKEKSNMSVADFYAGKSVFITGGTGFLGKVFIEKLLYSCPDIDKIYMLIREKKGQSIRERLTKIVDDPLFNRLKDKRPGDLGKIILIPGDITVPGLGISEENETILIEKVSVVIHSAATVKFNEPLATAWNVNVEGTRMIMALSRRMKRIEVFIHISTAYTNTNRAVIDEVLYPPPADINDVHQHVKNGVTEEETEKILNGRPNTYTFTKALTEHLVAENQSYMPTIIVRPSIVGAIKDDPIRGWLANWYGATGLSVFTAKGLNRVIYGHSNHVVDLIPVDYVANLVIVAGAKTYHSNEVTIYNSCSSSCNPITMKRLVGLFIDYTVKHKSYVMPLPGWYVYSNYRWLVFLVTLIFQVIPAYLGDIGRRLLGKNPRYYKLQNLVAQTQEAVHFFTSHTWEIKSKRTSELFSSLSLTDQRMFPCDANRIDWTDYITDYCSGVRQFLEKIK

SEQ ID NO: 40 - DNA, 1380 bp, Spodoptera exigua
  source 1..1380: mol_type = other DNA
atgacctaccgacagatcaacgagttcgacgccgagaagttcaccgccgctgctgtgcccacctcttacgtgtctgtgcccgacttctacgccggcaagtctatcttcattaccggcggcaccggattcctcggcaaggtgttcctcgagaagctgctgtactcttgcaaggacgtcgagactgtgtacatcctgatccgagagaagaagggcaagacccctcagcagcgagtcgaggacctgttcaacaagcccatcttctcgcgactgaagcaaaaggactctcagtgcatgaagaaggtgaccgccatcatcggcgacctgtctgagcccggcctgggcatctctaaggacgacgaggaactgctgctgcaaaaggtgtctgtggtgttccacgtggccgccaacgtgcagttctacaaggaattcaaggaaatcatcaacaccaacgtcggaggcaccaagtacgtgctgcagctgtgccagcgaatcaaggacatcaaggccttcgtgcacatctctaccgcctactgccacaccgaccagaaggtgctggaagaacgaatctaccctcctcctgccgaactgtctgaggtgctgaagttcctgcagcagccccagcacgacaagaagcagatcaaggaactgtttaagaagcagcccaactcttacaccttcgccaaggctctggccgagacttacattgccgagaactgcggacgagtgcccaccatcatcattcgaccctctattatctctgcctctctgaaggaacccctgcctggctgggtcgactcttggaacggcgccaccggcctgatcaccgcctcttacaacggcgctaaccgagtgctgctcggcgaaggcggcaacttcctggacctgattcctgtggacttcgtggccaacctggccatcgtggccgctgccaagtgcacctcttcgctgaaggtgtacaactgctgttcttctggctgcaaccctctgactctgaagcagctggtgtctcacatgaacaacgtgggcttcgacaagaacgtgtctatcatctttaccaacaacaaggcctctctgtctaccctgacattcttcctgcagaccactccttcttttaccgccgacatgttcctgcgagtgaccggaaagtctccccgatatatgaagatccagtctaagctgaccattgctcgaaacgccctgaacttcttcacctgtcactcttgggtgatgaaggccgacaactctcgacgactgtacgcttctctgtctctgcacgaccgacacactttcccttgcgaccccaccgacatcgactggaagaagtacatcaccatctacatcgagggaatcaaccagttcctgatgaagaagcgatcttag

SEQ ID NO: 41 - protein, 459 aa, Spodoptera exigua
MTYRQINEFDAEKFTAAAVPTSYVSVPDFYAGKSIFITGGTGFLGKVFLEKLLYSCKDVETVYILIREKKGKTPQQRVEDLFNKPIFSRLKQKDSQCMKKVTAIIGDLSEPGLGISKDDEELLLQKVSVVFHVAANVQFYKEFKEIINTNVGGTKYVLQLCQRIKDIKAFVHISTAYCHTDQKVLEERIYPPPAELSEVLKFLQQPQHDKKQIKELFKKQPNSYTFAKALAETYIAENCGRVPTIIIRPSIISASLKEPLPGWVDSWNGATGLITASYNGANRVLLGEGGNFLDLIPVDFVANLAIVAAAKCTSSLKVYNCCSSGCNPLTLKQLVSHMNNVGFDKNVSIIFTNNKASLSTLTFFLQTTPSFTADMFLRVTGKSPRYMKIQSKLTIARNALNFFTCHSWVMKADNSRRLYASLSLHDRHTFPCDPTDIDWKKYITIYIEGINQFLMKKRS

SEQ ID NO: 42 - DNA, 1362 bp, Trichoplusia ni
  source 1..1362: mol_type = other DNA
atgatcgccgtgacctctcacgagaacgaggcctctatcgtggacttctacgagggcaagtctgtgttcattaccggcggcaccggattcctcggcaaggtgttcatcgagaagctgctgtactgctgtcccggcctggtgaagatctacatgctgatccgagagaagaagggcctgaccatcaaggaacgaattgaccgattcgtggacgactctctgttcgaccgactcaagacccagcgacctaaggacctggacaagatcgtgctgatccccggcgacatcactgccccttctctgggcatctctcaagagcaagagaacatcctgatcgaagaggtgtctgtgatcatccactctgccgccaccatcaagttcaacgcccctctggaagaggcctggtctatcaacgtggacggcacccgacgaatcctggacctgtgccgaaacatgaagaaggtcgaggttttcctgcacatctctaccgcctacaccaacatcaaccgaaacgtgatcgaggaagtgctctaccctcctccagccgacattaaggaactggaaaagctggacaagcacaacgtgaccgagaagcagaccatgaagctgctcaacggcttccccaacacctacaccttcaccaagtctctgaccgagcacctggtggccgagaactgcgcctacatgcccaccatcatcgtgcgaccctctgtggtggccgccgtgctcgaggaccccatcaagggctggctggaaaactggtacggcgccaccggcatctccgtgttcaccgccaagggactgaaccgagtgttcctgggccacaagaacattattgtggacgtgattcccgccgactacgtggccaacctggtgatcattgccggcgctcgaaacaacaagtccaaggaactcaagatctataactgctgttcttctggctgcaaccctctgaccatcggcaacctgctgggcgccttcatcgacgacgccatccgacacaagacctacgctatgcccctgcctggctggttcatcttcacccgataccgactggtggctaccatcgtgaccttcatcttccaggtggtgcccgcctacctggccgacctgtaccgacgaattatcggcaagaagcccaagtacaccaagctgctctacctggtggtgcagacccgagtggccctggacttcttctcctctaactcttgggtgatgaaggccgaccgaatgcgagagctgtacgcctctctgtctaacaacgacaagcgaatgttcccttgcgaccccactcacatcaactggcccgagtacctgtctgactacggcgctggcgtgcgacgattcctcgagaagcgaaactag

SEQ ID NO: 43 - protein, 453 aa, Trichoplusia ni
MIAVTSHENEASIVDFYEGKSVFITGGTGFLGKVFIEKLLYCCPGLVKIYMLIREKKGLTIKERIDRFVDDSLFDRLKTQRPKDLDKIVLIPGDITAPSLGISQEQENILIEEVSVIIHSAATIKFNAPLEEAWSINVDGTRRILDLCRNMKKVEVFLHISTAYTNINRNVIEEVLYPPPADIKELEKLDKHNVTEKQTMKLLNGFPNTYTFTKSLTEHLVAENCAYMPTIIVRPSVVAAVLEDPIKGWLENWYGATGISVFTAKGLNRVFLGHKNIIVDVIPADYVANLVIIAGARNNKSKELKIYNCCSSGCNPLTIGNLLGAFIDDAIRHKTYAMPLPGWFIFTRYRLVATIVTFIFQVVPAYLADLYRRIIGKKPKYTKLLYLVVQTRVALDFFSSNSWVMKADRMRELYASLSNNDKRMFPCDPTHINWPEYLSDYGAGVRRFLEKRN

SEQ ID NO: 44 - DNA, 1608 bp, Plodia interpunctella
  source 1..1608: mol_type = other DNA
atgcgactggtgccctctgctctggacgtggtgctggccgccgagcgacgactggtccccatccacgaggctaccctgaagggcgactctgaggtgcagaagttctacgccggctctgtggtcctggtgaccggcggctctggcttcctgggcaagcagctgatcgagaagctgttccgaacctgcgacgtgaagaagctgtacatcctgctgcgagccaagaagggcaagtctatcggcgagcgactggactacattctgcgaaaccccgtgtacgacaccctgcgagagactcgaccctggttcgccgaccgaatcgaggccgtcgagggcgacattgcctcttaccgactgggcgtcgccgaccatatgtgggaccgactggtggacgaggtgaccgtggtgttccacggcgctgccaccatcaacttcgctgaccctctgcagctggccaccaacatcaacgtgaagggcaccctcgagatgctcgagttcggcaaggcctgcaagaacctccgatcttacgtgcacatctctaccgcctacaaccaggctctgaagaacaacgtgtcttgcgacatcctggaagagttccccaaggctcccctgtctcctaccactctggtggacctggccaaccgaaccgagactaaggtgctggacgagatgttccacaagtacctggcacctgagtaccccaacacctacgcctacaccaaggccgtggccgaagaggccgtgcgaaccatggccggcgacctgcctatctgcgtggtgcgaccctctatcgtgatccccgcctacactgagccctctgccggctgggtcgacaagaactctatctacggcgcctctggcatcatcatgggcctcggcctgggcgtgattcacaccctgatctctgaccccgacatcaagatcgacatcatccccgtggacatcgtgaacaacgccgtgatcgtggccggatgggagactgcccgacgacgagagcagggcgagactgacaccaagatctacaccgtgggatcttctaaccgaaaccacatcacctggcgatttctgaccaacgtgttcgacaacgagggccgaaagcacctgtcctctcaggccgtgtggtacgccttcgccgtgcagaccaagtctaagcacctctacctggctctggcctggctgctgcacttcatccccggctacatcgtggacggcgtgctgatgatccgaggcaagaagccccaggtttctaagatctaccgactgctgaacactatggccaccatcttctcgttcttcaccctccgaggctggaacttcaaggacgacaacctgctgcagctgtacaagtctctgtctaagaccgaccagcagatcttcaacatggacattgctaccctggacatgaaggaagtgatctacctctggcacattggcctgcgacgattctacctgaaggacaagctgaacgaccaagagaagtctatgaagaagcagttcttcctgcgaatcatcacctacaccttcattcctatctacctgtacttcctgttcaagatctcttgcttcgtgttctacggcctgtactacctgatcttctcttacctgatttga

SEQ ID NO: 45 - protein, 535 aa, Plodia interpunctella
MRLVPSALDVVLAAERRLVPIHEATLKGDSEVQKFYAGSVVLVTGGSGFLGKQLIEKLFRTCDVKKLYILLRAKKGKSIGERLDYILRNPVYDTLRETRPWFADRIEAVEGDIASYRLGVADHMWDRLVDEVTVVFHGAATINFADPLQLATNINVKGTLEMLEFGKACKNLRSYVHISTAYNQALKNNVSCDILEEFPKAPLSPTTLVDLANRTETKVLDEMFHKYLAPEYPNTYAYTKAVAEEAVRTMAGDLPICVVRPSIVIPAYTEPSAGWVDKNSIYGASGIIMGLGLGVIHTLISDPDIKIDIIPVDIVNNAVIVAGWETARRREQGETDTKIYTVGSSNRNHITWRFLTNVFDNEGRKHLSSQAVWYAFAVQTKSKHLYLALAWLLHFIPGYIVDGVLMIRGKKPQVSKIYRLLNTMATIFSFFTLRGWNFKDDNLLQLYKSLSKTDQQIFNMDIATLDMKEVIYLWHIGLRRFYLKDKLNDQEKSMKKQFFLRIITYTFIPIYLYFLFKISCFVFYGLYYLIFSYLI

SEQ ID NO: 46 - DNA, 1089 bp, Amyelois transitella
  source 1..1089: mol_type = other DNA
atggttccaaacaagggttcctctaacattttgccagaaactccagaaggtcagttcaagaaattgattgctccacaagctgaacctagaaagcacaaaatcgtttacagaaacttgttgaccttcggttactggcatttgtctgctgtttatggcttgtacttgtgtttcacttctgctaagagagccaccattattttcgccattttcatctacattatcgccaagattggtattactggtggtgctcatagattatgggctcatagagcttacaaagctaagttgccattggagatcttgctgttgattatgaactccattgctttccaagataccgttttgacttgggctagagatcatagattgcatcacaagtactctgatactgatgctgatccacataatccaactagaggttttttcttcgctcatatgggttgggtcttagttagaaaacatccagaagttttggctagaggtaaggctttgccaattgatgatttgaagaacaaccccttgttgaggttccaaaaaaagtacgccattccaatgattggcaccttgtgttttttgatgccaactttcgttccagtttacttctggggtgaatctatttctactgcctggaacatcaacctgttgagatacgttactaatctgaacgtcatcttcctgattaactcttgggctcatttgattggtaacaagccttacgataagaacattgctgctacccaatctatcccaatttccattgctactttaggtgaaggtttccataactaccatcatacttatccttgggattacaaggcttctgaattgggtaacaacaagattaacttgaccaccaagttcatcgattttttcgcttgggttggttgggcttacgatttgaaatctatgccatctgaaggtgttgctaaaagacgtgctaaaactggtgatggtactgatttgtggggtagaaaggttaagatcaacgaaaacatcgttaagagaaccccaccaattgtcttgataggtagagaagctaaagagggtaagcctatccctaaccctctcctcggtctcgattctacgtaa

SEQ ID NO: 47 - DNA, 1005 bp, Amyelois transitella
  source 1..1005: mol_type = other DNA
atggttccaaacaaaggttcctccgatgttttgtctgaacattctgaaccacaattcaccaagttgattgctccacaagctggtccaagaaagtacaaaatcgtttacagaaacttgctgaccttcggttattggcatttgtctgctgtttatggcttgtacttgtgtttcacttgtgctaagtgggctactattttgttcgctttcttcttgtacgttatcgccgaaattggtattactggtggtgctcatagattatgggctcatagaacttacaaagccaagatgccattggagatcttgttgttgattatgaactccattgccttccaagatactgcttttacttgggctagagatcatagattgcatcacaagtactctgatactgatgctgatccacataatgctactagaggtttcttctactctcatgttggttggttgttggttaagaaacacccagaagttaaggctagaggtaagttcttgtctttggatgacttgaagaacaaccccttgttgaagttccaaaagaagtacgccattttggtcattggcactttgtgttttttgatgccaactttcgttccagtttacttctggggtgaatctatttctactgcctggaacattaacttgttgaggtacgttatgaacctgaacatgacctttttggttaactccgctgctcatatttttggtaacaagccttacgataagtctatcgcctctgttcaaaacatctctgtttctttggctactttcggtgaaggtttccataactaccatcatacttatccatgggattacagagctgctgaattgggtaacaataggttgaatatgaccaccgccttcattgattttttcgcttggattggttgggcctacgatttgaaatctgttccacaagaggctattgctaagagatgtgctaaaactggtgatggtactgatatgtggggtagaaaaagacaccaccatcatcaccactaatgatag

SEQ ID NO: 48 - DNA, 1398 bp, Helicoverpa armigera
  source 1..1398: mol_type = other DNA
atggtcgttttaacttctaaagagacaaaaccttcagtagctgagttttatgcgggaaaatctgtttttattacgggtggcactggattccttggaaaggtattcatagagaaacttctgtatagctgtccagatatcgagaatatctacatgctcatacgagagaagaaaggtctttctgttagcgaaagaataaaacagttccttgatgacccgctctttaccagactaaaagacaaaagaccagctgacttagagaagattgtattaataccaggagatattactgctcctgacctgggcattaattctgaaaacgagaagatgcttatagagaaggtatcggtgattattcattcggctgctacggtgaagtttaatgagcctctccctacggcttggaagatcaacgtggaaggaaccagaatgatgctggctttgagtcgaagaatgaagcggattgaggttttcattcacatatcgacagcatacacgaacacaaacagggaagtggttgacgagatcctgtacccagctcctgctgatatcgaccaagttcatcagtatgtcaaagatggaatctctgaggaagacactgagaaaatactgaatggtcgtccaaatacgtacacgtttacgaaagcgctgactgagcatttagttgctgagaaccaagcctacgtacccactattatcgtcaggccgtcagtcgtggcagcaataaaagatgagccattaaaaggttggttaggcaactggtttggagcgactggtctcaccgtgttcaccgctaagggtctcaaccgagtcatctacggtcattctagctacatcgtagacctgattcctgtggattatgtcgctaatctggtgattgctgctggggctaagagtagcaagtcaactgagttgaaggtatacaactgctgcagcagctcctgcaatcccgtcactattggcacgctgatgagcatgtttgctgacgatgccatcaaacagaagtcgtatgctatgccgctaccggggtggtacatattcacgaaatataagtggctggttcttcttctgacatttctcttccaagttataccggcgtatgtcacagatctctccaggcacttgattgggaagagtccacggtacataaaactccaatcactagtaaatcaaacgcgctcttcaatcgacttcttcacgaatcactcctgggtgatgaaggcagacagagtgagagagctgtatgcgtctctttcccccgcagacaagtacctgtttccctgtgatcctacggacattaactggacacattacatacaagactactgttggggagtccgacattttttggagaaaaaaagctacgaagagcagaagctgatctccgaggaggacctgtaa

SEQ ID NO: 49 - DNA, 1689 bp, Amyelois transitella
  source 1..1689: mol_type = other DNA
atggctgccaagtgtgaaatcggtgtttctcaatttttcgccggtaagcacgttttcattactggtgctactggtttcatgggtaaagttttggtcgaaaggttgttgtctacttgtgctgatgttggtagaatctacttgttgatgagatccaagaaggatcactctccagaacaaagattgcaagaattgaaagcttcccaagccttcgataacttgagacaatctcaaccatctcagttggaaaagttgagaatagttgccggtgatattatgttgccaggtttgggtatttctcaagaaggtttggaagatctgcaagaggtttctgttgtttttcattcagctgctaccgtcaagtttaacgaagctttgaagattgccatcgagcagaatttgatgtccgttattaggttgttggaaatctgcgatagattgccacatatgcaagctttcgttcatgtttctactgcttactctaacccagaattgtctaccgttgaagaaagagtttatccaccaccagttccattggattacttgttggatttggctgctgaagattctcctttgttggataacgttgagtccttcatttctccaaagccaaactcttacactttcactaaggctatggctgaagctgctgttcaaagacatggttgtcaatattacccaatcgctatcgttagaccaactatcgttatctcctctctgaaacatccatttccaggttggttagaaaatgctaatggtccaactggtgttatttttggtgctggtagaggtttgttgagagttttcagatgtagacaatctgctttggctgatttgttgccagttgatattggtattgataccttgattgctgctgcctgggaagttgcttctgatagaccaccatctccaagagtttacaactgctctatttctgaaaacccaactacttggggtgatttgaagagagctattaacttgcataccagatcacaaccacaatgtggtgctgtttggtatccaggattgcatactgctgaaaatggcttcttgtacaaaatcttggaagtcttgttgcagactactcccttgtacattggtgaaattttggtcagaatcttgggctactctcacaagagattgaacttgattagagtctctacccagatgaagaacatgcaaaatgccatgaagtactttgctttgagagaatggcgtttccaatccgataatgtgagaagattgagagaaagaatgtccgatgttgacaagaggatctacaatttggatccatgttctatcgtttgggacgaacactattctaactacgtaaaaggtgtcaggttgtacctgttgagggaaaaagaattggatcaaccagctgctaaaaagaggttgaagaagttgtactacttgcactacggtctgtctgtttttatgccattcttgttgtttagagccttcgtcagaagaaagcacagacaaaacattttggaggttttgttcgccatcctgtctattttcaacgctatcttcagaaggtctacgcagaatatcttgtacatctacaaggtgtctctgacgttcttgaacgataacgttttcctgaacaaggtgttcaacgagtcctctgagaacctgtacttccaaggcgagcagaagctgatctccgaggaggacctgtgatagtga

SEQ ID NO: 50 - DNA, 1038 bp, Chilo suppressalis
  source 1..1038: mol_type = other DNA
atggccccgaattcaattcaaaatgatgaaggcttaggagtggaggtgcaaccggagagtcttacaccaccgaaacttacacaaatacggcataagatattgtacgttaatttaacgtatttcctgtattggcatctggcggcgccctatggaatttacctgtgttttacatcagtaaaatgggaaaccataattttcggttttgtcgtatttttggcggcagaactgggagtcactgccggcgcccacagactatggtcccacaagagctacaaggcgaaaaggccactggagatcatccttatgctgtttaactctatggccttccagaactccataatccactgggctagagaccatagactccaccataagtactgtgacaccgacgcggatccgtacaacgcctcaagagggttcttttactctcacgttggatggctgctgaccaagaaaaataaggaagtgttgaaaaggggcaagaccatagacgcttccgacttgttcaacaatccagtattgcagtttcagaagaagtacgcaatcccagtgttcgcgttgtgttgtttcattctgccaacagcaatacccatgtatgtatggggagagactttgaacaactcatggcatgccaacttgttgagatacaccataaacctgaacgtcacattcctcgtgaatagcgccgcacacatatggggctatcggccttacgacaaaagtatctacccagctcaaaacgtgtacgtgtctctggtgacattgggtgaaggattccacaactaccaccacgttttcccatgggattacaaaacggctgaactcggcaacaacaagttgaatgtaaccacttggtttatcaacttctttacgaagcttggctgggcttacgatctcaaaacggtgtcgaacgaaatagtgagagccagggcgatgagaaccggggatggcaaggatctgtggggatggaatgataaagacatcacggatatggataaggagcatacagatatattgtacagcaagacggaggattga

SEQ ID NO: 51 - protein, 345 aa, Chilo suppressalis
MAPNSIQNDEGLGVEVQPESLTPPKLTQIRHKILYVNLTYFLYWHLAAPYGIYLCFTSVKWETIIFGFVVFLAAELGVTAGAHRLWSHKSYKAKRPLEIILMLFNSMAFQNSIIHWARDHRLHHKYCDTDADPYNASRGFFYSHVGWLLTKKNKEVLKRGKTIDASDLFNNPVLQFQKKYAIPVFALCCFILPTAIPMYVWGETLNNSWHANLLRYTINLNVTFLVNSAAHIWGYRPYDKSIYPAQNVYVSLVTLGEGFHNYHHVFPWDYKTAELGNNKLNVTTWFINFFTKLGWAYDLKTVSNEIVRARAMRTGDGKDLWGWNDKDITDMDKEHTDILYSKTED

SEQ ID NO: 52 - DNA, 1056 bp, Plutella xylostella
  source 1..1056: mol_type = other DNA
atgtgtccaaagtctccagaacaagcttctgttgttgaagaaagggaagaaaagtctgccgaattcgaaaaattgattgctccacaagctggtccaagaaagtttaacatcgtttacttcaacttgctgaccttcggttattggcatttggctggtgcttatggtctgtacttgtgttttacttctgctaagttcgctaccgttttgttcgctattttgacttataccgctgccgaaattggtattactgctggtgctcatagattgtggtcacataaggcttacaaagctaaattgccactgcagattatcctgatgactttcaatactttggccttccaaaactccgctattgaatgggttagagatcacagattgcatcacaagtactctgatactgatgctgatccacataatgctactagaggtttcttctactctcatgttggttggttgttggttagaaaacactccgaagttaagaagagaggtaagaccattgatatgtccgacatgtactctaatccagtcttggcttttcaaaagaggtatatcgttccttgggttatcttggttactttcttgttgccaaccatcattccagtttacttgtggaatgaatctttgtggacttcttggcatgttaccatgttgagatacgttgctaatttgaacgctaccttcttggttaattctgctgctcatttgtggggttacaaaccatacgataagaacattatgccagctcagaacatctctgtttctttggctacttttggtgaaggtttccataactaccatcatgtttttccatgggattacaaggctgctgaattgggtaacaatagatacaacttgaccaccaagttcatcgatttctttgcttggattggttgggcttacgatttgaagtctgtttccgaagaattggtcgtcaacagaatgagaagaactggtgatggttctaacttatggggttggggtgataaggatatgactgaagaggatagaaatggtgctttgttgactaccaagtccgttgttgaatgatagtaa

SEQ ID NO: 53 - protein, 669 aa, Agrotis segetum
MNTMDGSPGRENQKSTRKISLPTVLCTPEPSDDRSDMKVNSFMSSDSKMSLPTNLGTPTSPLRLSSPISLPTSPCVKTSQTSSANSLPNPNPKPVTSSAATNNIVNSGSATGNPRNKCALQPGHSLMDWIRLGNSGKDLTGVGGRIRPVSPAELSTHNTLNDAWLAIRGRVYNITHYLPYHPGGPEELMRGAGMDATQLFDKVHPWVNYDSLLAKCLVGPLRFELPDAEELFDTSNPSPKSDRLREPSKAQELVRKSMENLANCITPVRKKITKGDDNTKGSPPSKIMQSLVQSADLPMSISRRAASSPVKKTEKSPDSPLPLRYDWIQTSTKLTIYVYTGHLANPGGCARITEGFLLIEVATNGWLRTLKIKPEAKLKEPLQLRVFAESGKIEITALKVDPSVWKGCGDVSVGVASHVSSPRSVECRVMEVSRVSHDTSLLSVCPRAGPVVVPLGHHVRVHRRIEGTECIRSYTPVGEGWGPADGTEVFSALRLAVKRYDSGALSPHLTALKVGDLITLSGPYGNFQLQKLKTVKSLYLIAAGTGVTPMLGLIRFMISRSNPRCERTHLLFFNKTEEDILFRENFEEMMKEDDRLKITHVLSEGSSSWSGHRGRIRDELLAETIGNVKCDDKCSHYACLCGPTEFTYAGLDLLKKHGFKDDCIHAFMG

SEQ ID NO: 54 - DNA, 1044 bp, Thaumetopoea pityocampa
  source 1..1044: mol_type = genomic DNA
atggcgccgaacacacgagagaacgagacgatatacgatgaagttgaacataagctggagaaactcgtgccgccgcaagcgggcccctggaactacaaaatagtttatctgaacctactgaccttctcttactggcttatcgccggcgcctacgggttatatttgtgttttacatcagcaaaatgggccacaattattttcgaattcatcttattctttttcgccgagatgggcatcacggcaggtgctcaccgtctttggacacataaatcttacaaagccaagttgcctttggaaattttcctcatggtgctgaattctgttgcgttccaaaacacggccactgactgggtgagagatcatcggctgcaccataagtacagcgacacggacgcggatcctcacaacgctgcgcgagggctgttcttctctcatgtcgggtggctgctcgtccggaagcacgacgaagtcaagaagcgcgggaagtttaccgatatgtcagatatatacaacaatcctgtgttgaagttccagaagaaatatgccataccgttcataggtgccgtttgttttatcttaccgacggtgatcccgatgtacttttggggcgagagtctcaacaacgcctggcacatctgtatacttcggtatgcgatgaacctcaacgtcacgttctcggtgaacagtctggcgcatatttggggcaataagccttacgacaaggatataaaaccggctcagaacttcggtgttacattagcgaccttcggcgaagggtttcacaactatcaccacgtgttcccttgggactatcgtacgtctgaactcggcgacaacaagttcaatttcacgacgaaattcataaatttctttgaacggatcggcttagcgtatgatctgaagactgtttcggatgacgttatcgcgcaaagagccaaaagaaccggtgatggtacacatctttgggattgtgccgataagaataataacgatgttgttcaaacgaaagcgcaaatagatactttatgtacaaaacatgaataa

SEQ ID NO: 55 - protein, 347 aa, Thaumetopoea pityocampa
MAPNTRENETIYDEVEHKLEKLVPPQAGPWNYKIVYLNLLTFSYWLIAGAYGLYLCFTSAKWATIIFEFILFFFAEMGITAGAHRLWTHKSYKAKLPLEIFLMVLNSVAFQNTATDWVRDHRLHHKYSDTDADPHNAARGLFFSHVGWLLVRKHDEVKKRGKFTDMSDIYNNPVLKFQKKYAIPFIGAVCFILPTVIPMYFWGESLNNAWHICILRYAMNLNVTFSVNSLAHIWGNKPYDKDIKPAQNFGVTLATFGEGFHNYHHVFPWDYRTSELGDNKFNFTTKFINFFERIGLAYDLKTVSDDVIAQRAKRTGDGTHLWDCADKNNNDVVQTKAQIDTLCTKHE

SEQ ID NO: 56 - protein, 342 aa, Notodonta dromedarius
MSSNTQPNNTLIEEVDENVGELQTRTRKLQIVYPNLITFGYWHIAGLYGLYLYFTSAKWATCIFGIILYTCAQIGITAGAHRLWAHRTYKAKLPLEILLLVFNSIAFQNTVIDWVRDHRLHHKHSDTDADPHNASRGFFFSHIGWLLVRKHDAVKKCGKRIDLSDIYNNPVLKFQKKYAIPFIGTMCFILPTLIPMYCWGETFTNAWHITILRYALNLNITWLINSVAHRWGNKPYDKSIVPVQNLSVSLISLGEGFHNYHHVFPWDYRTAELGNNTMNLTTKFIDFFAWIGWAYDLKTVPDHMIKQRVKRTGDGTNLWGWGDKNMSEEMKVGAKILFIKSE

SEQ ID NO: 57 - DNA, 1029 bp, Notodonta dromedarius
  source 1..1029: mol_type = other DNA
atgtcctctaacacccagcctaacaacaccctgatcgaagaggtggacgagaacgtgggcgagctgcagacccgaactcgaaagctgcagatcgtgtaccccaacctgatcaccttcggctactggcacattgccggcctgtacggactgtacctgtacttcacctctgccaagtgggccacctgtatcttcggcatcatcctgtacacctgtgctcagatcggcatcaccgctggcgcccaccgactgtgggctcaccgaacctacaaggccaagctgcccctcgagatcctgctgctggtgttcaactctatcgccttccagaacaccgtgattgactgggtgcgagatcaccgactgcaccacaagcactctgacaccgacgctgaccctcacaacgcctctcgaggcttcttcttctctcacatcggctggctgctggtccgaaagcacgacgccgtgaagaagtgcggcaagcgaatcgacctgtctgacatctacaacaaccccgtgctgaagttccagaagaagtacgctatccccttcatcggcaccatgtgcttcattctgcccactctgatccccatgtactgctggggcgagactttcaccaacgcctggcacatcaccatcctgcgatacgccctgaacctgaacatcacctggctgatcaactctgtggcccaccgatggggcaacaagccctacgacaagtctatcgtgcccgtgcagaacctgtctgtgtctctgatctctctcggcgagggcttccacaactaccaccacgtgttcccttgggactaccgaaccgccgagctgggcaacaacaccatgaacctgactaccaagttcatcgactttttcgcctggatcggctgggcctacgacctcaagaccgtgcctgaccacatgatcaagcagcgagtgaagcgaaccggcgacggcaccaacctgtggggctggggagacaagaacatgtctgaagagatgaaggtgggagccaagatcctgttcatcaagtctgagtag

SEQ ID NO: 58 - DNA, 1035 bp, Diatraea saccharalis
  source 1..1035: mol_type = other DNA
atggctcccaacctgatcaagaacgagggcgccttcgacgtggacatcaagcccgacgagctgcagcctcctaagctgtctaagatcaagcacaagatcctgtacgtgaacctgatctacttcctgtactggcacctggctgctccctacggcctgtacctgtgctgcacctctgccaagtgggccaccatcatcttcgccttcgtgatgttcctggccgccgagctgggcatcaccgctggcgcccaccgactgtggtcccacaagtcttacaaggccaagctgcccctcgagatcatcctgatgctgttcaactctatcgccttccagaactctatcatccactgggcccgagatcaccgactgcaccacaagtactctgacaccgacgctgaccctcacaacgcctctcgaggcttcttctactctcacgtcggctggctgatcaccaagaagaacaaggaagtcctgaagcgaggcaagaacatcgacgtgtctgacctgtacaacaaccccgtgctgcagttccagcgaaagtacgctgtgcccgtgttcgcctctttctgcttcattctgcccactctgatccccatgtacctctggggcgagtctttcaagaactcctggtacgtcaacctcctgcgatacgtgatcaacctgaacgtgaccttcctggtgaactctgccgctcacaagtggggctacaagccctacgagaagaacatcaaccccgctcagaacgtgtacgtgtctttcgctaccctcggcgagggcttccacaactaccaccacatctttccatgggactacaagaccgctgagctgggaaacaacaagctgaacgtcactacctggttcatcaactttttcgccaagatcggctgggcctacgacctcaagaccgtgtctaacgagatggtgcgagcccgagccatgcgaaccggcgacggcaacgacctgtggggctgggacgacgaggacattaccgacaaggacaaggaacacaccgacatcctgaacggcaagaccgactag

SEQ ID NO: 59 - protein, 344 aa, Diatraea saccharalis
MAPNLIKNEGAFDVDIKPDELQPPKLSKIKHKILYVNLIYFLYWHLAAPYGLYLCCTSAKWATIIFAFVMFLAAELGITAGAHRLWSHKSYKAKLPLEIILMLFNSIAFQNSIIHWARDHRLHHKYSDTDADPHNASRGFFYSHVGWLITKKNKEVLKRGKNIDVSDLYNNPVLQFQRKYAVPVFASFCFILPTLIPMYLWGESFKNSWYVNLLRYVINLNVTFLVNSAAHKWGYKPYEKNINPAQNVYVSFATLGEGFHNYHHIFPWDYKTAELGNNKLNVTTWFINFFAKIGWAYDLKTVSNEMVRARAMRTGDGNDLWGWDDEDITDKDKEHTDILNGKTD

SEQ ID NO: 60 - DNA, 1035 bp, Plodia interpunctella
  source 1..1035: mol_type = other DNA
atggtgcccaacaagggatcttctgtggtgctgaccgagaacgctgagccccagttcaagaagctggtcgctccccaggctggaccccgaaagtaccagatcgtgtaccgaaacctgctgaccttcggctactggcacctgtctgccatctacggcctgtacctgtgcttcacctctgccaagtgggctactattatcttcgccttcttcctgtacatgttcgccgagatcggcatcaccgctggcgcccaccgactgtggacccaccgagcctacaaggccaagctgcccctgcagatcctgctgatcatcatgaactctatcgccttccaggacaccgctctgacctggtgccgagatcaccgaatgcaccaccgatactctgacaccgacgctgaccctcacaacgctacccgaggcttcttctactctcacgtcggctggctgctggtgaagaagcaccccgaggtcaagacccgaggcaagtacattcccctggacgacctgcgaaacaaccccgtgctgcgattccagaagaagtacgctatccccttcgtgggcaccctgtgctttctgatgcccaccttcgtgcccgtgtacttctggggcgagtctatctctaccgcctggcacatcaacctgctgcgatacgtgaccaacctgaacgtgaccttcctggtgaactctgccgctcacctgatcggcaacaagccctacgaccgaactctggcctctgtgcagaacatccccgtgtctatcgccaccttcggcgagggcttccacaactaccaccacacttacccctgggactaccgaaccgccgagctgggcaacaaccgactgaacgtcactaccaagttcatcgactttttcgcctggctcggctgggcctacgacctcaagaccgtgcctcaagaggccgtcgagaagcgaatggcccgaaccggcgacggcaccgacctgtggggctacggcgaaaagaagcccgtcaaggacgttaaggacaactctgacgagacttactacgacttttag

SEQ ID NO: 61 - protein, 344 aa, Plodia interpunctella
MVPNKGSSVVLTENAEPQFKKLVAPQAGPRKYQIVYRNLLTFGYWHLSAIYGLYLCFTSAKWATIIFAFFLYMFAEIGITAGAHRLWTHRAYKAKLPLQILLIIMNSIAFQDTALTWCRDHRMHHRYSDTDADPHNATRGFFYSHVGWLLVKKHPEVKTRGKYIPLDDLRNNPVLRFQKKYAIPFVGTLCFLMPTFVPVYFWGESISTAWHINLLRYVTNLNVTFLVNSAAHLIGNKPYDRTLASVQNIPVSIATFGEGFHNYHHTYPWDYRTAELGNNRLNVTTKFIDFFAWLGWAYDLKTVPQEAVEKRMARTGDGTDLWGYGEKKPVKDVKDNSDETYYDF

SEQ ID NO: 62 - DNA, 1053 bp, Lobesia botrana
  source 1..1053: mol_type = genomic DNA
atggtgccccgcctagaagtaaatactggggtgaaaacagtggatgaagagcaattgccgtcaagggatgagaacaaaccgagaataagcaaactagtaaaaataaacatagtcagctttggatatctacatatagctgcactttatggattatatttatgcttcacatcggctaaatgggctacaattgggctcgctttctttttgctggtcgtcggtgaaatcggcatcacggcaggcgcccacaggttatggtcccacagaacctacaaagccaacagggcgttagaaatattactcatcattatgaactccgtggccttccaaaactcggccatccactggattcgggaccacagattacaccataaatacagcgacactaatgccgacccccacaatgcatccagaggatttttctattcacatataggctggttgctagtcaggaaacactctgacgtcataaacaaaggaaagatgattgacatgtctgatatatacaacaatccagttttaacattccaaaagaaatatgccattcccttcatcggcaccatatgcttcggtctacctaccctgataccgatgtactgctggggagagtctctgaacaacgcttggcacatcaacataatgcgatatgtcctgtgcctcaacattactttcctagtgaacagtgccgcgcatttcttcggtcagaaaccgtatgacaagaacattttaccgggacagaatatacttgtttcaatactcgctgttggcgaaggatttcataattaccaccacgtctttccgtgggactatcgcacggcagaactaggcaacaacatgttgaacttaacaacgaagtttatagacttcttcgcttggcttggttgggcgtacgagttaaaaactgtttcaactaatgcgattgatttaagagctaagaggactggcgacggaaccaacttgtggggttggggcgacaagcatatgacgcaagaagaaattgaggctagtgacattttgtttgttaacaaagaaaacgagaaaactcataggcattaa

SEQ ID NO: 63 - protein, 350 aa, Lobesia botrana
MVPRLEVNTGVKTVDEEQLPSRDENKPRISKLVKINIVSFGYLHIAALYGLYLCFTSAKWATIGLAFFLLVVGEIGITAGAHRLWSHRTYKANRALEILLIIMNSVAFQNSAIHWIRDHRLHHKYSDTNADPHNASRGFFYSHIGWLLVRKHSDVINKGKMIDMSDIYNNPVLTFQKKYAIPFIGTICFGLPTLIPMYCWGESLNNAWHINIMRYVLCLNITFLVNSAAHFFGQKPYDKNILPGQNILVSILAVGEGFHNYHHVFPWDYRTAELGNNMLNLTTKFIDFFAWLGWAYELKTVSTNAIDLRAKRTGDGTNLWGWGDKHMTQEEIEASDILFVNKENEKTHRH

SEQ ID NO: 64 - DNA, 1077 bp, Lobesia botrana
  source 1..1077: mol_type = other DNA
atggccccaaacaagaaggagatcgccgagcccgcccctgacatcgataatgagttccgggccctggtggctccccaggccggccccagaaagtacaccgtggtgtatatgacagtgctgaacttcctgtactggcacctgggcgggctgtacggcctgtatctgtgctttacctccgccaagtgggccacaatcttctttaatgcaatcctgcacgtgctggccgtgtggggaatcaccgccggcgcccaccggctgtggagccacagatcctataaggccaagctgccactccagatcctgctgatgctgatgcacacactggcctttcagaacccagcaatccactgggcacgggatcacagactgcaccacaagtacagcgacaccgatgccgaccctcacaatgccacacggggcttcttttattcccacatcggctggctgctggtgaggaagcaccctgaggccatcaagcgcggcaagaccatcgatgtgacagacatcatccagaaccccgtgctccagatccagaagaagtactatctgtacctgtctggcatgctgagcttcgtgctgcctgccctgatcccaatgttcttttggggcgagacactgaacaatgcccaccacgtgtctctgctgagactgatcatcgtgtttaacgtgacattctttgtgaatagcgccgcccacaagtacggcaacaagccctatgatatgaatatgtcccctacccagtctgtgacactgagcctggccaccctgggcgagtgtttccacaactaccaccacgtgttcccctttgactataggacagccgagctgggcaacaactacatcaatttcaccacaaagtttatcgatttctttgccgccatcggctgggcctacgacctgaagtatgcctccgacgatatgatcatgaagaggtctaaccgcaccggcgatggaacagacctgtggggaagggtgaagtccaccaacgacgagcaggcccctaagaacgtgtggggctggggcgatgaggacatggtggaggagcacaagaaggtgctgctgtctcagaatcagtga

SEQ ID NO: 65 - protein, 358 aa, Lobesia botrana
MAPNKKEIAEPAPDIDNEFRALVAPQAGPRKYTVVYMTVLNFLYWHLGGLYGLYLCFTSAKWATIFFNAILHVLAVWGITAGAHRLWSHRSYKAKLPLQILLMLMHTLAFQNPAIHWARDHRLHHKYSDTDADPHNATRGFFYSHIGWLLVRKHPEAIKRGKTIDVTDIIQNPVLQIQKKYYLYLSGMLSFVLPALIPMFFWGETLNNAHHVSLLRLIIVFNVTFFVNSAAHKYGNKPYDMNMSPTQSVTLSLATLGECFHNYHHVFPFDYRTAELGNNYINFTTKFIDFFAAIGWAYDLKYASDDMIMKRSNRTGDGTDLWGRVKSTNDEQAPKNVWGWGDEDMVEEHKKVLLSQNQ

SEQ ID NO: 66 - DNA, 1041 bp, Antheraea pernyi
  source 1..1041: mol_type = other DNA
atggctccctgcaccgagaaggaagatgagctgatcacctgtgagggctctatcgagaagctgatcgctccccaggccaacaagcgaaagcaccagatcgtgtacatgaacctgttcaccttcggctactggtacattgccggcctgtacggactgtacctgttcttcacctctaccaagtggtctactattatcctgaacatcttcctgatcattgcctctggcctgggcatcaccgtgggagcccaccgactgtgggctcacaagtgcttcaaggccaagctgcccctgcagatcatcctgatgattctgaacaccctggccttccagaacaccgccatcacctgggtgcgagatcaccgaatgcaccacaagttcaccgacaccgacgctgaccctcacaacgctacccgaggcttcttcttctctcacgtcggctggctgctggtgaagaagcaccccgagctgatgaagcgatctaagcacatcgacatgtctgacatctacggcaaccccgtgctgacctttcagaagaagtacgctttccctctggtcgccaccttcaccttcattctgcccaccgtggtgcctatctacttctgggacgagtctctgaacaacgcctggcacgccaccgtgttccgatacgcctacaacctgaacattacctttctggtgaactctgccgctcacatgtggggctaccgaccttacgacaagaacattcagcccactcagaacattttcaccactctgtgtaccttcggcgagggcttccacaactaccaccacacattcccttgggactaccgagccgccgagctgggcaacaactacctgaacctgtctaccaaggtgatcgacttcttcgcctggatcggctgggcctacgacctcaagaccgtgcctgccgacatcgtgaactctcgagccaagcgaaccggcgacggcaccgacctctggggcggcaaccacaagcacaccctggatgagaaggaaaaccgagagctggacgactacgtgcacaagtctgccttttag

SEQ ID NO: 67 - protein, 346 aa, Antheraea pernyi
MAPCTEKEDELITCEGSIEKLIAPQANKRKHQIVYMNLFTFGYWYIAGLYGLYLFFTSTKWSTIILNIFLIIASGLGITVGAHRLWAHKCFKAKLPLQIILMILNTLAFQNTAITWVRDHRMHHKFTDTDADPHNATRGFFFSHVGWLLVKKHPELMKRSKHIDMSDIYGNPVLTFQKKYAFPLVATFTFILPTVVPIYFWDESLNNAWHATVFRYAYNLNITFLVNSAAHMWGYRPYDKNIQPTQNIFTTLCTFGEGFHNYHHTFPWDYRAAELGNNYLNLSTKVIDFFAWIGWAYDLKTVPADIVNSRAKRTGDGTDLWGGNHKHTLDEKENRELDDYVHKSAF

SEQ ID NO: 68 - DNA, 1029 bp, Cadra cautella
  source 1..1029: mol_type = other DNA
atggtgccctacaagggatcttctaccgtgctgaccgaggaccagcctcagttcgagaagctggtggctccccaggctggaccccgaaagtacgagatcgtgtaccgaaacctgatcaccttcggctactggcacctggccgccatctacggcctgtacctgtgcttcaccaccgccaagtgggccactattatcttcgccctgttcctgtacaccattgccgagatcggcatcaccgctggcgcccaccgactgtggacccaccgagcctacaaggccaagctgcccctgcagatcctgctgatggtgatgaactctatcgccttccaggacaccgctctgacctggtgtcgagatcaccgaatgcaccaccgatactctgacaccgacgctgaccctcacaacgctacccgaggcttcttctactctcacgtcggctggctgctggtgaagaagcaccccgaggtcaaggcccgaggcaagtacattcccctggacgacctgctgaacaaccccgtgctgcgattccaaaagaagtacgctgtccccgtcgtgggcaccctgtgctttctgatgcccaccttcgtgcccgtgtacttctggggcgagactatctctaccgcctggcacatcaactttctgcgatacgtgatgaacctgaacatgacctttctggtgaactctgccgctcacatgttcggcaacaagccctacgaccgaactctggtgcccgtgcagaacgtggccgtgtctttcgccaccttcggagagggcttccacaactaccaccacacttacccctgggactaccgaaccgccgagctgggcaacaacaagctgaacatcaccactcacttcatcgactttttcgcctggatcggctgggcctacgacctcaagaccattcctcaggacgccatcgagaagcgaatgatccgaaccggcgacggcaccgacatgtggggcttcggtgagaagcgatttcgaaaggaagtggacaactctaacgagacttactctgacttttag

SEQ ID NO: 69 - protein, 342 aa, Cadra cautella
MVPYKGSSTVLTEDQPQFEKLVAPQAGPRKYEIVYRNLITFGYWHLAAIYGLYLCFTTAKWATIIFALFLYTIAEIGITAGAHRLWTHRAYKAKLPLQILLMVMNSIAFQDTALTWCRDHRMHHRYSDTDADPHNATRGFFYSHVGWLLVKKHPEVKARGKYIPLDDLLNNPVLRFQKKYAVPVVGTLCFLMPTFVPVYFWGETISTAWHINFLRYVMNLNMTFLVNSAAHMFGNKPYDRTLVPVQNVAVSFATFGEGFHNYHHTYPWDYRTAELGNNKLNITTHFIDFFAWIGWAYDLKTIPQDAIEKRMIRTGDGTDMWGFGEKRFRKEVDNSNETYSDF

SEQ ID NO: 70 - DNA, 1059 bp, Yponomeuta padella
  source 1..1059: mol_type = other DNA
atgcctccaaactctgccaactctgagcccctgctgcacgacgaggtgaaggaagaacagctcgagaagctggtggctccccaggctggaccccgaaagttccagatcgtgtaccccaacctgatcaccttcggctacggccacatctctctgctgtacggcgcctacctgatgttcaccttcgccaagtggcagaccatcgtgttcgcccacgtgatgatcattctgtctggcttcggcatcaccgctggcgcccaccgactgtgggctcaccgaacctacaaggccaagatgcccctgcagatcctgctgatgctgctgaacaccctggccttccagaacaccgccatggactgggtgcgagatcaccgactgcaccacaagtactctgacaccgacgctgaccctcacaacgctacccgaggcttcttctactctcacgtcggctggctgctggtgcgaaagcacaaggaagtgaagcgacgaggcaagctgatcgacctgtctgacatcgagaacaaccccgtgctgtctttccagaagaagtacgctattcccctggtgtctctggtgaccttcgtgatgcccaccgtgattcctatgtacttctggggcgagactctgaacaacgcctggcacgtggctaccctgtaccgatacgtgttcgtgctgaacgtgaccttcctggtgaactctgccgctcactactggggctgcaagccctacgagaagtctattctgccctctcagaaccacttcgtgtctctgttcgccctcggcgagggattccacaactaccaccacgtgttcccttgggactaccgaaccgccgagctgggcaacaacactctgaaccccactaccaagttcatcgactttttcgcctggatcggctgggcctacgacctgaaggctgtgcccgacgacatgatcaacaaccgaaagtctcgaaccggcgacggatcttctctgtggggctggggcgacaaggaccagcctaaggaagagatgaacgccgccatcatcaagtaccccgacgagaacgacaaggataagtag

SEQ ID NO: 71 - protein, 352 aa, Yponomeuta padella
MPPNSANSEPLLHDEVKEEQLEKLVAPQAGPRKFQIVYPNLITFGYGHISLLYGAYLMFTFAKWQTIVFAHVMIILSGFGITAGAHRLWAHRTYKAKMPLQILLMLLNTLAFQNTAMDWVRDHRLHHKYSDTDADPHNATRGFFYSHVGWLLVRKHKEVKRRGKLIDLSDIENNPVLSFQKKYAIPLVSLVTFVMPTVIPMYFWGETLNNAWHVATLYRYVFVLNVTFLVNSAAHYWGCKPYEKSILPSQNHFVSLFALGEGFHNYHHVFPWDYRTAELGNNTLNPTTKFIDFFAWIGWAYDLKAVPDDMINNRKSRTGDGSSLWGWGDKDQPKEEMNAAIIKYPDENDKDK

SEQ ID NO: 72 - DNA, 1362 bp, Heliothis virescens
  source 1..1362: mol_type = other DNA
atggttgtcttgacctccaaagaaactaagccatctgttgctgaattttacgctggtaagtctgttttcattactggtggtactggtttcttgggtaaggttttcattgaaaagttgttgtactcctgcccagatatcgttaacatctacatgttgatcagagaaaagaagggtttgtccgtttccgaaagaatcaagcaatttttggatgaccctttgttcaccagattgaaggacaaaagaccagctgatttggaaaagatcgttttgattccaggtgatattaccgctccagatttgggtattactgctgctaacgaaaagatgttgatcgaaaaggtttccgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagtttaccaatacgtcaaagaaggtatctccgaagaagataccgaaaaaatcttgaacggtagaccaaacacttacactttcactaaggctttgaccgaacatttggttgctgaaaatcaagcttacgttccaaccattatcgttagaccatcagttgttgctgccattaaggatgaaccattgaaaggttggttgggtaattggtttggtgctacaggtttgactgtttttactgctaagggtttgaacagagttatctacggtcattccaactacatcgttgatttgatcccagttgattacgttgccaacttggttattgctgctggtgctaaatctaacacctcttctgaattgaaggtctacaactgttgttcctcatcatgtaacccagttaagatcggtactttgatgtctatgtttgctgatgatgccatcaagcaaaagtcttatgctatgccattgccaggttggtacatttttactaagtacaagtggttggtcttgttgttgaccttcttgttccaagttattccagcctacattaccgatttgtcaagacacttggttggtaagagtccaagatatatcaagttgcaatccttggtcaatcaaaccagatcctccattgatttcttcaccaatcattcttgggttatgaaggccgatagagtcagagaattatacgcttctttgtctccagcagataagtacttgtttccatgtgatccagttaacatcaactggacccaatacttgcaagattactgttggggtgttagaaacttcttggaaaaaaagacttaa

SEQ ID NO: 73 - protein, 453 aa, Heliothis virescens
MVVLTSKETKPSVAEFYAGKSVFITGGTGFLGKVFIEKLLYSCPDIVNIYMLIREKKGLSVSERIKQFLDDPLFTRLKDKRPADLEKIVLIPGDITAPDLGITAANEKMLIEKVSVIIHSAATVKFNEPLPTAWKINVEGTRMMLALSRRMKRIEVFIHISTAYTNTNREVVDEILYPAPADIDQVYQYVKEGISEEDTEKILNGRPNTYTFTKALTEHLVAENQAYVPTIIVRPSVVAAIKDEPLKGWLGNWFGATGLTVFTAKGLNRVIYGHSNYIVDLIPVDYVANLVIAAGAKSNTSSELKVYNCCSSSCNPVKIGTLMSMFADDAIKQKSYAMPLPGWYIFTKYKWLVLLLTFLFQVIPAYITDLSRHLVGKSPRYIKLQSLVNQTRSSIDFFTNHSWVMKADRVRELYASLSPADKYLFPCDPVNINWTQYLQDYCWGVRNFLEKKT

SEQ ID NO: 74 - DNA, 1350 bp, Yponomeuta rorellus
  source 1..1350: mol_type = other DNA
atggtccaattgaaagaagattccgttgctgctttttacgccgaaaagtctattttcattactggtggtactggtttcttgggtaaggttttgattgaaaagttgttgtactcctgcaaggccgttgatcaaatctacgttttgatcagaaagaagaaggaccaaaccccatctgaaagaattgcccaattattggaatccgaattattctccagattgagaaaggatgatccatccgctttgaaaaaggttgttccagttgttggtgatttgaccatgcctaatttgggtttgtctgctgctgttgaagatttgattgtctctaaggttaccgttatcttccatgttgctgctactgttaagttcaacgaaagaatgaagaacgccttggttaacaacgttgaagctactagagaagtcatcaacttgtgccatagattggaaaaggttgatgccttcattcatgtttctaccgcttactctaataccgatcaaaaggtcgttgaagaaagagtttatccaccaccagctccattgtctgaagtttatgcttttgttaagaactacggtgacgacatggatatcatccaaaatttgttgaacggtagaccaaacacttacacctacacaaaagctttggctgaagatatcgtcttgaaagaacatggtggtattccaaccgctattatcagaccatctatcgttttgtccgttttaaaagaacctatcccaggttggttggataattggaatggtccaactggtttgttgcatgcttcttcacaaggtgttcattgctctatgttgggttctggttctaatgttgccgatttgattccagttgatatcgtcactaacttgatgatcgttgttgcttctagatgcagaaagtctaatggtttgaaggtctacaactcttgttctggtactaccaacccaattacttaccaagctttcaccaagatgttcttggattcctgtatttctagaggttggaacaaggttccattccctttgttgatttttgtcaagtgggctttcttgaacagagtcttgaagttcttgttggttatcgtcccattcttcttgatcgatgtctacttgagatttttcggtaagccaaactacatgagaatgatcacctacaccaagaaagctgaagatttgatgaccttcttcacctctcatgaatggcaattcaaggatggtaatgtcagagacttgatcaacatgatgtctccagaagatagaaagatcttctactgcgatcctgaagaaattcaatggaagccatacttcgatgattactgcgttggtgtctttaaatacttgttgaagagaaaggtttaa

SEQ ID NO: 75 - protein, 449 aa, Yponomeuta rorellus
MVQLKEDSVAAFYAEKSIFITGGTGFLGKVLIEKLLYSCKAVDQIYVLIRKKKDQTPSERIAQLLESELFSRLRKDDPSALKKVVPVVGDLTMPNLGLSAAVEDLIVSKVTVIFHVAATVKFNERMKNALVNNVEATREVINLCHRLEKVDAFIHVSTAYSNTDQKVVEERVYPPPAPLSEVYAFVKNYGDDMDIIQNLLNGRPNTYTYTKALAEDIVLKEHGGIPTAIIRPSIVLSVLKEPIPGWLDNWNGPTGLLHASSQGVHCSMLGSGSNVADLIPVDIVTNLMIVVASRCRKSNGLKVYNSCSGTTNPITYQAFTKMFLDSCISRGWNKVPFPLLIFVKWAFLNRVLKFLLVIVPFFLIDVYLRFFGKPNYMRMITYTKKAEDLMTFFTSHEWQFKDGNVRDLINMMSPEDRKIFYCDPEEIQWKPYFDDYCVGVFKYLLKRKV

SEQ ID NO: 76 - DNA, 1389 bp, Ostrinia nubilalis
  source 1..1389: mol_type = other DNA
atgtctgccaacaccatggaaaccgacgagcagttcacctacaactctcccatcgtgaacttctactctggcaagtctgtgttcgtgaccggcgctaccggcttcctgggcaccgtgctggtcgagaagctgctgttctcttgcaagggcatcaacaacatctacatcctgatcaagcagaccgaggacctgaccatcgaggcccgaatcctgaactacctgaactctaaggccttccaccgagtgaagaacactaaccccgagctgatgaagaagatcatccccatctgcggcaacctcgaggacaagaacctgggcatctctgactctgacatgaagaccctgctggaagaggtgtctatcgtgttccacgtggccgccaagctgctctttaagatgtctctgaccgccgctgtgaacatcaacaccaagcctaccgagcagctgatcgccatctgcaagaagatgcgacgaaaccccatcttcatctacgtgtcctctgcctactctaacgtgaacgagcagatcatcgacgagaaggtgtacaacaccggcgtgcccctcgagactatctacgacaccctggacaccgagaacacccgaattaccgacatcttcctggacaagcgacccaacacctacacctactccaaggctctggccgaggtggtcgtcgagaaggaattcgacgagtctgccgccatcgtgcgaccctctatcatcgtgtcctccattcgagagcccattccaggctggctgtctggctctcacggcttccctcgagtggtgggagccgcctgcaagggcctgctgctccgatggcacggcgacggcaccgtggtgtgcgacctgattcctgtggaccacgtcgccaacctgatcattgccgccgcttgggagtctaacgagcgacgactgatgggcaacaagggcgtgaaggtctacaactgttgttcttctctgcgaaaccctatcgacgtgatcaccgtggtcaagacctgcatcaagtaccgaaagtacttcggcacccgaaccatgtctatcttcacccctcgattcatcatgaagaagaactacttcatctacaagctgctgtacttcacctaccacactatccccgctgctattatcgacggcttcttctggctgaccggacgaacccctatcatgctcaagaccctcgacaagctgggcaagatctcttctgtgctcgagtactttacccaccaccagttcatctttctggactccaacgtgcgaggactgctgcgacgaatggaaggcaccgaccgacagaccttcaacttcgacgtgaccgagatcgagtgggagccctacctgcagaacttcgtgcgaggcattgccaacaactacgactactctatgtag

SEQ ID NO: 77 - protein, 462 aa, Ostrinia nubilalis
MSANTMETDEQFTYNSPIVNFYSGKSVFVTGATGFLGTVLVEKLLFSCKGINNIYILIKQTEDLTIEARILNYLNSKAFHRVKNTNPELMKKIIPICGNLEDKNLGISDSDMKTLLEEVSIVFHVAAKLLFKMSLTAAVNINTKPTEQLIAICKKMRRNPIFIYVSSAYSNVNEQIIDEKVYNTGVPLETIYDTLDTENTRITDIFLDKRPNTYTYSKALAEVVVEKEFDESAAIVRPSIIVSSIREPIPGWLSGSHGFPRVVGAACKGLLLRWHGDGTVVCDLIPVDHVANLIIAAAWESNERRLMGNKGVKVYNCCSSLRNPIDVITVVKTCIKYRKYFGTRTMSIFTPRFIMKKNYFIYKLLYFTYHTIPAAIIDGFFWLTGRTPIMLKTLDKLGKISSVLEYFTHHQFIFLDSNVRGLLRRMEGTDRQTFNFDVTEIEWEPYLQNFVRGIANNYDYSM

SEQ ID NO: 78 - DNA, 1377 bp, Agrotis segetum
  source 1..1377: mol_type = other DNA
atgcccgtgctgacctcgcgagaggacgagaagctgtctgtgcccgagttctacgccggcaagtctatcttcgtgaccggcggcaccggattcctcggcaaggtgttcattgagaagctgctctactgctgccccgacatcgacaagatctacatgctgatccgagagaagaagaacctgtctatcgacgagcgaatgtctaagttcctggacgaccctctgttctctcgactgaaggaagaacgacccggcgacctcgagaagatcgtgctgatccccggcgacatcaccgctcctaacctgggcctgtctgccgagaacgaacgaatcctgctcgagaaggtgtccgtgatcatcaactctgccgccaccgtgaagttcaacgagcccctgcctatcgcctggaagatcaacgtcgagggcacccgaatgctgctggccctgtctcgacgaatgaagcgaatcgaggtgtttatccacatctctaccgcctactctaacgcctcttctgaccgaatcgtggtggacgagattctgtaccccgctcctgccgacatggaccaggtgtaccagctcgtgaaggacggcgtgaccgaggaagagactgagcgactgctgaacggactgcccaacacctacaccttcaccaaggctctgaccgagcacctggtggccgagcaccagacctacgtgcccaccatcatcattcgaccctccgtggtggcctctatcaaggacgagcccatccgaggctggctgtgcaactggttcggcgccaccggcatctctgtgttcaccgccaagggcctgaaccgagtgctgctcggaaaggcctctaacatcgtggacgtgatccccgtggactacgtggccaacctggtgatcgtggctggcgccaagtctggcggccagaagtctgacgagctgaagatctataactgctgttcttctgactgcaaccccgtgactctgaagaagatcatcaaggaattcaccgaggacaccatcaagaacaagtctcacatcatgcctctgcctggctggttcgtgttcaccaagtacaagtggctgctgaccctcctgaccatcatcttccagatgctgcccatgtacctggccgacgtgtaccgagtcctgaccggcaagattccccggtacatgaagctgcaccacctggtcattcagacccgactgggaatcgacttcttcacctctcactcttgggtgatgaagaccgaccgagtgcgagagctgttcggctctctgtctctggccgagaagcacatgttcccttgcgacccctcttccatcgactggaccgactacctgcagtcttactgctacggcgtgcgacgattcctggaaaagaagaagtag

SEQ ID NO: 79 - protein, 458 aa, Agrotis segetum
MPVLTSREDEKLSVPEFYAGKSIFVTGGTGFLGKVFIEKLLYCCPDIDKIYMLIREKKNLSIDERMSKFLDDPLFSRLKEERPGDLEKIVLIPGDITAPNLGLSAENERILLEKVSVIINSAATVKFNEPLPIAWKINVEGTRMLLALSRRMKRIEVFIHISTAYSNASSDRIVVDEILYPAPADMDQVYQLVKDGVTEEETERLLNGLPNTYTFTKALTEHLVAEHQTYVPTIIIRPSVVASIKDEPIRGWLCNWFGATGISVFTAKGLNRVLLGKASNIVDVIPVDYVANLVIVAGAKSGGQKSDELKIYNCCSSDCNPVTLKKIIKEFTEDTIKNKSHIMPLPGWFVFTKYKWLLTLLTIIFQMLPMYLADVYRVLTGKIPRYMKLHHLVIQTRLGIDFFTSHSWVMKTDRVRELFGSLSLAEKHMFPCDPSSIDWTDYLQSYCYGVRRFLEKKK

SEQ ID NO: 80 - DNA, 1009 bp, Marinobacter algicola
  source 1..1009: mol_type = other DNA
atgtgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatggtgagtttcagaggcagcagcaattgccacgggctttgagcacacggccgggtgtggtcccattcccatcgacacaagacgccacgtcatccgaccagcactttttgcagtactaaccgcaggccacccagcagcagcagaacggtgcatccgcttcgggcgttctggagcagcttagaggcaagcatgtcttgattaccggtactacaggatttctgggaaaggtggttctggagaagctgatccgaaccgtgcctgacatcggtggtattcatctgctgattagaggcaacaagagacatcctgctgccagagaaagattcttgaacgaaatcgcctcttcctctgtgttcgagcggcttagacatgacgacaacgaagcctttgagactttcctggaggagcgtgtgcactgcatcaccggagaagtgaccgagtcgagatttggccttactcctgagcggttccgagcccttgctggccaagtggatgccttcatcaattccgccgcctctgttaacttcagagaggagctggacaaggcactcaagatcaacaccctgtgtctggagaacgtggctgctctggccgaacttaactccgctatggcagtgatccaagtttccacctgttacgtgaacggcaagaactctggacagatcaccgagtccgttatcaagcccgctggcgaatccatccccagatccacagatggctactacgagatcgaggagctggtccaccttctgcaagacaagatc

SEQ ID NO: 81 - protein, 512 aa, Marinobacter algicola
MATQQQQNGASASGVLEQLRGKHVLITGTTGFLGKVVLEKLIRTVPDIGGIHLLIRGNKRHPAARERFLNEIASSSVFERLRHDDNEAFETFLEERVHCITGEVTESRFGLTPERFRALAGQVDAFINSAASVNFREELDKALKINTLCLENVAALAELNSAMAVIQVSTCYVNGKNSGQITESVIKPAGESIPRSTDGYYEIEELVHLLQDKISDVKARYSGKVLEKKLVDLGIREANNYGWSDTYTFTKWLGEQLLMKALSGRSLTIVRPSIIESALEEPSPGWIEGVKVADAIILAYAREKVSLFPGKRSGIIDVIPVDLVANSIILSLAEALSGSGQRRIYQCCSGGSNPISLGKFIDYLMAEAKTNYAAYDQLFYRRPTKPFVAVNRKLFDVVVGGMRVPLSIAGKAMRLAGQNRELKVLKNLDTTRSLATIFGFYTAPDYIFRNDSLMALASRMGELDRVLFPVDARQIDWQLYLCKIHLGGLNRYALKERKLYSLRAADTRKKAA

SEQ ID NO: 82 - DNA, 1557 bp, Tyta alba
  source 1..1557: mol_type = other DNA
atggtgtctatccccgagtactacgagggcaagaacatcctgctgaccggcgccaccggcttcatgggcaaggtgctgctcgagaagctgctgcgatcttgccccaaggtgaaggccgtgtacgtgctggtgcgacacaaggccggacagacccctgaggctcgaatcgaggaaatcaccaactgcaagctgttcgaccgactgcgagatgagcagcccgacttcaaggccaagatcatcgtgatcacctctgagctgacccagcctgagctggacctgtctgagcccatcaaggaaaagctgatcgagcgaatcaacatcatcttccactgcgccgccaccgtgcgattcaacgagactctccgagatgccgtgcagctgaacgtgaccgctactcagcagctcctgttcctggctcagcgaatgaagaacctggaagtgttcatgcacgtgtctaccgcctacgcctactgcaaccgaaagcagatcgaagagatcgtgtaccctcctccagtggaccccaagaagctgattgactctctcgagtggatggacgacggcctggtgaacgacatcacccctaagctcatcggcgaccgacctaacacctacacttacaccaaggctctggccgagtacgtggtgcagcaagagggcgccaagctgaacaccgccatcattcgaccctctatcgtgggcgcctcttggaaggaaccctttcctggctggatcgacaacttcaacggcccctctggcctgttcattgccgccggaaagggcatcctgcgaaccatgcgagcctctaactctgccgtggccgacctggtgcctgtggacgtggtggtgaacaccactctggccgctgcctggtactctggcgtgaaccgacctcgaaacgtgatgatctacaactgcaccaccggcggcactaaccccttccactggggcgaagtgggctaccacatcaacctgaacttcaagatcaaccctctcgagaacgccgtgcgacaccccaactgttctctgcagtctaaccctctgctccatcagtactggaccgccgtgtctcacaccatgcctgcctttctgctggacctcctgctgcgactgaccggacacaagccctggatgatgaagaccatcactcgactgcacaaggccatgatgctcctcgagtacttcacctccaactcttggatctggaacaccgagaacatgaccatgctgatgaaccagctgaaccccgaggacaagaagaccttcaacttcgacgtgcgacagctgcactgggctgagtacatggaaaactactgcatgggcaccaagaagtacgtcctgaacgaggaaatgtctggactgcccgctgccagaaagcacctgaacaagctgcgaaacatccgatacggcttcaacaccgtgctggtcatcctgatctggcgaatcttcattgcccgatctcagatggcccgaaacatctggtacttcgtggtgtctctgtgctacaagttcctgtcttacttccgagcctcttctaccatgcgatactctaagctgtag

SEQ ID NO: 83 - protein, 515 aa, Tyta alba
MVSIPEYYEGKNILLTGATGFMGKVLLEKLLRSCPKVKAVYVLVRHKAGQTPEARIEEITNCKLFDRLRDEQPDFKAKIIVITSELTQPELDLSEPIKEKLIERINIIFHCAATVRFNETLRDAVQLNVTATQQLLFLAQRMKNLEVFMHVSTAYAYCNRKQIEEIVYPPPVDPKKLIDSLEWMDDGLVNDITPKLIGDRPNTYTYTKALAEYVVQQEGAKLNTAIIRPSIVGASWKEPFPGWIDNFNGPSGLFIAAGKGILRTMRASNSAVADLVPVDVVVNTTLAAAWYSGVNRPRNVMIYNCTTGGTNPFHWGEVGYHINLNFKINPLENAVRHPNCSLQSNPLLHQYWTAVSHTMPAFLLDLLLRLTGHKPWMMKTITRLHKAMMLLEYFTSNSWIWNTENMTMLMNQLNPEDKKTFNFDVRQLHWAEYMENYCMGTKKYVLNEEMSGLPAARKHLNKLRNIRYGFNTVLVILIWRIFIARSQMARNIWYFVVSLCYKFLSYFRASSTMRY

SEQ ID NO: 84 - DNA, 1407 bp, Chilo suppressalis
  source 1..1407: mol_type = other DNA
atggaaccctacaagtctgacgaggaaatcaagtctaagctgttctctcccgtggtgaacttctacaccggcaagtctgtgttcattaccggcgctaccggcttcctgggcaccgtgctgctcgagaagctgatgttcacctgtgctcacaacatcaagaacatctacatcctgatcaagcccaccgacggccagtctatcgacgagaagatgtctaagttcttcgactctcgagccttcgagcgactgcgagagcacaaccccaacttccgatctaagatcattcccctgaccggcgacatcaccaagaagtctatcggcctgtctgagaacgacatcttcattctgcgaaaggaagtgtctgtcgttttccactctgccgccgacacctctttccagctgtctctgtctgaggccatcatcatcaacaccaaggccaccgaggaactgctgaagatctgcaaggacatgcaccagctgaaggctttcgtgtacgtgtctaccgcctactctaactgcaaccgacctatcattgacgagaaggtgtaccctaccgacgtgtctctcgagactgtgtacgagctgctggaatactctaagtctgacaagctgatcgagttcctgttcaacggacgacccaacgcctacacctactccaaggctctgtcggaggaactggtgcagaactacggcaacatcatcccctctatcatcattcgaccctccatcatcacctcttctatcaaggaaccctatcctggctggctgtctggctggaacggcctgaaccaggtgatcctgtctggcatgaagggctacctccgatgctggttcgccgacgactcttgtatcgccgacactatccccgtggactacaccgccaacgtgatgatcgtgtctgcctgggacgcccacgagcgacgactgaagaacgacaagcagctcaaggtgttcaactgctgctctggcctgcagaaccccatcgacaccggaaacatgatgtctatctgcctcgagcacaacaagaagcacgagaaggacaagtccaaggccaacaccatgttcatcatccgaaagaacttttacgtgtacttcttctacttcctggtgctgcatctgatccccgctctgattatcgacgtgttctactttctgctgggccgagagatgctgatgtgtaccaacctgaagaagatcaagcgattctcttcgatcctgaaggtgttctgcttcaaccagtttctgttcatcgacaagaacgtccgacgactgcaccaggctctgaacgagactgacaaggtgctgttcgacttcgacgtgcgaaacatccagtggcgagtgtacctgaaggacttcgtgatcgccctgaaggaatacgacaagacctctcgaaccgtgaagatttag

SEQ ID NO: 85 - protein, 468 aa, Chilo suppressalis
MEPYKSDEEIKSKLFSPVVNFYTGKSVFITGATGFLGTVLLEKLMFTCAHNIKNIYILIKPTDGQSIDEKMSKFFDSRAFERLREHNPNFRSKIIPLTGDITKKSIGLSENDIFILRKEVSVVFHSAADTSFQLSLSEAIIINTKATEELLKICKDMHQLKAFVYVSTAYSNCNRPIIDEKVYPTDVSLETVYELLEYSKSDKLIEFLFNGRPNAYTYSKALSEELVQNYGNIIPSIIIRPSIITSSIKEPYPGWLSGWNGLNQVILSGMKGYLRCWFADDSCIADTIPVDYTANVMIVSAWDAHERRLKNDKQLKVFNCCSGLQNPIDTGNMMSICLEHNKKHEKDKSKANTMFIIRKNFYVYFFYFLVLHLIPALIIDVFYFLLGREMLMCTNLKKIKRFSSILKVFCFNQFLFIDKNVRRLHQALNETDKVLFDFDVRNIQWRVYLKDFVIALKEYDKTSRTVKI

SEQ ID NO: 86 - DNA, 1449 bp, Bombus lapidarius
  source 1..1449: mol_type = other DNA
atgtctcaccaccaccatcaccacggctctaacaccaagctgaacgagaacgagatcaacgagaagctgcgaaacgtgaactctatcggcggcttctacgccggcaccggcatcctgatcaccggcggcaccggattcgtcggcaagggcctgctcgagaagctcatccgaacctgctctcacattgccgccatcttcatcctgattcgacccaagcgaaaccagaccatcgagcagcgattcaagaagatcatcgacgaccccatcttcgacggcgtgcgagcacagaaccccgccattttctacaagatccacctggtcgagggcgacgtgaccctgcctgacctgggcctgctgcaaaaggaccgagacatgctgattgagaacgtgaacatcgtgttccacattgctgccaccatcaacttccaccagcctctggacatgatcgtgaacgtcaacgtgaagggcaccgccaacatcatcaagctgtgcaaggaactgaagcacgtgatctctgtggtgtacgtgtctaccgcctactctaaccccaacctgtctgacatcgaggaaaaggtctacactaccaacttggacccctctctggtgatggacatctgcgaccgacaggacaaggaactcatcaacctgatcgaggaacgaatcctcaagactcaccccaacacctacaccttcaccaagaacctggccgagcagaccatctctaacaactctaagggcctgaccgtggccatcgtgcgaccctctatcatctcttcgtccctgaaggaaccctttcctggctggctggtgtctttcgccggccagtctggcatcttcaagaacatcggcaacggcatggccaaggtgctgctcggacgaggcgacgtcatctccgacatcgtgcccgtggactacgtggtggacgccatcatgtgcgccgcctggcacgtcaccctgcagatcgacaacaacgtcaaggtgtacaactgcacctcttctgctcgacccattaagctgggcgagattgtgaacatcttcgtcgagtgttctcgagagatccccatgaagaacaccctgtggtatccctcttgcaccatcgtggccaaccgattcgtgtacaacgtgctgaacatcctgctgaacgtgctgcccgccttcgccgtggacatcttcctgcgactgcgaggcggcaagcccatggctatgaacatgaacaagtactacaacaagctggtggtcgccacctcttacttcaactctaacgagtggtctttcaagcgagacaacattgccgacatgatcaacaaggtgaacaccctcgaggacggcaacatcgtgaagctggacctgcaggacatggtgtggcgaaagtacattgccaactacctggccggcatcaagaagttcattctgaaggaagatcccaagtctatcaacgctgctccctaa

SEQ ID NO: 87 - protein, 482 aa, Bombus lapidarius
MSHHHHHHGSNTKLNENEINEKLRNVNSIGGFYAGTGILITGGTGFVGKGLLEKLIRTCSHIAAIFILIRPKRNQTIEQRFKKIIDDPIFDGVRAQNPAIFYKIHLVEGDVTLPDLGLLQKDRDMLIENVNIVFHIAATINFHQPLDMIVNVNVKGTANIIKLCKELKHVISVVYVSTAYSNPNLSDIEEKVYTTNLDPSLVMDICDRQDKELINLIEERILKTHPNTYTFTKNLAEQTISNNSKGLTVAIVRPSIISSSLKEPFPGWLVSFAGQSGIFKNIGNGMAKVLLGRGDVISDIVPVDYVVDAIMCAAWHVTLQIDNNVKVYNCTSSARPIKLGEIVNIFVECSREIPMKNTLWYPSCTIVANRFVYNVLNILLNVLPAFAVDIFLRLRGGKPMAMNMNKYYNKLVVATSYFNSNEWSFKRDNIADMINKVNTLEDGNIVKLDLQDMVWRKYIANYLAGIKKFILKEDPKSINAAP

SEQ ID NO: 88 - DNA, 1365 bp, Spodoptera littoralis
  source 1..1365: mol_type = other DNA
atggtggtgctgacctctaaggaaaagtctaacatgtctgtggccgacttctacgccggcaagtctgtgttcattaccggcggcaccggattcctcggcaaggtgttcatcgagaagctgctgtactcttgccccgacatcgacaagatctacatgctgatccgagagaagaagggccagtctatccgagagcgactgaccaagatcgtggacgaccctctgttcaaccgactgaaggacaagcgacccggcgacctgggaaagatcgtgctgatccccggcgacatcaccgtgcctggcctgggcatctctgaggaaaacgagactatcctgaccgagaaggtgtctgtggtgatccactctgccgccaccgtgaagttcaacgagcccctggccaccgcctggaacgtgaacgtcgagggcacccgaatgatcatggccctgtctcgacgaatgaagcgaatcgaggtgtttatccacatctctaccgcctacaccaacaccaaccgagccgtgatcgacgaggtgctgtaccctcctcctgccgacatcaacgacgtgcaccagcacgtgaagaacggcgtgaccgaggaagagactgagaagatcctgaacggacgacccaacacctacaccttcaccaaggctctgaccgagcacctggtggccgagaaccagtcttacatgcccaccatcatcgtgcgaccctctatcgtgggcgccatcaaggacgaccccatccgaggctggctggccaactggtacggcgccaccggcctgtccgtgttcaccgccaagggcctgaaccgagtgatctacggccactctaaccacgtcgtggatctgattcccgtggactacgtggccaacctggtgatcgtggctggcgccaagacctaccactccaacgaggtgaccatctacaactcttgttcttcttcttgcaaccccatcaccatgaagcgactggtgggcctgttcattgactacaccgtcaagcacaagtcttacgtgatgcccctgcctggctggtacgtgtactctaactacaagtggctggtgttcctggtgaccgtgatcttccaggtgatccccgcctacctgggcgacatcggccgacgactgctgggcaagaaccctcggtactacaagctgcagaacctggtcgctcagacccaagaggccgtgcacttcttcacctctcacacctgggagattaagtctaagcgaacctctgagctgttctcttctctgtccctgaccgaccagcgaatgttcccctgcgacgccaaccgaatcgactggaccgactacatcaccgactactgctctggcgtgcgacagttcctcgagaagatcaagtag

SEQ ID NO: 89 - protein, 454 aa, Spodoptera littoralis
MVVLTSKEKSNMSVADFYAGKSVFITGGTGFLGKVFIEKLLYSCPDIDKIYMLIREKKGQSIRERLTKIVDDPLFNRLKDKRPGDLGKIVLIPGDITVPGLGISEENETILTEKVSVVIHSAATVKFNEPLATAWNVNVEGTRMIMALSRRMKRIEVFIHISTAYTNTNRAVIDEVLYPPPADINDVHQHVKNGVTEEETEKILNGRPNTYTFTKALTEHLVAENQSYMPTIIVRPSIVGAIKDDPIRGWLANWYGATGLSVFTAKGLNRVIYGHSNHVVDLIPVDYVANLVIVAGAKTYHSNEVTIYNSCSSSCNPITMKRLVGLFIDYTVKHKSYVMPLPGWYVYSNYKWLVFLVTVIFQVIPAYLGDIGRRLLGKNPRYYKLQNLVAQTQEAVHFFTSHTWEIKSKRTSELFSSLSLTDQRMFPCDANRIDWTDYITDYCSGVRQFLEKIK

SEQ ID NO: 90 - DNA, 1377 bp, Agrotis ipsilon
  source 1..1377: mol_type = other DNA
atggccgtgatcatctcccgagaggaagagaagctgtctgtccccgagttctacgccggcaagtctatcttcattaccggcggcaccggattcctcggcaaggtgttcatcgagaagctgctctactcttgccccgacatcgacaagatctacatgctgatccgagagaagaagaacctctctatcgacgagcgaatgaccatgttcctggacgaccctctgttctctcgactgaaggaaaagcgacccggcgacgtcgagaagatcgtgctgatccccggcgacatctcttctcccaacctgggcctgtctgccgagaacgaacgaatcctgatcgagaacgtgtctgtgatcatccactctgccgccaccatcaagttcaacgagcccctgcctatcgcctggaagatcaacgtcgagggcacccgaatgctgatggacctgtctcgacgaatgaagcgaatcaaggtgtttatccacatctctaccgcctactctaacgccaactctgagcgagccgccgtggaagagattctgtaccccgctcctgccgacatggaccaggtgtaccagctcgtgaaggacggcgtgaccgaggaagaaaccgagatcctgctgaacggactgcccaacacctacaccttcaccaaggctctggccgagcacctggccgctgagcaccaggtgcacgtgcccaccgtgattattcgaccctctatcgtgggctctatcaaggacgagcccatccgaggctggctgtgcaactggttcggcgccaccggcatctctgtgttcaccgccaagggcctgaaccgagtgctgctcggaaaggcctctaacatcgtggacgtgatccccgtggactacgtggccaacctggtgatcgtggctggcgccaagaacggcggcgagaagtctgaggaactgaagatctataactgctgttcttctgactgcaaccccgtgaccgtgaagaagatcctgaaggaattcatcgacgacaccattaagaacaagtctcacatcatgcctctgcctggctggttcgtgttcaccaagtacaagtggctgatgaccctgctgaccatcatcttccagatgatccccatgtacctggccgacgtgtaccgagtcctgatgggcaagaaccctcggtacatgaagctgcaccacctggtcattcagacccgactggtgatcaacttcttcaccttccactcttgggtgatgaagaccgatcgagcccgagagctgttcggctctctgtctcccgttgagaagcacatgttcccttgggacccctctggcatcgactggaccgagtacctgcagtcttactgctacggcgtgcgacacttcctcgagaagcgaaagtag

SEQ ID NO: 91 - protein, 458 aa, Agrotis ipsilon
MAVIISREEEKLSVPEFYAGKSIFITGGTGFLGKVFIEKLLYSCPDIDKIYMLIREKKNLSIDERMTMFLDDPLFSRLKEKRPGDVEKIVLIPGDISSPNLGLSAENERILIENVSVIIHSAATIKFNEPLPIAWKINVEGTRMLMDLSRRMKRIKVFIHISTAYSNANSERAAVEEILYPAPADMDQVYQLVKDGVTEEETEILLNGLPNTYTFTKALAEHLAAEHQVHVPTVIIRPSIVGSIKDEPIRGWLCNWFGATGISVFTAKGLNRVLLGKASNIVDVIPVDYVANLVIVAGAKNGGEKSEELKIYNCCSSDCNPVTVKKILKEFIDDTIKNKSHIMPLPGWFVFTKYKWLMTLLTIIFQMIPMYLADVYRVLMGKNPRYMKLHHLVIQTRLVINFFTFHSWVMKTDRARELFGSLSPVEKHMFPWDPSGIDWTEYLQSYCYGVRHFLEKRK

SEQ ID NO: 92 - DNA, 1383 bp, Spodoptera frugiperda
  source 1..1383: mol_type = other DNA
atggtggtgctgacctctaaggaaaagtctaacatgtctgtggccgacttctacgccggcaagtctgtgttcattaccggcggcaccggattcctcggcaaggtgttcatcgagaagctgctgtactcttgccccgacatcgacaagatctacatgctgatccgagagaagaagggccagtctatccgagagcgaatgaccaagatcgtggctgaccctctgttcaaccgactgaaggacaagcgacccggcgacctggacaagattgtgctggtgcccggcgacatcaccgtgcctggcctgggcatctctgaggaaaacgagactatcctgaccgacaaggtgtctgtggtgatccactctgccgccaccgtgaagttcaacgagcccctggccaccgcctggaacatcaacgtcgagggcaccaagatgatcatggccctgtctcgacgaatgaagcgaatcgaggtgtttatccacatctctaccgcctacaccaacaccaaccgagccgtgatcgacgaggtgctgtaccctcctcctgccgacatcaacgaggtccaccagcacgtgaagaacggcgtgaccgaggaagagactgagaagatcctgaacggacgacccaacacctacaccttcaccaaggctctgaccgagcacctggtggccgagaaccaggcctacatgcccaccatcatcgtgcgaccctctatcgtgggcgccatcaaggacgaccccatccgaggctggctgtctaactggtacggcgccaccggcctgaccgtgttcaccgccaagggcctgaaccgagtgatccacggacaccccaaccacatcgtggatctgatccccgtggactacgtggccaacctggtgatcgtggccggtgccaagacctaccactctaacgaggtgtctatctacaactgctgttcttcttcttgcaaccccatcaccatgaagcgactggtgggcctgttcattgacgacgccgtgaagcacaagtcttacgtgatgcccctgcctggctggtacgtgtactctaactaccgatggctggtgttcctgatcaccctgatcttccaggtgatccccgccttcctgggcgacatcggccgacgactgctgggcaagtctccccggtactacaagctgcagaacctggtcgctcagacccaagaggccgtgcacttcttcacctctcacacctgggagattaagtctaagcgaacccgagagctgttctcttccctgtcgctgaccgaccagcgaatgttcccctgcgacgccaaccgaatcggctggaccgactatatcaccgactactcttccggcgtgtacattgagaagctcctccactcttgtaagaagatcgacaagtag

SEQ ID NO: 93 - protein, 460 aa, Spodoptera frugiperda
MVVLTSKEKSNMSVADFYAGKSVFITGGTGFLGKVFIEKLLYSCPDIDKIYMLIREKKGQSIRERMTKIVADPLFNRLKDKRPGDLDKIVLVPGDITVPGLGISEENETILTDKVSVVIHSAATVKFNEPLATAWNINVEGTKMIMALSRRMKRIEVFIHISTAYTNTNRAVIDEVLYPPPADINEVHQHVKNGVTEEETEKILNGRPNTYTFTKALTEHLVAENQAYMPTIIVRPSIVGAIKDDPIRGWLSNWYGATGLTVFTAKGLNRVIHGHPNHIVDLIPVDYVANLVIVAGAKTYHSNEVSIYNCCSSSCNPITMKRLVGLFIDDAVKHKSYVMPLPGWYVYSNYRWLVFLITLIFQVIPAFLGDIGRRLLGKSPRYYKLQNLVAQTQEAVHFFTSHTWEIKSKRTRELFSSLSLTDQRMFPCDANRIGWTDYITDYSSGVYIEKLLHSCKKIDK

SEQ ID NO: 94 - DNA, 1353 bp, Spodoptera frugiperda
  source 1..1353: mol_type = other DNA
atgtctaaggccaactgctctaacgtggcctcgttctacgccggcaagtctgtgttcatcaccggcggcaccggcttcgtgggcaagaccctgatcgagaagctgctgtactcttgctctggcatcgacaagatctacgtgctggtgcgagacaagtacggcaagcaggcctctgagcgactggcccgagtgacctctactcccgtgttcgaccgactcaagacctctaagtctgaggaactgaagaagatcgtcgtcatctctggcgacatcacccgagagaacctgggcctgcaagaggacgtgctcaagaccctcgagaacgaggtgtctgtggtgttccacctggccgccaccgtggccttcaagctgcccctgaaggacgccatgcgaatcaacgtgaacggcaccgagaacgtgatcgagctgtgccgacgaatgaagaagctgcaggccttcgtccacgtgtctaccgccttcaccaactctgaccgaaaggaaatcaacgagatggtgtaccccatgcctatcaacctggaagaggcccgacacgtggcctacacctactctcacgacgacgtgatcgtgtctgccttcatgggaaagaagcccaacacctacaccttctccaaggccttcgccgagcagcaggtcatgaagcagtgcgaggacctgcctaccgccatcattcgaccctctatcgtgatgtcctctgtgaaggaaccctgtcctggctggatcgacacctggaacggctctaccggcctgttcgtgggaatgcccgctggcatcctgcgagtggtgctcggacgaggatctaacgtgaccgacctggtgcctgtggacatcgtgaccaacctgatcgtggtggccgctaccgagtgccagaagtctaaggaaatcaaggtctacaactgctgcaccggcacttctaaccccatcacctgtgacgaggctaccaccatctctcgaaaggtggccctgaagtactctctgaacgagctgccctggcctttcctgatgtttaccccttctgtgttcctgtataccctgatcaccttcctgctgcagatcgtgcccgcctacctgatcgacctgtggtgcatcctgaccggccgaaaggctacccagatcaagctgcagcgacgacttaagaaggtggtcgacgccgtcaagttctttctgctgaacgagtggaagttctctgacgacaacattcgagatctgcttaagaccatgtctcccgtggacaaggaaatcttcaacttcgacgtcaagaccatcaactgggagacttgcatcgaggactacatcctgggagcccgaaagtacctgctgaagtctgagaagtag

SEQ ID NO: 95 - protein, 450 aa, Spodoptera frugiperda
MSKANCSNVASFYAGKSVFITGGTGFVGKTLIEKLLYSCSGIDKIYVLVRDKYGKQASERLARVTSTPVFDRLKTSKSEELKKIVVISGDITRENLGLQEDVLKTLENEVSVVFHLAATVAFKLPLKDAMRINVNGTENVIELCRRMKKLQAFVHVSTAFTNSDRKEINEMVYPMPINLEEARHVAYTYSHDDVIVSAFMGKKPNTYTFSKAFAEQQVMKQCEDLPTAIIRPSIVMSSVKEPCPGWIDTWNGSTGLFVGMPAGILRVVLGRGSNVTDLVPVDIVTNLIVVAATECQKSKEIKVYNCCTGTSNPITCDEATTISRKVALKYSLNELPWPFLMFTPSVFLYTLITFLLQIVPAYLIDLWCILTGRKATQIKLQRRLKKVVDAVKFFLLNEWKFSDDNIRDLLKTMSPVDKEIFNFDVKTINWETCIEDYILGARKYLLKSEK

SEQ ID NO: 96 - DNA, 1407 bp, Plutella xylostella
  source 1..1407: mol_type = other DNA
atgaagctgggcaccttcggcaacatggccgccaacggcggctctgtgtgcgacttctacgccaactctaccgtgttcattaccggcggcaccggattcctcggcaaggtgctgatcgagaagctgctgcgatcttgccgaaagatcgacaagatctacgtcctgctgcgaggcaccaccaacaagaccgctcaagagcgactcgaggacctgtctcacgaggtcctgttcgagaaggtgcgacaggacgaccccaacatcatgaagaagctgatccccgtcaacggcgacctgactctgcccaacctgggcatgaacaacgaggactactctctgatcgtgaagaacgtgtctatcgtgttccacgccgctgcctctatccgattcaacctgaagatcaaggaagccctgcactgcaacgtcgagggcaccaagaaggtgatcgagctttgccacaaggcccgatgcatcaaggccttcgtgcacatctctaccgcctactctaacaccgacaagaccttcgtgaaggaagaggtgtaccccgctcctgagaacctggataagatctatgtgatcatcggccagaacggcgacgacaccgaggccgacgacctgacctcttctcgacccaacacctacacctacgccaaggccgtggccgagtacgaggtgctgcgaaaccacggatctgtgcccaccgccatcattcgaccctctatcgtgacccctatctggcgagatcccactcctggctggctggacaagtggtctggcggcacccctatcatcttcggcctgtgcaagggcctgctccgatgcctggacacccagccttctaacgtgatcgacttcatccccgccgacattgccgtgaacctgtccatcgtggccgctgccaagtgccacgtggacacccctaagatccgagtgtacaactgctgctctggccctacaaaccccatgaccttcaagatgctgtctgactactgccacgagaactctatcaagcacggcctgcactggttcccttatcctaagatcttcctgtctaccaaccgagtgttcctgaagattgtgcgactggtgctgcagttcctgcctctgttcctggccgacatgtggctgaagattcgaggcaagcagccccgattcgtgaagatgctggaaaagctgaccaaggtggaaaagaccctgcacttcttcatcaagaaccagtggcagttcgagaaccacaacgttgaggccctgatctctaccctgtctccttcggaccgagtgcagttcccttgcgacccctctgagatctgctggaagacccacttcgaggattactgcctgggcatcgacaagtacctgatgaacacctctgtgaagtctgccaacatcaactctggcatgtacaagtag

SEQ ID NO: 97 - protein, 468 aa, Plutella xylostella
MKLGTFGNMAANGGSVCDFYANSTVFITGGTGFLGKVLIEKLLRSCRKIDKIYVLLRGTTNKTAQERLEDLSHEVLFEKVRQDDPNIMKKLIPVNGDLTLPNLGMNNEDYSLIVKNVSIVFHAAASIRFNLKIKEALHCNVEGTKKVIELCHKARCIKAFVHISTAYSNTDKTFVKEEVYPAPENLDKIYVIIGQNGDDTEADDLTSSRPNTYTYAKAVAEYEVLRNHGSVPTAIIRPSIVTPIWRDPTPGWLDKWSGGTPIIFGLCKGLLRCLDTQPSNVIDFIPADIAVNLSIVAAAKCHVDTPKIRVYNCCSGPTNPMTFKMLSDYCHENSIKHGLHWFPYPKIFLSTNRVFLKIVRLVLQFLPLFLADMWLKIRGKQPRFVKMLEKLTKVEKTLHFFIKNQWQFENHNVEALISTLSPSDRVQFPCDPSEICWKTHFEDYCLGIDKYLMNTSVKSANINSGMYK

SEQ ID NO: 98 - DNA, 1572 bp, Plodia interpunctella
  source 1..1572: mol_type = other DNA
atggctccctctgtgcgagagttctacaagggcaagaacatcttcattaccggcggcaccggattcctcggcaaggccctgatcgagaagctgctgcgatcttgccccgacgtgtctaccatctacctgctgctccgacagaagaagggcctgtcctctgacgagcgactgcgagagctgtgcaacaacaaggtgttcgacgtggtgcgagagcagaaccccgaggtgttcaagaagctgcgactgatctctggcgacattctcgaggaccgactgggcctgtctgccgaggacgagcgagaactgcgagaacgatgcaacatcgtgttccactctgccgcctgcgtgcgattcgaccaaaagctgaaggacgccgtgcacatgaacaccaccggcactctgcgagtgctcgagctggccgagactatggaacatctcgaggtgtttctgcacctgtctaccgcctactgccgatgccacctggacgtgctggaagagaagtcttaccccgctgtgcacgaccctcgacgagtgatggaactggtcgagtggatggacgacgacacccagaactacctcgagcctaagctgatctcttctgagcccaacacctactcttacaccaaggccatcaccgagcagctggtggaagagtaccaagagaagttccccattgccattgctcgaccctctatcgtgatcgccgcctggaaggaacccattcctggctgggtcgacaacatgaacggccccaccggcctgctgatcggctctggcaagggcgccgtgcgatctatgcactgcgagcccgccttcgccgccgacgtgctgcccgtggacgtgaccgtgaacacctgtctgctggccgcctgtgccaccgccaccgaccgacctaaggaagtggtgttctacaacgtgaccctgtctgaggtgatcaagattacctggggcgagatcatcgagctgggcaagaagtggctggctatctaccctcacactctggccctgtggcaccccggtggcaccatcaagtcttactactttcagcaccagctgtgcgtgctgttttaccatctgatccccgcctacctggtggacggactgctgttcctgctgggaaagaaaaccttcctggtcaacgcccagaagcgaatctctcacggactgtctgtgctgcagtactacaccaccaagcagtggcacttcgtcaacaccaacttccgatctctgcgaaaccgaatctctaaggaagatgacgagatcttcttcactgacgtgtcgcccctggactgcgaggacttcctgaagaactacatcctgggagcccgatattacgtgtgcaaggaagatccctctactctgccccgagccagaaagctgtaccgaatccgattcatcgtggaccagatcttccgactcctgttctacttcctgatcttctggctgctgtactcctacaaggacttcttcgtgacctctgttgagaagctggactctgccctgaagtctctgacccctatcaacgtgaaggccgacgtgtgtgagctgaacgaactgaacgaggtgctgtag

SEQ ID NO: 99 - protein, 523 aa, Plodia interpunctella
MAPSVREFYKGKNIFITGGTGFLGKALIEKLLRSCPDVSTIYLLLRQKKGLSSDERLRELCNNKVFDVVREQNPEVFKKLRLISGDILEDRLGLSAEDERELRERCNIVFHSAACVRFDQKLKDAVHMNTTGTLRVLELAETMEHLEVFLHLSTAYCRCHLDVLEEKSYPAVHDPRRVMELVEWMDDDTQNYLEPKLISSEPNTYSYTKAITEQLVEEYQEKFPIAIARPSIVIAAWKEPIPGWVDNMNGPTGLLIGSGKGAVRSMHCEPAFAADVLPVDVTVNTCLLAACATATDRPKEVVFYNVTLSEVIKITWGEIIELGKKWLAIYPHTLALWHPGGTIKSYYFQHQLCVLFYHLIPAYLVDGLLFLLGKKTFLVNAQKRISHGLSVLQYYTTKQWHFVNTNFRSLRNRISKEDDEIFFTDVSPLDCEDFLKNYILGARYYVCKEDPSTLPRARKLYRIRFIVDQIFRLLFYFLIFWLLYSYKDFFVTSVEKLDSALKSLTPINVKADVCELNELNEVL

SEQ ID NO: 100 - DNA, 1596 bp, Plodia interpunctella
  source 1..1596: mol_type = other DNA
atgaccgtgaacacccgaccttctaccaacatggaatctaacatcatctctgagcgaaactacgtgcgagactgcaccggcatcgacgtgaacatgaacgagaagcgactgacctctgtgcagcagttctacaacggcaagaacgtgctgattaccggcgctaccggcttcctgggcaagatcctggtcgagaagctgctccgatgctgtcccggcgtcgagaacctgtacctgctggtgcgacagaagaagggcaaggacatctacacccgaatcgacgagatcttcgacgaccccgtgttcgacctgctgaaggaacagcaccccaagttccgacacaaggtggtggtggtccccgccgactgcgaggtggccggcctgggactgaccctgaccgaccgacagatgctgaccgagaaggtgaacatcattttccactctgccgccaccgtgaagttcgacgagcacctccgagccgctctgaacaccaacgtgcgagcccctctgcacctcctgcgactggcccgagacatgaaggacctggacgtcctgatgcacatctctaccgcctactctaactctcatctgccccacatcgaggaacgattctacccctgcgaggccgactgtgagcagctgcaccgaatgatcgacaagctgaccgacggcgagatcgacgctctgctgcccaccatcctcggcgcttggcccaacacctacaccttcaccaaggctctggccgagaaggaactgcgagagaacgccggcgacctgcctgtgggcatcttccgacctgccatcgtgacctctacctacaaggaacccatcaacaactggctggacaacatgtacggccccactggcgtggccgtgggcaccgccaccggaatcctgcgaaccctgcagtgcgaccccaacgtgaccgccgacctggtgcctgtggaccacgtcgtgaactgcctggtggtggccgctcagaaggtcaacgccgcctaccagacctccacacctcctcgagagcctccaatcttcaactacgtgtcctctaccgagaaccgaatgacctggggcgacttcatgagacagaacattgcccgactggacaagacccctttctctaacgccatctggtatctgtctctgcgactcacccgatctggcgcccgacaccgagtgtactccctgctgctgcatctgctgcctgccgctctggctgacgccctggccttctgcctgggaaagaagcccaagatgctgaaggtctaccgaaaggtgcacaagctgtcctctgtgctgtcttacttctgtaccaccgagctgactttctgcaaccgaaacactcgacagctgtgggagtctacctctgacgaggacaagaagattttccccttctctatgcgggacgtgaactgggacgagttctttgaagagtacatcgtgggcatgcgacgacacctgttcaaggacggtgacgacaccctgcctcaggctcgaatcaagtggaagcgactctactacctgcaccagatcgtgaagatcctgttctgtgccctggccgtgtacgccctgtggtctatctttctgcgactgtggtag

SEQ ID NO: 101 - protein, 531 aa, Plodia interpunctella
MTVNTRPSTNMESNIISERNYVRDCTGIDVNMNEKRLTSVQQFYNGKNVLITGATGFLGKILVEKLLRCCPGVENLYLLVRQKKGKDIYTRIDEIFDDPVFDLLKEQHPKFRHKVVVVPADCEVAGLGLTLTDRQMLTEKVNIIFHSAATVKFDEHLRAALNTNVRAPLHLLRLARDMKDLDVLMHISTAYSNSHLPHIEERFYPCEADCEQLHRMIDKLTDGEIDALLPTILGAWPNTYTFTKALAEKELRENAGDLPVGIFRPAIVTSTYKEPINNWLDNMYGPTGVAVGTATGILRTLQCDPNVTADLVPVDHVVNCLVVAAQKVNAAYQTSTPPREPPIFNYVSSTENRMTWGDFMRQNIARLDKTPFSNAIWYLSLRLTRSGARHRVYSLLLHLLPAALADALAFCLGKKPKMLKVYRKVHKLSSVLSYFCTTELTFCNRNTRQLWESTSDEDKKIFPFSMRDVNWDEFFEEYIVGMRRHLFKDGDDTLPQARIKWKRLYYLHQIVKILFCALAVYALWSIFLRLW

SEQ ID NO: 102 - DNA, 1569 bp, Manducta sexta
  source 1..1569: mol_type = other DNA
atggtgccccgacctgtgtctccctacgctcgagagccccagattcctcagttctacgccggcaagtgcatcttcatcaccggcgccaccggcttcatgggcaaggtgctgatcgagcgactgctgtggacctgtcctgacatcggcaagctgtacctgctgatgcgacagaagcgagaggtttctcccgagaagcgactgtctcagctgaagcagtctcaggtgttcgacaacatccgagagaagtgcccctctcagctggacaagctctacatcatccacggcgacaccaccgagcctaacctgggcatctctctggactctatgtctctgctgaacgaggtgtctatcgtgttccacggcgctgctaccctgaagttcgacgaggtgctgggcaagtctgtggaccagaacgtgcgatctgtgatgcgactgctcgacatctgcgaccgactgcctaacatccaggccttcatccacgtgtctaccgcctactgcaacgccgacctgatcaccgtggaagagaaggtctacacctctcctgtgtcgctggaccaggtgctggccctgaccgactctgtgcccgacgagctgatggccgagatcaccgacaagttcattgctcccaagcctaacacctacaccttcaccaaggctctggccgagaacgccgtcgagcagcaccgaaaccgaggctaccccgtggccatcttccgacctaccatcgtgatctctgccctgcagcacccctttccaggctggatcgagaacctgaacggcccctctggcgtggtggtcggagccggcaagggcctgctgcacgtgttctgctgcaagaactctgccaaggccgacctgctgcctgtggacattgccatcgacaccctgattgccgtggcctgggagactgccctcgacaagctgtctgaggtgcgagtgtacaactgctctaccatcgacaaccccgtgacctggcgagagttcgagactaccatccgacgagagctgcgaattcaccccttcgacaaggccttctggtatccctctggcaccgtgatcgagtctaagtacctgcagaaggctatcgccctgctgacccagactctgcccctgcacctggccgagtacaccgtgcgactgttcggcatcaagacccgactgtctttcattaccgtgaaccagcgactgtgcgccatgtctgacgtgctgcagttcttctcgctgcgagagtggatcttcaagaaggacaacgtccgacgactgcaggctcgactgtcgccccgagatgccgagatctacaacctggatcctcagaccatcaactggaccgagcacttcatcaacttcgtgaagggcacccgaaagtatctgctgaaggaaaaggaccaggacatcggcgaggccaagcgacacatcagacgaatgtactacgtgcaccacggcttcatcctgtttctgctggtgctggtgttccgatacgccatgcagaaccccatcatccgatccatcgtgtacggcaccctgaagctgctgttctctgccttcaacgccgcctacggccgaatctga

SEQ ID NO: 103 - protein, 522 aa, Manducta sexta
MVPRPVSPYAREPQIPQFYAGKCIFITGATGFMGKVLIERLLWTCPDIGKLYLLMRQKREVSPEKRLSQLKQSQVFDNIREKCPSQLDKLYIIHGDTTEPNLGISLDSMSLLNEVSIVFHGAATLKFDEVLGKSVDQNVRSVMRLLDICDRLPNIQAFIHVSTAYCNADLITVEEKVYTSPVSLDQVLALTDSVPDELMAEITDKFIAPKPNTYTFTKALAENAVEQHRNRGYPVAIFRPTIVISALQHPFPGWIENLNGPSGVVVGAGKGLLHVFCCKNSAKADLLPVDIAIDTLIAVAWETALDKLSEVRVYNCSTIDNPVTWREFETTIRRELRIHPFDKAFWYPSGTVIESKYLQKAIALLTQTLPLHLAEYTVRLFGIKTRLSFITVNQRLCAMSDVLQFFSLREWIFKKDNVRRLQARLSPRDAEIYNLDPQTINWTEHFINFVKGTRKYLLKEKDQDIGEAKRHIRRMYYVHHGFILFLLVLVFRYAMQNPIIRSIVYGTLKLLFSAFNAAYGRI

SEQ ID NO: 104 - protein, 578 aa, Bombus terrestris
MKKSLSKNQRKSDDTETKDIRGKMEEIDVKSSSSTNSMTNNLALLPSGVGRMKQLQGAKMVNQTGSSSSGSATGNPRNKTALAPGHSLMDWIRLGNSGVDLTGVGGVPRVVTLSELATHNKQDDAWIAIRGIVFNVTRYMDFHPGGVNELMRGVGKDATKLFENVHAWVNYQSILQKCVVGRLSRGSVSGTTSSPTENPVSSTSNCSSATLNLITSCTTHSVSQENVSDASSLNIKMDWRQTSDSLTLSYQTTRYYPGLCYQLSRINDSKLMYKLVFASGSITHELELTAEIQWPPVCIKNFETREITFTFMKQKQELWKSYGEQMVCREMNRDNRTYREYQVLTNKQLSKLVHLLVVRAKDFLQIVPIGRHVEAMLNVMGTEVSRLYTPVPPCLHPDDMAPNYKSDCLCFMIKKYPNGALSPSITALQIGETLRLSNALGDFTVESCDGYSINHMIAGGTGLTAMLGIIQRALARRSVKTINLLNFNKDEDNMFYVKELEKASADNKLKVTHILSQAGDAWTGRRGTLSDDLLQDLVGTSNPDAFVYTCGPQEFIQLAKNSLQKLNWKPFQMYEFDD

SEQ ID NO: 105 - protein, 678 aa, Lobesia botrana
MADPPAEDNTPSRNGSLKISIPTVLCTPESSENSDILCKTKSLLSSESKLSLPSNLGTPTSPLRLSSPLSLPASPTPFMKVTQPPPEKVTSGQMNSPPSTIVLQSGPKPPLANTGSATGNPRNKCALQPGHSLMDWIRLGNSGKDLTGVGGRMRPVSPDELASHNSQNDAWLAIRGRVYNITHYLPYHPGGPEELMRGAGIDATQLFDKVHPWVNYDSLLAKCLVGPLRLERPDAEELFGPSTPSPKTENRLREPSKAQELVRKSMENLANCITPVRKKISAKSEESVKGSPPSKIMQSLIQSSDLPVSISRRAAANPVKTSEKTDVAPSPLRFDWIQTSTKLTLSIYTGPLVNPGVCARIIEGILFVEVATNGWVRTLKLVPEAKLQDSLQLRVFAESGKIEVVAQKVEPGVWKGCGDAVSGAASHVPTPRSVVCRVSRVTRVSHDTVLLSLATPAQALVAPLGHHVRVHRTISDVECVRSYTPVGEGWTSDGERDSAIHLAVKGYETGALSPHLVALQPDDEVTVSGPYGNFELQKLRGVKEICFVAAGTGITPMLGLLKFILPRSNPRCERVHLLFFNKTEEDILFRQNFEEIAKEDDRFSVTHVLSDASSSWSGRRGRITSDLLTQVVGKDSLKCREQCVHFACVCGPTDFTHTTIDLLKKLHMKDSCIHAFMG

SEQ ID NO: 106 - DNA, 1566 bp, Amyelois transitella
  source 1..1566: mol_type = other DNA
atggtggcccgacgagagtctgtgtctgctcccgtgattccccagttctacgccggacgatctatcttcatcaccggcgccaccggcttcatgggcaaggtgctgatcgagcgaatcctggccacctgtcctgacgtggaccgactgtacctgctggtgcgacacaagaaggaagtcacccctgagcgacgactgcagcagctgattgagtctcccgtgttcgaccccatccgagcttctaacccctctcagctggacaagctgtctatgctggccggcgacatcacccgacctgacctgggcatgacccaagagtctctgatgcgactgcaagaggtgtctatcgtgttccactctgccgccactctgaagttcgacgaggccatgatgatggccgtcgagcagaacgtgctgtctgtgatccgactgctcgagatctgcgaccgactgcctaagatgcaggccttcgtgcacgtgtctaccgcctactctaacgccgagctgtctgaggtcgaggaacacgtgtaccctccaaaggtgcccctcgagaagctgctggccattgccgagtctgtccccgacgagctgatgaccgagatcacccctaagttcattgctcccaagcctaacacctacaccttcaccaagtctatggccgagcacgtggtgcagcagcacggcaacaagggctaccccgtggccatcttccgacctaccatcgtgatctcttccgtgaagcacccctttccaggctggatcgagaacctgaacggcccctctggcgtggtggctgctgccggcaagggcctgctgcacgtgttctgctgccgaggcgacgcccacgccgacatgctgcccgtggacgtggccattgacaccctgatcgccgctgcctgggagactgccatcgacaagcccaacgaggcccgagtgtacaactgcaccactcgagagaaccccaccaagtggatcgacttcgagcacgccctgcgaaagtacctggtcgagtaccctctggacaaggccttctggtatccctctggtgccgctatcgacaacacctacgctcaaaaggccatggaactgttcacccagaccgtgcctctgcacatcgtggcctacaccgtgcgactgctgggcatcaagatgcagatgaacctgatcaccgtgtctcaccgactgcaggccatgaacaaggtcctgaagttcttcgcccagcgagagtgggtgttccacaacgagaacgtgaagcgactgcgaaaccgactgacccctcaggacgccgccatctacaacctggatcctaagtctttcaactgggacgagcactactgcaacttcatcaagggcacccgaaagtatctgctgaaggaaaaggaacaggacctggaagaggcccgaaagcacgtgcgacgaatgttctacgtgcactactctgtgctgctgttcgtggtggtcctgctgctgcgattcgccctgaacaacaacttcgtgcgacagttcatctacggcgtggtgcgaaccctgaccgctgccttcggcgccgtgttctctaagatcgtgtag

SEQ ID NO: 107 - protein, 521 aa, Amyelois transitella
MVARRESVSAPVIPQFYAGRSIFITGATGFMGKVLIERILATCPDVDRLYLLVRHKKEVTPERRLQQLIESPVFDPIRASNPSQLDKLSMLAGDITRPDLGMTQESLMRLQEVSIVFHSAATLKFDEAMMMAVEQNVLSVIRLLEICDRLPKMQAFVHVSTAYSNAELSEVEEHVYPPKVPLEKLLAIAESVPDELMTEITPKFIAPKPNTYTFTKSMAEHVVQQHGNKGYPVAIFRPTIVISSVKHPFPGWIENLNGPSGVVAAAGKGLLHVFCCRGDAHADMLPVDVAIDTLIAAAWETAIDKPNEARVYNCTTRENPTKWIDFEHALRKYLVEYPLDKAFWYPSGAAIDNTYAQKAMELFTQTVPLHIVAYTVRLLGIKMQMNLITVSHRLQAMNKVLKFFAQREWVFHNENVKRLRNRLTPQDAAIYNLDPKSFNWDEHYCNFIKGTRKYLLKEKEQDLEEARKHVRRMFYVHYSVLLFVVVLLLRFALNNNFVRQFIYGVVRTLTAAFGAVFSKIV

SEQ ID NO: 108 - DNA, 1572 bp, Amyelois transitella
  source 1..1572: mol_type = other DNA
atggcctcttgcgtcggcggagccttccacgctcaagagtacctgcctgtggccgacttctacgccgacaagtctatcttcgtgaccggcggcaccggcttcatgggcaaggtgctggtcgagaagctgctgcgatcttgccccaagatcaagaagatctacctgctgatgcgacccaagcgaggccaggacgtgtcctctcgactgaccgagctgacccagtctcctctgttcgagactctgcgaaaggaacgaccctctgagctgtctaagatcgtgcccatcgtgggcgacatcaccgagcctgagctgggcatctctcccgccgaccaggccatgctgtgccaaaaggtgtctgtggtgttccactctgccgccaccgtgaagttcgacgagaagctcaagctgtctgtgaccatcaacatgctgggcacccagcagctggtgcagctgtgccaccgaatgctgggactcgaggccctggtgcacgtgtctaccgcctactgcaactgcgagcgagagcgagtcgaggaaaccgtgtacgcccctcctgctcaccccgagcacgtggtgaccctggtccagactctgcccgacgacctggtggaccgaatcacccctgacctggttggcgaccgacctaacacctacaccttcaccaaggctctggccgaggacatgctgatcaaggaatgcggcaacttgcccgtggccatcgtgcgaccctctatcgtgctgtcctctctgcgagagcccgtgaagggctgggtcgacaactggaacggccccaacggcatcattgccgccgtcggcaagggcgtgttccgatctatgctcggaaccggcgctcgagtggccgatctggtgcccgtggacaccgtgatcaacctgatgatcgtgtgcgcctggcgaacccacctccgacgaggcgagggcgtcgtggtgtacaactgctgcaccggccagcagaaccccatcacctggcagcgattcgtcaagacctctttcaagtacatgcgaaagcacccattctctgaggtggtgtggttccccggtggcgacattaccaacaaccgactgaagcacaacatcctgtctctgctccagcacaaggctcccgccatcgtgatggacctggtgtctcgagcctctggcaacaagcccgtgatggtgcgagtgcagaacaagctcgagaaggccgctgcctgcctcgagtacttcaccactcgacagtgggccttctctgacgacaacgtgcaggccctgtgctctgccctgtctgtcgaggaccgacgaatcttcgacttcaacgtgcgaaacatcgactgggacgcctacatcgagtcttacgtgctgggaatccgacgattcctgttcaaggaatctcccgacactctgcccaagtcgcgagccctgctgcgacgactgcacatcgtgcacaccctgactcaggtgaccaccgtgtttctgctgtggcgatttctgttctctcagtcgtctaccctgcgatctatctggcgacgaattctcgagttcatcacccgaattttccgactgctggccattgcctag

SEQ ID NO: 109 - protein, 523 aa, Amyelois transitella
MASCVGGAFHAQEYLPVADFYADKSIFVTGGTGFMGKVLVEKLLRSCPKIKKIYLLMRPKRGQDVSSRLTELTQSPLFETLRKERPSELSKIVPIVGDITEPELGISPADQAMLCQKVSVVFHSAATVKFDEKLKLSVTINMLGTQQLVQLCHRMLGLEALVHVSTAYCNCERERVEETVYAPPAHPEHVVTLVQTLPDDLVDRITPDLVGDRPNTYTFTKALAEDMLIKECGNLPVAIVRPSIVLSSLREPVKGWVDNWNGPNGIIAAVGKGVFRSMLGTGARVADLVPVDTVINLMIVCAWRTHLRRGEGVVVYNCCTGQQNPITWQRFVKTSFKYMRKHPFSEVVWFPGGDITNNRLKHNILSLLQHKAPAIVMDLVSRASGNKPVMVRVQNKLEKAAACLEYFTTRQWAFSDDNVQALCSALSVEDRRIFDFNVRNIDWDAYIESYVLGIRRFLFKESPDTLPKSRALLRRLHIVHTLTQVTTVFLLWRFLFSQSSTLRSIWRRILEFITRIFRLLAIA

SEQ ID NO: 110 - DNA, 1389 bp, Ostrinia furnacalis
  source 1..1389: mol_type = other DNA
atgtctgccaacaccatggaaaccgacgagcagttcacctacaactctcccatcgtgaacttctactctggcaagtctgtgttcgtgaccggcgctaccggcttcctgggcaccgtgctggtcgagaagctgctgttctcttgcaagggcatcaacaacatctacatcctgatcaagcagaccgaggacctgaccatcgaggcccgaatcctgaactacctgaactctaaggccttccaccgagtgaagaacactaaccccgagctgatgaagaagatcatccccatctgcggcaacctcgaggacaagaacctgggcatctctgactctgacatgaagaccctgctggaagaggtgtctatcgtgttccacgtggccgccaagctgatcttcaagatgtctctgaccgccgctgtgaacatcaacaccaagcctaccgagcagctgatcgccatctgcaagaagatgcgacgaaaccccatcttcatctacgtgtcctctgcctactctaacgtgaacgagcagatcatcgacgagaaggtgtactctaccggcgtgcccctcgagactatctacgacaccctggacgccaagaacacccgactgatggacatcttcctggacaagcgacccaacacctacacctactccaaggctctggccgaggtggtcgtcgagaaggaattcgacgagtctgccgccatcgtgcgaccctctatcattgcctcttctattcgagagcccattcctggctggctgtctggctctcacggcttccctcgagtggtcgaggccgcctgcaagggcctgctgctccgatggcacggcgacggcaccgtggccttcgacgtgatccccgtggaccacgtcgccaacctgatcattgccgccgcttgggagtctaacgagcgacgactcatgggcaacaagggcgtgaaggtctacaactgctgctctggcctgcgaaaccctatcgacgtgtctaccgtgatgaacacctgtatcaagtaccgaaagtacttcggcacccgaaccatgtctatcatcacccctcgattcattatgaagaagaactacttcctgtacaagctgctgtacttcacctaccacactatccccgctgctattatcgacggcttcttctggctgaccggacgaacccctatgatgctgaacaccctgcacaagctccgaaagctgtcctctgtgctcgagtactttaccctgcaccagtttctgtttctggactccaacgtgcgaggactgctgcgacgaatggaatctaccgaccgacagaccttcaacttcgacgtcaccgagatcgagtgggagccctacctgcagaacttcgtgcgaggcattgccaacaactacaactattctatgtag

SEQ ID NO: 111 - protein, 462 aa, Ostrinia furnacalis
MSANTMETDEQFTYNSPIVNFYSGKSVFVTGATGFLGTVLVEKLLFSCKGINNIYILIKQTEDLTIEARILNYLNSKAFHRVKNTNPELMKKIIPICGNLEDKNLGISDSDMKTLLEEVSIVFHVAAKLIFKMSLTAAVNINTKPTEQLIAICKKMRRNPIFIYVSSAYSNVNEQIIDEKVYSTGVPLETIYDTLDAKNTRLMDIFLDKRPNTYTYSKALAEVVVEKEFDESAAIVRPSIIASSIREPIPGWLSGSHGFPRVVEAACKGLLLRWHGDGTVAFDVIPVDHVANLIIAAAWESNERRLMGNKGVKVYNCCSGLRNPIDVSTVMNTCIKYRKYFGTRTMSIITPRFIMKKNYFLYKLLYFTYHTIPAAIIDGFFWLTGRTPMMLNTLHKLRKLSSVLEYFTLHQFLFLDSNVRGLLRRMESTDRQTFNFDVTEIEWEPYLQNFVRGIANNYNYSM

SEQ ID NO: 112 - DNA, 1392 bp, Ostrinia zag
  source 1..1392: mol_type = other DNA
atgtctgccaacaccgtcgaaaccgacgagcagttcaccgacaacaactctcccatcgtgaacttctactctggcaagtctgtgttcgtgaccggcgctaccggcttcctgggcaccgtgctggtcgagaagctgctgttctcttgcaagggcatcaacaacatctacatcctgatcaagcagaccgaggacctgaccatcgaggcccgaatcctgaactacctgaactctaaggccttccaccgagtgaagaacactaaccccgagctgatgaagaagatcatccccatctgcggcaacctcgaggacaagaacctgggcatctctgactctgacatgaagaccctgctggaagaggtgtctatcgtgttccacctggccgccaagctgctcttcaagatgtctctgaccgccgctgtgaacatcaacaccaagcctaccgagcagctgatcgccatctgcaagaagatgcgacgaaaccccatcttcatctacgtgtcctctgcctactctaacgtgaacaagcagatcatcgacgagaaggtgtactctaccggcgtgcccctcgagactatctacgacaccctgaacaccgagaacacccgaatcaccgacatcctgctggacaagcgacccaacacctacacctactccaaggctctggccgaggtggtcgtcgagaaggaattcgacgagtctgccgccatcgtgcgaccctctatcattgtgtcttctattcgagagcccattcctggctggatgtctggctctcacggcttccctcgagtggtgggagccgcctgcgagggcctgctgctccgatggcacggcgactctaccgtggtgttcgacgtgatccccgtggaccacgtcgccaacctgatcattgccgccgcttgggagtctaacgagcgacgactcatgggcaacaagggcgtgaaggtctacaactgctgctctggcctgcgaaaccctatcgacgtgtctaccatcatgaacacctgtctgaagtaccgaaagtacttcggcacccgaaccctgtctatcttcacccctcgattcattgtgaagaagaactacttcatctacaagctgctgtacttcacctaccacactatccccgctgctattgtggacggcttcttctggctgacccgacgaacccctatgatgctgaacaccctgcacaagctctctaagctgtcctctgtgctcgagtactttaccctgcaccagtttctgtttctggactccaacgtgcgaggactgctgcgacgaatggaatctaccgaccgacagaccttcaacttcgacgtcaccgagatcgagtgggagccctacctgcagaactgtgtgcgaggaatcgccaacaactgcgactattctatgtag

SEQ ID NO: 113 - protein, 463 aa, Ostrinia zag
MSANTVETDEQFTDNNSPIVNFYSGKSVFVTGATGFLGTVLVEKLLFSCKGINNIYILIKQTEDLTIEARILNYLNSKAFHRVKNTNPELMKKIIPICGNLEDKNLGISDSDMKTLLEEVSIVFHLAAKLLFKMSLTAAVNINTKPTEQLIAICKKMRRNPIFIYVSSAYSNVNKQIIDEKVYSTGVPLETIYDTLNTENTRITDILLDKRPNTYTYSKALAEVVVEKEFDESAAIVRPSIIVSSIREPIPGWMSGSHGFPRVVGAACEGLLLRWHGDSTVVFDVIPVDHVANLIIAAAWESNERRLMGNKGVKVYNCCSGLRNPIDVSTIMNTCLKYRKYFGTRTLSIFTPRFIVKKNYFIYKLLYFTYHTIPAAIVDGFFWLTRRTPMMLNTLHKLSKLSSVLEYFTLHQFLFLDSNVRGLLRRMESTDRQTFNFDVTEIEWEPYLQNCVRGIANNCDYSM

SEQ ID NO: 114 - DNA, 1389 bp, Ostrinia zea
  source 1..1389: mol_type = other DNA
atggccgccaacaccatcgaaaccgacgagcagttcaccgacaactctcccatcgtgaacttctactctggcaagtctgtgttcgtgaccggcgctaccggcttcgtgggcaccgtgctggtcgagaagctgctgttctcttgcaagggcatcaacaacatctacatcctgatcaagcagaccgaggacctgaccatcgaggcccgaatcctgaactacctgaactctaagcccttccaccgagtgaagaacactaaccccgagctgctgaagaagatcatccccatctgcggcaacctcgaggacaagaacctgggcatctctgactctgacatgaagaccctgctggaagaggtgtctatcgtgttccacgtggccgccaagctgctcttcaagatgaacctgaccgccgctgtgaacatcaacaccaagcctaccgagcagctgatcgccatctgcaagaagatgcgacgaaaccccatcttcatctacgtgtcctctgcctactctaacgtgaacgagcagatcatcgacgagaaggtgtactctacccgagtgcccctcgagactgtgtacgacaccctggacatcgagaacacccgaattaccgacatcttcctggacaagcgacccaacacctacacctactccaaggctctggccgaggtggtcgtcgagaaggaattcaacgagtctgccgccatcgtgcgaccctctatcattctgtcttctattcgagagcccattcctggctggctgtctggctctcacggcttccctcgagtggtcggagccgcctgcaagggcctgctgctccgatggcacggcgacggcaccgtggtgtgcgacgtgatccccgtggaccacgtcgccaacctgtctattgccgtggcctgggagtctaacgagcgacgactcatgggcaacaagggcgtgaaggtctacaactgctgctcttctctgcgaaaccctatcgacgtgtctaccgtgctgaacacctgtctgaagtaccgaaagtacttcggcacccgaaccatgtctatcttcacccctcgattcattatgaagaagaactacttcctgtacaagctgctgtacttcacctaccacactatccccgctgctattatcgacggcttcttctggctgaccggacgaacccctatcatgctcaagaccctggacaagctccgaaagctgtcctctgtgctcgagtactttacccaccaccagtttctgtttctggactccaacgtgcgaggactgctgcgacgaatggaaggcaccgaccgacagaccttcaacttcgacgtcaccgagatcgagtgggagccctacctgcagaacttcgtgcgaggcattgccaacaactacgacaactctatgtag

SEQ ID NO: 115 - protein, 462 aa, Ostrinia zea
MAANTIETDEQFTDNSPIVNFYSGKSVFVTGATGFVGTVLVEKLLFSCKGINNIYILIKQTEDLTIEARILNYLNSKPFHRVKNTNPELLKKIIPICGNLEDKNLGISDSDMKTLLEEVSIVFHVAAKLLFKMNLTAAVNINTKPTEQLIAICKKMRRNPIFIYVSSAYSNVNEQIIDEKVYSTRVPLETVYDTLDIENTRITDIFLDKRPNTYTYSKALAEVVVEKEFNESAAIVRPSIILSSIREPIPGWLSGSHGFPRVVGAACKGLLLRWHGDGTVVCDVIPVDHVANLSIAVAWESNERRLMGNKGVKVYNCCSSLRNPIDVSTVLNTCLKYRKYFGTRTMSIFTPRFIMKKNYFLYKLLYFTYHTIPAAIIDGFFWLTGRTPIMLKTLDKLRKLSSVLEYFTHHQFLFLDSNVRGLLRRMEGTDRQTFNFDVTEIEWEPYLQNFVRGIANNYDNSM

SEQ ID NO: 116 - DNA, 1563 bp, Spodoptera exigua
  source 1..1563: mol_type = other DNA
atggtgccccgacctgctcctcagttccccacacctcctctgatccccgagtacttcgccggacgagaggtgctgatcaccggcgccaccggcttcatgggcaaggtgctggtcgagcgactgctgtggacctgtcctgacatcggccgactgcatctgctgatgcgacacaagcgagatgtggctcccgacaagcgactggccctgctgaagcagtctcaggtgttcgacgtggtgcgagaacgatgtccccagcagctggacaagctgtgcatggtgcccggcgacgtgaccaagcgacgattcggcttcgaccagtctgccctgaaccagctcaaccaggtgtctgtggtgttccactctgccgccactctgaagttcgacgagcccctgtctgtggccgccgagcagaacgtgcgacccgtgctgaccctgatggacatctgcgaccagctgcctaacatgcaggtcctggtgcacgtgtctaccgcctactctaacgccgagctggccgaggtcgaggaacgagtgtaccccgctcctgtgactcccgagcacctcctggctctggtggacgctctgcccgcctctatgctgcaagagatcacccctcgactgatcgctcccaagcctaacacctacaccttcaccaaggccgtggccgagtctgccgtgtctgagcgagccgtgaccgctcgatacgcctgcgccatcttccgacctaccatcgtggtgtcctcgctgcgacacccctttccaggctggatcgagaacctgaacggcccctctggcgtggtggctggcgccggaaagggcctgctgcgagtgctccgatgtggcgcccagcgacgagccgacatgatgcccgtcgacatctgcatcgacaccctgatcgccgtggcctgggagactggaatcgacaacctgcgagaggcccgagtctaccagtgcgcctcttcttctcacgccgccacctggggccagttccgagagcgaatgctgcgactggtccgagagcaccccttcgacaacgtgctgtggtatccctacggcgtgatctgcgagaacaccgtggtgcaaaaggtgctcgaggccgtgctgcagaccgctcctctgtgcgtggcccactgtgtggcccgagcctgcggcctgaagcagaagccctctctctggaccgcctgtaagcgactgcaggccatgaaccaggctctgcagttcttcgctacccgacactggtctttccgaactacccgagtgcagcagctcgcccaccgactgcaccccgacgaccagaagctgtacaacctccgacctgaaaccatcgactgggagcagcactgcgtggacttcgtgaagggcgctcgacgatacctgcttcgagagcgagatgacgacatccacgctgcccgacgacgaatgcgaatcctgtacctgatccacaaggctaccctgctgttcgccattttcaccatgtgccgactgaccatccgaactgctcccgccatcctgtggggcgtcgccgctctgacccgacagcgaaacaagtctgctctgctgtcttag

SEQ ID NO: 117 - protein, 520 aa, Spodoptera exigua
MVPRPAPQFPTPPLIPEYFAGREVLITGATGFMGKVLVERLLWTCPDIGRLHLLMRHKRDVAPDKRLALLKQSQVFDVVRERCPQQLDKLCMVPGDVTKRRFGFDQSALNQLNQVSVVFHSAATLKFDEPLSVAAEQNVRPVLTLMDICDQLPNMQVLVHVSTAYSNAELAEVEERVYPAPVTPEHLLALVDALPASMLQEITPRLIAPKPNTYTFTKAVAESAVSERAVTARYACAIFRPTIVVSSLRHPFPGWIENLNGPSGVVAGAGKGLLRVLRCGAQRRADMMPVDICIDTLIAVAWETGIDNLREARVYQCASSSHAATWGQFRERMLRLVREHPFDNVLWYPYGVICENTVVQKVLEAVLQTAPLCVAHCVARACGLKQKPSLWTACKRLQAMNQALQFFATRHWSFRTTRVQQLAHRLHPDDQKLYNLRPETIDWEQHCVDFVKGARRYLLRERDDDIHAARRRMRILYLIHKATLLFAIFTMCRLTIRTAPAILWGVAALTRQRNKSALLS

SEQ ID NO: 118 - DNA, 1386 bp, Trichoplusia ni
  source 1..1386: mol_type = other DNA
atgttcacctccaccaacaacatccgaaccgaggacgacgtgaacgccgagcacgtcgagtctatcgccgacttctacgccggcaagtctgtgttcattaccggcgtgaccggcttcctcggcaaggtgtacctcgagaagctgctgtacacctgtaaggacatcgacaaggtgttcatcctgatccgaaacaagaagggcgacgacgtcggcaagcgaattgagaagctcctggactcttctgtgttctctcgactgaagtctcagcgaccccaggacatcaagaagatcgtccccgtctacggcgacatggacaagtctgagctgggcctgtcctctgaggaccaagagcgactgatcaacgaggtgtctgtggtgttccacgtggccgcctctgtgaacctggacgccgacctgcgatcttctatggctaccaacttcttcggcacctcttacgtgctgaagctgtgccaccgaatgaagaagatcaaggttttcgtctacgtgtctaccgcctactgcaactctaccctgaaggtgctggaagagaaggtctaccccgtgcaagaggaactggaagaggtcctgaagtacctggaagaaccccacctggaccgagatcgagtgaagaagctgttgaacggacgacccaacgcctacgctctgtctaaggccctggccgagcactacgtggcccagaaccacggtgacatccccatcattttcattcgaccctctatcgtgatctcttcgtctaaggaacccgtgcctggctgggtcgactctttccagggcgccaccgctctgatcaccgccgtcctgaagggcgtgaaccgagtgatctacggcaacaagaacaacgtgctggacctgattcctgtggactacgtgtccaacctgtctatcgtggccgctgctcgagtcaagtcctctaaggaagtgaccgtctacaactgctgcacctcttctatcaagcccatcactctgaaggaactggcctctcagatcacccgacgaaaggtcgaggacaagcagcgattcctgttcctgcctaccgtgttcttctctacccactggctggtggtgatcattctgaccttcgtgatgcagatcatgccctcgtttattgccgaccagttcctgtacttcaccggaaacaagcctatgtacatgaagatccagtctaaggtgatcaacggccgaaacgtcctgaactactacactaacaactcttgggtgttcaagtccgtggcctctcgacgactgctggacaccctgtctctgtctgacaagcgactgttcccttgctacccctctgacctgaactgggacaagtactttaagacctacttcgacggaatcgagaacttcctgctgaagaagtag

SEQ ID NO: 119 - protein, 461 aa, Trichoplusia ni
MFTSTNNIRTEDDVNAEHVESIADFYAGKSVFITGVTGFLGKVYLEKLLYTCKDIDKVFILIRNKKGDDVGKRIEKLLDSSVFSRLKSQRPQDIKKIVPVYGDMDKSELGLSSEDQERLINEVSVVFHVAASVNLDADLRSSMATNFFGTSYVLKLCHRMKKIKVFVYVSTAYCNSTLKVLEEKVYPVQEELEEVLKYLEEPHLDRDRVKKLLNGRPNAYALSKALAEHYVAQNHGDIPIIFIRPSIVISSSKEPVPGWVDSFQGATALITAVLKGVNRVIYGNKNNVLDLIPVDYVSNLSIVAAARVKSSKEVTVYNCCTSSIKPITLKELASQITRRKVEDKQRFLFLPTVFFSTHWLVVIILTFVMQIMPSFIADQFLYFTGNKPMYMKIQSKVINGRNVLNYYTNNSWVFKSVASRRLLDTLSLSDKRLFPCYPSDLNWDKYFKTYFDGIENFLLKK

SEQ ID NO: 120 - DNA, 1581 bp, Trichoplusia ni
  source 1..1581: mol_type = other DNA
atggcctcttgcctgtctggcggccactacgtgcccggctctcaagaatacgtccccgtggccgagttctacgccgacaagtctgtgttcgtgaccggcggcaccggcttcatgggcaaggtgctggtcgagaagctgctgcgatcttgccccaagatcaagaagatctacctgctgatgcgacccaagcgaggccaggacgtggcctctcgactgaccgagctgacccagtctcctctgttcgagactctgcgacgagagcgaccccaagagctgaacaagatcgtgcccatcgtgggcgacatcaccgagcctgagctgggcatctctgccgccgaccagaccatgctgtgccaaaaggtgtctgtggtgttccactccgccgccaccgtgaagttcgacgagaagctcaagctgtctgtgaccatcaacatgctgggcacccagcagctggtgcagctgtgccaccgaatgctgtctctcgaggccctggtgcacgtgtctaccgcctactgcaactgtgagcgagagcgagtcgaggaaaccgtgtacgcccctcctgctcaccccgagcacgtggtgaccctggtccagactctgcccgacgagctggtggaccgaatcacccctgacctggtgggcgaccgacctaacacctacaccttcaccaaggctctggccgaggacatgctgatcaaggaatctggcaacctgcctgtgtctatcgtgcgaccctctattgtgctgtcctctctgcgagagcccgtgaagggctgggtcgacaactggaacggccccaacggcatcattgccgccgtcggcaagggcatcttccgaaccatgctcggaaccggcaccaaggtggccgatctggtgcccgtggacaccgtgatcaacctgatgatcgtgtgcgcctggcgaacccacctccgacgaggcgagggcgtcgtggtgtacaactgctgcaccggccagcagaaccccatcacctggcagcgattcgtcaagacctctttcaagtacatgcgaaagcaccccttcaacgaggtggtgtggtatcccggcggagacatcacctctaaccgactgtggcacggcgccctgtctctgctccagcatcgagcccctgccgctctgatggacctggccgcctctgcctctggcaagaagcccatgatggtgcgagtgcagaacaagctcgagaaggccgctgcctgcctcgagtacttcaccactcgacagtgggccttcgccgacgacaacgtgcaggccctgtgttcttcgctgtctcctgaggaccgacgaaccttcgacttcaacgtgcgaaacatcaactgggacgcctacatcgagtcttacgtgctgggaatccgacgattcctgtttaaggaatctcccgacactctgccccagtctcgagccgtgctgcgacgactgcacatcgtgcacattctgacccaggtggctaccgtgttctttctgtggcgatttctgttctctcgatctaacgccctgcgaaacctgtggcgacgagtgctcgagctgctgacccgagccgctcgactgctggccattgcctag

SEQ ID NO: 121 - protein, 526 aa, Trichoplusia ni
MASCLSGGHYVPGSQEYVPVAEFYADKSVFVTGGTGFMGKVLVEKLLRSCPKIKKIYLLMRPKRGQDVASRLTELTQSPLFETLRRERPQELNKIVPIVGDITEPELGISAADQTMLCQKVSVVFHSAATVKFDEKLKLSVTINMLGTQQLVQLCHRMLSLEALVHVSTAYCNCERERVEETVYAPPAHPEHVVTLVQTLPDELVDRITPDLVGDRPNTYTFTKALAEDMLIKESGNLPVSIVRPSIVLSSLREPVKGWVDNWNGPNGIIAAVGKGIFRTMLGTGTKVADLVPVDTVINLMIVCAWRTHLRRGEGVVVYNCCTGQQNPITWQRFVKTSFKYMRKHPFNEVVWYPGGDITSNRLWHGALSLLQHRAPAALMDLAASASGKKPMMVRVQNKLEKAAACLEYFTTRQWAFADDNVQALCSSLSPEDRRTFDFNVRNINWDAYIESYVLGIRRFLFKESPDTLPQSRAVLRRLHIVHILTQVATVFFLWRFLFSRSNALRNLWRRVLELLTRAARLLAIA

SEQ ID NO: 122 - DNA, 1353 bp, Trichoplusia ni
  source 1..1353: mol_type = other DNA
atgtcctcgctgaagggccccaaggtggcctcgttctacaccggcaagaccgtgttcattaccggcggaaccggcttcctgggcaagaccctgatcgagaagctgctgtactcttgctctggcatcgacaagatgtacattctggtgcgagacaagcgaggcaacgccgctcacgagcgactggcccgaatcaccaacactcccgccttcgaccgactgaagggctctcgaaacgaggacctgaagaagatcaccgtcatcaccggcgacattgctcgacccaacctgggactgcccgacgacgtgctgaagcgactcgaggacgaggtgtctatcgtgttccacctggccgccaccgtggccttcaacctgagactgaaggacgccatgggcatcaacatcaacggcaccgaagaggtggtgaagctgtgccaccgaatgaagaagctgcaggccttcgtccacgtgtctaccgccttctctaacgccgaccgacaggacatcgaagagaaggtgtaccccatgcctgtgactcttgaggacgcccgacacgtcgccgagatgtactctcacgacgacgaggtgatctctcagttcctgggaaagaagcccaacacctacaccttttctaaggccctggctgaggacctggtgatgaagcagtgtcaggacctgcctaccgccatcgtgcgaccctctatcgtgatgtcctctatcaaggaaccctgtcctggctggatcgacacctggtctggctctaccggcctgttcgtgggaatgcccgctggcgtcctgaaggtggtcaagggcaagggctctaacgtgaccgacctggtgcctgtggacatcgtgaccaactgcctgatcgtggtcgccaccgagtgccacaagaccaacgagatcaaggtgtacaactgctgcaccggcacttctaaccccatcacctctaactacgcctctgctatcgcccgacgagtgaccctgcgatactctctgaacgagctgccctggcctttcctggtgttcacctcttctgtgatcatctacaccctgatcaccttcatcctgcagactatccccgccttcctgatcgacatgtggtgcgtgctgaccggaaagaaggctacccagctgaagctccagtctcgactcaagaagaacatcgaggtcgtcaagttcttcctgatgaacgagtggaagttctctgacggcaacgtgcgaaacctgttcaagtctctgtctcccgaggacaaggaaaccttcaacttcgacgtgaagtgcatccactgggagaagtctatcgaggactacatcctgggagcccgaaagtacctgctgaagtacgagaagtag

SEQ ID NO: 123 - protein, 450 aa, Trichoplusia ni
MSSLKGPKVASFYTGKTVFITGGTGFLGKTLIEKLLYSCSGIDKMYILVRDKRGNAAHERLARITNTPAFDRLKGSRNEDLKKITVITGDIARPNLGLPDDVLKRLEDEVSIVFHLAATVAFNLRLKDAMGININGTEEVVKLCHRMKKLQAFVHVSTAFSNADRQDIEEKVYPMPVTLEDARHVAEMYSHDDEVISQFLGKKPNTYTFSKALAEDLVMKQCQDLPTAIVRPSIVMSSIKEPCPGWIDTWSGSTGLFVGMPAGVLKVVKGKGSNVTDLVPVDIVTNCLIVVATECHKTNEIKVYNCCTGTSNPITSNYASAIARRVTLRYSLNELPWPFLVFTSSVIIYTLITFILQTIPAFLIDMWCVLTGKKATQLKLQSRLKKNIEVVKFFLMNEWKFSDGNVRNLFKSLSPEDKETFNFDVKCIHWEKSIEDYILGARKYLLKYEK

SEQ ID NO: 124 - DNA, 1353 bp, Chrysodeixis includens
  source 1..1353: mol_type = other DNA
atgtcctcgctgaagggccccaaggtggcctcgttctacaccggcaagtctgtgttcattaccggcggaaccggcttcctgggcaagaccctgatcgagaagctgctgtactcttgctctggcatcgacaagctgtacatcctggtgcgagacaagcgaggctacgccgctcacgagcgactggcccgaatcaccaacactcccgccttcgaccgactgaagggcacccgaaacgaggacctgaagaagatcaccgtcatcaccggcgacattgctcgacccaacctgggactgcccgacgacatcctgaagcgactcgaggacgaggtgtctgtggtgttccacctggccgccaccgtggccttcaacctgagactgaaggacgccatgggcatcaacatcaacggcaccgaagaggtggtgaagctgtgccaccgaatgaagaagctgcaggccttcgtccacgtgtctaccgccttctctaactctgaccgattcgagatcgaagagaaggtgtaccccatgcctgtgactcttgaggacgcccgacacgtggccgagatgtactctcacgacgacgaggtgattacccagttcctgggaaagaagcccaacacctacaccttttctaaggccctggctgaggacctggtgatgaagcagtgtcaggacctgcctgccgccatcgtgcgaccctctatcgtgatgtcctctatcaaggaaccctgtcctggctggatcgacacctggtctggctctaccggcctgttcgtgggaatgcccgctggcgtgctgaaggtggtcaagggcaagggctctaacgtgaccgacctggtgcctgtggatatcgtgaccaacttcctgatcgtggtcgccaccgagtgccaaaagaccaacgagatcaaggtgtacaactgctgcaccggcacttctaaccccatcacctctaactacgcctctgctatcgcccgacgagtgaccctgcgatactctctgaacgagctgccctggcctttcctggtgttcacctcttctctgattgtgtacaccctgattaccttcatgctgcagactatccccgccttcctgattgacctgtggtgcgtgctgaccggcaagaaggctacccagctgaagctccagtctcgactcaagaagaacatcgaggtcgtcaagttcttcctgctgaacgagtggaagttctgcgacgagaacgtgcgaaacctgttcaagtctctgtctcccgaggacaaggaaaccttcaacttcgacgtgaagtctatcaactgggagaagtccatcgaggactacattctgggagcccgaaagtacctgctgaagtacgagaagtag

SEQ ID NO: 125 - protein, 450 aa, Chrysodeixis includens
MSSLKGPKVASFYTGKSVFITGGTGFLGKTLIEKLLYSCSGIDKLYILVRDKRGYAAHERLARITNTPAFDRLKGTRNEDLKKITVITGDIARPNLGLPDDILKRLEDEVSVVFHLAATVAFNLRLKDAMGININGTEEVVKLCHRMKKLQAFVHVSTAFSNSDRFEIEEKVYPMPVTLEDARHVAEMYSHDDEVITQFLGKKPNTYTFSKALAEDLVMKQCQDLPAAIVRPSIVMSSIKEPCPGWIDTWSGSTGLFVGMPAGVLKVVKGKGSNVTDLVPVDIVTNFLIVVATECQKTNEIKVYNCCTGTSNPITSNYASAIARRVTLRYSLNELPWPFLVFTSSLIVYTLITFMLQTIPAFLIDLWCVLTGKKATQLKLQSRLKKNIEVVKFFLLNEWKFCDENVRNLFKSLSPEDKETFNFDVKSINWEKSIEDYILGARKYLLKYEK

SEQ ID NO: 126 - DNA, 1386 bp, Chrysodeixis includens
  source 1..1386: mol_type = other DNA
atgttcgcctctaccgacggcggacgatctgacgaggaaacccctcagcagcacgagtctatcgccgacttctacgccggcaagtctgtgttcattaccggcgtgaccggcttcctcggcaaggcctacctcgagaagctgctgtactcttgcaaggacatcgacaaggtgttcgtgctgatccgaaacaagcgaggcgacgacgtgggcaagcgaattgagaagctcctggactcctctgtgttctctcgactgcgatctcaccgacctgaggacctgaagaagatcacccctgtctacggcgacattgacaagtctgagctgggcctgtctctggccgaccaagagcgactgatcaaggaagtgtctgtcgttttccacgtggccgcctctgtgaacctggacgccgacctcaagacctctctggtgaccaactacttcggcaccactcacgtgctgaagctgtgccaccgaatgaagaagctgaaggttttcgtctacgtgtctaccgcctactgcaacaccaccgtgaaggtgctggaagagaaggtgtaccctctgcctgtcgagctggacgaggtggtgaagatcatggaacagccccacctggactctaaccgagtcaagaagttgctgaacggacgacccaacacctacgctctgtctaaggccctggccgagcactacattgccgagaaccacggtgacatccccgtgattttcattcgaccctctatcgtgacctcttctgctcgagagcccgctcctggctgggccgactctttccagggcgccaccgctctgatcaccgcctgctggaagcgagtgaaccgagtgatctacggcaacggcgacaacatcatcgatctgatccccgtggactacgtgtccaacctgtctatcgtggccgctgccaaggtgaagtctaccaccgagatcgccgtgtacaactcttgcacctcttctatcaagcccatgactctgaagggaatcgccgagcagatcgagcgagtcaacaccgagaacaagcagggctctatcttcctgcctactattttcttcacctcctcttggctgttcgtgatcatgctgaccttcgtgctgcagctgctcccctcttttattgccgacctgttcctgtacatcaccggcaacaagcctatgtacatgaagatccagtctaaggtgctgcacggccgacacatcctgaactactacaccaacgactcttgggtgttcaagtctgactcttctcagcgactgcaggactctctgtctcccgccgacaagcgactgttcccctgctgtccctctgacatcaagtgggacgagtactttaacacctacttttgcggcatcgacaactacctgctcaagcgaaagtag

SEQ ID NO: 127 - protein, 461 aa, Chrysodeixis includens
MFASTDGGRSDEETPQQHESIADFYAGKSVFITGVTGFLGKAYLEKLLYSCKDIDKVFVLIRNKRGDDVGKRIEKLLDSSVFSRLRSHRPEDLKKITPVYGDIDKSELGLSLADQERLIKEVSVVFHVAASVNLDADLKTSLVTNYFGTTHVLKLCHRMKKLKVFVYVSTAYCNTTVKVLEEKVYPLPVELDEVVKIMEQPHLDSNRVKKLLNGRPNTYALSKALAEHYIAENHGDIPVIFIRPSIVTSSAREPAPGWADSFQGATALITACWKRVNRVIYGNGDNIIDLIPVDYVSNLSIVAAAKVKSTTEIAVYNSCTSSIKPMTLKGIAEQIERVNTENKQGSIFLPTIFFTSSWLFVIMLTFVLQLLPSFIADLFLYITGNKPMYMKIQSKVLHGRHILNYYTNDSWVFKSDSSQRLQDSLSPADKRLFPCCPSDIKWDEYFNTYFCGIDNYLLKRK

SEQ ID NO: 128 - DNA, 1569 bp, Cydia pomonella
  source 1..1569: mol_type = other DNA
atggtggctccccgagatcgacgaggcgctctgattccccagttcttcgccggacgatctgtgctgatcaccggcgccaccggcttcgtcggcaaggtgctggtcgagcgactgctgtctacctgtcctgacatctctaccctgcatctgctgctgcgagtgaagaagggcgctaccccttctgagcgactgaaggaactcaaggaatctcaggtgttcgacgtggtgcgacagcgaaacccctctcagctggacaagctgcgagttcttgagggtgacgttgctgctcctggtcttggtcttgctgctgacgctgctcttactcttcaggacgtgtctgtggtgttccactctgccgccactctgaagttcgacgaggaactgcgaaaggccgtggaccagaacgtgcgatctgtgatgcgactgctcgagctgtgcgacaagctccccaacatgcaggccttcatccacgtgtctaccgcctactctaacgccgagctgtcctctgtcgaggaacgagtgtaccctcctcctgctcctctggaagccgtgctggccctgaccgaccacgtgcctgatcctactctggccggcatcacccaccagtacattgctcccaagcctaacacctacaccttcaccaaggctctggccgagactgtggttgagcgacacggcaacactggctaccccatctctatcttccgacctaccatcgtgatctctgcccagaagcacccctttccaggctggatcgagaacttcaacggcccctctggcgtggtcgtggccaccggcaagggcctgctgcacgtgttccccaccaagtgcggcgtgcacaccgactttctgcccgtggacattgccgtggacaccctgatcgccgtggcctgggagactgccgtcgaccactctaaggaagtgcgagtctacaactgctctaccggcgctaaccccaccaccttcgaggacttcgagcgaaacctgcgacgagagactacccgacatcctctggacggcgccctgtggtatccctctggcaccgccgtgcagaccaagtgggctcgaaccctgctcgagttcctgctgcagaccgtgcctctgcacctggccgagtacaccgctcgaatgatcggcctcaagacccctgtgaacctgattaccgccaacaagcgactgaccgccatgaccgacgctctcgagttctttgctacccgagagttcaacttcgagtgtgagcgagtgcgagccctgcaccaccgactgtcgcctgaggacaagaagatctacaacctggacgtgatgtctatcaactgggacgaccactacgccgacttcgtgaagggcacccgaaagtacctgctgaacgagaaggaacaggacctcgagaaggcccgacgacacatgcacaagatgtggtttctgcacaaggtcctgaagctggtgaccctgctgctgttcctgcgaatcctggtgcagaaccgatacgcccgatctttcgtgtacggcaccctccgactgctgctctcgctgttcggcgccgtgtaccatcgactggtgcacgcctag

SEQ ID NO: 129 - protein, 522 aa, Cydia pomonella
MVAPRDRRGALIPQFFAGRSVLITGATGFVGKVLVERLLSTCPDISTLHLLLRVKKGATPSERLKELKESQVFDVVRQRNPSQLDKLRVLEGDVAAPGLGLAADAALTLQDVSVVFHSAATLKFDEELRKAVDQNVRSVMRLLELCDKLPNMQAFIHVSTAYSNAELSSVEERVYPPPAPLEAVLALTDHVPDPTLAGITHQYIAPKPNTYTFTKALAETVVERHGNTGYPISIFRPTIVISAQKHPFPGWIENFNGPSGVVVATGKGLLHVFPTKCGVHTDFLPVDIAVDTLIAVAWETAVDHSKEVRVYNCSTGANPTTFEDFERNLRRETTRHPLDGALWYPSGTAVQTKWARTLLEFLLQTVPLHLAEYTARMIGLKTPVNLITANKRLTAMTDALEFFATREFNFECERVRALHHRLSPEDKKIYNLDVMSINWDDHYADFVKGTRKYLLNEKEQDLEKARRHMHKMWFLHKVLKLVTLLLFLRILVQNRYARSFVYGTLRLLLSLFGAVYHRLVHA

SEQ ID NO: 130 - DNA, 1572 bp, Grapholita molesta
  source 1..1572: mol_type = other DNA
atggtggctccccgagagcgacgatctggcgctctgattccccagttcttcgccggacgatctgtgctgatcaccggcgccaccggcttcgtcggcaaggtgctggtcgagcgactgctgtctacctgtcctgacatctctaacctgcatctgctgctgcgagtgaagaagggcgtcacccctgccgagcgactcaagaccctgaaggaatctcaggtgttcgacgtggtgcgacagcgaaaccccgctcagctcgagaagctgcgagttcttgagggtgacgttgctgctcctggtcttggtcttgctgctgacgctgctcttcaccttcaggacgtgtctgtggtgttccactctgccgccactctgaagttcgacgaggaactgcgaaaggccgtggaccagaacgtgcgatctgtgatccgactgctcgagctgtgcgacaagctgcccaacatgcaggccttcatccacgtgtctaccgcctactctaacgccgagctgtcctctgtggccgagcgagtgtaccctcctcctgctcctgtggacgccgtgctggccctgaccgaccacgtgcctgatcctactctggccggcatcacccaccagtacattgctcccaagcctaacacctacaccttcaccaaggctctggccgagaacgtgatcctgcgacacggcaacaccggctacgccatctctatcttccgacctaccatcgtgatctctgccatgaagcacccctatcctggctggatcgagaacctgaacggcccctctggcgtgatcgtggccaccggcaagggcctgctgcacgtgttccccatcaagtgcggcgtgcacaccgactttctgcccgtggacattgccgtggacaccctgatcgccgtggcctgggagactgccatcgaccagtctaaggacgtgcgagtctacaactgctctaccggcgacaaccccaccaccttcgaggacttcgagactatgctgcgacgagaggtgactcgacaccccatggacggcgccctgtggtatccctctggacgagccgtgcagtctaagtgggcccgaaccgctctcgagttcctgctgcagaccgtgcctctccacctggccgagtacaccgctcgaatgatcggcgtcaagacccctgtgaacctgattaccgccaacaagcgactgtccgccatgaccgacgctctggaattcttcgctacccgagagttccacttcgagtctgagcgagtgcgagccctgcacgaccgactgtcgcctgaggaccgagacatctacaacctggacgtgatgaccatcaactgggacgagcactacgccgacttcgtgaagggcacccgaaagtacctgctgcaagagaaggaccaggacctcgagaaggcccgacgacacatgaacaagatgtggctgctgcacaacgtgttcaagttcgtgaccctgctgatcttcctgcgactgctcatgcagaaccgatacgtgcgagccttcgtgttcggcaccctgagactgctgctgtctctgttcaacaccgtgtacaaccgactggtgcacgcctag

SEQ ID NO: 131 - protein, 523 aa, Grapholita molesta
MVAPRERRSGALIPQFFAGRSVLITGATGFVGKVLVERLLSTCPDISNLHLLLRVKKGVTPAERLKTLKESQVFDVVRQRNPAQLEKLRVLEGDVAAPGLGLAADAALHLQDVSVVFHSAATLKFDEELRKAVDQNVRSVIRLLELCDKLPNMQAFIHVSTAYSNAELSSVAERVYPPPAPVDAVLALTDHVPDPTLAGITHQYIAPKPNTYTFTKALAENVILRHGNTGYAISIFRPTIVISAMKHPYPGWIENLNGPSGVIVATGKGLLHVFPIKCGVHTDFLPVDIAVDTLIAVAWETAIDQSKDVRVYNCSTGDNPTTFEDFETMLRREVTRHPMDGALWYPSGRAVQSKWARTALEFLLQTVPLHLAEYTARMIGVKTPVNLITANKRLSAMTDALEFFATREFHFESERVRALHDRLSPEDRDIYNLDVMTINWDEHYADFVKGTRKYLLQEKDQDLEKARRHMNKMWLLHNVFKFVTLLIFLRLLMQNRYVRAFVFGTLRLLLSLFNTVYNRLVHA

SEQ ID NO: 132 - protein, 326 aa, synthetic construct
MVPNKGSSDVLSEHSEPQFTKLIAPQAEPRKHKIVYRNLLTFGYWHLSAVYGLYLCFTSAKRATIIFAIFIYIIAKIGITGGAHRLWAHRAYKAKLPLEILLLIMNSIAFQDTVLTWARDHRLHHKYSDTDADPHNPTRGFFFAHMGWVLVRKHPEVLARGKALPIDDLKNNPLLRFQKKYAIPMIGTLCFLMPTFVPVYFWGESISTAWNINLLRYVTNLNVIFLINSWAHLIGNKPYDKNIAATQSIPISIATLGEGFHNYHHTYPWDYKASELGNNKINLTTKFIDFFAWVGWAYDLKSVPQEAIAKRCAKTGDGTDMWGRKR

SEQ ID NO: 133 - protein, 345 aa, synthetic construct
MVPYKGSSTVLTEDQPQFEKLIAPQAEPRKHKIVYRNLLTFGYWHLSAVYGLYLCFTSAKRATIIFAIFIYIIAKIGITGGAHRLWAHRAYKAKLPLEILLLIMNSIAFQDTVLTWARDHRLHHKYSDTDADPHNPTRGFFFAHMGWVLVRKHPEVLARGKALPIDDLKNNPLLRFQKKYAIPMIGTLCFLMPTFVPVYFWGESISTAWNINLLRYVTNLNVIFLINSWAHLIGNKPYDKNIAATQSIPISIATLGEGFHNYHHTYPWDYKASELGNNKINLTTKFIDFFAWVGWAYDLKSVSEELVVNRMRRTGDGSNLWGWGDKDMTEEDRNGALLTTKSVVE

SEQ ID NO: 134 - protein, 346 aa, synthetic construct
MCPKSPEQASVVEEREEKSAEFEKLIAPQAEPRKHKIVYRNLLTFGYWHLSAVYGLYLCFTSAKRATIIFAIFIYIIAKIGITGGAHRLWAHRAYKAKLPLEILLLIMNSIAFQDTVLTWARDHRLHHKYSDTDADPHNPTRGFFFAHMGWVLVRKHPEVLARGKALPIDDLKNNPLLRFQKKYAIPMIGTLCFLMPTFVPVYFWGESISTAWNINLLRYVTNLNVIFLINSWAHLIGNKPYDKNIAATQSIPISIATLGEGFHNYHHTYPWDYKASELGNNKINLTTKFIDFFAWVGWAYDLKTIPQDAIEKRMIRTGDGTDMWGFGEKRFRKEVDNSNETYSDF

SEQ ID NO: 135 - DNA, 1017 bp, synthetic construct
  source 1..1017: mol_type = other DNA
atggtgcccaacaagggttcttccgacgtcctgtctgagcactccgagccccagttcaccaagctgattgctccccaggctgagccccgaaagcacaagatcgtgtaccgaaacctgctgaccttcggatactggcacctgtctgccgtctacggtctgtacctgtgtttcacctctgccaagcgagctaccattatcttcgccatcttcatctacatcatcgctaagatcggcattaccggcggagcccaccgactgtgggctcaccgagcctacaaggccaagctgcccctggagatcctgctgctgattatgaactctatcgctttccaggacaccgtgctgacctgggctcgagatcaccgactgcaccacaagtactctgacaccgacgctgaccctcacaaccctacccgaggtttcttcttcgcccacatgggctgggtgctcgtccgaaagcaccccgaggtgctggcccggggaaaggctctgcccatcgacgacctgaagaacaaccccctgctgcgattccagaagaagtacgctatccccatgattggcaccctgtgtttcctgatgcccaccttcgtgcccgtctacttctggggtgagtctatttctaccgcctggaacatcaacctgctgcgatacgtgaccaacctgaacgtgatcttcctgatcaactcctgggctcacctgatcggcaacaagccctacgacaagaacattgccgccactcagtctatccccatctccatcgctaccctcggagagggtttccacaactaccaccacacctacccttgggactacaaggcttctgagctgggcaacaacaagatcaacctgaccaccaagttcattgacttcttcgcctgggtcggatgggcttacgacctgaagtccgtcccccaggaagccatcgctaagcgatgcgctaagaccggcgacggaaccgacatgtggggacgaaagcgatagggcgacggaaccgacatgtggggacgaaagcgatag

SEQ ID NO: 136 - DNA, 1038 bp, synthetic construct
  source 1..1038: mol_type = other DNA
atggtgccctacaagggatcttctaccgtgctgaccgaggaccagcctcagttcgagaagctgattgctccccaggctgagccccgaaagcacaagatcgtgtaccgaaacctgctgaccttcggatactggcacctgtctgccgtctacggtctgtacctgtgtttcacctctgccaagcgagctaccattatcttcgccatcttcatctacatcatcgctaagatcggcattaccggcggagcccaccgactgtgggctcaccgagcctacaaggccaagctgcccctggagatcctgctgctgattatgaactctatcgctttccaggacaccgtgctgacctgggctcgagatcaccgactgcaccacaagtactctgacaccgacgctgaccctcacaaccctacccgaggtttcttcttcgcccacatgggctgggtgctcgtccgaaagcaccccgaggtgctggcccggggaaaggctctgcccatcgacgacctgaagaacaaccccctgctgcgattccagaagaagtacgctatccccatgattggcaccctgtgtttcctgatgcccaccttcgtgcccgtctacttctggggtgagtctatttctaccgcctggaacatcaacctgctgcgatacgtgaccaacctgaacgtgatcttcctgatcaactcctgggctcacctgatcggcaacaagccctacgacaagaacattgccgccactcagtctatccccatctccatcgctaccctcggagagggtttccacaactaccaccacacctacccttgggactacaaggcttctgagctgggcaacaacaagatcaacctgaccaccaagttcattgacttcttcgcctgggtcggatgggcttacgacctgaagtctgtgtctgaggaactggtggtgaaccgaatgcgacgaaccggcgacggctctaacctctggggctggggcgacaaggacatgaccgaagaggaccgaaacggcgccctgctcaccaccaagtccgtggtcgagtag

SEQ ID NO: 137 - DNA, 1041 bp, synthetic construct
  source 1..1041: mol_type = other DNA
atgtgccccaagtctcccgagcaggcctctgtggtcgaggaacgagaggaaaagtctgccgagttcgagaagctgattgctccccaggctgagccccgaaagcacaagatcgtgtaccgaaacctgctgaccttcggatactggcacctgtctgccgtctacggtctgtacctgtgtttcacctctgccaagcgagctaccattatcttcgccatcttcatctacatcatcgctaagatcggcattaccggcggagcccaccgactgtgggctcaccgagcctacaaggccaagctgcccctggagatcctgctgctgattatgaactctatcgctttccaggacaccgtgctgacctgggctcgagatcaccgactgcaccacaagtactctgacaccgacgctgaccctcacaaccctacccgaggtttcttcttcgcccacatgggctgggtgctcgtccgaaagcaccccgaggtgctggcccggggaaaggctctgcccatcgacgacctgaagaacaaccccctgctgcgattccagaagaagtacgctatccccatgattggcaccctgtgtttcctgatgcccaccttcgtgcccgtctacttctggggtgagtctatttctaccgcctggaacatcaacctgctgcgatacgtgaccaacctgaacgtgatcttcctgatcaactcctgggctcacctgatcggcaacaagccctacgacaagaacattgccgccactcagtctatccccatctccatcgctaccctcggagagggtttccacaactaccaccacacctacccttgggactacaaggcttctgagctgggcaacaacaagatcaacctgaccaccaagttcattgacttcttcgcctgggtcggatgggcttacgacctgaagaccattcctcaggacgccatcgagaagcgaatgatccgaaccggcgacggcaccgacatgtggggcttcggtgagaagcgatttcgaaaggaagtggacaactctaacgagacttactctgacttttag

SEQ ID NO: 138 - DNA, 1038 bp, Chilo suppressalis
  source 1..1038: mol_type = other DNA
atggctcccaactctatccagaacgacgaaggcctgggcgtcgaggtgcagcccgagtctctgacccctcctaagctgacccagatccgacacaagatcctgtacgtgaacctgacctacttcctgtactggcacctggctgctccctacggcatctacctgtgcttcacctctgtgaagtgggagactatcatcttcggcttcgtggtgttcctggccgccgagctgggcgtgaccgctggcgcccacagactgtggtcccacaagtcttacaaggccaagcgacccctcgagatcatcctgatgctgttcaactctatggccttccagaactctatcatccactgggcccgagatcaccgactgcaccacaagtactgcgacaccgacgctgacccctacaacgcctctcgaggcttcttctactctcacgtcggctggctgctgaccaagaagaacaaggaagtcctgaagcgaggcaagaccatcgacgcctctgacctgttcaacaaccccgtgctgcagttccagaagaagtacgctatccccgttttcgccctgtgctgcttcattctgcccaccgctattcccatgtacgtgtggggcgagactctgaacaactcttggcacgccaacctgctgcgatacaccatcaacctgaacgtgaccttcctggtgaactctgccgctcacatctggggctaccgaccttacgacaagtctatctaccccgctcagaacgtgtacgtgtctctggtgaccctcggcgagggcttccacaactaccaccacgtgttcccttgggactacaagaccgctgagctgggaaacaacaagctgaacgtcactacctggttcatcaactttttcaccaagctcggctgggcctacgacctcaagaccgtgtctaacgagatcgtgcgagcccgagccatgcgaaccggcgacggcaaggacctgtggggctggaacgacaaggacatcaccgacatggacaaggaacacactgacatcctgtactctaagaccgaggactag

(19) United States
(12) Patent Application Publication
(10) Pub. No.: US 2024/0287555 A1
(43) Pub. Date: Aug. 29, 2024

(54) METHODS AND YEAST CELLS FOR PRODUCTION OF DESATURATED COMPOUNDS

(71) Applicant: FMC Agricultural Solutions A/S, Harboøre (DK)

(72) Inventors: Carina Holkenbrink, Harboøre (DK); Irina Borodina, Harboøre (DK); Baojian Ding, Lund (SE); Christer Löfstedt, Lund (SE)

(21) Appl. No.: 18/570,271

(22) PCT Filed: Jul. 1, 2022
(86) PCT No.: PCT/EP2022/068311

§ 371 Date: Dec. 14, 2023

(30) Foreign Application Priority Data
Jul. 2, 2021 (EP) 21183459.3
Mar. 9, 2022 (EP) 22161120.5

(51) Int. Cl. C12P 7/6409; C12N 9/02; C12P 7/6436

(52) U.S. Cl. CPC C12P 7/6409; C12N 9/0008; C12N 9/0071; C12P 7/6436; C12Y 102/01084; C12Y 114/19; C12Y 114/19005

(57) ABSTRACT

The present invention relates to yeast cells capable of producing Δ13 desaturated fatty acyl-CoAs and optionally desaturated fatty alcohols, said yeast cells expressing heterologous Δ13 desaturases. Said compounds are precursors of sex pheromone components of several insects, such as the navel orangeworm Amyelois transitella, the grape leaffolder Desmia funeralis, Cedar processionary moth Thaumetopoea bonjeani, the iron prominent Notodonta dromedarius, and others.

### TECHNICAL FIELD

The present invention relates to yeast cells capable of producing 413 desaturated fatty acyl-CoAs and optionally desaturated fatty alcohols, said yeast cells expressing heterologous 413 desaturases. Said compounds are precursors of sex pheromone components of several insects, such as the navel orangeworm Amyelois transitella, the grape leaffolder Desmia funeralis, Cedar processionary moth Thaumetopoea bonjeani, the iron prominent Notodonta dromedarius, and others.

### BACKGROUND

Integrated Pest Management (IPM) is expected to play a major role for both increasing the crop yield and for minimizing environmental impact and enabling organic food production. IPM employs alternative pest control methods, such as mating disruption using pheromones, mass trapping using pheromones, beneficial insects, etc.

Pheromones constitute a group of diverse chemicals that insects (like other organisms) use to communicate with individuals of the same species in various contexts, including mate attraction, alarm, trail marking and aggregation. Insect pheromones associated with long-range mate finding are already used in agriculture and forestry applications for monitoring and control of pests, as a safe and environmentally friendly alternative to pesticides. The biological production of pheromones for use pest control is advantageous over chemical synthesis in respect to price, specificity, and environmental impact.

Pheromones of interest are (Z11, Z13)-hexadecadien-1-ol, (Z11, Z13)-hexadecadienal and (Z11, Z13)-hexadecadien-1-ol acetate. These compounds are either alone or in a mixture sex pheromone components of a number of insects, such as the navel orangeworm Amyelois transitella, the grape leaffolder Desmia funeralis, Cedar processionary moth Thaumetopoea bonjeani, the iron prominent Notodonta dromedarius and others. These moths are pests of significant economic importance in storage and horticultural crops.

There remains a need for microbial cell factories which can produce Δ13 desaturated fatty alcohols and derivatives with high titers.

### SUMMARY

The invention is as defined in the claims.

The present disclosure provides yeast cells capable of producing 413 desaturated fatty acyl-CoAs and optionally 413 desaturated fatty alcohols, said yeast cells expressing a heterologous 413 desaturase. In particular, the present disclosure provides yeast cells capable of producing (Z11, Z13)-hexadecadienoic acid or (Z11, Z13)-hexadecadien-1-ol, the precursors of the pheromones (Z11, Z13)-hexadecadienal and (Z11, Z13)-hexadecadien-1-ol acetate. (Z11, Z13)-hexadecadien-1-ol, (Z11, Z13)-hexadecadienal and (Z11, Z13)-hexadecadien-1-olol acetate are sex pheromone components of several insects, such as the navel orangeworm Amyelois transitella, the grape leaffolder Desmia funeralis, Cedar processionary moth Thaumetopoea bonjeani, the iron prominent Notodonta dromedarius, and others. (Z11, Z13)-hexadecadien-1-ol acetate is also known and referred to as (Z11, Z13)-hexadecadien-1-yl acetate.

Thus, provided herein is a yeast cell capable of producing a desaturated fatty acyl-coenzyme A (fatty acyl-CoA) having n′ double bond(s), wherein at least one of said double bond(s) is at position 13, wherein said yeast cell expresses a heterologous Δ13 fatty acyl-CoA desaturase, said desaturase being capable of introducing a double bond at position 13 in a saturated or desaturated fatty acyl-CoA, preferably a desaturated fatty acyl-CoA, having a carbon chain length of at least 14 and having n double bond(s), wherein n and n′ are integers, wherein 0≤n≤3 and wherein 1≤n′≤4.

Also provided herein is a method for producing a desaturated fatty acyl-CoA having n′ double bond(s), wherein at least one of said double bond(s) is at position 13, in a yeast cell, said method comprising the steps of:
i) providing a yeast cell as disclosed herein,
ii) incubating said yeast cell in a medium under conditions allowing expression of a heterologous Δ13 desaturase, said desaturase being capable of introducing a double bond at position 13 in a saturated or desaturated fatty acyl-CoA, preferably a desaturated fatty-acyl-CoA, having a carbon chain length of at least 14 and having n double bonds,
wherein n and n′ are integers,
wherein 0≤n≤3 and wherein 1≤n′≤4,
thereby producing a desaturated fatty acyl-CoA having n′ double bond(s), wherein at least one of said double bond(s) is at position 13.

Further provided herein is a nucleic acid or a system of nucleic acids for modifying a yeast cell, said nucleic acid or system of nucleic acids comprising at least one polynucleotide encoding a heterologous Δ13 desaturase said desaturase being capable of introducing a double bond at position 13 in a saturated or desaturated fatty acyl-CoA having a carbon chain length of at least 14 and having n double bonds thereby producing a desaturated fatty acyl-CoA having n′ double bond(s), wherein at least one of said double bond(s) is at position 13,
wherein n and n′ are integers,
wherein 0≤n≤3 and wherein 1≤n′≤4.

Thus, provided herein is a yeast cell capable of producing a desaturated fatty acyl-coenzyme A (fatty acyl-CoA) having n′ double bond(s), wherein at least one of said double bond(s) is at position 13,
wherein said yeast cell expresses a heterologous Δ13 fatty acyl-CoA desaturase, said desaturase being capable of introducing a double bond at position 13 in a saturated fatty acyl-CoA or a desaturated fatty acyl-CoA having a carbon chain consisting solely of single and double bonds, preferably a desaturated fatty acyl-CoA, wherein said fatty acyl-CoA has a carbon chain length of at least 14 and has n double bond(s),
wherein n and n′ are integers,
wherein 0≤n≤3 and wherein 1≤n′≤4.

Also provided herein is a method for producing a desaturated fatty acyl-CoA having n′ double bond(s), wherein at least one of said double bond(s) is at position 13, in a yeast cell, said method comprising the steps of:
i) providing a yeast cell as disclosed herein,
ii) incubating said yeast cell in a medium under conditions allowing expression of a heterologous Δ13 desaturase, said desaturase being capable of introducing a double bond at position 13 in a saturated fatty-acyl-CoA or a desaturated fatty acyl-CoA having a carbon chain consisting solely of single and double bonds, preferably a desaturated fatty-acyl-CoA, wherein said fatty acyl-CoA has a carbon chain length of at least 14 and has n double bonds,
wherein n and n′ are integers,
wherein 0≤n≤3 and wherein 1≤n′≤4,
thereby producing a desaturated fatty acyl-CoA having n′ double bond(s), wherein at least one of said double bond(s) is at position 13.

Further provided herein is a nucleic acid or a system of nucleic acids for modifying a yeast cell, said nucleic acid or system of nucleic acids comprising at least one polynucleotide encoding a heterologous Δ13 desaturase said desaturase being capable of introducing a double bond at position 13 in a saturated fatty acyl-CoA or a desaturated fatty acyl-CoA having a carbon chain consisting solely of single and double bonds, preferably a desaturated fatty-acyl-CoA, wherein said fatty acyl-CoA has a carbon chain length of at least 14 and has n double bonds, thereby producing a desaturated fatty acyl-CoA having n′ double bond(s), wherein at least one of said double bond(s) is at position 13,
wherein n and n′ are integers,
wherein 0≤n≤3 and wherein 1≤n′≤4.

Also provided herein is a method of monitoring the presence of pest or disrupting the mating of pest, said method comprising the steps of:
i) producing (Z11, Z13)-hexadecadien-1-ol and/or a Z13-fatty alcohol by the method according to any one of items 55 to 64;
ii) converting the Z13-fatty alcohol and/or (Z11, Z13)-hexadecadien-1-ol into a Z13-fatty alcohol acetate and/or (Z11, Z13)-hexadecadien-1-ol acetate, respectively;
iii) converting the Z13-fatty alcohol and/or (Z11, Z13)hexadecadien-1-ol into a Z13-fatty aldehyde and/or (Z11, Z13)-hexadecadienal, respectively;
iv) recovering said Z13-fatty alcohol, (Z11, Z13)-hexadecadien-1-ol, Z13-fatty alcohol acetate, (Z11, Z13)-hexadecadien-1-ol acetate, Z13-fatty aldehyde, and/or (Z11, Z13)-hexadecadienal;
v) formulating said Z13-fatty alcohol, (Z11, Z13)-hexadecadien-1-ol, Z13-fatty alcohol acetate, (Z11, Z13)-hexadecadien-1-ol acetate, Z13-fatty aldehyde and/or (Z11, Z13)-hexadecadienal as a pheromone composition; and employing said pheromone composition as an integrated pest management composition.

Further provided herein is a pheromone composition obtainable by a method comprising the following steps:
i) producing (Z11, Z13)-hexadecadien-1-ol and/or a Z13-fatty alcohol by the method as disclosed herein;
ii) converting the Z13-fatty alcohol and/or (Z11, Z13)-hexadecadien-1-ol into a Z13-fatty alcohol acetate and/or (Z11, Z13)-hexadecadien-1-ol acetate, respectively;
iii) converting the Z13-fatty alcohol and/or (Z11, Z13)-hexadecadien-1-ol into a Z13-fatty aldehyde and/or (Z11, Z13)-hexadecadienal, respectively;
iv) recovering said Z13-fatty alcohol, (Z11, Z13)-hexadecadien-1-ol, Z13-fatty alcohol acetate, (Z11, Z13)-hexadecadien-1-ol acetate, Z13-fatty aldehyde, and/or (Z11, Z13)-hexadecadienal;
v) formulating said Z13-fatty alcohol, (Z11, Z13)-hexadecadien-1-ol, Z13-fatty alcohol acetate, (Z11, Z13)-hexadecadien-1-ol acetate, Z13-fatty aldehyde, and/or (Z11, Z13)-hexadecadienal as a pheromone composition.

Further provided herein is a pheromone composition comprising a Z13-fatty alcohol, (Z11, Z13)-hexadecadien-1-ol, a Z13-fatty alcohol acetate, (Z11, Z13)-hexadecadien-1-ol acetate, a Z13-fatty aldehyde and/or (Z11, Z13)-hexadecadienal, wherein said pheromone composition comprises at least 20% biobased carbon, such as at least 30% biobased carbon, such as at least 40% biobased carbon, such as at least 50% biobased carbon, such as at least 60% biobased carbon, such as at least 70% biobased carbon, such as at least 75% biobased carbon, such as at least 80% biobased carbon, such as at least 85% biobased carbon, such as at least 90% biobased carbon, such as at least 95% biobased carbon, such as 100% biobased carbon.

Also provided herein is a pheromone compound selected from the group consisting of a Z13-fatty alcohol, (Z11, Z13)-hexadecadien-1-ol, a Z13-fatty alcohol acetate, (Z11, Z13)-hexadecadien-1-ol acetate, a Z13-fatty aldehyde and/or (Z11, Z13)-hexadecadienal, wherein said pheromone compound comprises at least 20% biobased carbon, such as at least 30% biobased carbon, such as at least 40% biobased carbon, such as at least 50% biobased carbon, such as at least 60% biobased carbon, such as at least 70% biobased carbon, such as at least 75% biobased carbon, such as at least 80% biobased carbon, such as at least 85% biobased carbon, such as at least 90% biobased carbon, such as at least 95% biobased carbon, such as 100% biobased carbon.

Further provided herein is a pheromone composition comprising a Z13-fatty alcohol, (Z11, Z13)-hexadecadien-1-ol, a Z13-fatty alcohol acetate, (Z11, Z13)-hexadecadien-1-ol acetate, a Z13-fatty aldehyde and/or (Z11, Z13)-hexadecadienal, wherein said pheromone composition has a radioactive ¹⁴C level of at least 20%, such as at least 30% biobased carbon, such as at least 40% biobased carbon, such as at least 50% biobased carbon, such as at least 60% biobased carbon, such as at least 70% biobased carbon, such as at least 75% biobased carbon, such as at least 80% biobased carbon, such as at least 85% biobased carbon, such as at least 90% biobased carbon, such as at least 95% biobased carbon, such as 100% biobased carbon.

Also provided herein is a pheromone compound selected from the group consisting of a Z13-fatty alcohol, (Z11, Z13)-hexadecadien-1-ol, a Z13-fatty alcohol acetate, (Z11, Z13)-hexadecadien-1-ol acetate, a Z13-fatty aldehyde and/or (Z11, Z13)-hexadecadienal, wherein said pheromone compound has a radioactive ¹⁴C level of at least 20%, such as at least 30% biobased carbon, such as at least 40% biobased carbon, such as at least 50% biobased carbon, such as at least 60% biobased carbon, such as at least 70% biobased carbon, such as at least 75% biobased carbon, such as at least 80% biobased carbon, such as at least 85% biobased carbon, such as at least 90% biobased carbon, such as at least 95% biobased carbon, such as 100% biobased carbon.

Also provided herein is the use of an Amyelois transitella desaturase in a method for introducing a double bond at position 13 in a saturated or desaturated fatty acyl-CoA, optionally wherein the Amyelois transitella desaturase is AtrAATQ (SEQ ID NO: 1) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

Provided herein is also the use of an Notodonta dromedarius desaturase in a method for introducing a double bond at position 13 in a saturated or desaturated fatty acyl-CoA, optionally wherein the Notodonta dromedarius desaturase is NdDes1 (SEQ ID NO: 56) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

### DESCRIPTION OF DRAWINGS

FIG. 1. Pathway for production of (Z11, Z13)-hexadecadien-1-ol (Z11,Z13-16:OH) in yeast. Hexadecanoyl-CoA (16:CoA) is converted to (Z11)-hexadecenoyl-CoA (Z11-16:CoA) by the action of a Δ11 desaturase. A Δ13 desaturase in turn converts Z11-16:CoA to (Z11, Z13)-hexadecadienoyl-CoA (Z11,Z13-16:CoA), which is then reduced to (Z11,Z13)-hexadecadien-1-ol (Z11,Z13-16:OH) by the action of a fatty acyl-CoA reductase. Z11,Z13-16:OH can be optionally chemically or enzymatically oxidized into Z11,Z13-hexadecadienal (Z11,Z13-16:Ald) or chemically or enzymatically acetylated into (Z11, Z13)-hexadecadien-1-ol acetate (Z11,Z13-16:OAc).

FIG. 2. Representative GC chromatograms of (A) extracts of control strain ST10615, (B) extracts of strain ST10750 expressing AtrAATQ, and (C) Z11,Z13-16:Me analytical standard. Peak at retention time (RT) 10.95 min corresponds to methyl palmitate (16:Me), RT=11.2 min to methyl cis-9-hexadecenoate (Z9-16:Me), RT=11.3 min to methyl cis-11-hexadecenoate (Z11-16:Me) and RT=13.15 min to methyl (Z,Z)-11,13-hexadecadienoate (Z11,Z13-16:Me). The mass spectra of the (D) extract of ST10750 and (E) Z11,Z13-16:Me analytical standard at retention time 13.15 min. Arbitrary units.

FIG. 3. Representative GC chromatograms (A) extracts of strains ST11304 to ST11306 and Z11,Z13-16:OH analytical standard. RT=10.6 min corresponds to hexadecanol (16:OH), RT=11.05 min to (Z9)-hexadecenol (Z9-16:OH), RT=11.25 min to (Z11)-hexadecenol (Z11-16:OH), RT=13.7 to octadecanol (18:OH) and RT=13.75 min to (Z11,Z13)-hexadecadien-1-ol (Z11,Z13-16:OH) (B) mass spectra of extract of ST11306 and (C) Z11,Z13-16:OH analytical standard at retention time 13.75 min.

FIG. 4. Representative GC chromatograms (A) extracts of strains ST12613 (control) and ST12611 (expressing NdDes1) and Z11,Z13-16:Me analytical standard, and mass spectra of (B) extract of ST12611 and (C) Z11,Z13-16:Me analytical standard at retention time 13.4 min.

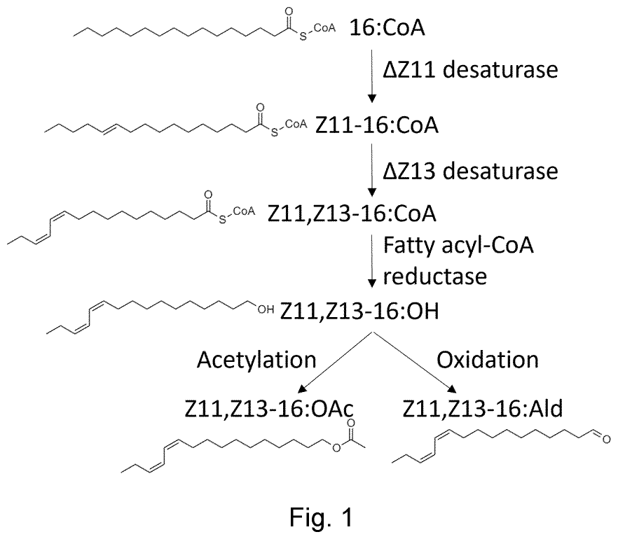

### DETAILED DESCRIPTION

### Definitions

Heterologous Δ13 desaturase: the term “heterologous Δ13 desaturase” as used herein refers to a desaturase, which is different from “further heterologous desaturase”. The heterologous desaturase is capable of introducing a double bond at position 13 in a saturated or desaturated fatty acyl-coenzyme A (fatty acyl-CoA).

A further heterologous desaturase: the term “a further heterologous desaturase” as used in herein refers to a desaturase which is different from “a heterologous desaturase”. The further heterologous desaturase is capable of introducing a double bond at any other position than position 13 in a saturated or desaturated fatty acyl-CoA. The further heterologous desaturase may be native to the same organism as the heterologous Δ13 desaturase, or it may be native to a different organism than the heterologous Δ13 desaturase, and catalyses a different reaction than the heterologous Δ13 desaturase.

Biopesticide: the term “biopesticide” is a contraction of ‘biological pesticide’ and refers to several types of pest management intervention: through predatory, parasitic, or chemical relationships. In the EU, biopesticides have been defined as “a form of pesticide based on micro-organisms or natural products”. In the US, they are defined by the EPA as “including naturally occurring substances that control pests (biochemical pesticides), microorganisms that control pests (microbial pesticides), and pesticidal substances produced by plants containing added genetic material (plant-incorporated protectants) or PIPs”. The present invention relates more particularly to biopesticides comprising natural products or naturally occurring substances. In the present context, these are manufactured by cultivating and concentrating naturally occurring organisms and/or their metabolites including bacteria and other microbes, fungi, nematodes, proteins, etc. These compounds are considered to be important components of integrated pest management (IPM) programmes, and have received much practical attention as substitutes to synthetic chemical plant protection products (PPPs). The Manual of Biocontrol Agents (2009: formerly the Biopesticide Manual) gives a review of the available biological insecticide (and other biology-based control) products.

Desaturated: the term “desaturated” will be herein used interchangeably with the term “unsaturated” and refers to a compound containing one or more double or triple carbon-carbon bonds, preferably a double carbon-carbon bond. The following nomenclature is used herein throughout: a Δi desaturated compound, where i is an integer, refers to a compound having a double or triple carbon-carbon bond at position i of the carbon chain. The carbon chain length is thus at least equal to i+1. For example, a 413 desaturated compound refers to a compound having a double or triple carbon-carbon bond between carbon 13 and carbon 14, and is herein referred to as a carbon chain having a carbon-carbon bond at position 13 and having a carbon chain length of 14 or more. The double or triple bond can be in an E configuration or in a Z configuration. Thus, herein, an Ei or a Zi desaturated compound will refer to a compound having a double carbon-carbon bond in an E configuration or in a Z configuration, respectively, at position i of the carbon chain, i.e. between position i and position i+1 of the carbon chain, wherein said desaturated compound has a total length at least equal to i+1. For example, a Z13 desaturated fatty alcohol has a desaturation at position 13 in a Z configuration (i.e. a double bond between carbon atom 13 and carbon atom 14), and has a carbon chain length of 14 or more.

Derived from: the term “derived from” when referring to a polypeptide or a polynucleotide derived from an organism means that said polypeptide or polynucleotide is native to said organism, i.e. that it is naturally found in said organism.

Z13-fatty acyl-CoA having at least one double bond at position 13: the term “Z13-fatty acyl-CoA having at least one double bond at position 13” as used herein, means that the Z13-fatty acyl-CoA has one double bond at position 13. In some embodiments of the invention, the Z13-fatty acyl-CoA only has the double bond at position 13. In other embodiments of the invention, the Z13-fatty acyl-CoA has additional double bonds beside the double bond at position 13. The additional double bonds can be at any position other than position 13. One example hereof is a Z13-fatty acyl-CoA having a double bond at position 13 and an additional double bond at any other position, such as at position 11.

Fatty acid: the term “fatty acid” refers herein to a carboxylic acid having a long aliphatic chain, i.e. an aliphatic chain between 14 and 30 carbon atoms, such as 14, 15, 16, 17, 18, 19, 20, 21, 22, 23, 24, 25, 26, 27, 28, 29 or 30 carbon atoms. Most naturally occurring fatty acids are unbranched. They can be saturated, or desaturated.

Fatty acyl-CoA: the term will herein be used interchangeably with “fatty acyl-CoA ester”, and refers to compounds of general formula R—CO—SCOA, where R is a fatty carbon chain having a carbon chain length of 14 to 30 carbon atoms, such as 14, 15, 16, 17, 18, 19, 20, 21, 22, 23, 24, 25, 26, 27, 28, 29 or 30 carbon atoms. The fatty carbon chain is joined to the —SH group of CoA by a thioester bond. Fatty acyl-CoAs can be saturated or desaturated, depending on whether the fatty acid which it is derived from is saturated or desaturated.

Fatty alcohol: the term “fatty alcohol” refers herein to an alcohol derived from a fatty acyl-CoA, having a carbon chain length of 14 to 30 carbon atoms, such as 14, 15, 16, 17, 18, 19, 20, 21, 22, 23, 24, 25, 26, 27, 28, 29 or 30 carbon atoms. Fatty alcohols can be saturated or desaturated.

Fatty alcohol acetate: the term refers to an acetate having a fatty carbon chain, i.e. an aliphatic chain between 14 and 30 carbon atoms, such as 14, 15, 16, 17, 18, 19, 20, 21, 22, 23, 24, 25, 26, 27, 28, 29 or 30 carbon atoms. Fatty acyl acetates can be saturated or desaturated.

Fatty aldehyde: the term refers herein to an aldehyde derived from a fatty acyl-CoA, having a carbon chain length of 14 to 30 carbon atoms, such as 14, 15, 16, 17, 18, 19, 20, 21, 22, 23, 24, 25, 26, 27, 28, 29 or 30 carbon atoms. Fatty aldehydes can be saturated or desaturated.

Functional variant: the term refers herein to functional variants of an enzyme, which retain at least some of the activity of the parent enzyme. Thus, a functional variant of an acyl-CoA oxidase, a desaturase, an alcohol-forming fatty acyl-CoA reductase, an alcohol dehydrogenase, an aldehyde-forming fatty acyl-CoA reductase, an acetyltransferase, or an NAD(P)H cytochrome b5 oxidoreductase (Ncb5or) can catalyse the same conversion as the acyl-CoA oxidase, the desaturase, the alcohol-forming fatty acyl-CoA reductase, the alcohol dehydrogenase, the aldehyde-forming fatty acyl-CoA reductase, or the acetyltransferase, respectively, from which they are derived, although the efficiency of reaction may be different, e.g. the efficiency is decreased or increased compared to the parent enzyme or the substrate specificity is modified.

Heterologous: the term “heterologous” when referring to a polypeptide, such as a protein or an enzyme, or to a polynucleotide, shall herein be construed to refer to a polypeptide or a polynucleotide which is not naturally present in a wild type cell. For example, the term “heterologous Δ11 desaturase” when applied to Saccharomyces cerevisiae refers to a Δ11 desaturase which is not naturally present in a wild type S. cerevisiae cell, e.g. a Δ11 desaturase derived from Spodoptera litura.

Identity/similarity: the terms identity and similarity, with respect to a polynucleotide (or polypeptide), are defined herein as the percentage of nucleic acids (or amino acids) in the candidate sequence that are identical, homologous or similar, respectively, to the residues of a corresponding native nucleic acids (or amino acids), after aligning the sequences and introducing gaps, if necessary, to achieve the maximum percent identity/similarity, and considering any conservative substitutions according to the NCIUB rules (https://iubmb.qmul.ac.uk/misc/naseq.html; NC-IUB, Eur J Biochem (1985)) as part of the sequence identity. In particular, the percentage of similarity refers to the percentage of residues conserved with similar physiochemical properties. Neither 5′ or 3′ extensions nor insertions (for nucleic acids) or N′ or C′ extensions nor insertions (for polypeptides) result in a reduction of identity, similarity or homology. Methods and computer programs for the alignments are well known in the art. Generally, a given similarity between two sequences implies that the identity between these sequences is at least equal to the similarity; for example, if two sequences are 70% similar to one another, they cannot be less than 70% identical to one another—but could be sharing 80% identity.

Increased activity: the term “increased activity” herein refers to an increase in activity of a given peptide, such as a protein or an enzyme. The increase in activity can be measured using methods known in the art, such as for example using enzyme assays to measure the increase in activity of an enzyme. In some cases, the increase in activity results in higher production of the compound or compounds which the enzyme is generating, i.e. the product. Thus, increased activity of an enzyme may be measured by measuring the amount, such as the concentration, of said product. If an enzyme has increased activity, the concentration of product will be higher compared the concentration of product generated in similar or identical conditions by the same enzyme which does not have increased activity, e.g. the parent enzyme or unmodified enzyme. If the enzyme with increased activity is expressed inside a cell, the product can be measured as the product titer, i.e. the amount of product said cell has produced, and can be compared to the titer or amount of the same product obtained in similar or identical conditions from a cell expressing the parent or unmodified enzyme but otherwise having an identical or similar genotype as the cell expressing the enzyme with increased activity.

n double bonds: the term “n double bonds” used herein refer to the number of double bonds present in a fatty acyl-CoA. A fatty acyl-CoA having n double bonds has 0-3 double bond(s). Thus, a fatty acyl-CoA having n double bonds can be a saturated fatty acyl-CoA having 0 double bonds or a desaturated fatty acyl-CoA having one or more double bonds, such as 1, 2 or 3 double bonds. The n double bonds may be at any position other than position 13.

n′ double bonds: the term “n′ double bonds” as used herein refers to the number of double bonds present in a fatty acyl-CoA. A fatty acyl-CoA having n′ double bonds has 1-4 double bond(s). Thus a fatty acyl-CoA having n′ double bonds can be a desaturated fatty acyl-CoA having one or more double bonds, such as 1, 2, 3 or 4 double bonds, whereof one of the double bonds is at position 13.

Native: the term “native” when referring to a polypeptide, such as a protein or an enzyme, or to a polynucleotide, shall herein be construed to refer to a polypeptide or a polynucleotide which is naturally present in a wild type cell.

Pest: as used herein, the term ‘pest’ shall refer to an organism, in particular an animal such as an insect, detrimental to humans or human concerns, in particular in the context of agriculture or livestock production. A pest is any living organism which is invasive or prolific, detrimental, troublesome, noxious, destructive, a nuisance to either plants or animals, human or human concerns, livestock, human structures, wild ecosystems etc. The term often overlaps with the related terms vermin, weed, plant and animal parasites and pathogens. It is possible for an organism to be a pest in one setting but beneficial, domesticated or acceptable in another.

Pheromone: pheromones are naturally occurring compounds. Lepidopteran pheromones are designated by an unbranched aliphatic chain (between 9 and 18 carbons, such as 9, 10, 11, 12, 13, 14, 15, 16, 17 or 18 carbon atoms) ending in an alcohol, aldehyde or acetate functional group and containing up to 3 double bonds in the aliphatic backbone, such as 1, 2 or 3 double bonds. Thus, desaturated fatty alcohols, desaturated fatty aldehydes and desaturated fatty alcohol acetates are typically comprised in pheromones. Pheromone compositions may be produced chemically or biochemically, for example as described herein. Pheromones thus comprise desaturated fatty alcohols, desaturated fatty aldehydes and/or desaturated fatty alcohol acetates, such as can be obtained by the methods and cells described herein.

Reduced activity: the term “reduced activity” may herein refer to a total or a partial loss of activity of a given peptide, such as a protein or an enzyme. In some cases, peptides are encoded by essential genes, which cannot be deleted. In these cases, activity of the peptide can be reduced by methods known in the art, such as down-regulation of transcription or translation, inhibition of the peptide. In other cases, the peptide is encoded by a non-essential gene, and the activity may be reduced or it may be completely lost, e.g. as a consequence of a deletion of the gene encoding the peptide. In order to reduce, whether partially or totally, the activity of a given peptide, methods known in the art include not only mutations in the genes encoding said peptide, but also mutation of genes encoding regulatory factors involved in transcription or translation of the gene encoding said peptide, e.g. mutation of transcription factor genes or of transcription repressor genes resulting in increased or decreased expression of said transcription factors or repressors, which in turn reduce transcription levels from the gene encoding the peptide; truncation or mutation of the native promoter of the gene, for example to remove transcription factor binding sites or to render them inaccessible to said transcription factors; replacement of the native promoter with a weaker promoter, leading to reduced transcription of the coding sequence encoding the peptide; truncation or mutation of the native terminator of the gene, or replacement of the native terminator of the gene with another terminator sequence; mutation of the Kozak sequence. Other methods involve regulation at the RNA level, and include RNA interference systems such as Dicer or Argonaute, RNA silencing methods, introduction of CRISPR/Cas systems resulting in targeted RNA degradation. Regulation at the protein level is also envisaged, e.g. by using inhibitors or protein degradation sequences. The listed methods may be inducible, i.e. they may be activated in a transient manner as known in the art.

Saturated: the term “saturated” refers to a compound which is devoid of double or triple carbon-carbon bonds.

Specificity: the specificity of an enzyme towards a given substrate is the preference exhibited by this enzyme to catalyse a reaction starting from said substrate. In the present disclosure, a desaturase and/or a fatty acyl-CoA reductase having a higher specificity towards hexadecanoyl-CoA (palmitoyl-CoA) than towards tetradecanoyl-CoA (myristoyl-CoA) preferably catalyse a reaction with hexadecanoyl-CoA than with hexadecanoyl-CoA as a substrate. Methods to determine the specificity of a desaturase or a fatty acyl-CoA reductase are known in the art. For example, specificity of a given desaturase in a given cell expressing it can be determined by incubating said cell in a solution comprising methyl palmitate for up to 48 hours, followed by extraction and esterification of the products with methanol. The profiles of the resulting fatty acid methyl esters can then be determined by GC-MS. Desaturases with higher specificity towards palmitoyl-CoA and low specificity towards myristoyl-CoA, for example, will result in higher concentration of (Z)11-C16:Me than (Z)11-C14:Me. For example, specificity of a given reductase in a given cell can be determined by incubating cells that express said reductase in a solution comprising methyl ester of (Z)11-palmitate for up to 48 hours, followed by extraction and analysis of the resulting fatty alcohols by GC-MS. Reductases with higher specificity towards (Z)11-C16:CoA and low specificity towards (Z)11-C14:CoA will result in higher concentration of (Z)11-C16:OH than (Z)11-C14:OH. Reductases can also deploy a specificity towards double-unsaturated fatty acyl-CoA and low specificity towards mono-unsaturated fatty acyl-CoA. This will result in a higher ratio of Z11,Z13-C16:OH to (Z)11-C16:OH.

Titer: the titer of a compound refers herein to the produced concentration of a compound. When the compound is produced by a cell, the term refers to the total concentration produced by the cell, i.e. the total amount of the compound divided by the volume of the culture medium. This means that, particularly for volatile compounds, the titer includes the portion of the compound which may have evaporated from the culture medium, and it is thus determined by collecting the produced compound from the fermentation broth and from potential off-gas from the fermenter.

### Production of Z13 Desaturated Compounds

The present inventors have discovered that expression of heterologous Δ13 desaturases in yeast cells results in the production of fatty acyl-CoA having a double bond at position 13. The heterologous Δ13 desaturases disclosed herein are capable of introducing a double bond at position 13, and optionally a double bond at a position other than position 13, in desaturated or saturated fatty acyl-CoAs having a carbon chain length of at least 14. The starting compound in which the desaturase may introduce the double bond at position 13 may be a saturated compound, or a desaturated compound already comprising a double bond at a position which is not position 13. Preferably, the starting compound is a desaturated fatty acyl-CoA comprising a double bond at position 11. The Δ13 desaturase may be able to introduce additional double bonds besides the double bond at position 13.

Thus, provided herein is a yeast cell capable of producing a desaturated fatty acyl-CoA having n′ double bond(s), wherein at least one of said double bond(s) is at position 13, wherein said yeast cell expresses a Δ13 heterologous fatty acyl-CoA desaturase, said desaturase being capable of introducing a double bond at position 13 in a saturated or desaturated fatty acyl-CoA having a carbon chain length of at least 14 and having n double bond(s),
wherein n and n′ are integers,
wherein 0≤n≤3 and wherein 1≤n′≤4.

Also provided herein is a yeast cell expressing a heterologous Δ13 desaturase, said desaturase being capable of introducing a double bond at position 13 in a saturated or desaturated fatty acyl-CoA having a carbon chain length of at least 14 and having n double bond(s) thereby producing a desaturated fatty acyl-CoA having n′ double bond(s), wherein at least one of said double bond(s) is at position 13,
wherein n and n′ are integers,
wherein 0≤n≤3 and wherein 1≤n′≤4.

In preferred embodiments, the saturated or desaturated fatty acyl-CoA having a carbon chain length of at least 14 and having n double bond(s) is a desaturated fatty acyl-CoA having a carbon chain length of 16, such as (Z11)-hexadecenoyl-CoA.

Further provided herein is a yeast cell capable of producing (Z11, Z13)-hexadecadienoyl-CoA, said yeast cell expressing
i) at least one further heterologous desaturase capable of introducing a first double bond at position 11 in hexadecanoyl-CoA, thereby converting said hexadecanoyl-CoA to (Z11)-hexadecenoyl-CoA; and
ii) at least one heterologous Δ13 desaturase capable of introducing a second double bond at position 13 in (Z11)-hexadecenoyl-CoA, thereby converting (Z11)-hexadecenoyl-CoA into (Z11, Z13)-hexadecadienoyl-CoA.

Also provided herein is a yeast cell capable of producing a desaturated fatty acyl-CoA, said yeast cell expressing AtrAATQ (SEQ ID NO: 1) or NdDes1 (SEQ ID NO: 56) capable of introducing a cis-double bond at position 13 in a fatty acyl-CoA, thereby converting the fatty acyl-CoA into a desaturated Z13-fatty acyl-CoA.

### Desaturase

In the present invention, the terms ‘fatty acyl-CoA desaturase’, ‘desaturase’, ‘fatty acyl desaturase’ and ‘FAD’ will be used interchangeably. The term generally refers to an enzyme capable of introducing at least one double bond in E/Z confirmations in an acyl-CoA having a chain length of 8, 9, 10, 11, 12, 13, 14, 15, 16, 17, 18, 19, 20, 21 or 22 carbon atoms. The double bond may be introduced in any position. For example, a desaturase introducing a double bond in position 13 is termed Δ13 desaturase. In some embodiments, the desaturases disclosed herein are capable of introducing double bonds at several positions of a fatty acyl-CoA, such as for example at position 13 and at position 11.

### Desaturases Catalyse the Reaction:

Fatty acyl-CoA+2 ferrocytochrome b5+O(2)+2H(+)<=>desaturated fatty acyl-CoA+2 ferricytochrome b5+2 H2O)O

Provided herein are yeast cells expressing heterologous Δ13 desaturases and optionally other heterologous desaturases.

### Δ13 Desaturase

Disclosed herein is a 413 desaturase capable of introducing a double bond at position 13 of a fatty acyl-CoA having a carbon chain length of 14 or more, which when expressed in a yeast cell allows said yeast cell to produce a desaturated fatty acyl-CoA having a double bond in position 13. The heterologous Δ13 desaturase may be capable of introducing a double bond at position 13 in a desaturated or saturated fatty acyl-CoA. In other words, the Δ13 desaturase may be capable of introducing a double bond at position 13 of a fatty acyl-CoA having no double bond, or one or more double bonds at one or more positions other than position 13, for example at one or more positions selected from the group consisting of position 8, 9, 10, 11, 12, 14, 15, 16, 17, 18, 19, 20 and 21. In one embodiment, the Δ13 desaturase is capable of introducing a double bond at position 13 of a fatty acyl-CoA having no double bond or having at least one double bond at position 11. In preferred embodiments, the heterologous Δ13 desaturase is capable of introducing a double bond at position 13 of a desaturated fatty acyl-CoA, such as a fatty acyl-CoA having a double bond at position 11. In preferred embodiments, the desaturated fatty acyl-CoA may have a carbon chain consisting solely of single and double bonds. In preferred embodiments, the saturated fatty acyl-CoA or desaturated fatty acyl-CoA used as a substrate and the desaturated fatty acyl-CoA having n′ double bonds have the same carbon chain length.

In some embodiments, the Δ13 desaturase is capable of introducing at least one double bond at position 13 and is also capable of introducing at least one additional double bond at an additional position, such as at any other position than position 13, of a fatty acyl-CoA. In other words, the Δ13 desaturase may be capable of introducing a double bond at position 13 and at least one more position selected from the group consisting of position 8, 9, 10, 11, 12, 14, 15, 16, 17, 18, 19, 20 and 21. In one embodiment, the Δ13 desaturase is capable of introducing a double bond at position 13 and at position 11 of a fatty acyl-CoA.

In one embodiment, the heterologous Δ13 desaturase is native to an organism of a genus selected from the group consisting of Amyelois, Desmia, Thaumetopoea and Notodonta. In one embodiment, the heterologous Δ13 desaturase is native to an organism of a species selected from the group consisting of Amyelois transitella, Desmia funeralis, Thaumetopoea bonjeani and Notodonta dromedarius. In a preferred embodiment, the heterologous Δ13 desaturase is an Amyelois desaturase, such as an Amyelois transitella desaturase. In another preferred embodiment, the heterologous Δ13 desaturase is a Notodonta desaturase, such as a Notodonta dromedarius desaturase.

In one embodiment, the Δ13 desaturase is an Amyelois Δ13 desaturase. In one embodiment, the desaturase is an Amyelois transitella Δ13 desaturase, such as the Δ13 desaturase as set forth in SEQ ID NO: 1 (AtrAATQ). In some embodiments, the Δ13 desaturase is a functional variant of an Amyelois Δ13 desaturase, a functional variant of an Amyelois transitella Δ13 desaturase, or a functional variant of the Δ13 desaturase as set forth in SEQ ID NO: 1 (AtrAATQ), having at least 60% identity or similarity thereto, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity or similarity to the Δ13 desaturase as set forth in SEQ ID NO: 1. Without being bound by theory, the AtrAATQ 413 desaturase introduces a single double bond in a fatty acyl-CoA having a carbon chain length of at least 14, wherein said single double bond is at position 13.

In one embodiment, the Δ13 desaturase is a Notodonta Δ13 desaturase. In one embodiment, the desaturase is a Notodonta dromedarius Δ13 desaturase, such as the Δ13 desaturase as set forth in SEQ ID NO: 56 (NdDes1). In some embodiments, the Δ13 desaturase is a functional variant of a Notodonta Δ13 desaturase, a functional variant of a Notodonta dromedarius Δ13 desaturase, or a functional variant of the Δ13 desaturase as set forth in SEQ ID NO: 56 (NdDes1), having at least 60% identity or similarity thereto, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity or similarity to the Δ13 desaturase as set forth in SEQ ID NO: 56. Without being bound by theory, the NdDes1 Δ13 desaturase introduces a single double bond in a fatty acyl-CoA having a carbon chain length of at least 14, wherein said single double bond is at position 13.

The gene encoding the heterologous Δ13 desaturase may be codon-optimized for the yeast cell expressing the desaturase, as is known in the art. Methods to determine whether the desaturase is expressed in the cell are known to the person of skill in the art, and include for instance detection of a given product from a given substrate, as detailed herein above and as illustrated in the examples below.

Further provided herein is the use of an Amyelois transitella desaturase in a method for introducing at least one double bond at position 13 in a saturated or desaturated fatty acyl-CoA. In some embodiments, the Amyelois transitella desaturase introduces a single double bond in the fatty acyl-CoA, which is at position 13. In some embodiments, the desaturated fatty acyl-CoA has a carbon chain consisting solely of single and double bonds. In some embodiments, the Amyelois transitella desaturase is AtrAATQ (SEQ ID NO: 1) or a functional variant thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

Also provided herein is the use of a Notodonta dromedarius desaturase in a method for introducing at least one double bond at position 13 in a saturated or desaturated fatty acyl-CoA. In some embodiments, the Notodonta dromedarius desaturase introduces a single double bond in the fatty acyl-CoA, which is at position 13. In some embodiments, the desaturated fatty acyl-CoA has a carbon chain consisting solely of single and double bonds. In some embodiments, the Notodonta dromedarius desaturase is NdDes1 (SEQ ID NO: 56) or a functional variant thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

In some embodiments, the functional variant of the Δ13 desaturase comprises or consists of an amino acid sequence from a desaturase, such as SEQ ID NO: 1 (AtrAATQ) or SEQ ID NO: 56 (NdDes1), wherein at least one amino acid residue has been deleted, inserted or substituted with another amino acid residue. The deletion, insertion, or substitution may be found in the C-terminal or N-terminal part of the sequence.

In some embodiments, at least one of amino acid residues 1 to 50, such as 1 to 40, such as 1 to 30, such as 1 to 20, of the sequence, such as SEQ ID NO: 1 (AtrAATQ) or SEQ ID NO: 56 (NdDes1), have been deleted, inserted or substituted with another amino acid residue.

In some embodiments, at least one of amino acid residues 250 to 400, such as 260 to 390, such as 270 to 380, such as 280 to 370, such as 290 to 350, such as 300 to 350, of the sequence, such as SEQ ID NO: 1 (AtrAATQ) or SEQ ID NO: 56 (NdDes1), have been deleted, inserted or substituted with another amino acid residue. In some embodiments, at least one of amino acid residues 290 to 400, such as 290 to 390, such as 290 to 380, such as 290 to 370, such as 290 to 350, of the sequence, such as SEQ ID NO: 1 (AtrAATQ) or SEQ ID NO: 56 (NdDes1), have been deleted, inserted or substituted with another amino acid residue. In some embodiments, at least one of amino acid residues 302 to 348 of the sequence, such as SEQ ID NO: 1 (AtrAATQ) or SEQ ID NO: 56 (NdDes1), have been deleted, inserted or substituted with another amino acid residue.

In some embodiments, at least one of amino acid residues 250 to 342, such as 260 to 342, such as 270 to 342, such as 280 to 342, such as 290 to 342, such as 300 to 342, of the sequence set forth in SEQ ID NO: 56 (NdDes1), have been deleted, inserted or substituted with another amino acid residue. In some embodiments, at least one of amino acid residues 290 to 342, such as 290 to 342, such as 290 to 342, such as 290 to 342, such as 290 to 342, of the sequence set forth in SEQ ID NO: 56 (NdDes1), have been deleted, inserted or substituted with another amino acid residue. In some embodiments, at least one of amino acid residues 300 to 342 of the sequence set forth in SEQ ID NO: 56 (NdDes1), have been deleted, inserted or substituted with another amino acid residue.

The skilled person will understand that the termini of many enzymes, such as some desaturases, may be changed with respect to their native sequences, while the function of said enzymes are preserved, i.e. without the enzymes loose their activity. In contrast, changing the residues comprising the catalytic site and/or residues of importance to the catalytic activity of an enzyme may not be possible without disturbing or changing the function and/or catalytic activity of said enzyme. With regards to the termini of an enzyme, the skilled person will understand, that it may be important to maintain the flexibility, hydrophobicity and/or hydrophilicity of the termini in order to preserve the features and/or function of said enzyme. For example, if the enzyme is anchored, also known as embedded and/or inserted, such as to the membrane, for example to a membrane protein, via at least one of its termini, if may be important to maintain the same or a similar hydrophobility of its terminus.

In some embodiments, the desaturated fatty acyl-CoA has a carbon length of 14 or more, such as 15, such as 16, such as 17 or more and/or the saturated fatty acid methyl ester has a carbon length of 14 or more, such as 15, such as 16, such as 17 or more.

In one embodiment, the method is performed in vitro, for example by providing a desaturase as described above, in particular AtrAATQ as set forth in SEQ ID NO: 1 or NdDes1 as set forth in SEQ ID NO: 56, or a functional variant thereof having at least 70% similarity or identity thereto, and contacting said desaturase with a saturated or desaturated fatty acyl-CoA. The desaturase may be purified from a cell expressing said desaturase as is known in the art. In one embodiment, the method is performed in vivo, preferably in a yeast cell as defined herein in the section “Yeast cell”.

In some embodiments, the Δ13 desaturase is a fusion protein, such as a chimeric protein or a mosaic protein, comprising amino acid sequences from at least two different proteins such as two different desaturases, such as three different desaturases. Preferably, at least one of the amino acid sequences comprised in such Δ13 desaturase fusion protein is derived from a Δ13 desaturase. Thus, in some embodiments, the Δ13 desaturase disclosed herein is a fusion protein comprising or consisting of an amino acid sequence from a 413 desaturase and at least one amino acid sequence, such as two amino acid sequences, or more, from at least one other desaturase, such as a Δ11 desaturase. In other embodiments, the Δ13 desaturase fusion protein comprises or consists of an amino acid sequence from a 413 desaturase and at least two amino acid sequences from at least one other desaturase, such as a Δ11 desaturase, two Δ11 desaturases, or more. In further other embodiments, the Δ13 desaturase disclosed herein is a fusion protein comprising an amino acid sequence derived from a 413 desaturase and an amino acid sequence derived from another Δ13 desaturase, such that the fusion protein comprises or consists of amino acid sequences from two different Δ13 desaturases.

In some embodiments, the Δ13 desaturase and the at least one other desaturase, such as the Δ11 or Δ13 desaturase, are derived from the same species. In other words, the Δ13 desaturase fusion protein comprises or consists of amino acid sequences from a Δ13 desaturase and at least one other desaturase which are native to the same species, such as an amino acid sequence from a Δ13 desaturase and an amino acid sequence from a Δ11 desaturase, wherein said Δ13 desaturase and Δ11 desaturase are both derived from Amyelois transitella. In other embodiments, the Δ13 desaturase and the at least one other desaturase are derived from two different species, three different species, or more different species. In other words, the Δ13 desaturase fusion protein comprises or consists of amino acid sequences from a 413 desaturase and at least one other desaturase which are native to at least two different species, such as an amino acid sequence from a Plutella xylostella Δ11 desaturase or a Cadra cautella Δ11 desaturase, and an amino acid sequence from an Amyelois transitella Δ13 desaturase.

In some embodiments, the Δ13 desaturase fusion protein comprises or consists of an amino acid sequence from one desaturase, wherein at least one of the termini of said one desaturase has been replaced, such as exchanged with a terminus of another desaturase, such as the corresponding terminus of another desaturase. The corresponding terminus of the N-terminus of said one desaturase herein refers to the N-terminus of said another desaturase, and the same terminology applies to the C-terminus. The skilled person will understand that the termini of many enzymes, such as some desaturases, may be changed with respect to their native sequences, while the function of said enzymes are preserved, i.e. without the enzymes loose their activity. In contrast, changing the residues comprising the catalytic site and/or residues of importance to the catalytic activity of an enzyme may not be possible without disturbing or changing the function and/or catalytic activity of said enzyme. With regards to the termini of an enzyme, the skilled person will understand, that it may be important to maintain the flexibility, hydrophobicity and/or hydrophilicity of the termini in order to preserve the features and/or function of said enzyme. For example, if the enzyme is anchored, also known as embedded and/or inserted, such as to the membrane, for example to a membrane protein, via at least one of its termini, if may be important to maintain the same or a similar hydrophobility of its terminus.

Thus, in some embodiments, the Δ13 desaturase fusion protein comprises or consists of an amino acid sequence from a 413 desaturase, that is stripped from its native N- and/or C-terminus, and fused to the N- and/or C-terminus of another desaturase, such as another Δ13 desaturase or a Δ11 desaturase. In some embodiments, the Δ13 desaturase is a fusion protein comprising or consisting of a part of the amino acid sequence of a 413 desaturase, wherein 60 N-terminus amino acid residues of said Δ13 desaturase have been deleted or substituted by an amino acid sequence from at least one other desaturase, such as wherein 50 N-terminus amino acid residues of said Δ13 desaturase have been deleted or substituted by an amino acid sequence from at least one other desaturase, such as wherein 40 N-terminus amino acid residues of said Δ13 desaturase have been deleted or substituted by an amino acid sequence from at least one other desaturase, such as wherein 35 N-terminus amino acid residues of said Δ13 desaturase have been deleted or substituted by an amino acid sequence from at least one other desaturase, such as wherein 30 N-terminus amino acid residues of said Δ13 desaturase have been deleted or substituted by an amino acid sequence from at least one other desaturase, such as wherein 25 N-terminus amino acid residues of said Δ13 desaturase have been deleted or substituted by an amino acid sequence from at least one other desaturase, such as wherein 20 N-terminus amino acid residues of said Δ13 desaturase have been deleted or substituted by an amino acid sequence from at least one other desaturase, such as wherein 15 N-terminus amino acid residues of said Δ13 desaturase have been deleted or substituted by an amino acid sequence from at least one other desaturase, such as wherein 10 N-terminus amino acid residues of said Δ13 desaturase have been deleted or substituted by an amino acid sequence from at least one other desaturase, such as wherein 5 N-terminus amino acid residues of said Δ13 desaturase have been deleted or substituted by an amino acid sequence from at least one other desaturase, such as wherein between 1 and 55 N-terminus amino acid residues of said Δ13 desaturase have been deleted or substituted by an amino acid sequence from at least one other desaturase, such as wherein between 5 and 45 N-terminus amino acid residues of said Δ13 desaturase have been deleted or substituted by an amino acid sequence from at least one other desaturase, such as wherein between 10 and 40 N-terminus amino acid residues of said Δ13 desaturase have been deleted or substituted by an amino acid sequence from at least one other desaturase, such as wherein between 15 and 35 N-terminus amino acid residues of said Δ13 desaturase have been deleted or substituted by an amino acid sequence from at least one other desaturase, such as wherein between 20 and 35 N-terminus amino acid residues of said Δ13 desaturase have been deleted or substituted by an amino acid sequence from at least one other desaturase, such as wherein between 25 and 35 N-terminus amino acid residues of said Δ13 desaturase have been deleted or substituted by an amino acid sequence from at least one other desaturase,
and/or
wherein 50 C-terminus amino acid residues of said Δ13 desaturase have been deleted or substituted by an amino acid sequence from at least one other desaturase, such as wherein 40 C-terminus amino acid residues of said Δ13 desaturase have been deleted or substituted by an amino acid sequence from at least one other desaturase, such as wherein 35 C-terminus amino acid residues of said Δ13 desaturase have been deleted or substituted by an amino acid sequence from at least one other desaturase, such as wherein 30 C-terminus amino acid residues of said Δ13 desaturase have been deleted or substituted by an amino acid sequence from at least one other desaturase, such as wherein 25 C-terminus amino acid residues of said Δ13 desaturase have been deleted or substituted by an amino acid sequence from at least one other desaturase, such as wherein 20 C-terminus amino acid residues of said Δ13 desaturase have been deleted or substituted by an amino acid sequence from at least one other desaturase, such as wherein 15 C-terminus amino acid residues of said Δ13 desaturase have been deleted or substituted by an amino acid sequence from at least one other desaturase, such as wherein 10 C-terminus amino acid residues of said Δ13 desaturase have been deleted or substituted by an amino acid sequence from at least one other desaturase, such as wherein between 1 and 55 C-terminus amino acid residues of said Δ13 desaturase have been deleted or substituted by an amino acid sequence from at least one other desaturase, such as wherein between 5 and 45 C-terminus amino acid residues of said Δ13 desaturase have been deleted or substituted by an amino acid sequence from at least one other desaturase, such as wherein between 10 and 40 C-terminus amino acid residues of said Δ13 desaturase have been deleted or substituted by an amino acid sequence from at least one other desaturase, such as wherein between 15 and 35 C-terminus amino acid residues of said Δ13 desaturase have been deleted or substituted by an amino acid sequence from at least one other desaturase, such as wherein between 20 and 35 C-terminus amino acid residues of said Δ13 desaturase have been deleted or substituted by an amino acid sequence from at least one other desaturase, such as wherein between 25 and 35 C-terminus amino acid residues of said Δ13 desaturase have been deleted or substituted by an amino acid sequence from at least one other desaturase. In preferred embodiments, said Δ13 desaturase is selected from the Δ13 desaturases set forth in SEQ ID NO: 1 (AtrAATQ) or SEQ ID NO: 56 (NdDes1).

Preferably, in the Δ13 desaturase fusion protein, the C- or N-terminus residues which are deleted are substituted with an equal or near equal amount of amino acid residues from the other desaturase, such that for example in a Δ13 desaturase fusion protein where 30 N-terminus amino acids have been deleted, said 30 amino acids have been substituted with in the range of 20 to 40 N-terminus amino acid residues from another desaturase, such as in the range of 25 to 35 N-terminus amino acid residues from another desaturase, such as in the range of 28 to 32 N-terminus amino acid residues from another desaturase, such as from a 411 desaturase. Thus, for example in a 413 desaturase fusion protein where 30 C-terminus amino acids have been deleted, said 30 amino acids have been substituted with in the range of 20 to 40 C-terminus amino acid residues from another desaturase, such as in the range of 25 to 35 C-terminus amino acid residues from another desaturase, such as in the range of 28 to 32 C-terminus amino acid residues from another desaturase, such as from a 411 desaturase.

The N-terminus residues are herein defined as the first residues of the amino acid sequence of a protein, such that deleting or replacing X amino acid residues of the N-terminus of a protein refers to deleting or replacing the X first amino acids of said protein. In other words, deleting or replacing 30 amino acid residues of the N-terminus of a protein of 300 amino acids refers to deleting or replacing amino acid number 1 to 30 of said protein.

The C-terminus residues are herein defined as the last residues of the amino acid sequence of a protein, such that deleting or replacing X amino acid residues of the C-terminus of a protein refers to deleting or replacing the X last amino acids of said protein. In other words, deleting or replacing 30 amino acid residues of the C-terminus of a protein of 300 amino acids refers to deleting or replacing amino acid number 271 to 300 of said protein.

In some embodiments, the functional variant of the Δ13 desaturase comprises or consists of an amino acid sequence from a desaturase, such as SEQ ID NO: 1 (AtrAATQ) or SEQ ID NO: 56 (NdDes1), wherein at least one amino acid residue has been deleted, inserted or substituted with another amino acid residue. The deletion, insertion, or substitution may be found in the C-terminus or N-terminus part of the sequence.

In some embodiments, at least one of the N-terminus amino acid residues 1 to 50, such as 1 to 40, such as 1 to 30, such as 1 to 20, of the sequence, such as of a sequence selected from SEQ ID NO: 1 (AtrAATQ) and SEQ ID NO: 56 (NdDes1), have been deleted, inserted or substituted with another amino acid residue.

In some embodiments, at least one of the C-terminus amino acid residues 250 to 400, such as 260 to 390, such as 270 to 380, such as 280 to 370, such as 290 to 350, such as 300 to 350, of the sequence, such as of a sequence selected from SEQ ID NO: 1 (AtrAATQ) and SEQ ID NO: 56 (NdDes1), have been deleted, inserted or substituted with another amino acid residue. In some embodiments, at least one of amino acid residues 290 to 400, such as 290 to 390, such as 290 to 380, such as 290 to 370, such as 290 to 350, of the sequence, such as SEQ ID NO: 1 (AtrAATQ) and/or SEQ ID NO: 56 (NdDes1), have been deleted, inserted or substituted with another amino acid residue. In some embodiments, at least one of amino acid residues 302 to 348 of the sequence, such as SEQ ID NO: 1 (AtrAATQ) and/or SEQ ID NO: 56 (NdDes1), have been deleted, inserted or substituted with another amino acid residue.

In some embodiments, the Δ13 desaturase is a fusion protein comprising or consisting of a part of the amino acid sequence of the Amyelois transitella desaturase AtrAATQ set forth in SEQ ID NO: 1, wherein the amino acid residues 1 to 20 comprising the N-terminus and/or amino acid residues 302 to 348 comprising the C-terminus are deleted or replaced by an amino acid sequence from at least one other desaturase.

In some embodiments, the functional variant of a Δ13 desaturase comprises or consists of an amino acid sequence from an Amyelois transitella Δ13 desaturase, and at least one amino acid sequence from an Amyelois transitella Δ11 desaturase. In one embodiment, the functional variant of a Δ13 desaturase comprises or consists of a part of the amino acid sequence of the Amyelois transitella desaturase AtrAATQ set forth in SEQ ID NO: 1, and at least one part of the amino acid sequence of the Amyelois transitella desaturase Desat16 set forth in SEQ ID NO: 7. In some embodiments, the functional variant of the Δ13 desaturase consists of a variant of AtrAATQ (SEQ ID NO: 1), wherein amino acid residues 1-20 of the N-terminus and/or amino acid residues 302-348 of the C-terminus have been substituted with amino acid residues 1-20 of the N-terminus and amino acid residues 302-326 of the C-terminus of Desat16 (SEQ ID NO: 7), respectively. Thus, in some embodiments, the 413 desaturase is a functional variant of a 413 desaturase consisting of the sequence set forth in SEQ ID NO: 132 (Desat83).

In some embodiments, the functional variant of a 413 desaturase comprises or consists of an amino acid sequence from an Amyelois transitella Δ13 desaturase, and an amino acid sequence from a Cadra cautella Δ11 desaturase and an amino acid from a Plutella xylostella Δ11 desaturase. In one embodiment, the functional variant of a 413 desaturase comprises or consists of part of the amino acid sequence of the Cadra cautella desaturase Desat70 set forth in SEQ ID NO: 69, part of the amino acid sequence of the Plutella xylostella desaturase Desat45 set forth in SEQ ID NO: 33, and/or part of the amino acid sequence of the Amyelois transitella desaturase AtrAATQ set forth in SEQ ID NO: 1. In some embodiments, the functional variant of the Δ13 desaturase consists of a variant of AtrAATQ (SEQ ID NO: 1), wherein amino acid residues 1-20 of the N-terminus and amino acid residues 302-348 of the C-terminus have been substituted with amino acid residues 1-19 of the N-terminus of Desat70 (SEQ ID NO: 69) and amino acid residues 305-349 of the C-terminus of Desat45 (SEQ ID NO: 33), respectively. Thus, in some embodiments, the Δ13 desaturase is a functional variant of a Δ13 desaturase consisting of the sequence set forth in SEQ ID NO: 133 (Desat84).

In some embodiments, the functional variant of a Δ13 desaturase comprises or consists of an amino acid sequence from an Amyelois transitella Δ13 desaturase, and an amino acid sequence from a Cadra cautella Δ11 desaturase and an amino acid from a Plutella xylostella Δ11 desaturase. In one embodiment, the functional variant of a 413 desaturase comprises or consists of part of the amino acid sequence of the Cadra cautella desaturase Desat70 set forth in SEQ ID NO: 69, part of the amino acid sequence of the Plutella xylostella desaturase Desat45 set forth in SEQ ID NO: 33, and/or part of the amino acid sequence of the Amyelois transitella desaturase AtrAATQ set forth in SEQ ID NO: 1. In some embodiments, the functional variant of the Δ13 desaturase consists of a variant of AtrAATQ (SEQ ID NO: 1), wherein amino acid residues 1-20 of the N-terminus and amino acid residues 302-348 of the C-terminus have been substituted with amino acid residues 1-23 of the N-terminus of Desat45 (SEQ ID NO: 33) and amino acid residues 301-342 of the C-terminus of Desat70 (SEQ ID NO: 69), respectively. Thus, in some embodiments, the Δ13 desaturase is a functional variant of a 413 desaturase consisting of the sequence set forth in SEQ ID NO: 134 (Desat85).

The present yeast cells may express at least one heterologous Δ13 desaturase. In some embodiments, the cell expresses one heterologous Δ13 desaturase. It may however be desirable to express several heterologous Δ13 desaturases, such as at least two heterologous Δ13 desaturases, which may be identical or different. Alternatively, it may be desirable to express several copies of the nucleic acid encoding the at least one heterologous Δ13 desaturase, such as at least two copies, such as at least three copies or more. In other embodiments, the cell expresses at least two heterologous Δ13 desaturases, for example three heterologous Δ13 desaturases.

### Further Desaturase

The yeast cell provided herein may, in addition to the heterologous Δ13 desaturase, express at least one further desaturase which is not a Δ13 desaturase, said further desaturase may be an endogenous, also known as a native or homologous desaturase, or a heterologous desaturase. In other words, the yeast cell expresses at least one native desaturase which is not a 413 desaturase. In preferred embodiments, however, the further desaturase is a heterologous desaturase. In other words, the yeast cell provided herein may, in addition to the heterologous Δ13 desaturase, express at least one further heterologous desaturase which is not a 413 desaturase. In one embodiment, the further heterologous desaturase is capable of introducing at least one double bond at any position which is not position 13 in a saturated or desaturated fatty acyl-CoA having a carbon chain length of at least 14. In other words, the further desaturase may be capable of introducing at least one double bond at a position selected from the group consisting of position 8, 9, 10, 11, 12, 14, 15, 16, 17, 18, 19, 20 and 21 in a fatty acyl-CoA. In a preferred embodiment, said fatty acyl-CoA is a saturated fatty acyl-CoA, for example a hexadecanoyl-CoA. In another embodiment, said fatty acyl-CoA is a desaturated fatty acyl-CoA having a double bond at position 13. In yet another embodiment, said fatty acyl-CoA is a desaturated fatty acyl-CoA having a double bond at position 13 and at a position other than position 13, such as for example at position 13 and at position 11. In preferred embodiments, the desaturated fatty acyl-CoA may have a carbon chain consisting solely of single and double bonds.

Thus, in one embodiment, the yeast cell is capable of expressing at least one further heterologous desaturase which is a heterologous 45 desaturase. In another embodiment, the yeast cell is capable of expressing at least one further heterologous desaturase which is a heterologous Δ6 desaturase. In another embodiment, the yeast cell is capable of expressing at least one further heterologous desaturase which is a heterologous 47 desaturase. In another embodiment, the yeast cell is capable of expressing at least one further heterologous desaturase which is a heterologous Δ8 desaturase. In another embodiment, the yeast cell is capable of expressing at least one further heterologous desaturase which is a heterologous 49 desaturase. In another embodiment, the yeast cell is capable of expressing at least one further heterologous desaturase which is a heterologous Δ10 desaturase. In another embodiment, the yeast cell is capable of expressing at least one further heterologous desaturase which is a heterologous Δ11 desaturase. In another embodiment, the yeast cell is capable of expressing at least one further heterologous desaturase which is a heterologous Δ12 desaturase. In another embodiment, the yeast cell is capable of expressing at least one further heterologous desaturase which is a heterologous Δ14 desaturase. In another embodiment, the yeast cell is capable of expressing at least one further heterologous desaturase which is a heterologous Δ15 desaturase. In another embodiment, the yeast cell is capable of expressing at least one further heterologous desaturase which is a heterologous Δ16 desaturase. In another embodiment, the yeast cell is capable of expressing at least one further heterologous desaturase which is a heterologous Δ17 desaturase. In another embodiment, the yeast cell is capable of expressing at least one further heterologous desaturase which is a heterologous Δ18 desaturase. In another embodiment, the yeast cell is capable of expressing at least one further heterologous desaturase which is a heterologous Δ19 desaturase. In another embodiment, the yeast cell is capable of expressing at least one further heterologous desaturase which is a heterologous 420 desaturase. In another embodiment, the yeast cell is capable of expressing at least one further heterologous desaturase which is a heterologous Δ21 desaturase. In preferred embodiments, the further desaturase is a 411 desaturase.

The further heterologous desaturase may be native to any type of organism which is different from the yeast cell in which it is expressed according to the present methods. In another embodiment, the heterologous desaturase is native to an insect, such as of the Lepidoptera order, such as of the genus Grapholita, Helicoverpa, Lampronia, Lobesia, Manducta, Ostrinia, Plutella, Thalassiosira, Thaumetopoea, Tribolium, Trichoplusia, Spodoptera, Amyelois, Chilo, Diatraea, Plodia, Antheraea, Cadra or Yponomeuta, such as Grapholita molesta, Helicoverpa assulta, Helicoverpa zea, Lampronia capitella, Lobesia botrana, Manducta sexta, Ostrinia furnacalis, Ostrinia nubilalis, Plutella xylostella, Spodoptera exigua, Spodoptera littoralis, Spodoptera litura, Thalassiosira pseudonana, Thaumetopoea pityocampa, Tribolium castaneum, Trichoplusia ni, Amyelois transitella, Chilo suppressalis, Diatraea saccharalis, Plodia interpunctella, Antheraea pernyi, Cadra cautella, or Yponomeuta padella.

In a preferred embodiment, the further heterologous desaturase is a Spodoptera desaturase, such as a Spodoptera litura desaturase, a Spodoptera littoralis desaturase, or a Spodotera exigua desaturase. In another embodiment, the further heterologous desaturase is a Plutella desaturase, such as a Plutella xylostella desaturase. In another embodiment, the further heterologous desaturase is a Helicoverpa desaturase, such as a Helicoverpa zea desaturase. In another embodiment, the further heterologous desaturase is an Amyelois desaturase, such as an Amyelois transitella desaturase. In another embodiment, the further heterologous desaturase is an Agrotis desaturase, such as an Agrotis segetum desaturase. In another embodiment, the further heterologous desaturase is a Trichoplusia desaturase, such as a Trichoplusia ni desaturase. In another embodiment, the further heterologous desaturase is an Amyelois desaturase, such as an Amyelois transitella desaturase. In another embodiment, the further heterologous desaturase is a Chilo desaturase, such as a Chilo suppressalis desaturase. In another embodiment, the further heterologous desaturase is a Diatraea desaturase, such as a Diatraea saccharalis desaturase. In another embodiment, the further heterologous desaturase is a Plodia desaturase, such as a Plodia interpunctella desaturase. In another embodiment, the further heterologous desaturase is an Antheraea desaturase, such as an Antheraea pernyi desaturase. In another embodiment, the further heterologous desaturase is a Cadra desaturase, such as a Cadra cautella desaturase. In another embodiment, the further heterologous desaturase is an Yponomeuta desaturase, such as an Yponomeuta padelladesaturase. In another embodiment, the further heterologous desaturase is a Lobesia desaturase, such as a Lobesia botrana desaturase.

In one embodiment, the further heterologous desaturase is a Spodoptera desaturase. In one embodiment, the desaturase is a Spodoptera litura desaturase, such as the desaturase as set forth in SEQ ID NO: 3 (Desat38). In one embodiment, the desaturase is a Spodoptera littoralis desaturase, such as the desaturase as set forth in SEQ ID NO: 5 (Desat20). In one embodiment, the desaturase is a Spodoptera exigua desaturase, such as the desaturase as set forth in SEQ ID NO: 31 (Desat37). In some embodiments, the desaturase is a functional variant of a Spodoptera desaturase, such as a functional variant of a Spodoptera litura desaturase, of a Spodoptera littoralis desaturase or of a Spodoptera exigua desaturase, such as a functional variant of a Spodoptera littoralis desaturase as set forth in SEQ ID NO: 3 (Desat38), a functional variant of the Spodoptera littoralis desaturase as set forth in SEQ ID NO: 5 (Desat20), or a functional variant of the Spodoptera exigua desaturase as set forth in SEQ ID NO: 31 (Desat37) having at least 60% identity or similarity thereto.

In one embodiment, the further heterologous desaturase is an Amyelois desaturase. In one embodiment, the desaturase is an Amyelois transitella desaturase, such as the desaturase as set forth in SEQ ID NO: 7 (Desat16). In some embodiments, the desaturase is a functional variant of an Amyelois desaturase, a functional variant of an Amyelois transitella desaturase, or a functional variant of the desaturase as set forth in SEQ ID NO: 7 (Desat16), having at least 60% identity or similarity thereto.

In one embodiment, the further heterologous desaturase is an Agrotis desaturase. In one embodiment, the desaturase is an Agrotis segetum desaturase, such as the desaturase as set forth in SEQ ID NO: 9 (Desat19). In some embodiments, the desaturase is a functional variant of an Agrotis desaturase, a functional variant of a Agrotis segetum desaturase, or a functional variant of the desaturase as set forth in SEQ ID NO: 9 (Desat19), having at least 60% identity or similarity thereto.

In one embodiment, the further heterologous desaturase is a Trichoplusia desaturase. In one embodiment, the desaturase is a Trichoplusia ni desaturase, such as the desaturase as set forth in SEQ ID NO: 11 (Desat21). In some embodiments, the desaturase is a functional variant of a Trichoplusia desaturase, a functional variant of a Trichoplusia ni desaturase, or a functional variant of the desaturase as set forth in SEQ ID NO: 11 (Desat21), having at least 60% identity or similarity thereto.

In one embodiment, the further heterologous desaturase is a Plutella desaturase. In one embodiment, the desaturase is a Plutella xylostella desaturase, such as the desaturase as set forth in SEQ ID NO: 33 (Desat45). In some embodiments, the desaturase is a functional variant of a Plutella desaturase, a functional variant of a Plutella xylostella desaturase, or a functional variant of the desaturase as set forth in SEQ ID NO: 33 (Desat45), having at least 60% identity or similarity thereto.

In one embodiment, the further heterologous desaturase is a Helicoverpa desaturase. In one embodiment, the desaturase is a Helicoverpa zea desaturase, such as the desaturase as set forth in SEQ ID NO: 29 (Desat51). In some embodiments, the desaturase is a functional variant of a Helicoverpa desaturase, a functional variant of a Helicoverpa zea desaturase, or a functional variant of the desaturase as set forth in SEQ ID NO: 29 (Desat51), having at least 60% identity or similarity thereto.

In one embodiment, the further heterologous desaturase is a Chilo desaturase. In one embodiment, the desaturase is a Chilo suppressalis desaturase, such as the desaturase as set forth in SEQ ID NO: 51 (Desat44). In some embodiments, the desaturase is a functional variant of a Chilo desaturase, a functional variant of a Chilo suppressalis desaturase, or a functional variant of the desaturase as set forth in SEQ ID NO: 51 (Desat44), having at least 60% identity or similarity thereto.

In one embodiment, the further heterologous desaturase is a Diatraea desaturase. In one embodiment, the desaturase is a Diatraea saccharalis desaturase, such as the desaturase as set forth in SEQ ID NO: 59 (Desat63). In some embodiments, the desaturase is a functional variant of a Diatraea desaturase, a functional variant of a Diatraea saccharalis desaturase, or a functional variant of the desaturase as set forth in SEQ ID NO: 59 (Desat63), having at least 60% identity or similarity thereto.

In one embodiment, the further heterologous desaturase is a Plodia desaturase. In one embodiment, the desaturase is a Plodia interpunctella desaturase, such as the desaturase as set forth in SEQ ID NO: 61 (Desat65). In some embodiments, the desaturase is a functional variant of a Plodia desaturase, a functional variant of a Plodia interpunctella desaturase, or a functional variant of the desaturase as set forth in SEQ ID NO: 61 (Desat65), having at least 60% identity or similarity thereto.

In one embodiment, the further heterologous desaturase is a Lobesia desaturase. In one embodiment, the desaturase is a Lobesia botrana desaturase, such as the desaturase as set forth in SEQ ID NO: 63 (Desat71) or SEQ ID NO: 65 (Desat79). In some embodiments, the desaturase is a functional variant of a Lobesia desaturase, a functional variant of a Lobesia botrana desaturase, or a functional variant of the desaturase as set forth in SEQ ID NO: 63 (Desat71) or SEQ ID NO: 65 (Desat79), having at least 60% identity or similarity thereto.

In one embodiment, the further heterologous desaturase is an Antheraea desaturase. In one embodiment, the desaturase is an Antheraea pernyi desaturase, such as the desaturase as set forth in SEQ ID NO: 67 (Desat72). In some embodiments, the desaturase is a functional variant of a Antheraea desaturase, a functional variant of a Antheraea pernyi desaturase, or a functional variant of the desaturase as set forth in SEQ ID NO: 67 (Desat72), having at least 60% identity or similarity thereto.

In one embodiment, the further heterologous desaturase is a Cadra desaturase. In one embodiment, the desaturase is a Cadra cautella desaturase, such as the desaturase as set forth in SEQ ID NO: 69 (Desat70). In some embodiments, the desaturase is a functional variant of a Cadra desaturase, a functional variant of a Cadra cautella desaturase, or a functional variant of the desaturase as set forth in SEQ ID NO: 69 (Desat70), having at least 60% identity or similarity thereto.

In one embodiment, the further heterologous desaturase is an Yponomeuta desaturase. In one embodiment, the desaturase is an Yponomeuta padella desaturase, such as the desaturase as set forth in SEQ ID NO: 71 (Desat73). In some embodiments, the desaturase is a functional variant of an Yponomeuta desaturase, a functional variant of an Yponomeuta padella desaturase, or a functional variant of the desaturase as set forth in SEQ ID NO: 71 (Desat73), having at least 60% identity or similarity thereto.

The term “variant thereof having at least 60% identity or similarity” in relation to a given enzyme shall be understood to refer to variants having 60% identity or similarity or more to said enzyme, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity or similarity to the enzyme, or more.

The yeast cell to be modified may express a native desaturase, which may have a negative impact on the production of desaturated fatty acyl-CoA and/or desaturated fatty alcohol. Accordingly, if the cell to be modified expresses such a native desaturase, the organism may be modified so that activity of the native desaturase is reduced or absent.

To ensure lack or at least reduction of activity of a native desaturase, methods known in the art can be employed. The gene encoding the native desaturase may be deleted or partly deleted in order to ensure that the native desaturase is not expressed. Alternatively, the gene may be mutated so that the native desaturase is expressed but lacks activity, e.g. by mutation of the catalytic site of the enzyme. Alternatively, translation of mRNA to an active protein may be prevented by methods such as silencing RNA or siRNA. Alternatively, the cell may be incubated in a medium comprising an inhibitor which inhibits activity of the native desaturase. A compound inhibiting transcription of the gene encoding the native desaturase may also be provided so that transcription is inactivated when said compound is present. Other methods known in the art may be employed.

Inactivation of the native desaturase may thus be permanent or long-term, i.e. the modified cell exhibits reduced or no activity of the native desaturase in a stable manner, or it may be transient, i.e. the modified cell may exhibit activity of the native desaturase for periods of time, but this activity can be suppressed or reduced for other periods of time.

In some embodiments, the native yeast desaturase is replaced with a heterologous desaturase. The heterologous desaturase may for example be a desaturase which does not generate undesired by-products, or generates less undesired by-products compared to the native yeast desaturase when tested under the same conditions. Such undesired by-products may for example be (Z9)-hexadecenoyl-CoA (Z9-16:CoA) and/or (Z9)-hexadecenol (Z9-16:OH). It has been found that replacing the Yarrowia lipolytica OLE1 desaturase with a heterologous desaturase from Puccinia graminis or

Arxula adeninivorans leads to reduced production of the by-product Z9-16:OH (Tsakraklides et al., 2018).

The heterologous desaturases replacing the native yeast desaturase may be specific to C18 compounds, such as octadecanoyl-CoA, and make octadecenoyl-CoA as the main product (Tsakraklides et al., 2018).

In some embodiments, the further desaturase may be a native desaturase. It will be understood by the skilled person that the features described anywhere herein, in particular in this section, applying to the further heterologous desaturase may also apply to said native desaturase, for example if said further heterologous desaturase is a Δ11 desaturase is may be substituted with a native Δ11 desaturase.

In some embodiments, the further desaturase may be a fusion protein, such as a chimeric protein or a mosaic protein, comprising or consisting of a plurality of desaturases, such as at least two desaturases, such as at least three desaturases, or more. The further desaturase fusion protein may constructed and comprise similar features as the Δ13 desaturases fusion protein described herein above in the section “Δ13 desaturase”.

In some embodiments, the functional variant of the further desaturase comprises or consists of an amino acid sequence from a desaturase, such as a Spodoptera desaturase, such as a Spodoptera litura desaturase, for example Desat38 as set forth in SEQ ID NO: 3, or such as a Spodoptera littoralis desaturase, for example Desat20 as set forth in SEQ ID NO: 5, or such as a Spodoptera exigua desaturase, for example Desat37 as set forth in SEQ ID NO: 31; an Amyelois desaturase, such as an Amyelois transitella desaturase, for example Desat16 as set forth in SEQ ID NO: 7, an Agrotis desaturase, such as an Agrotis segetum desaturase, for example Desat19 as set forth in SEQ ID NO: 9; a Trichoplusia desaturase, such as a Trichoplusia ni desaturase, for example Desat21 as set forth in SEQ ID NO: 11; a Plutella desaturase, such as a Plutella xylostella desaturase, for example Desat45 as set forth in SEQ ID NO: 33; a Helicoverpa desaturase, such as a Helicoverpa zea desaturase, for example Desat51 as set forth in SEQ ID NO: 29; a Chilo desaturase, such as a Chilo suppressalis desaturase, for example Desat44 as set forth in SEQ ID NO: 51; a Diatraea desaturase, such as a Diatraea saccharalis desaturase, for example Desat63 as set forth in SEQ ID NO: 59; a Plodia desaturase, such as a Plodia interpunctella desaturase, for example Desat65 as set forth in SEQ ID NO: 61; a Lobesia desaturase, such as a Lobesia botrana desaturase, for example Desat71 as set forth in SEQ ID NO: 63 or Desat79 as set forth in SEQ ID NO: 65; an Antheraea desaturase, such as an Antheraea pernyi desaturase, for example Desat72 as set forth in SEQ ID NO: 67; a Cadra desaturase, such as a Cadra cautella desaturase, for example Desat70 as set forth in SEQ ID NO: 69; and/or a Yponomeuta desaturase, such as a Yponomeuta padella desaturase, for example Desat73 as set forth in SEQ ID NO: 71, wherein at least one amino acid residue has been deleted, inserted or substituted with another amino acid residue. The deletion, insertion, or substitution may be found in the C-terminal or N-terminal part of the sequence.

In some embodiments, at least one of amino acid residues 1 to 50, such as 1 to 40, such as 1 to 30, such as 1 to 20, of the sequence, such as SEQ ID NO: 3 (Desat38), SEQ ID NO: 5 (Desat20), SEQ ID NO: 31 (Desat37), SEQ ID NO: 7 (Desat16), SEQ ID NO: 9 (Desat19), SEQ ID NO: 11 (Desat21), SEQ ID NO: 33 (Desat45), SEQ ID NO: 29 (Desat51), SEQ ID NO: 51 (Desat44), SEQ ID NO: 59 (Desat63), SEQ ID NO: 61 (Desat65), SEQ ID NO: 63 (Desat71), SEQ ID NO: 65 (Desat79), SEQ ID NO: 67 (Desat72), SEQ ID NO: 69 (Desat70), and/or SEQ ID NO: 71 (Desat73), have been deleted, inserted or substituted with another amino acid residue.

In some embodiments, at least one of amino acid residues 250 to 400, such as 260 to 390, such as 270 to 380, such as 280 to 370, such as 290 to 350, such as 300 to 350, of the sequence, such as SEQ ID NO: 3 (Desat38), SEQ ID NO: 5 (Desat20), SEQ ID NO: 31 (Desat37), SEQ ID NO: 7 (Desat16), SEQ ID NO: 9 (Desat19), SEQ ID NO: 11 (Desat21), SEQ ID NO: 33 (Desat45), SEQ ID NO: 29 (Desat51), SEQ ID NO: 51 (Desat44), SEQ ID NO: 59 (Desat63), SEQ ID NO: 61 (Desat65), SEQ ID NO: 63 (Desat71), SEQ ID NO: 65 (Desat79), SEQ ID NO: 67 (Desat72), SEQ ID NO: 69 (Desat70), and/or SEQ ID NO: 71 (Desat73), have been deleted, inserted or substituted with another amino acid residue. In some embodiments, at least one of amino acid residues 290 to 400, such as 290 to 390, such as 290 to 380, such as 290 to 370, such as 290 to 350, of the sequence, such as SEQ ID NO: 3 (Desat38), SEQ ID NO: 5 (Desat20), SEQ ID NO: 31 (Desat37), SEQ ID NO: 7 (Desat16), SEQ ID NO: 9 (Desat19), SEQ ID NO: 11 (Desat21), SEQ ID NO: 33 (Desat45), SEQ ID NO: 29 (Desat51), SEQ ID NO: 51 (Desat44), SEQ ID NO: 59 (Desat63), SEQ ID NO: 61 (Desat65), SEQ ID NO: 63 (Desat71), SEQ ID NO: 65 (Desat79), SEQ ID NO: 67 (Desat72), SEQ ID NO: 69 (Desat70), and/or SEQ ID NO: 71 (Desat73), have been deleted, inserted or substituted with another amino acid residue. In some embodiments, at least one of amino acid residues 302 to 348 of the sequence, such as SEQ ID NO: 3 (Desat38), SEQ ID NO: 5 (Desat20), SEQ ID NO: 31 (Desat37), SEQ ID NO: 7 (Desat16), SEQ ID NO: 9 (Desat19), SEQ ID NO: 11 (Desat21), SEQ ID NO: 33 (Desat45), SEQ ID NO: 29 (Desat51), SEQ ID NO: 51 (Desat44), SEQ ID NO: 59 (Desat63), SEQ ID NO: 61 (Desat65), SEQ ID NO: 63 (Desat71), SEQ ID NO: 65 (Desat79), SEQ ID NO: 67 (Desat72), SEQ ID NO: 69 (Desat70), and/or SEQ ID NO: 71 (Desat73), have been deleted, inserted or substituted with another amino acid residue.

The skilled person will understand that the termini of many enzymes, such as some desaturases, may be changed with respect to their native sequences, while the function of said enzymes are preserved, i.e. without the enzymes loose their activity. In contrast, changing the residues comprising the catalytic site and/or residues of importance to the catalytic activity of an enzyme may not be possible without disturbing or changing the function and/or catalytic activity of said enzyme. With regards to the termini of an enzyme, the skilled person will understand, that it may be important to maintain the flexibility, hydrophobicity and/or hydrophilicity of the termini in order to preserve the features and/or function of said enzyme. For example, if the enzyme is anchored, also known as embedded and/or inserted, such as to the membrane, for example to a membrane protein, via at least one of its termini, if may be important to maintain the same or a similar hydrophobility of its terminus.

### Δ13 Desaturase and Further Desaturase

Any of the above Δ13 desaturases can be expressed together with any of the above further desaturases.

Thus, in some embodiments, the cell expresses:
an Amyelois Δ13 desaturase, such as an Amyelois transitella Δ13 desaturase, for example AtrAATQ as set forth in SEQ ID NO: 1, and
a further desaturase selected from: a Spodoptera desaturase, such as a Spodoptera litura desaturase, for example Desat38 as set forth in SEQ ID NO: 3, or such as a Spodoptera littoralis desaturase, for example Desat20 as set forth in SEQ ID NO: 5, or such as a Spodoptera exigua desaturase, for example Desat37 as set forth in SEQ ID NO: 31; an Amyelois desaturase, such as an Amyelois transitella desaturase, for example Desat16 as set forth in SEQ ID NO: 7, an Agrotis desaturase, such as an Agrotis segetum desaturase, for example Desat19 as set forth in SEQ ID NO: 9; a Trichoplusia desaturase, such as a Trichoplusia ni desaturase, for example Desat21 as set forth in SEQ ID NO: 11; a Plutella desaturase, such as a Plutella xylostella desaturase, for example Desat45 as set forth in SEQ ID NO: 33; a Helicoverpa desaturase, such as a Helicoverpa zea desaturase, for example Desat51 as set forth in SEQ ID NO: 29;
a Chilo desaturase, such as a Chilo suppressalis desaturase, for example Desat44 as set forth in SEQ ID NO: 51; a Diatraea desaturase, such as a Diatraea saccharalis desaturase, for example Desat63 as set forth in SEQ ID NO: 59; a Plodia desaturase, such as a Plodia interpunctella desaturase, for example Desat65 as set forth in SEQ ID NO: 61; a Lobesia desaturase, such as a Lobesia botrana desaturase, for example Desat71 as set forth in SEQ ID NO: 63 or Desat79 as set forth in SEQ ID NO: 65; an Antheraea desaturase, such as an Antheraea pernyi desaturase, for example Desat72 as set forth in SEQ ID NO: 67; a Cadra desaturase, such as a Cadra cautella desaturase, for example Desat70 as set forth in SEQ ID NO: 69; a Yponomeuta desaturase, such as a Yponomeuta padella desaturase, for example Desat73 as set forth in SEQ ID NO: 71;
or functional variants thereof having at least 60% identity or similarity thereto.

Thus, in some embodiments, the cell expresses:
a Notodonta Δ13 desaturase, such as a Notodonta dromedarius Δ13 desaturase, for example NdDes1 as set forth in SEQ ID NO: 56, and
a further desaturase selected from: a Spodoptera desaturase, such as a Spodoptera litura desaturase, for example Desat38 as set forth in SEQ ID NO: 3, or such as a Spodoptera littoralis desaturase, for example Desat20 as set forth in SEQ ID NO: 5, or such as a Spodoptera exigua desaturase, for example Desat37 as set forth in SEQ ID NO: 31; an Amyelois desaturase, such as an Amyelois transitella desaturase, for example Desat16 as set forth in SEQ ID NO: 7, an Agrotis desaturase, such as an Agrotis segetum desaturase, for example Desat19 as set forth in SEQ ID NO: 9; a Trichoplusia desaturase, such as a Trichoplusia ni desaturase, for example Desat21 as set forth in SEQ ID NO: 11; a Plutella desaturase, such as a Plutella xylostella desaturase, for example Desat45 as set forth in SEQ ID NO: 33; a Helicoverpa desaturase, such as a Helicoverpa zea desaturase, for example Desat51 as set forth in SEQ ID NO: 29; a Chilo desaturase, such as a Chilo suppressalis desaturase, for example Desat44 as set forth in SEQ ID NO: 51; a Diatraea desaturase, such as a Diatraea saccharalis desaturase, for example Desat63 as set forth in SEQ ID NO: 59; a Plodia desaturase, such as a Plodia interpunctella desaturase, for example Desat65 as set forth in SEQ ID NO: 61; a Lobesia desaturase, such as a Lobesia botrana desaturase, for example Desat71 as set forth in SEQ ID NO: 63 or Desat79 as set forth in SEQ ID NO: 65; an Antheraea desaturase, such as an Antheraea pernyi desaturase, for example Desat72 as set forth in SEQ ID NO: 67; a Cadra desaturase, such as a Cadra cautella desaturase, for example Desat70 as set forth in SEQ ID NO: 69; a Yponomeuta desaturase, such as a Yponomeuta padella desaturase, for example Desat73 as set forth in SEQ ID NO: 71;
or functional variants thereof having at least 60% identity or similarity thereto.

It will be understood that a functional variant of a desaturase, such as a Δ13 desaturase and/or a further desaturase, having at least 60% identity or similarity to a given FAR or desaturase as detailed above may have at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity or similarity to the desaturase.

### Increased C16 Specificity

Many desirable pheromone compounds have a carbon chain length of 16 or longer, such as 17, such as 18, such as 19, such as 20, such as 22, such as 24 and longer. It may thus be of interest to direct the reaction towards the production of pheromone compounds having a carbon chain length of 16 or longer. In some embodiments, the yeast cell disclosed herein expresses a desaturase having a higher specificity towards hexadecanoyl-CoA, heptadecanoyl-CoA, octadecanoyl-CoA, nonadecanoyl-CoA, icosanoyl-CoA, docosanoyl-CoA or tetracosanoyl-CoA, than towards tetradecanoyl-CoA and/or a fatty acyl-CoA reductase having a higher specificity towards desaturated hexadecanoyl-CoA (i.e. hexadecenoyl-CoA), desaturated heptadecanoyl-CoA (i.e. heptadecenoyl-CoA), desaturated octadecanoyl-CoA (i.e. octadecenoyl-CoA), desaturated nonadecanoyl-CoA (i.e. nonadecenoyl-CoA), desaturated icosanoyl-CoA (i.e. icosenoyl-CoA), desaturated docosanoyl-CoA (i.e. docosenoyl-CoA) and desaturated tetracosanoyl-CoA (i.e. tetracosenoyl-CoA), than towards desaturated tetradecanoyl-CoA (i.e. tetradecanoyl-CoA). In other words, the desaturase is more specific for substrates having a carbon chain length of 16 or longer, than for substrates having a carbon chain length of 14.

Expression of such desaturases (and of any of the reductases described herein) in the yeast cell increases the fraction of total desaturated fatty alcohols having a carbon chain length of 16 or longer, particularly compared to the fraction of total desaturated fatty alcohols having a carbon chain length of 14. Desaturases which have the required specificity are in particular desaturases native to Helicoverpa, such as Helicoverpa zea, or variants thereof having at least 60% identity thereto.

Such desaturases preferentially catalyse desaturation of substrates having a carbon chain length of 16 or longer, such as 17, such as 18, such as 19, such as 20, such as 22, such as 24 or longer when expressed in a yeast cell.

In such cells, the ratio of desaturated hexadecanoyl-CoA, desaturated heptadecanoyl-CoA, desaturated octadecanoyl-CoA, desaturated nonadecanoyl-CoA, desaturated icosanoyl-CoA, desaturated docosanoyl-CoA, or desaturated tetracosanoyl-CoA to desaturated tetradecanoyl-CoA, is of at least 0.1, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

When the heterologous desaturase is expressed with a fatty acyl-CoA reductase, as described in detail below, the yeast cell produces desaturated fatty alcohols. In some embodiments, the produced desaturated fatty alcohols may have a carbon chain consisting solely of single and double bonds.

In such embodiments, the titre of desaturated fatty alcohols derived from desaturated fatty acyl-CoAs, having a carbon chain length of 16 or longer, such as desaturated hexadecanoyl-CoA, desaturated heptadecanoyl-CoA, desaturated octadecanoyl-CoA, desaturated nonadecanoyl-CoA, desaturated icosanoyl-CoA, desaturated docosanoyl-CoA, and desaturated tetracosanoyl-CoA is of at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, or more.

In some embodiments, the titre of desaturated fatty alcohols derived from desaturated fatty acyl-CoA having a chain length of 16 or longer is at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, or more.

In some embodiments, desaturated fatty alcohols are yielded comprising at least 1% of a desaturated fatty alcohol having a chain length of 16, such as at least 1.5%, such as at least 2%, such as at least 2.5%, such as at least 3%, such as at least 3.5%, such as at least 4%, such as at least 4.5%, such as at least 5%, such as at least 7.5%, such as at least 10%, or more. In some embodiments, the yeast cell produces desaturated fatty alcohols having a range of carbon chain lengths, including desaturated fatty alcohols having a carbon chain length of 14 and desaturated fatty alcohols having a carbon chain length of 16. In such embodiments, the proportion of desaturated fatty alcohols having a carbon chain length of 16 relative to the sum of desaturated fatty alcohols having a carbon chain length of 14 and desaturated fatty alcohols having a carbon chain length of 16 is at least 5%, such as at least 7.5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 65%, such as at least 70%, such as at least 75%, or more.

These desaturases have been found to preferentially catalyse desaturation of C16 substrates when expressed in a yeast cell.

In such cells, the ratio of desaturated hexadecanoyl-CoA to desaturated tetradecanoyl-CoA is of at least 0.1, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

When the heterologous desaturase is expressed with a fatty acyl-CoA reductase, as described in detail below, the yeast cell produces desaturated fatty alcohols.

In such embodiments, the titre of desaturated fatty alcohols derived from the desaturated hexadecanoyl-CoA is of at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, or more.

In some embodiments, the titre of desaturated fatty alcohols derived from desaturated hexadecanoyl-CoA having a chain length of 16 is at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, or more.

In some embodiments, desaturated fatty alcohols are yielded comprising at least 1% of a desaturated fatty alcohol having a chain length of 16, such as at least 1.5%, such as at least 2%, such as at least 2.5%, such as at least 3%, such as at least 3.5%, such as at least 4%, such as at least 4.5%, such as at least 5%, such as at least 7.5%, such as at least 10%, or more. In some embodiments, the yeast cell produces desaturated fatty alcohols having a range of carbon chain lengths, including desaturated fatty alcohols having a carbon chain length of 14 and desaturated fatty alcohols having a carbon chain length of 16. In such embodiments, the proportion of desaturated fatty alcohols having a carbon chain length of 16 relative to the sum of desaturated fatty alcohols having a carbon chain length of 14 and desaturated fatty alcohols having a carbon chain length of 16 is at least 5%, such as at least 7.5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 65%, such as at least 70%, such as at least 75%, or more.

How to test whether a given desaturase has the required specificity can be done as described herein.

In some embodiments, the desaturase is selected from the groups consisting of:
i) a 411 desaturase having at least 60% identity to the Δ11 desaturase from Spodoptera litura as set forth in SEQ ID NO: 3 (Desat38);
ii) a Δ11 desaturase having at least 60% identity to the Δ11 desaturase from Spodoptera littoralis as set forth in SEQ ID NO: 5 (Desat20);
iii) a desaturase having at least 60% identity to the Δ11 desaturase from Amyelois transitella as set forth in SEQ ID NO: 7 (Desat16);
iv) a desaturase having at least 60% identity to the Δ11 desaturase from Agrotis segetum as set forth in SEQ ID NO: 9 (Desat19);
v) a Δ11 desaturase having at least 60% identity to the Δ11 desaturase from Trichoplusia ni as set forth in SEQ ID NO: 11 (Desat21);
vi) a Δ11 desaturase having at least 60% identity to the Δ11 desaturase from Spodoptera exigua as set forth in SEQ ID NO: 31 (Desat37);
vii) a Δ11 desaturase having at least 60% identity to the Δ11 desaturase from Plutella xylostella as set forth in SEQ ID NO: 33 (Desat45);
viii) a Δ11 desaturase having at least 60% identity to the Δ11 desaturase from Helicoverpa zea as set forth in SEQ ID NO: 29 (Desat51);
ix) a Δ11 desaturase having at least 60% identity to the Δ11 desaturase from Chilo suppressalis as set forth in SEQ ID NO: 51 (Desat44);
x) a Δ11 desaturase having at least 60% identity to the Δ11 desaturase from Diatraea saccharalis as set forth in SEQ ID NO: 59 (Desat63);
xi) a Δ11 desaturase having at least 60% identity to the Δ11 desaturase from Plodia interpunctella as set forth in SEQ ID NO: 61 (Desat65);
xii) a Δ11 desaturase having at least 60% identity to the Δ11 desaturase from Lobesia botrana as set forth in SEQ ID NO: 63 (Desat71) and/or SEQ ID NO: 65 (Desat79);
xiii) a Δ11 desaturase having at least 60% identity to the Δ11 desaturase from Antheraea pernyi as set forth in SEQ ID NO: 67 (Desat72);
xiv) a Δ11 desaturase having at least 60% identity to the Δ11 desaturase from Cadra cautella as set forth in SEQ ID NO: 69 (Desat70);
XV) a Δ11 desaturase having at least 60% identity to the Δ11 desaturase from Yponomeuta padella as set forth in SEQ ID NO: 71 (Desat73);
   and functional variants thereof having at least 60% identity or similarity thereto, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity or similarity thereto.

### Fatty Acyl-CoA Reductase

Further provided herein is a yeast cell expressing a fatty acyl-CoA reductase (FAR), said yeast cell being capable of converting at least a part of the (Z13)-fatty acyl-CoA or (Z11, Z13)-fatty acyl-CoA into the corresponding fatty alcohol. For example, the yeast cell may be capable of converting at least part of (Z11, Z13)-hexadecadienoyl-CoA into (Z11, Z13)-hexadecadien-1-ol. In some embodiments, the produced corresponding fatty alcohol(s) may have a carbon chain consisting solely of single and double bonds.

The terms ‘fatty acyl-CoA reductase’ and ‘FAR’ will be used herein interchangeably. The term ‘heterologous FAR’ refers to a FAR which is not naturally expressed by the organism, such as by the yeast cell.

### FARs Catalyze the Two-Step Reaction:

Acyl-CoA+2 NADPH<=>CoA+alcohol+2 NADP(+)

wherein in a first step, the fatty acyl-CoA is reduced to a fatty aldehyde, before the fatty aldehyde is further reduced into a fatty alcohol in a second step. The fatty acyl-CoA may be a desaturated or a saturated fatty acyl-CoA. In some embodiments, the desaturated fatty acyl-CoA may have a carbon chain consisting solely of single and double bonds.

The FARs capable of catalyzing such reaction are alcohol-forming fatty acyl-CoA reductases with an EC number 1.2.1.84.

In one embodiment, the FAR is native to an organism of a genus selected from the group consisting of Agrotis, Amyelois, Bicyclus, Bombus, Chilo, Cydia, Helicoverpa, Heliothis, Lobesia, Ostrinia, Plodia, Plutella, Spodoptera, Trichoplusia, Tyta, Yponomeuta, Ostrinia, Marinobacter, Manducta, Chrysodeixis, and Grapholita, such as Agrotis ipsilon, Agrotis segetum, Amyelois transitella, Bicyclus anynana, Bombus lapidarius, Chilo suppressalis, Cydia pomonella, Helicoverpa armigera, Helicoverpa assulta, Heliothis subflexa, Heliothis virescens, Lobesia botrana, Ostrinia nubilalis, Plodia interpunctella, Plutella xylostella, Spodoptera exigua, Spodoptera frugiperda, Spodoptera littoralis, Spodoptera litura, Trichoplusia ni, Tyta alba, Yponomeuta rorellus, Ostrinia nubilalis, Ostrinia furnacalis, Ostrinia zag, Ostrinia zea, Marinobacter algicola, Manducta sexta, Chrysodeixis includes, and Grapholita molesta, especially Spodoptera exigua, Helicoverpa armigera, Spodoptera litura, Amyelois transitella and Agrotis ipsilon.

In one embodiment, the FAR is selected from the group consisting of FAR1 (SEQ ID NO: 13), FAR4 (SEQ ID NO: 19), FAR6 (SEQ ID NO: 21), FAR33 (SEQ ID NO: 15), FAR16 (SEQ ID NO: 37), FAR17 (SEQ ID NO: 41), FAR19 (SEQ ID NO: 39), FAR28 (SEQ ID NO: 45), FAR32 (SEQ ID NO: 35), FAR38 (SEQ ID NO: 43), FAR5 (SEQ ID NO: 73), FAR8 (SEQ ID NO: 75), FAR9 (SEQ ID NO: 77), FAR12 (SEQ ID NO: 79), FAR42 (SEQ ID NO: 81), FAR25 (SEQ ID NO: 83), FAR13 (SEQ ID NO: 85), FAR14 (SEQ ID NO: 87), FAR15 (SEQ ID NO: 89), FAR18 (SEQ ID NO: 91), FAR20 (SEQ ID NO: 93), FAR22 (SEQ ID NO: 95), FAR27 (SEQ ID NO: 97), FAR29 (SEQ ID NO: 99), FAR30 (SEQ ID NO: 101), FAR43 (SEQ ID NO: 103), FAR34 (SEQ ID NO: 107), FAR35 (SEQ ID NO: 109), FAR44 (SEQ ID NO: 111), FAR48 (SEQ ID NO: 113), FAR49 (SEQ ID NO: 115), FAR45 (SEQ ID NO: 117), FAR40 (SEQ ID NO: 119), FAR41 (SEQ ID NO: 121), FAR51 (SEQ ID NO: 123), FAR52 (SEQ ID NO: 125), FAR47 (SEQ ID NO: 127), FAR46 (SEQ ID NO: 129), and FAR50 (SEQ ID NO: 131) or functional variants thereof having at least 60% identity or similarity thereto, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity or similarity thereto.

In one embodiment, the heterologous FAR is a Helicoverpa FAR. In one embodiment, the FAR is a Helicoverpa armigera FAR. In one embodiment, the FAR is a Helicoverpa armigera FAR, such as the FAR as set forth in SEQ ID NO: 13 (FAR1). In some embodiments, the FAR is a functional variant of a Helicoverpa FAR, or a functional variant of a Helicoverpa armigera FAR, such as a functional variant of the FAR set forth in SEQ ID NO: 13 (FAR1), having at least 60% identity thereto. In one embodiment, the FAR is a Helicoverpa assulta FAR. In one embodiment, the FAR is a Helicoverpa assulta FAR, such as the FAR as set forth in SEQ ID NO: 21 (FAR6). In some embodiments, the FAR is a functional variant of a Helicoverpa FAR, or a functional variant of a Helicoverpa assulta FAR, such as a functional variant of the FAR set forth in SEQ ID NO: 21 (FAR6), having at least 60% identity thereto.

In one embodiment, the heterologous FAR is an Amyelois FAR. In one embodiment, the FAR is an Amyelois transitella FAR. In one embodiment, the FAR is an Amyelois transitella FAR, such as the FAR as set forth in SEQ ID NO: 15 (FAR33), SEQ ID NO: 107 (FAR34) or SEQ ID NO: 109 (FAR35). In some embodiments, the FAR is a functional variant of an Amyelois FAR, or a functional variant of an Amyelois transitella FAR, such as a functional variant of the FAR set forth in SEQ ID NO: 15 (FAR33), SEQ ID NO: 107 (FAR34) or SEQ ID NO: 109 (FAR35), having at least 60% identity thereto.

In one embodiment, the heterologous FAR is a Heliothis FAR. In one embodiment, the FAR is a Heliothis subflexa FAR. In one embodiment, the FAR is a Heliothis subflexa FAR, such as the FAR as set forth in SEQ ID NO: 19 (FAR4). In one embodiment, the FAR is a Heliothis virescens FAR, such as the FAR as set forth in SEQ ID NO: 73 (FAR5). In some embodiments, the FAR is a functional variant of an Heliothis FAR, or a functional variant of an Heliothis subflexa FAR, such as a functional variant of the FAR set forth in SEQ ID NO: 19 (FAR4), or SEQ ID NO: 73 (FAR5), having at least 60% identity thereto.

In one embodiment, the heterologous FAR is a Plodia FAR. In one embodiment, the FAR is a Plodia interpunctella FAR. In one embodiment, the FAR is a Plodia interpunctella FAR, such as the FAR as set forth in SEQ ID NO: 45 (FAR28), SEQ ID NO: 35 (FAR32), SEQ ID NO: 99 (FAR29) or SEQ ID NO: 101 (FAR30). In some embodiments, the FAR is a functional variant of a Plodia FAR, or a functional variant of a Plodia interpunctella FAR, such as a functional variant of the FAR set forth in SEQ ID NO: 45 (FAR28), SEQ ID NO: 35 (FAR32), SEQ ID NO: 99 (FAR29) or SEQ ID NO: 101 (FAR30), having at least 60% identity thereto.

In one embodiment, the heterologous FAR is a Spodoptera FAR. In one embodiment, the FAR is a Spodoptera exigua FAR, such as the FAR as set forth in SEQ ID NO: 37 (FAR16), SEQ ID NO: 41 (FAR17) or SEQ ID NO: 117 (FAR45). In one embodiment, the FAR is a Spodoptera litura FAR, such as the FAR as set forth in SEQ ID NO: 39 (FAR19). In one embodiment, the FAR is a Spodoptera littoralis FAR, such as the FAR as set forth in SEQ ID NO: 89 (FAR15). In one embodiment, the FAR is a Spodoptera frugiperda FAR, such as the FAR as set forth in SEQ ID NO: 93 (FAR20) or SEQ ID NO: 95 (FAR22). In some embodiments, the FAR is a functional variant of a Spodoptera FAR, a functional variant of a Spodoptera exigua FAR, a functional variant of a Spodoptera litura FAR, a functional variant of a Spodoptera littoralis FAR, a functional variant of a Spodoptera frugiperda FAR, a functional variant of the FAR set forth in SEQ ID NO: 37 (FAR16), SEQ ID NO: 41 (FAR17), SEQ ID NO: 117 (FAR45), SEQ ID NO: 39 (FAR19), SEQ ID NO: 89 (FAR15), SEQ ID NO: 93 (FAR20) or SEQ ID NO: 95 (FAR22), having at least 60% identity thereto.

In one embodiment, the heterologous FAR is a Trichoplusia FAR. In one embodiment, the FAR is a Trichoplusia ni FAR. In one embodiment, the FAR is a Trichoplusia ni FAR, such as the FAR as set forth in SEQ ID NO: 43 (FAR38), SEQ ID NO: 119 (FAR40), SEQ ID NO: 121 (FAR41) or SEQ ID NO: 123 (FAR51). In some embodiments, the FAR is a functional variant of a Trichoplusia FAR, or a functional variant of a Trichoplusia ni FAR, such as a functional variant of the FAR set forth in SEQ ID NO: 43 (FAR38), SEQ ID NO: 119 (FAR40), SEQ ID NO: 121 (FAR41) or SEQ ID NO: 123 (FAR51), having at least 60% identity thereto.

In one embodiment, the heterologous FAR is an Yponomeuta FAR. In one embodiment, the FAR is an Yponomeuta rorellus FAR. In one embodiment, the FAR is an Yponomeuta rorellus FAR, such as the FAR as set forth in SEQ ID NO: 75 (FAR8). In some embodiments, the FAR is a functional variant of an Yponomeuta FAR, or a functional variant of an Yponomeuta rorellus FAR, such as a functional variant of the FAR set forth in SEQ ID NO: 75 (FAR8), having at least 60% identity thereto.

In one embodiment, the heterologous FAR is an Ostrinia FAR. In one embodiment, the FAR is an Ostrinia nubilalis FAR, such as the FAR as set forth in SEQ ID NO: 77 (FAR9). In one embodiment, the FAR is an Ostrinia nubilalis (E-strain) FAR. In one embodiment, the FAR is an Ostrinia furnacalis FAR, such as the FAR as set forth in SEQ ID NO: 111 (FAR44). In one embodiment, the FAR is an Ostrinia zag FAR, such as the FAR as set forth in SEQ ID NO: 113 (FAR48). In one embodiment, the FAR is an Ostrinia zea FAR, such as the FAR as set forth in SEQ ID NO: 115 (FAR49). In some embodiments, the FAR is a functional variant of an Ostrinia FAR, an Ostrinia nubilalis FAR, an Ostrinia nubilalis (E-strain), an Ostrinia furnacalis FAR, an Ostrinia zag FAR, an Ostrinia zea FAR, or a functional variant of the FAR set forth in SEQ ID NO: 77 (FAR9), SEQ ID NO: 111 (FAR44), SEQ ID NO: 113 (FAR48) or SEQ ID NO: 115 (FAR49), having at least 60% identity thereto.

In one embodiment, the heterologous FAR is an Agrotis FAR. In one embodiment, the FAR is an Agrotis segetum FAR. In one embodiment, the FAR is an Agrotis segetum FAR, such as the FAR as set forth in SEQ ID NO: 79 (FAR12). In one embodiment, the FAR is an Agrotis ipsilon FAR. In one embodiment, the FAR is an Agrotis ipsilon FAR, such as the FAR as set forth in SEQ ID NO: 91 (FAR18). In some embodiments, the FAR is a functional variant of an Agrotis FAR, an Agrotis segetum FAR, an Agrotis ipsilon FAR or a functional variant of the FAR set forth in SEQ ID NO: 79 (FAR12) or SEQ ID NO: 91 (FAR18), having at least 60% identity thereto.

In one embodiment, the heterologous FAR is a Marinobacter FAR. In one embodiment, the FAR is a Marinobacter algicola FAR. In one embodiment, the FAR is a Marinobacter algicola FAR, such as the FAR as set forth in SEQ ID NO: 81 (FAR42). In some embodiments, the FAR is a functional variant of a Marinobacter FAR, or a functional variant of a Marinobacter algicola FAR, such as a functional variant of the FAR set forth in SEQ ID NO: 81 (FAR42), having at least 60% identity thereto.

In one embodiment, the heterologous FAR is a Tyta FAR. In one embodiment, the FAR is a Tyta alba FAR. In one embodiment, the FAR is a Tyta alba FAR, such as the FAR as set forth in SEQ ID NO: 83 (FAR25). In some embodiments, the FAR is a functional variant of a Tyta FAR, or a functional variant of a Tyta alba FAR, such as a functional variant of the FAR set forth in SEQ ID NO: 83 (FAR25), having at least 60% identity thereto.

In one embodiment, the heterologous FAR is a Chilo FAR. In one embodiment, the FAR is a Chilo suppressalis FAR. In one embodiment, the FAR is a Chilo suppressalis FAR, such as the FAR as set forth in SEQ ID NO: 85 (FAR13). In some embodiments, the FAR is a functional variant of a Chilo FAR, or a functional variant of a Chilo suppressalis FAR, such as a functional variant of the FAR set forth in SEQ ID NO: 85 (FAR13), having at least 60% identity thereto.

In one embodiment, the heterologous FAR is a Bombus FAR. In one embodiment, the FAR is a Bombus lapidarius FAR. In one embodiment, the FAR is a Bombus lapidarius FAR, such as the FAR as set forth in SEQ ID NO: 87 (FAR14). In some embodiments, the FAR is a functional variant of a Bombus FAR, or a functional variant of a Bombus lapidarius FAR, such as a functional variant of the FAR set forth in SEQ ID NO: 87 (FAR14), having at least 60% identity thereto.

In one embodiment, the heterologous FAR is a Plutella FAR. In one embodiment, the FAR is a Plutella xylostella FAR. In one embodiment, the FAR is a Plutella xylostella FAR, such as the FAR as set forth in SEQ ID NO: 97 (FAR27). In some embodiments, the FAR is a functional variant of a Plutella FAR, or a functional variant of a Plutella xylostella FAR, such as a functional variant of the FAR set forth in SEQ ID NO: 97 (FAR27), having at least 60% identity thereto.

In one embodiment, the heterologous FAR is a Manducta FAR. In one embodiment, the FAR is a Manducta sexta FAR. In one embodiment, the FAR is a Manducta sexta FAR, such as the FAR as set forth in SEQ ID NO: 103 (FAR43). In some embodiments, the FAR is a functional variant of a Manducta FAR, or a functional variant of a Manducta sexta FAR, such as a functional variant of the FAR set forth in SEQ ID NO: 103 (FAR43), having at least 60% identity thereto.

In one embodiment, the heterologous FAR is a Chrysodeixis FAR. In one embodiment, the FAR is a Chrysodeixis includens FAR. In one embodiment, the FAR is a Chrysodeixis includens FAR, such as the FAR as set forth in SEQ ID NO: 125 (FAR52) or SEQ ID NO: 127 (FAR47). In some embodiments, the FAR is a functional variant of a Chrysodeixis FAR, or a functional variant of a Chrysodeixis includens FAR, such as a functional variant of the FAR set forth in SEQ ID NO: 125 (FAR52) or SEQ ID NO: 127 (FAR47), having at least 60% identity thereto.

In one embodiment, the heterologous FAR is a Cydia FAR. In one embodiment, the FAR is a Cydia pomonella FAR. In one embodiment, the FAR is a Cydia pomonella FAR, such as the FAR as set forth in SEQ ID NO: 129 (FAR46). In some embodiments, the FAR is a functional variant of a Cydia FAR, or a functional variant of a Cydia pomonella FAR, such as a functional variant of the FAR set forth in SEQ ID NO: 129 (FAR46), having at least 60% identity thereto.

In one embodiment, the heterologous FAR is a Grapholita FAR. In one embodiment, the FAR is a Grapholita molesta FAR. In one embodiment, the FAR is a Grapholita molesta FAR, such as the FAR as set forth in SEQ ID NO: 131 (FAR50). In some embodiments, the FAR is a functional variant of a Grapholita FAR, or a functional variant of a Grapholita molesta FAR, such as a functional variant of the FAR set forth in SEQ ID NO: 131 (FAR50), having at least 60% identity thereto.

The term “variant thereof having at least 60% identity or similarity” in relation to a given enzyme shall be understood to refer to variants having 60% identity or similarity or more to said enzyme, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity or similarity to the enzyme, or more.

The present cells may express at least one heterologous FAR. In some embodiments, the cell expresses one heterologous FAR. It may however be desirable to express several heterologous FARs, such as at least two heterologous FARs, which may be identical or different. Alternatively, it may be desirable to express several copies of the nucleic acid encoding the at least one heterologous FAR, such as at least two copies, at least three copies or more. In other embodiments, the cell expresses at least two heterologous FARs, for example three heterologous FARs.

For example, the cell may express two copies of FAR1 or a variant thereof; or one copy of FAR1 and one copy of FAR 16; or two copies of FAR1, one copy of FAR 16 and one copy of FAR19.

### Desaturases and FARs

Any of the above FARs can be expressed together with any desaturase, in particular any of the desaturases described herein.

In some embodiments, the cell expresses:
an Amyelois Δ13 desaturase, such as an Amyelois transitella Δ13 desaturase, for example AtrAATQ as set forth in SEQ ID NO: 1, and
a further desaturase selected from: a Spodoptera desaturase, such as a Spodoptera litura desaturase, for example Desat38 as set forth in SEQ ID NO: 3, or such as a Spodoptera littoralis desaturase, for example Desat20 as set forth in SEQ ID NO: 5, or such as a Spodoptera exigua desaturase, for example Desat37 as set forth in SEQ ID NO: 31; an Amyelois desaturase, such as an Amyelois transitella desaturase, for example Desat16 as set forth in SEQ ID NO: 7, an Agrotis desaturase, such as an Agrotis segetum desaturase, for example Desat19 as set forth in SEQ ID NO: 9; a Trichoplusia desaturase, such as a Trichoplusia ni desaturase, for example Desat21 as set forth in SEQ ID NO: 11; a Plutella desaturase, such as a Plutella xylostella desaturase, for example Desat45 as set forth in SEQ ID NO: 33; a Helicoverpa desaturase, such as a Helicoverpa zea desaturase, for example Desat51 as set forth in SEQ ID NO: 29; a Chilo desaturase, such as a Chilo suppressalis desaturase, for example Desat44 as set forth in SEQ ID NO: 51; a Diatraea desaturase, such as a Diatraea saccharalis desaturase, for example Desat63 as set forth in SEQ ID NO: 59; a Plodia desaturase, such as a Plodia interpunctella desaturase, for example Desat65 as set forth in SEQ ID NO: 61; a Lobesia desaturase, such as a Lobesia botrana desaturase, for example Desat71 as set forth in SEQ ID NO: 63 or Desat79 as set forth in SEQ ID NO: 65; an Antheraea desaturase, such as an Antheraea pernyi desaturase, for example Desat72 as set forth in SEQ ID NO: 67; a Cadra desaturase, such as a Cadra cautella desaturase, for example Desat70 as set forth in SEQ ID NO: 69; a Yponomeuta desaturase, such as a Yponomeuta padella desaturase, for example Desat73 as set forth in SEQ ID NO: 71; and/or
a FAR selected from: a Helicoverpa FAR, such as a Helicoverpa armigera FAR, for example FAR1 as set forth in SEQ ID NO: 13, or a Helicoverpa assulta FAR, for example FAR6 as set forth in SEQ ID NO: 21; a Heliothis FAR, such as a Heliothis subflexa FAR, for example FAR4 as set forth in SEQ ID NO: 19, or a Heliothis virescens FAR, for example FAR5 as set forth in SEQ ID NO: 73; an Amyelois FAR, such as an Amyelois transitella FAR, for example FAR33 as set forth in SEQ ID NO: 15, FAR34 as set forth in SEQ ID NO: 107 or FAR35 as set forth in SEQ ID NO: 109; a Plodia FAR, such as a Plodia interpunctella FAR, for example FAR28 as set forth in SEQ ID NO: 45, FAR32 as set forth in SEQ ID NO: 35, FAR29 as set forth in SEQ ID NO: 99 or FAR30 as set forth in SEQ ID NO: 101; a Spodoptera FAR, such as a Spodoptera exigua FAR, for example FAR16 as set forth in SEQ ID NO: 37, FAR17 as set forth in SEQ ID NO: 41, or FAR45 as set forth in SEQ ID NO: 117, or such as a Spodoptera litura FAR, for example FAR19 as set forth in SEQ ID NO: 39, or such as a Spodoptera littoralis FAR, for example FAR15 as set forth in SEQ ID NO: 89, or such as a Spodoptera frugiperda FAR, for example FAR20 as set forth in SEQ ID NO: 93 or FAR22 as set forth in SEQ ID NO: 95; a Trichoplusia FAR, such as a Trichoplusia ni FAR, for example FAR38 as set forth in SEQ ID NO: 43, FAR40 as set forth in SEQ ID NO: 119, FAR41 as set forth in SEQ ID NO: 121 or FAR51 as set forth in SEQ ID NO: 123; an Yponomeuta FAR, such as an Yponomeuta rorellus FAR, for example FAR8 as set forth in SEQ ID NO: 75; an Ostrinia FAR, such as an Ostrinia nubilalis FAR, for example FAR9 as set forth in SEQ ID NO: 77, or such as an Ostrinia furnacalis FAR, for example FAR44 as set forth in SEQ ID NO: 111, or such as an Ostrinia zag FAR, for example FAR48 as set forth in SEQ ID NO: 113, or such as an Ostrinia zea FAR, for example FAR49 as set forth in SEQ ID NO: 115; an Agrotis FAR, such as an Agrotis segetum FAR, for example FAR 12 as set forth in SEQ ID NO: 79, or such as an Agrotis ipsilon FAR, for example FAR18 as set forth in SEQ ID NO: 91; a Marinobacter FAR, such as a Marinobacter algicola FAR, for example FAR42 as set forth in SEQ ID NO: 81; a Tyta FAR, such as a Tyta alba FAR, for example FAR25 as set forth in SEQ ID NO: 83; a Chilo FAR, such as a Chilo suppressalis FAR, for example FAR13 as set forth in SEQ ID NO: 85; a Bombus FAR, such as a Bombus lapidarius FAR, for example FAR14 as set forth in SEQ ID NO: 87; a Plutella FAR, such as a Plutella xylostella FAR, for example FAR27 as set forth in SEQ ID NO: 97; a Manducta FAR, such as a Manducta sexta FAR, for example FAR43 as set forth in SEQ ID NO: 103; a Chrysodeixis FAR, such as a Chrysodeixis includens FAR, for example FAR52 as set forth in SEQ ID NO: 125 and/or FAR47 as set forth in SEQ ID NO: 127; a Cydia FAR, such as a Cydia pomonella FAR, for example FAR46 as set forth in SEQ ID NO: 129; a Grapholita FAR, such as a Grapholita molesta FAR, for example FAR50 as set forth in SEQ ID NO: 131;
   or functional variants thereof having at least 60% identity or similarity thereto.

In some embodiments, the cell expresses:
a Notodonta Δ13 desaturase, such as a Notodonta dromedarius Δ13 desaturase, for example NdDes1 as set forth in SEQ ID NO: 56, and
a further desaturase selected from: a Spodoptera desaturase, such as a Spodoptera litura desaturase, for example Desat38 as set forth in SEQ ID NO: 3, or such as a Spodoptera littoralis desaturase, for example Desat20 as set forth in SEQ ID NO: 5, or such as a Spodoptera exigua desaturase, for example Desat37 as set forth in SEQ ID NO: 31; an Amyelois desaturase, such as an Amyelois transitella desaturase, for example Desat16 as set forth in SEQ ID NO: 7, an Agrotis desaturase, such as an Agrotis segetum desaturase, for example Desat19 as set forth in SEQ ID NO: 9; a Trichoplusia desaturase, such as a Trichoplusia ni desaturase, for example Desat21 as set forth in SEQ ID NO: 11; a Plutella desaturase, such as a Plutella xylostella desaturase, for example Desat45 as set forth in SEQ ID NO: 33; a Helicoverpa desaturase, such as a Helicoverpa zea desaturase, for example Desat51 as set forth in SEQ ID NO: 29; a Chilo desaturase, such as a Chilo suppressalis desaturase, for example Desat44 as set forth in SEQ ID NO: 51; a Diatraea desaturase, such as a Diatraea saccharalis desaturase, for example Desat63 as set forth in SEQ ID NO: 59; a Plodia desaturase, such as a Plodia interpunctella desaturase, for example Desat65 as set forth in SEQ ID NO: 61; a Lobesia desaturase, such as a Lobesia botrana desaturase, for example Desat71 as set forth in SEQ ID NO: 63 or Desat79 as set forth in SEQ ID NO: 65; an Antheraea desaturase, such as an Antheraea pernyi desaturase, for example Desat72 as set forth in SEQ ID NO: 67; a Cadra desaturase, such as a Cadra cautella desaturase, for example Desat70 as set forth in SEQ ID NO: 69; a Yponomeuta desaturase, such as a Yponomeuta padella desaturase, for example Desat73 as set forth in SEQ ID NO: 71; and/or
a FAR selected from: a Helicoverpa FAR, such as a Helicoverpa armigera FAR, for example FAR1 as set forth in SEQ ID NO: 13, or a Helicoverpa assulta FAR, for example FAR6 as set forth in SEQ ID NO: 21; a Heliothis FAR, such as a Heliothis subflexa FAR, for example FAR4 as set forth in SEQ ID NO: 19, or a Heliothis virescens FAR, for example FAR5 as set forth in SEQ ID NO: 73; an Amyelois FAR, such as an Amyelois transitella FAR, for example FAR33 as set forth in SEQ ID NO: 15, FAR34 as set forth in SEQ ID NO: 107 or FAR35 as set forth in SEQ ID NO: 109; a Plodia FAR, such as a Plodia interpunctella FAR, for example FAR28 as set forth in SEQ ID NO: 45, FAR32 as set forth in SEQ ID NO: 35, FAR29 as set forth in SEQ ID NO: 99 or FAR30 as set forth in SEQ ID NO: 101; a Spodoptera FAR, such as a Spodoptera exigua FAR, for example FAR16 as set forth in SEQ ID NO: 37, FAR17 as set forth in SEQ ID NO: 41, or FAR45 as set forth in SEQ ID NO: 117, or such as a Spodoptera litura FAR, for example FAR19 as set forth in SEQ ID NO: 39, or such as a Spodoptera littoralis FAR, for example FAR15 as set forth in SEQ ID NO: 89, or such as a Spodoptera frugiperda FAR, for example FAR20 as set forth in SEQ ID NO: 93 or FAR22 as set forth in SEQ ID NO: 95; a Trichoplusia FAR, such as a Trichoplusia ni FAR, for example FAR38 as set forth in SEQ ID NO: 43, FAR40 as set forth in SEQ ID NO: 119, FAR41 as set forth in SEQ ID NO: 121 or FAR51 as set forth in SEQ ID NO: 123; an Yponomeuta FAR, such as an Yponomeuta rorellus FAR, for example FAR8 as set forth in SEQ ID NO: 75; an Ostrinia FAR, such as an Ostrinia nubilalis FAR, for example FAR9 as set forth in SEQ ID NO: 77, or such as an Ostrinia furnacalis FAR, for example FAR44 as set forth in SEQ ID NO: 111, or such as an Ostrinia zag FAR, for example FAR48 as set forth in SEQ ID NO: 113, or such as an Ostrinia zea FAR, for example FAR49 as set forth in SEQ ID NO: 115; an Agrotis FAR, such as an Agrotis segetum FAR, for example FAR12 as set forth in SEQ ID NO: 79, or such as an Agrotis ipsilon FAR, for example FAR18 as set forth in SEQ ID NO: 91; a Marinobacter FAR, such as a Marinobacter algicola FAR, for example FAR42 as set forth in SEQ ID NO: 81; a Tyta FAR, such as a Tyta alba FAR, for example FAR25 as set forth in SEQ ID NO: 83; a Chilo FAR, such as a Chilo suppressalis FAR, for example FAR13 as set forth in SEQ ID NO: 85; a Bombus FAR, such as a Bombus lapidarius FAR, for example FAR14 as set forth in SEQ ID NO: 87; a Plutella FAR, such as a Plutella xylostella FAR, for example FAR27 as set forth in SEQ ID NO: 97; a Manducta FAR, such as a Manducta sexta FAR, for example FAR43 as set forth in SEQ ID NO: 103; a Chrysodeixis FAR, such as a Chrysodeixis includens FAR, for example FAR52 as set forth in SEQ ID NO: 125 and/or FAR47 as set forth in SEQ ID NO: 127; a Cydia FAR, such as a Cydia pomonella FAR, for example FAR46 as set forth in SEQ ID NO: 129; a Grapholita FAR, such as a Grapholita molesta FAR, for example FAR50 as set forth in SEQ ID NO: 131;
   or functional variants thereof having at least 60% identity or similarity thereto.

It will be understood that a functional variant of a FAR or a desaturase, such as a 413 desaturase and/or a further desaturase, having at least 60% identity or similarity to a given FAR or desaturase as detailed above may have at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity or similarity to the FAR or the desaturase.

The yeast cell expressing said Δ13 desaturase, further desaturase, and FAR is capable of producing (Z11, Z13)-hexadecadien-1-ol.

### Yeast Cell

The present invention provides a yeast cell which has been modified or engineered to produce desaturated compounds, in particular desaturated fatty acyl-CoAs and desaturated fatty alcohols having at least one double bond at position 13. Some of these are components of pheromones. The yeast cell disclosed herein thus provides an improved platform for environment-friendly pheromone production.

Accordingly, provided herein is a yeast cell capable of producing a desaturated fatty acyl-CoA having n′ double bond(s), wherein at least one of said double bond(s) is at position 13, wherein said yeast cell expresses a heterologous Δ13 desaturase, said desaturase being capable of introducing a double bond at position 13 in a saturated or desaturated fatty acyl-CoA, preferably in a desaturated fatty acyl-CoA, having a carbon chain length of at least 14 and having n double bond(s), wherein n and n′ are integers, wherein 0≤n≤3 and wherein 1≤n′≤4.

In some embodiments, the desaturated fatty acyl-CoA may have a carbon chain consisting solely of single and double bonds.

In preferred embodiments, the saturated fatty acyl-CoA or desaturated fatty acyl-CoA used as a substrate and the desaturated fatty acyl-CoA having n′ double bonds have the same carbon chain length.

Provided herein is also a yeast cell capable of producing a desaturated fatty acyl-CoA having n′ double bond(s), wherein at least one of said double bond(s) is at position 13, wherein said yeast cell expresses a heterologous Δ13 desaturase, said desaturase being capable of introducing a double bond at position 13 in a saturated fatty acyl-CoA or desaturated fatty acyl-CoA having a carbon chain consisting solely of single and double bonds, preferably in a desaturated fatty acyl-CoA, wherein said fatty acyl-CoA has a carbon chain length of at least 14 and has n double bond(s), wherein n and n′ are integers, wherein 0≤n≤3 and wherein 1≤n′≤4.

The heterologous Δ13 desaturase may be as disclosed herein in the section “Δ13 desaturase”. For example, the Δ13 desaturase may be an Amyelois Δ13 desaturase, such as an Amyelois transitella Δ13 desaturase, such as the Δ13 desaturase as set forth in SEQ ID NO: 1 (AtrAATQ), or a functional variant thereof having at least 60% similarity or identity thereto. In some embodiments, the Δ13 desaturase may be a Notodonta Δ13 desaturase, such as a Notodonta dromedarius Δ13 desaturase, such as the Δ13 desaturase as set forth in SEQ ID NO: 56 (NdDes1), or a functional variant thereof having at least 60% similarity or identity thereto.

In one embodiment, the yeast cell disclosed herein expresses a further desaturase that may be as disclosed herein in the section “Further desaturase”. For example, the further desaturase may be a desaturase capable of introducing a double bond in a saturated or desaturated fatty acid at a position other than position 13. In some embodiments, the desaturase is a Spodoptera desaturase, such as a Spodoptera litura desaturase, for example Desat38 as set forth in SEQ ID NO: 3, or such as a Spodoptera littoralis desaturase, for example Desat20 as set forth in SEQ ID NO: 5, or such as a Spodoptera exigua desaturase, for example Desat37 as set forth in SEQ ID NO: 31; an Amyelois desaturase, such as an Amyelois transitella desaturase, for example Desat16 as set forth in SEQ ID NO: 7; an Agrotis desaturase, such as an Agrotis segetum desaturase, for example Desat19 as set forth in SEQ ID NO: 9; a Trichoplusia desaturase, such as a Trichoplusia ni desaturase, for example Desat21 as set forth in SEQ ID NO: 11; a Plutella desaturase, such as a Plutella xylostella desaturase, for example Desat45 as set forth in SEQ ID NO: 33; a Helicoverpa desaturase, such as a Helicoverpa zea desaturase, for example Desat51 as set forth in SEQ ID NO: 29; a Chilo desaturase, such as a Chilo suppressalis desaturase, for example Desat44 as set forth in SEQ ID NO: 51; a Diatraea desaturase, such as a Diatraea saccharalis desaturase, for example Desat63 as set forth in SEQ ID NO: 59; a Plodia desaturase, such as a Plodia interpunctella desaturase, for example Desat65 as set forth in SEQ ID NO: 61; a Lobesia desaturase, such as a Lobesia botrana desaturase, for example Desat71 as set forth in SEQ ID NO: 63 or Desat79 as set forth in SEQ ID NO: 65; an Antheraea desaturase, such as an Antheraea pernyi desaturase, for example Desat72 as set forth in SEQ ID NO: 67; a Cadra desaturase, such as a Cadra cautella desaturase, for example Desat70 as set forth in SEQ ID NO: 69; a Yponomeuta desaturase, such as a Yponomeuta padella desaturase, for example Desat73 as set forth in SEQ ID NO: 71; or functional variants thereof having at least 60% similarity or identity thereto.

The yeast cell expressing a heterologous Δ13 desaturase and a further desaturase is capable of producing a double desaturated fatty acyl-CoA with a double bond at position 13 and a double bond at one other position. In one embodiment, the yeast cell expressing a heterologous Δ13 desaturase and a further heterologous desaturase is capable of producing a fatty acyl-CoA with a double bond at position 13 and at position 11, such as for example (Z11, Z13)-hexadecadienoyl-CoA. In some embodiments, the produced desaturated fatty acyl-CoA may have a carbon chain consisting solely of single and double bonds.

In one embodiment, the yeast cell disclosed herein further expresses a FAR that may be as disclosed herein in the section “Fatty acyl-CoA reductase”. For example, the FAR may be a Helicoverpa FAR, such as a Helicoverpa armigera FAR, for example FAR1 as set forth in SEQ ID NO: 13, or a Helicoverpa assulta FAR, for example FAR6 as set forth in SEQ ID NO: 21; a Heliothis FAR, such as a Heliothis subflexa FAR, for example FAR4 as set forth in SEQ ID NO: 19, or a Heliothis virescens FAR, for example FAR5 as set forth in SEQ ID NO: 73; an Amyelois FAR, such as Amyelois transitella FAR, for example FAR33 as set forth in SEQ ID NO: 15, FAR34 as set forth in SEQ ID NO: 107 or FAR35 as set forth in SEQ ID NO: 109; a Plodia FAR, such as a Plodia interpunctella FAR, for example FAR28 as set forth in SEQ ID NO: 45 or FAR32 as set forth in SEQ ID NO: 35, FAR29 as set forth in SEQ ID NO: 99 or FAR30 as set forth in SEQ ID NO: 101; a Spodoptera FAR, such as a Spodoptera exigua FAR, for example FAR 16 as set forth in SEQ ID NO: 37, FAR17 as set forth in SEQ ID NO: 41, or FAR45 as set forth in SEQ ID NO: 117, or such as a Spodoptera litura FAR, for example FAR19 as set forth in SEQ ID NO: 39, or such as a Spodoptera littoralis FAR, for example FAR15 as set forth in SEQ ID NO: 89, or such as a Spodoptera frugiperda FAR, for example FAR20 as set forth in SEQ ID NO: 93 or FAR22 as set forth in SEQ ID NO: 95; a Trichoplusia FAR, such as a Trichoplusia ni FAR, for example FAR38 as set forth in SEQ ID NO: 43, FAR40 as set forth in SEQ ID NO: 119, FAR41 as set forth in SEQ ID NO: 121 or FAR51 as set forth in SEQ ID NO: 123; an Yponomeuta FAR, such as an Yponomeuta rorellus FAR, for example FAR8 as set forth in SEQ ID NO: 75; an Ostrinia FAR, such as an Ostrinia nubilalis FAR, for example FAR9 as set forth in SEQ ID NO: 77, or such as an Ostrinia furnacalis FAR, for example FAR44 as set forth in SEQ ID NO: 111, or such as an Ostrinia zag FAR, for example FAR48 as set forth in SEQ ID NO: 113, or such as an Ostrinia zea FAR, for example FAR49 as set forth in SEQ ID NO: 115; an Agrotis FAR, such as an Agrotis segetum FAR, for example FAR12 as set forth in SEQ ID NO: 79, or such as an Agrotis ipsilon FAR, for example FAR18 as set forth in SEQ ID NO: 91; a Marinobacter FAR, such as a Marinobacter algicola FAR, for example FAR42 as set forth in SEQ ID NO: 81; a Tyta FAR, such as a Tyta alba FAR, for example FAR25 as set forth in SEQ ID NO: 83; a Chilo FAR, such as a Chilo suppressalis FAR, for example FAR13 as set forth in SEQ ID NO: 85; a Bombus FAR, such as a Bombus lapidarius FAR, for example FAR14 as set forth in SEQ ID NO: 87; a Plutella FAR, such as a Plutella xylostella FAR, for example FAR27 as set forth in SEQ ID NO: 97; a Manducta FAR, such as a Manducta sexta FAR, for example FAR43 as set forth in SEQ ID NO: 103; a Chrysodeixis FAR, such as a Chrysodeixis includens FAR, for example FAR52 as set forth in SEQ ID NO: 125 and/or FAR47 as set forth in SEQ ID NO: 127; a Cydia FAR, such as a Cydia pomonella FAR, for example FAR46 as set forth in SEQ ID NO: 129; a Grapholita FAR, such as a Grapholita molesta FAR, for example FAR50 as set forth in SEQ ID NO: 131, or functional variants thereof having at least 60% similarity or identity thereto.

The yeast cell expressing a heterologous Δ13 desaturase, a further desaturase, and a FAR is capable of producing double desaturated fatty alcohols, such as for example (Z11, Z13)-hexadecadien-1-ol. In other words, the yeast cell expressing a heterologous Δ13 desaturase, a further desaturase, and a FAR is capable of converting at least a part of the (Z11, Z13)-hexadecadienoyl-CoA produced by the heterologous Δ13 desaturase and the further heterologous desaturase into (Z11, Z13)-hexadecadien-1-ol by the action of the FAR.

In some embodiments, the genes encoding the heterologous Δ13 desaturase, the further heterologous desaturase or the FAR have been codon optimized for said yeast cell.

In one embodiment, at least one of the genes encoding the heterologous Δ13 desaturase, the further heterologous desaturase or the FAR is present in a high copy number in the yeast cell.

In one embodiment, at least one of the genes encoding the heterologous Δ13 desaturase, the further heterologous desaturase or the FAR is under the control of an inducible promoter.

In one embodiment, at least one of the genes encoding the heterologous Δ13 desaturase, the further heterologous desaturase or the FAR are each independently comprised within the genome of the cell or within a vector comprised within the yeast cell.

In one embodiment, the yeast cell belongs to a genus selected from Saccharomyces, Pichia, Yarrowia, Kluyveromyces, Candida, Rhodotorula, Rhodosporidium, Cryptococcus, Trichosporon and Lipomyces, optionally wherein the yeast cell belongs to a species selected from Saccharomyces cerevisiae, Saccharomyces boulardi, Pichia pastoris, Kluyveromyces marxianus, Candida tropicalis, Cryptococcus albidus, Lipomyces lipofera, Lipomyces starkeyi, Rhodosporidium toruloides, Rhodotorula glutinis, Trichosporon pullulan and Yarrowia lipolytica, preferably the yeast cell is a Yarrowia lipolytica cell or a Saccharomyces cerevisiae cell.

In some embodiments, the yeast cell comprises a nucleic acid or a system of nucleic acids, as described in the section “Nucleic acid”.

The yeast cell according to the present invention may be comprised in a fermentation broth, a fermentation system, and/or a catalytic system. In other words, a fermentation broth, a fermentation system and/or a catalytic system may comprise the yeast cell according to the present invention.

In one embodiment, the yeast cell further expresses a heterologous NAD(P)H cytochrome b5 oxidoreductase (Ncb5or). The term ‘NAD(P)H cytochrome b5 oxidoreductase’ and ‘Ncb5or’ will be used herein interchangeably. The term ‘heterologous Ncb5or’ refers to an Ncb5or which is not naturally expressed by the organism, such as by the cell.

Ncb5or is an oxidoreductase acting on NADH or NADPH, with a heme protein as acceptor. It contains functional domains similar to cytochrome b5, cytochrome b5 reductase and CHORD-SGT1 (Deng, et al., 2010). Ncb5ors catalyze the reaction:

2 Fe³⁺+NAD(P)H<=>2 Fe²⁺+H++NAD(P)+

Ncb5ors capable of catalyzing such reaction have EC number 1.6.2.2.

Expression of one or more Ncb5ors in a cell expressing a desaturase and/or a FAR has a positive effect on the activity of the desaturase and/or FAR, as it results in an increase in titer of desaturated fatty acyl-CoA and/or desaturated fatty alcohol. Thus, a yeast cell expressing a desaturase and/or a FAR and one or more Ncb5ors is capable of producing said compounds with a higher titer compared to a yeast cell expressing the same desaturase and/or FAR but no heterologous Ncb5or when cultivated in the same conditions.

The Ncb5or may be native to a plant, an insect or a mammal, such as Homo sapiens. In one embodiment, the Ncb5or is native to an insect, such as an insect of the genus Agrotis, Amyelois, Aphantopus, Arctia, Bicyclus, Bombus, Bombyx, Chilo, Cydia, Danaus, Drosophila, Eumeta, Galleria, Helicoverpa, Heliothis, Hyposmocoma, Leptidea, Lobesia, Manduca, Operophtera, Ostrinia, Papilio, Papilio, Papilio, Pieris, Plutella, Spodoptera, Trichoplusia, and Vanessa. In one embodiment, the Ncb5or is native to an insect selected from Agrotis segetum, Amyelois transitella, Aphantopus hyperantus, Arctia plantaginis, Bicyclus anynana, Bombus terrestris, Bombyx mandarina, Bombyx mori, Chilo suppressalis, Cydia pomonella, Danaus plexippus, Drosophila grimshawi, Drosophila melanogaster, Eumeta japonica, Galleria mellonella, Helicoverpa armigera, Heliothis virescens, Hyposmocoma kahamanoa, Leptidea sinapis, Lobesia botrana, Manduca sexta, Operophtera brumata, Ostrinia furnacalis, Papilio machaon, Papilio polytes, Papilio xuthus, Pieris rapae, Plutella xylostella, Spodoptera frugiperda, Spodoptera litura, Trichoplusia ni, and Vanessa tameamea.

In one embodiment, the Ncb5or is selected from the group of Ncb5ors set forth in the table “Ncb5ors” in the section “Sequence overview” in patent application PCT/EP2022/062641 entitled “Improved methods and cells for increasing enzyme activity and production of insect pheromones”, filed by the same applicant on 10 May 2022.

Thus, in one embodiment, the Ncb5or is selected from the group of Ncb5ors set forth in SEQ ID NO: 23, SEQ ID NO: 24, SEQ ID NO: 25, SEQ ID NO: 26, SEQ ID NO: 27, SEQ ID NO: 53, SEQ ID NO: 104 and SEQ ID NO: 105, or functional variants having at least 70% identity or similarity thereto, such as at least 75% identity, such as at least 80% identity, such as at least 85% identity, such as at least 90% identity, such as at least 95% identity or similarity thereto.

In some embodiments, the yeast cell has reduced activity of one or proteins as disclosed in WO 2018/109163 and in European patent 3555268, in particular in the section entitled “Reduced activity of Hfd1, Hfd4, Pex10, Fao1, GPAT or a homologue thereof”. For example, the yeast cell may have a mutation resulting in reduced activity (i.e. downregulation) of Pex10, Hfd1, Hfd4, Fao1 and/or GPAT. Preferably, the yeast cell has at least one mutation resulting in reduced activity of at least Fao1 and one or more of Hfd1, Hfd4, Pex10 and/or GPAT. Such mutations may increase the production of desaturated fatty alcohol and/or desaturated fatty alcohol acetate in a yeast cell expressing a heterologous desaturase and a heterologous fatty acyl-CoA reductase.

Thus, in one embodiment, the yeast cell has a mutation leading to partial or total loss of activity of one or more of Hfd1, Hfd4, GPAT, Fao1, Pex10, such as having at least a mutation leading to partial or total loss of activity of Fao1 and one or more of Hfd1, Hfd4, Pex10 or GPAT.

In one embodiment, the yeast cell has a mutation in PEX10 (accession number: XP_501311.1) and at least one of HFD1 (accession number: XP_505802.1), HFD4 (accession number: XP_500380.1X), FAO1 (accession number: XP_500864.1) and/or GPAT (accession number: XP_501275.1), or a homologue thereof having at least 60% identity thereto.

In one embodiment, the yeast cell has a mutation in FAO1 (accession number: XP_500864.1) and at least one of HFD1 (accession number: XP_505802.1), HFD4 (accession number: XP_500380.1X), PEX10 (accession number: XP_501311.1) and/or GPAT (accession number: XP_501275.1), or a homologue thereof having at least 60% identity thereto.

In one embodiment, HFD1 (accession number: XP_505802.1), HFD4 (accession number: XP_500380.1X), PEX10 (accession number: XP_501311.1) and/or FAO1 (accession number: XP_500864.1) or homologue thereof having at least 60% identify thereto is deleted or mutated, resulting in partial loss of total loss of activity of Hfd1, Hfd4, Pex10, and/or Fao1, and/or GPAT or a homologue thereof having at least 60% identity thereto is mutated, resulting in reduced activity of GPAT.

In one embodiment, the yeast cell comprises a mutation in at least one POX gene, such as a POX gene selected from the group consisting of POX1 (accession number: O74934.1), POX2 (accession number: XP_505264.1), POX3 (accession number: XP_503244.1), POX4 (accession number: XP_504475.1), POX5 (accession number: XP_502199.1), and POX6 (accession number: XP_503632.1).

In one embodiment, the yeast cell comprises a deletion or mutation in POX1 (accession number: 074934.1), POX2 (accession number: XP_505264.1), POX3 (accession number: XP_503244.1), POX4 (accession number: XP_504475.1), POX5 (accession number: XP_502199.1), and/or POX6 (accession number: XP_503632.1) or a homologue thereof having at least 60% identify thereto, resulting in partial loss of total loss of activity of Pox1, Pox2, Pox3, Pox4, Pox5 and/or Pox6.

The yeast cell may be further engineered to express any type of protein. Expression of such other proteins may improve the production, such as the titer, of the compounds disclosed herein. In one embodiment, the yeast cell expresses:
i) a heterologous cytochrome b5;
ii) a heterologous cytochrome b5 reductase;
iii) a hemoglobin;
iv) a heterologous thioesterase gene; and/or
v) of a fusion protein of a fatty acyl synthase and of a thioesterase.

The native genes of the yeast cell may also be engineered. In one embodiment, the yeast cell has an inactivation or modification of:
i) native elongase(s) resulting in total or partial loss of activity;
ii) thioesterases(s) resulting in total or partial loss of activity; and/or
iii) activity of native fatty aldehyde dehydrogenase(s), fatty alcohol oxidase(s), peroxisome biogenesis factor and/or fatty acyl synthase(s).

In some embodiments, the yeast cell is further modified so that the availability of fatty acyls having a chain length of 16 is increased or further increased. For instance, the fatty acid synthase complex may be engineered so that formation of hexadecanoyl-CoA is increased. The fatty acid synthase complex (EC 2.3.1.86) consists of two subunits, Fas1 (beta subunit) and Fas2 (alpha subunit). The alpha subunit comprises a ketoacyl synthase domain (a “binding pocket”) which is hypothesized to be involved in determining the length of the synthesized fatty acids.

Accordingly, in order to direct the metabolic flux towards production of desaturated fatty alcohols, acetates or aldehydes having a chain length of 16 carbons (C16), the yeast cell may further express a fatty acyl synthase variant having a modified ketone synthase domain. Without being bound by theory, it is hypothesized that the modified ketone synthase domain results in a modified binding pocket, which thus more readily accommodates medium length substrates such as C16 substrates, thereby producing a higher proportion of C16 products.

In one embodiment, the yeast cell is a yeast cell as described herein, wherein the cell further expresses a modified fatty acid synthase complex. In one embodiment, the fatty acid synthase complex is modified by mutating the gene encoding the alpha subunit of the complex. In some embodiments, the mutation is in the gene encoding FAS2. In one embodiment, the yeast cell is a Yarrowia lipolytica yeast cell and the mutation may result in modification of one or more of residue 1220 (11220), residue 1217 (M1217) or residue 1226 (M1226) of Yarrowia lipolytica FAS2, resulting in a variant FAS2. The skilled person will know how to design such mutations.

Preferably, the mutation results in an 11220F variant, an 11220W variant, an 11220Y variant or an 11220H variant. In a specific embodiment, the mutation results in an 11220F variant. In some embodiments, the mutation results in an M1217F variant, an M1217W variant, an M1217Y variant or an M1217H variant. In other embodiments, the mutation results in an M1226F variant, an M1226W variant, an M1226Y variant or an M1226H variant. Yeast cells with more than one of the above mutations are also contemplated, such as two mutations or three mutations at residue 11220, M1217 or M1226.

In one embodiment, the yeast cell is further modified to increase the availability of hexadecanoyl-CoA and/or (Z11)-hexadecenoyl-CoA.

The fatty acyl-CoAs produced by the cell may be further converted by the cell to the corresponding fatty acids. Said fatty acids may be free fatty acids or part of triacylglycerides (lipids).

In one embodiment, the yeast cell is capable of producing a Z13-fatty acid and/or (Z11, Z13)-hexadecadienoic acid with a titer of at least 0.005 mg/L, such as at least 0.01 mg/L, such as at least 0.02 mg/L, such as at least 0.03 mg/L, such as at least 0.05 mg/L, such as at least 0.06 mg/L, such as at least 0.075 mg/L, such as at least 0.1 mg/L, such as at least 0.2 mg/L, such as at least 0.3 mg/L, such as at least 0.4 mg/L, such as at least 0.5 mg/L, such as at least 0.6 mg/L, such as at least 0.7 mg/L, such as at least 0.8 mg/L, such as at least 0.9 mg/L, such as at least 1 mg/L, such as at least 2 mg/L, such as at least 3 mg/L, such as at least 4 mg/L, such as at least 5 mg/L, such as at least 10 mg/L, such as at least 15 mg/L, such as at least 20 mg/L, such as at least 25 mg/L, such as at least 30 mg/L, such as at least 35 mg/L, such as at least 50 mg/L, such as at least 60 mg/L, such as at least 70 mg/L, such as at least 80 mg/L, such as at least 90 mg/L, such as at least 100 mg/L, such as at least 200 mg/L, such as at least 300 mg/L, such as at least 400 mg/L, such as at least 500 mg/L, such as at least 750 mg/L, such as at least 1 g/L, such as at least 2 g/L, such as at least 5 g/L, such as at least 10 g/L, such as at least 20 g/L, such as at least 50 g/L, such as at least 100 g/L, or more.

In one embodiment, the yeast cell is capable of producing a Z13-fatty alcohol and/or (Z11, Z13)-hexadecadien-1-ol with a titer of at least 0.005 mg/L, such as at least 0.01 mg/L, such as at least 0.02 mg/L, such as at least 0.03 mg/L, such as at least 0.05 mg/L, such as at least 0.06 mg/L, such as at least 0.075 mg/L, such as at least 0.1 mg/L, such as at least 0.2 mg/L, such as at least 0.3 mg/L, such as at least 0.4 mg/L, such as at least 0.5 mg/L, such as at least 0.6 mg/L, such as at least 0.7 mg/L, such as at least 0.8 mg/L, such as at least 0.9 mg/L, such as at least 1 mg/L, such as at least 2 mg/L, such as at least 3 mg/L, such as at least 4 mg/L, such as at least 5 mg/L, such as at least 10 mg/L, such as at least 15 mg/L, such as at least 20 mg/L, such as at least 25 mg/L, such as at least 30 mg/L, such as at least 35 mg/L, such as at least 50 mg/L, such as at least 60 mg/L, such as at least 70 mg/L, such as at least 80 mg/L, such as at least 90 mg/L, such as at least 100 mg/L, such as at least 200 mg/L, such as at least 300 mg/L, such as at least 400 mg/L, such as at least 500 mg/L, such as at least 750 mg/L, such as at least 1 g/L, such as at least 2 g/L, such as at least 5 g/L, such as at least 10 g/L, such as at least 20 g/L, such as at least 50 g/L, such as at least 100 g/L, or more.

The yeast cell disclosed herein expressing a heterologous Δ13 desaturase, optionally a further heterologous desaturase, and further optionally a heterologous FAR is capable of producing desaturated fatty acyl-CoAs and/or desaturated fatty alcohols with a carbon chain length of at least 14 having a double bond at position 13 and optionally other double bond(s) at position(s) other than position 13. In some embodiments, the desaturated fatty acyl-CoAs and/or desaturated fatty alcohols may have a carbon chain consisting solely of single and double bonds.

In some embodiments, the produced desaturated fatty acyl-CoAs and/or desaturated fatty alcohols may have a carbon chain consisting solely of single and double bonds.

Thus, provided herein are desaturated fatty acyl-CoAs and desaturated fatty alcohols obtainable according to the methods presented herein.

Also provided herein is a yeast cell capable of producing a pheromone compound according the invention, wherein the yeast cell expresses a heterologous Δ13 fatty acyl-CoA desaturase.

In some embodiments, the yeast cell expresses a further heterologous desaturase.

In some embodiments, the yeast cell further expresses at least one alcohol-forming fatty acyl-CoA reductase (FAR).

Further provided herein is the use of a desaturated fatty acyl-CoA and/or a desaturated fatty alcohol obtainable according to the methods presented herein. Such compounds can be used for example for monitoring the presence of pest or disrupting the presence of pest; for example they can be formulated as a pheromone composition.

### Desaturated Fatty Acyl-CoAs

In one embodiment, the yeast cell expresses a heterologous Δ13 desaturase, and optionally a further heterologous desaturase, said yeast cell being capable of producing a desaturated fatty acyl-CoA having a double bond at position 13, and optionally a double bond at a position other than position 13. In some embodiments, the desaturated fatty acyl-CoA may have a carbon chain consisting solely of single and double bonds.

Thus, the yeast cell disclosed herein is capable of producing a desaturated fatty acyl-CoA having n′ double bond(s), wherein at least one of said double bond(s) is at position 13, wherein said yeast cell expresses a heterologous Δ13 desaturase, said desaturase being capable of introducing a double bond at position 13 in a saturated or desaturated fatty acyl-CoA, preferably a desaturated fatty acyl-CoA, having a carbon chain length of at least 14 and having n double bond(s), wherein n and n′ are integers, wherein 0≤n≤3 and wherein 1≤n′≤4.

Thus, the yeast cell disclosed herein is capable of producing a desaturated fatty acyl-CoA having n′ double bond(s), wherein at least one of said double bond(s) is at position 13, wherein said yeast cell expresses a heterologous Δ13 desaturase, said desaturase being capable of introducing a double bond at position 13 in a saturated fatty acyl-CoA or desaturated fatty acyl-CoA having a carbon chain consisting solely of single and double bonds, preferably a desaturated fatty acyl-CoA, wherein said fatty acyl-CoA has a carbon chain length of at least 14 and has n double bond(s), wherein n and n′ are integers, wherein 0≤n≤3 and wherein 1≤n′≤4.

In one embodiment the yeast cell expresses a further desaturase as disclosed herein in the section “Further desaturase”.

In one embodiment, n=0. In other words, the yeast cell is capable of introducing up to 4 double bonds, such as 1, 2, 3, or 4 double bonds, into a saturated fatty acyl-CoA having no double bonds. Thus, said yeast cell is capable of producing a desaturated fatty acyl-CoA having 1, 2, 3, or 4 double bonds. In a preferred embodiment, the yeast cell is capable of introducing a double bond into hexadecanoyl-CoA, thereby converting said hexadecanoyl-CoA to (Z13)-hexadecenoyl-CoA.

In one embodiment, n=1. In other words, the yeast is capable of introducing up to 3 double bonds, such as 1, 2 or 3 double bonds into a desaturated fatty acyl-CoA having one double bond at a position other than position 13, such as at position 8, 9, 10, 11, 12, 14, 15, 16, 17, 18, 19, 20, 21 or 22. Thus, said yeast cell is capable of producing a desaturated fatty acyl-CoA having 2, 3 or 4 double bonds, preferably a desaturated fatty acyl-CoA having 2 double bonds. In one embodiment, the yeast cell is capable of introducing a double bond into a desaturated fatty acyl-CoA having a double bond at position 11. In a preferred embodiment, the yeast cell is capable of introducing a double bond into (Z11)-hexadecenoyl-CoA, thereby converting said (Z11)-hexadecenoyl-CoA into (Z11, Z13)-hexadecadienoyl-CoA.

In one embodiment, n=2. In other words, the yeast cell is capable of introducing up to 2 double bonds, such as 1 or 2 double bonds, into a desaturated fatty acyl-CoA having two double bonds at a position other than position 13, such as a double bond in at least two of positions 8, 9, 10, 11, 12, 14, 15, 16, 17, 18, 19, 20, 21 or 22. Thus, said yeast cell is capable of producing a desaturated fatty acyl-CoA having 3 or 4 double bonds.

In one embodiment, n=3. In other words, the yeast is capable of introducing one double bond into a desaturated fatty acyl-CoA having three double bonds at a position other than position 13, such as one double bond at least three of positions 8, 9, 10, 11, 12, 14, 15, 16, 17, 18, 19, 20, 21 or 22. Thus, said yeast cell is capable of producing a desaturated fatty acyl-CoA having 4 double bonds.

In some embodiments, the desaturated fatty acyl-CoA(s) has a carbon chain consisting solely of single and double bonds. In other embodiments, the produced desaturated fatty acyl-CoA(s) has a carbon chain consisting solely of single and double bonds.

In preferred embodiments, the saturated fatty acyl-CoA or desaturated fatty acyl-CoA used as a substrate and the desaturated fatty acyl-CoA having n′ double bonds have the same carbon chain length.

In one embodiment, n′=1 and n=0. In other words, the yeast cell is capable of converting a saturated fatty acyl-CoA with no double bond into a desaturated fatty acyl-CoA with one double bond at position 13.

In one embodiment, n′=2 and n=0. In other words, the yeast cell is capable of converting a saturated fatty acyl-CoA into a desaturated fatty acyl-CoA with two double bonds, wherein one double bond is at position 13.

In one embodiment, n′=3 and n=0. In other words, the yeast cell is capable of converting a saturated fatty acyl-CoA into a desaturated fatty acyl-CoA with three double bonds, wherein one double bond is at position 13.

In one embodiment, n′=4 and n=0. In other words, the yeast cell is capable of converting a saturated fatty acyl-CoA into a desaturated fatty acyl-CoA with four double bonds, wherein one double bond is at position 13.

In a preferred embodiment, n′=2 and n=1. In other words, the yeast cell is capable of converting a desaturated fatty acyl-CoA with one double bond into a desaturated fatty acyl-CoA with two double bonds, wherein one double bond is at position 13.

In one embodiment, n′=3 and n=1. In other words, the yeast cell is capable of converting a desaturated fatty acyl-CoA with one double bond into a desaturated fatty acyl-CoA with three double bonds, wherein one double bond is at position 13.

In one embodiment, n′=4 and n=1. In other words, the yeast cell is capable of converting a desaturated fatty acyl-CoA with one double bond into a desaturated fatty acyl-CoA with four double bonds, wherein one double bond is at position 13.

In one embodiment, n′=3 and n=2. In other words, the yeast cell is capable of converting a desaturated fatty acyl-CoA with two double bonds into a desaturated fatty acyl-CoA with three double bonds, wherein one double bond is at position 13.

In one embodiment, n′=4 and n=2. In other words, the yeast cell is capable of converting a desaturated fatty acyl-CoA with two double bonds into a desaturated fatty acyl-CoA with four double bonds, wherein one double bond is at position 13.

In one embodiment, n′=4 and n=3. In other words, the yeast cell is capable of converting a desaturated fatty acyl-CoA with three double bonds into a desaturated fatty acyl-CoA with four double bonds, wherein one double bond is at position 13.

In one embodiment, the saturated or desaturated fatty acyl-CoA having a carbon chain length of at least 14 and having n double bonds, has a carbon chain length of at least 15, such as at least 16, such as at least 17, such as at least 18, such as at least 19, such as at least 20, such as at least 21, such as at least 22, such as at least 23, such as at least 24. In a preferred embodiment, the saturated or desaturated fatty acyl-CoA having a carbon chain length of at least 14 and having n double bonds, has a carbon chain length of 14, 15, 16, 17, 18, 19 or 20.

In one embodiment, the saturated or desaturated fatty acyl-CoA having a carbon chain length of at least 14 and having n double bonds has a length of 16 and is hexadecanoyl-CoA or (Z11)-hexadecenoyl-CoA. In a preferred embodiment, the saturated or desaturated fatty acyl-CoA having a carbon chain length of at least 14 and having n double bonds has a length of 16 and is (Z11)-hexadecenoyl-CoA.

In one embodiment, the desaturated fatty acyl-CoA having n′ double bond(s) has a carbon chain length of at least 14, such as at least 15, such as at least 16, such as at least 17, such as at least 18, such as at least 19, such as at least 20, such as at least 21, such as at least 22, such as at least 23. In one embodiment, the desaturated fatty acyl-CoA having n′ double bond(s) has a carbon chain length of at most 20, such as at most 19, such as at most 18, such as at most 17, such as at most 16, such as at most 15, such as at most 14. In a preferred embodiment, the desaturated fatty acyl-CoA having n′ double bond(s) has a carbon chain length of 14, 15, 16, 17, 18, 19 or 20.

In one embodiment, the desaturated fatty acyl-CoA having n′ double bond(s) has one double bond, and is (Z13)-hexadecenoyl-CoA.

In one embodiment, the desaturated fatty acyl-CoA having n′ double bond(s) has two double bonds, and is (Z11, Z13)-hexadecadienoyl-CoA.

Thus, also provided herein is (Z13)-hexadecenoyl-CoA and/or (Z11, Z13)-hexadecadienoyl-CoA obtainable according to the methods presented herein.

### Desaturated Fatty Alcohols

In one embodiment, the yeast cell expressing a Δ13 desaturase, and optionally a further desaturase, such as a further heterologous desaturase, also expresses a FAR as presented herein in the section “Fatty acyl-CoA reductase”. Said yeast cell may be capable of producing a desaturated fatty alcohol having a double bond at position 13, and optionally a double bond at a position other than position 13, such as at position 11. In some embodiments, the desaturated fatty alcohol may have a carbon chain consisting solely of single and double bonds.

Said FAR, which is necessary for producing the desaturated fatty alcohol, can be any FAR described herein in the section “Fatty acyl-CoA reductase”, and said desaturated fatty alcohol can be any desaturated fatty alcohol corresponding to the fatty acyl-CoAs described herein in the section “Desaturated fatty acyl-CoAs”.

In one embodiment, the desaturated fatty alcohol is a (Z13)-fatty alcohol, such as (Z13)-hexadecen-1-ol. In one embodiment, the desaturated fatty alcohol is (Z11, Z13)-hexadecadien-1-ol.

Thus, further provided herein is a (Z13)-fatty alcohol, such as (Z13)-hexadecen-1-ol, and/or (Z11, Z13)-hexadecadien-1-ol obtainable by the methods presented herein.

Method for production of a desaturated fatty acyl-CoA Provided herein is a method for producing a desaturated fatty acyl-CoA having n′ double bond(s), wherein at least one of said double bond(s) is at position 13 in a yeast cell, said method comprising the steps of:
i) providing a yeast cell as defined herein,
ii) incubating said yeast cell in a medium under conditions allowing expression of a heterologous Δ13 desaturase, said desaturase being capable of introducing a double bond at position 13 in a saturated or desaturated fatty acyl-CoA, preferably a desaturated fatty acyl-CoA, having a carbon chain length of at least 14 and having n double bonds,
wherein n and n′ are integers,
wherein 0≤n≤3 and wherein 1≤n′≤4,
thereby producing a desaturated fatty acyl-CoA having n′ double bond(s), wherein at least one of said double bond(s) is at position 13.

In some embodiments, the desaturated fatty acyl-CoA(s) may have a carbon chain consisting solely of single and double bonds.

In preferred embodiments, the saturated fatty acyl-CoA or desaturated fatty acyl-CoA used as a substrate and the desaturated fatty acyl-CoA having n′ double bonds have the same carbon chain length.

Provided herein is also a method for producing a desaturated fatty acyl-CoA having n′ double bond(s), wherein at least one of said double bond(s) is at position 13 in a yeast cell, said method comprising the steps of:
i) providing a yeast cell as defined herein,
ii) incubating said yeast cell in a medium under conditions allowing expression of a heterologous Δ13 desaturase, said desaturase being capable of introducing a double bond at position 13 in a saturated fatty acyl-CoA or a desaturated fatty acyl-CoA having a carbon chain consisting solely of single and double bonds, preferably a desaturated fatty acyl-CoA, wherein said fatty acyl-CoA has a carbon chain length of at least 14 and has n double bonds,
wherein n and n′ are integers,
wherein 0≤n≤3 and wherein 1≤n′≤4,
thereby producing a desaturated fatty acyl-CoA having n′ double bond(s), wherein at least one of said double bond(s) is at position 13.

In one embodiment, the heterologous Δ13 desaturase is as defined herein in the section “Δ13 desaturase”. For example, the Δ13 desaturase may be an Amyelois Δ13 desaturase, such as an Amyelois transitella Δ13 desaturase, such as the Δ13 desaturase as set forth in SEQ ID NO: 1 (AtrAATQ) or a functional variant thereof having at least 60% identity or similarity thereto. In some embodiments, the Δ13 desaturase may be a Notodonta Δ13 desaturase, such as a Notodonta dromedarius Δ13 desaturase, such as the Δ13 desaturase as set forth in SEQ ID NO: 56 (NdDes1), or a functional variant thereof having at least 60% similarity or identity thereto.

In one embodiment, the further heterologous desaturase is as defined herein in the section “Further desaturase”. For example, the further desaturase may be a desaturase capable of introducing a double bond in a saturated or desaturated fatty acid at a position other than position 13. In some embodiments, the desaturase is a Spodoptera desaturase, such as a Spodoptera litura desaturase, for example Desat38 as set forth in SEQ ID NO: 3, or a Spodoptera littoralis desaturase, for example Desat20 as set forth in SEQ ID NO: 5, or such as a Spodoptera exigua desaturase, for example Desat37 as set forth in SEQ ID NO: 31; an Amyelois desaturase, such as an Amyelois transitella desaturase, for example Desat16 as set forth in SEQ ID NO: 7, an Agrotis desaturase, such as an Agrotis segetum desaturase, for example Desat19 as set forth in SEQ ID NO: 9; a Trichoplusia desaturase, such as a Trichoplusia ni desaturase, for example Desat21 as set forth in SEQ ID NO: 11; a Plutella desaturase, such as a Plutella xylostella desaturase, for example Desat45 as set forth in SEQ ID NO: 33; a Helicoverpa desaturase, such as a Helicoverpa zea desaturase, for example Desat51 as set forth in SEQ ID NO: 29; a Chilo desaturase, such as a Chilo suppressalis desaturase, for example Desat44 as set forth in SEQ ID NO: 51; a Diatraea desaturase, such as a Diatraea saccharalis desaturase, for example Desat63 as set forth in SEQ ID NO: 59; a Plodia desaturase, such as a Plodia interpunctella desaturase, for example Desat65 as set forth in SEQ ID NO: 61; a Lobesia desaturase, such as a Lobesia botrana desaturase, for example Desat71 as set forth in SEQ ID NO: 63 or Desat79 as set forth in SEQ ID NO: 65; an Antheraea desaturase, such as an Antheraea pernyi desaturase, for example Desat72 as set forth in SEQ ID NO: 67; a Cadra desaturase, such as a Cadra cautella desaturase, for example Desat70 as set forth in SEQ ID NO: 69; a Yponomeuta desaturase, such as a Yponomeuta padella desaturase, for example Desat73 as set forth in SEQ ID NO: 71; or functional variants thereof having at least 60% similarity or identity thereto.

The saturated or desaturated fatty acyl-CoA having a carbon chain length of at least 14 and having n double bond(s) may be as defined herein in the section “Desaturated fatty acyl-CoA”. In one embodiment, the saturated or desaturated fatty acyl-CoA having a carbon chain length of at least 14 and having n double bonds, is hexadecanoyl-CoA or (Z11)-hexadecenoyl-CoA. In other embodiments, the desaturated fatty acyl-CoA may have a carbon chain consisting solely of single and double bonds.

The desaturated fatty acyl-CoA having n′ double bond(s) may be as defined herein in the section “Desaturated fatty acyl-CoA”. In one embodiment, the desaturated fatty acyl-CoA having n′ double bond(s) is (Z13)-hexadecenoyl-CoA and/or (Z11, Z13)-hexadecadienoyl-CoA. In other embodiments, the desaturated fatty acyl-CoA may have a carbon chain consisting solely of single and double bonds.

In one embodiment, the method yields a desaturated fatty acyl-CoA having n′ double bond(s), such as (Z13)-hexadecenoyl-CoA and/or (Z11, Z13)-hexadecadienoyl-CoA, at a titer of at least 0.005 mg/L, such as at least 0.01 mg/L, such as at least 0.02 mg/L, such as at least 0.03 mg/L, such as at least 0.05 mg/L, such as at least 0.06 mg/L, such as at least 0.075 mg/L, such as at least 0.1 mg/L, such as at least 0.2 mg/L, such as at least 0.3 mg/L, such as at least 0.4 mg/L, such as at least 0.5 mg/L, such as at least 0.6 mg/L, such as at least 0.7 mg/L, such as at least 0.8 mg/L, such as at least 0.9 mg/L, such as at least 1 mg/L, such as at least 2 mg/L, such as at least 3 mg/L, such as at least 4 mg/L, such as at least 5 mg/L, such as at least 10 mg/L, such as at least 15 mg/L, such as at least 20 mg/L, such as at least 25 mg/L, such as at least 30 mg/L, such as at least 35 mg/L, such as at least 50 mg/L, such as at least 60 mg/L, such as at least 70 mg/L, such as at least 80 mg/L, such as at least 90 mg/L, such as at least 100 mg/L, such as at least 200 mg/L, such as at least 300 mg/L, such as at least 400 mg/L, such as at least 500 mg/L, such as at least 750 mg/L, such as at least 1 g/L, such as at least 2 g/L, such as at least 5 g/L, such as at least 10 g/L, such as at least 20 g/L, such as at least 50 g/L, such as at least 100 g/L, or more.

In one embodiment, the yeast cell further expresses at least one alcohol-forming fatty acyl-CoA reductase (FAR) capable of converting at least a part of (Z13)-hexadecenoyl-CoA and/or (Z11, Z13)-hexadecadienoyl-CoA into (Z13)-hexadecen-1-ol and/or (Z11, Z13)-hexadecadien-1-ol, respectively.

The FAR may be as defined herein in the section “Fatty acyl-CoA reductase”. For example, the FAR may be a Helicoverpa FAR, such as a Helicoverpa armigera FAR, for example FAR1 as set forth in SEQ ID NO: 13, or a Helicoverpa assulta FAR, for example FAR6 as set forth in SEQ ID NO: 21; a Heliothis FAR, such as a Heliothis subflexa FAR, for example FAR4 as set forth in SEQ ID NO: 19, or a Heliothis virescens FAR, for example FAR5 as set forth in SEQ ID NO: 73; an Amyelois FAR, such as an Amyelois transitella FAR, for example FAR33 as set forth in SEQ ID NO: 15, FAR34 as set forth in SEQ ID NO: 107 or FAR35 as set forth in SEQ ID NO: 109; a Plodia FAR, such as a Plodia interpunctella FAR, for example FAR28 as set forth in SEQ ID NO: 45 or FAR32 as set forth in SEQ ID NO: 35, FAR29 as set forth in SEQ ID NO: 99 or FAR30 as set forth in SEQ ID NO: 101; a Spodoptera FAR, such as a Spodoptera exigua FAR, for example FAR16 as set forth in SEQ ID NO: 37, FAR17 as set forth in SEQ ID NO: 41, or FAR45 as set forth in SEQ ID NO: 117, or such as a Spodoptera litura FAR, for example FAR19 as set forth in SEQ ID NO: 39, or such as a Spodoptera littoralis FAR, for example FAR15 as set forth in SEQ ID NO: 89, or such as a Spodoptera frugiperda FAR, for example FAR20 as set forth in SEQ ID NO: 93 or FAR22 as set forth in SEQ ID NO: 95; a Trichoplusia FAR, such as a Trichoplusia ni FAR, for example FAR38 as set forth in SEQ ID NO: 43, FAR40 as set forth in SEQ ID NO: 119, FAR41 as set forth in SEQ ID NO: 121 or FAR51 as set forth in SEQ ID NO: 123; an Yponomeuta FAR, such as an Yponomeuta rorellus FAR, for example FAR8 as set forth in SEQ ID NO: 75; an Ostrinia FAR, such as an Ostrinia nubilalis FAR, for example FAR9 as set forth in SEQ ID NO: 77, or such as an Ostrinia furnacalis FAR, for example FAR44 as set forth in SEQ ID NO: 111, or such as an Ostrinia zag FAR, for example FAR48 as set forth in SEQ ID NO: 113, or such as an Ostrinia zea FAR, for example FAR49 as set forth in SEQ ID NO: 115; an Agrotis FAR, such as an Agrotis segetum FAR, for example FAR12 as set forth in SEQ ID NO: 79, or such as an Agrotis ipsilon FAR, for example FAR18 as set forth in SEQ ID NO: 91; a Marinobacter FAR, such as a Marinobacter algicola FAR, for example FAR42 as set forth in SEQ ID NO: 81; a Tyta FAR, such as a Tyta alba FAR, for example FAR25 as set forth in SEQ ID NO: 83; a Chilo FAR, such as a Chilo suppressalis FAR, for example FAR13 as set forth in SEQ ID NO: 85; a Bombus FAR, such as a Bombus lapidarius FAR, for example FAR14 as set forth in SEQ ID NO: 87; a Plutella FAR, such as a Plutella xylostella FAR, for example FAR27 as set forth in SEQ ID NO: 97; a Manducta FAR, such as a Manducta sexta FAR, for example FAR43 as set forth in SEQ ID NO: 103; a Chrysodeixis FAR, such as a Chrysodeixis includens FAR, for example FAR52 as set forth in SEQ ID NO: 125 and/or FAR47 as set forth in SEQ ID NO: 127; a Cydia FAR, such as a Cydia pomonella FAR, for example FAR46 as set forth in SEQ ID NO: 129; a Grapholita FAR, such as a Grapholita molesta FAR, for example FAR50 as set forth in SEQ ID NO: 131, or functional variants thereof having at least 60% similarity or identity thereto.

In one embodiment, the method yields (Z11, Z13)-hexadecadien-1-ol at a titer of at least 0.005 mg/L, such as at least 0.01 mg/L, such as at least 0.02 mg/L, such as at least 0.03 mg/L, such as at least 0.05 mg/L, such as at least 0.06 mg/L, such as at least 0.075 mg/L, such as at least 0.1 mg/L, such as at least 0.2 mg/L, such as at least 0.3 mg/L, such as at least 0.4 mg/L, such as at least 0.5 mg/L, such as at least 0.6 mg/L, such as at least 0.7 mg/L, such as at least 0.8 mg/L, such as at least 0.9 mg/L, such as at least 1 mg/L, such as at least 2 mg/L, such as at least 3 mg/L, such as at least 4 mg/L, such as at least 5 mg/L, such as at least 10 mg/L, such as at least 15 mg/L, such as at least 20 mg/L, such as at least 25 mg/L, such as at least 30 mg/L, such as at least 35 mg/L, such as at least 50 mg/L, such as at least 60 mg/L, such as at least 70 mg/L, such as at least 80 mg/L, such as at least 90 mg/L, such as at least 100 mg/L, such as at least 200 mg/L, such as at least 300 mg/L, such as at least 400 mg/L, such as at least 500 mg/L, such as at least 750 mg/L, such as at least 1 g/L, such as at least 2 g/L, such as at least 5 g/L, such as at least 10 g/L, such as at least 20 g/L, such as at least 50 g/L, such as at least 100 g/L, or more.

In one embodiment, the method further comprises the step of recovering the Z13-fatty alcohol and/or the (Z11, Z13)-hexadecadien-1-ol.

In one embodiment, the Z13-fatty alcohol and/or the (Z11, Z13)-hexadecadien-1-ol is further modified into a Z13-fatty alcohol acetate and/or (Z11, Z13)-hexadecadien-1-ol acetate, respectively.

In one embodiment, the conversion of the Z13-fatty alcohol and/or the (Z11, Z13)-hexadecadien-1-ol to a Z13-fatty alcohol acetate and/or (Z11, Z13)-hexadecadien-1-ol acetate is performed in vitro.

In one embodiment the conversion of the Z13-fatty alcohol and/or the (Z11, Z13)-hexadecadien-1-ol to a Z13-fatty alcohol acetate and/or (Z11, Z13)-hexadecadien-1-ol acetate is performed in vivo by further expressing in the cell an acetyltransferase capable of converting the Z13-fatty alcohol and/or the (Z11, Z13)-hexadecadien-1-ol to a Z13-fatty alcohol acetate and/or (Z11, Z13)-hexadecadien-1-ol acetate, respectively.

In one embodiment the method further comprises a step of converting the Z13-fatty alcohol and/or the (Z11, Z13)-hexadecadien-1-ol to a (Z13)-fatty aldehyde and/or (Z11, Z13)-hexadecadienal. In one embodiment, the conversion to an aldehyde is a chemical or an enzymatic conversion.

Compounds of the present disclosure may be oxidized as described herein, by methods known to the skilled person or as described in European patent application EP21190097.2 filed on 6 Aug. 2021 by same applicant. In one embodiment, the Z13-fatty alcohol and/or the (Z11, Z13)-hexadecadien-1-ol is chemically oxidized into a (Z13)-fatty aldehyde and/or (Z11, Z13)-hexadecadienal.

Compounds of the present disclosure may be acetylated as described herein or by methods known to the skilled person. In one embodiment, the Z13-fatty alcohol and/or the (Z11, Z13)-hexadecadien-1-ol is chemically acetylated into a Z13-fatty alcohol acetate and/or (Z11, Z13)-hexadecadien-1-ol acetate.

In one embodiment, the method further comprises the steps of recovering and formulating the Z13-fatty alcohol, such as the (Z13)-hexadecen-1-ol, the (Z11, Z13)-hexadecadien-1-ol, the Z13-fatty alcohol acetate, the (Z11, Z13)-hexadecadien-1-ol acetate, the (Z13)-fatty aldehyde and/or the (Z11, Z13)-hexadecadienal in a pheromone composition as defined herein in the section “Pheromone composition”.

Thus, provided herein is a method for producing (Z11, Z13)-hexadecadien-1-ol, said method comprising the steps of:
i) providing a yeast cell as disclosed herein,
ii) incubating said yeast cell in a medium under conditions allowing expression of:
   a. a further desaturase, said desaturase being capable of introducing a double bond at position 11 in hexadecanoyl-CoA, thereby converting at least part of hexadecanoyl-CoA to (Z11)-hexadecenoyl-CoA;
   b. a heterologous Δ13 desaturase, said desaturase being capable of introducing a double bond at position 13 in (Z11)-hexadecenoyl-CoA, thereby converting at least part of (Z11)-hexadecenoyl-CoA to (Z11, Z13)-hexadecadienoyl-CoA;
   c. a FAR, said FAR being capable of converting at least part of (Z11, Z13)-hexadecadienoyl-CoA into (Z11, Z13)-hexadecadien-1-ol;
iii) optionally, recovering the (Z11, Z13)-hexadecadien-1-ol;
iv) optionally, converting the (Z11, Z13)-hexadecadien-1-ol into (Z11, Z13)-hexadecadien-1-ol acetate;
v) optionally, converting the (Z11, Z13)-hexadecadien-1-ol into (Z11, Z13)-hexadecadienal;
vi) optionally, formulating the (Z11, Z13)-hexadecadien-1-ol, the (Z11, Z13)-hexadecadien-1-ol acetate, and/or the (Z11, Z13)-hexadecadienal into a pheromone composition.

### Nucleic Acid

Provided herein is a nucleic acid or a system of nucleic acids for modifying a yeast cell, said nucleic acid or system comprising at least one polynucleotide encoding a heterologous Δ13 desaturase, said desaturase being capable of introducing a double bond at position 13 in a saturated or desaturated fatty acyl-CoA having a carbon chain length of at least 14 and having n double bonds thereby producing a desaturated fatty acyl-CoA having n′ double bond(s), wherein at least one of said double bond(s) is at position 13,
wherein n and n′ are integers,
wherein 0≤n≤3 and wherein 1≤n′≤4.

Provided herein is also a nucleic acid or a system of nucleic acids for modifying a yeast cell, said nucleic acid or system of nucleic acids comprising at least one polynucleotide encoding a heterologous Δ13 desaturase said desaturase being capable of introducing a double bond at position 13 in a saturated fatty acyl-CoA or a desaturated fatty acyl-CoA having a carbon chain consisting solely of single and double bonds, preferably a desaturated fatty-acyl-CoA, wherein said fatty acyl-CoA has a carbon chain length of at least 14 and has n double bonds, thereby producing a desaturated fatty acyl-CoA having n′ double bond(s), wherein at least one of said double bond(s) is at position 13,
wherein n and n′ are integers,
wherein 0≤n≤3 and wherein 1≤n′≤4.

In one embodiment, the heterologous Δ13 desaturase is encoded by a nucleic acid having at least 60% identity to the nucleic acid encoding an Amyelois transitella Δ13 desaturase, as set forth in SEQ ID NO: 2 and/or SEQ ID NO: 46, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% identity thereto.

In one embodiment, the heterologous Δ13 desaturase is encoded by a nucleic acid having at least 60% identity to the nucleic acid encoding an Notodonta dromedarius 413 desaturase, as set forth in SEQ ID NO: 57, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% identity thereto.

In one embodiment, the heterologous Δ13 desaturase is encoded by a nucleic acid having at least 60% identity to the nucleic acid encoding a Δ13 desaturase fusion protein, as set forth in SEQ ID NO: 135, SEQ ID NO: 136 and/or SEQ ID NO: 137, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% identity thereto.

In one embodiment, the nucleic acid or the system of nucleic acids further comprises a polynucleotide encoding a further heterologous desaturase capable of introducing a double bond at any position which is not position 13 in a saturated or desaturated fatty acyl-CoA having a carbon chain length of at least 14 and having n double bonds. In some embodiments, the desaturated fatty acyl-CoA may have a carbon chain consisting solely of single and double bonds. In preferred embodiments, the saturated fatty acyl-CoA or desaturated fatty acyl-CoA used as a substrate and the desaturated fatty acyl-CoA having n′ double bonds have the same carbon chain length.

In some embodiments, the further heterologous desaturase is encoded by a nucleic acid having at least 60% identity to a nucleic acid selected from the group of nucleic acids as set forth in SEQ ID NO: 4, SEQ ID NO: 6, SEQ ID NO: 8, SEQ ID NO: 10, SEQ ID NO: 12, SEQ ID NO: 28, SEQ ID NO: 30, SEQ ID NO: 32, SEQ ID NO: 47, SEQ ID NO: 52, SEQ ID NO: 50, SEQ ID NO: 138, SEQ ID NO: 58, SEQ ID NO: 60, SEQ ID NO: 62, SEQ ID NO: 64, SEQ ID NO: 66, SEQ ID NO: 68, and SEQ ID NO: 70, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% identity thereto.

In one embodiment, the further heterologous desaturase is encoded by a nucleic acid having at least 60% identity to the nucleic acid encoding a Spodoptera litura desaturase, as set forth in SEQ ID NO: 4.

In one embodiment, the further heterologous desaturase is encoded by a nucleic acid having at least 60% identity to the nucleic acid encoding a Spodoptera littoralis desaturase, as set forth in SEQ ID NO: 6.

In one embodiment, the further heterologous desaturase is encoded by a nucleic acid having at least 60% identity to the nucleic acid encoding a Spodoptera exigua desaturase, as set forth in SEQ ID NO: 30.

In one embodiment, the further heterologous desaturase is encoded by a nucleic acid having at least 60% identity to the nucleic acid encoding an Amyelois transitella desaturase, as set forth in SEQ ID NO: 8 and/or SEQ ID NO: 47.

In one embodiment, the further heterologous desaturase is encoded by a nucleic acid having at least 60% identity to the nucleic acid encoding an Agrotis segetum desaturase, as set forth in SEQ ID NO: 10.

In one embodiment, the further heterologous desaturase is encoded by a nucleic acid having at least 60% identity to the nucleic acid encoding a Trichoplusia ni desaturase, as set forth in SEQ ID NO: 12.

In one embodiment, the further heterologous desaturase is encoded by a nucleic acid having at least 60% identity to the nucleic acid encoding a Plutella xylostella desaturase, as set forth in SEQ ID NO: 32 and/or SEQ ID NO: 52.

In one embodiment, the further heterologous desaturase is encoded by a nucleic acid having at least 60% identity to the nucleic acid encoding a Helicoverpa zea desaturase, as set forth in SEQ ID NO: 28.

In one embodiment, the further heterologous desaturase is encoded by a nucleic acid having at least 60% identity to the nucleic acid encoding a Chilo suppressalis desaturase, as set forth in SEQ ID NO: 50 and/or SEQ ID NO: 138.

In one embodiment, the further heterologous desaturase is encoded by a nucleic acid having at least 60% identity to the nucleic acid encoding a Diatraea saccharalis desaturase, as set forth in SEQ ID NO: 58.

In one embodiment, the further heterologous desaturase is encoded by a nucleic acid having at least 60% identity to the nucleic acid encoding a Plodia interpunctella desaturase, as set forth in SEQ ID NO: 60.

In one embodiment, the further heterologous desaturase is encoded by a nucleic acid having at least 60% identity to the nucleic acid encoding a Lobesia botrana desaturase, as set forth in SEQ ID NO: 62 and/or SEQ ID NO: 64.

In one embodiment, the further heterologous desaturase is encoded by a nucleic acid having at least 60% identity to the nucleic acid encoding an Antheraea pernyi desaturase, as set forth in SEQ ID NO: 66.

In one embodiment, the further heterologous desaturase is encoded by a nucleic acid having at least 60% identity to the nucleic acid encoding a Cadra cautella desaturase, as set forth in SEQ ID NO: 68.

In one embodiment, the further heterologous desaturase is encoded by a nucleic acid having at least 60% identity to the nucleic acid encoding an Yponomeuta padella desaturase, as set forth in SEQ ID NO: 70.

In one embodiment, the nucleic acid or system of nucleic acids further comprises a polynucleotide encoding a FAR capable of converting at least a part of the double desaturated (Z11, Z13)-hexadecadienoyl-CoA into (Z11, Z13)-hexadecadien-1-ol.

In some embodiments, the FAR is encoded by a nucleic acid having at least 60% identity to a nucleic acid selected from the group of FARs set forth in SEQ ID NO: 14, SEQ ID NO: 16, SEQ ID NO: 20, SEQ ID NO: 22, SEQ ID NO: 36, SEQ ID NO: 38, SEQ ID NO: 40, SEQ ID NO: 42, SEQ ID NO: 44, SEQ ID NO: 48, SEQ ID NO: 49, SEQ ID NO: 72, SEQ ID NO: 74, SEQ ID NO: 76, SEQ ID NO: 110, SEQ ID NO: 112, SEQ ID NO: 114, SEQ ID NO: 78, SEQ ID NO: 90, SEQ ID NO: 80, SEQ ID NO: 82, SEQ ID NO: 84, SEQ ID NO: 86, SEQ ID NO: 88, SEQ ID NO: 92, SEQ ID NO: 94, SEQ ID NO: 116, SEQ ID NO: 96, SEQ ID NO: 98, SEQ ID NO: 100, SEQ ID NO: 102, SEQ ID NO: 106, SEQ ID NO: 108, SEQ ID NO: 118, SEQ ID NO: 120, SEQ ID NO: 122, SEQ ID NO: 124, SEQ ID NO: 126, SEQ ID NO: 128, and SEQ ID NO: 130, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% identity thereto.

In one embodiment, the FAR is encoded by a nucleic acid having at least 60% identity to the nucleic acid encoding a Helicoverpa armigera FAR, as set forth in SEQ ID NO: 14 and/or SEQ ID NO: 48.

In one embodiment, the FAR is encoded by a nucleic acid having at least 60% identity to the nucleic acid encoding a Helicoverpa assulta FAR, as set forth in SEQ ID NO: 22.

In one embodiment, the FAR is encoded by a nucleic acid having at least 60% identity to the nucleic acid encoding a Heliothis subflexa FAR, as set forth in SEQ ID NO: 20.

In one embodiment, the FAR is encoded by a nucleic acid having at least 60% identity to the nucleic acid encoding an Amyelois transitella FAR, as set forth in SEQ ID NO: 16, SEQ ID NO: 14, SEQ ID NO: 106 or SEQ ID NO: 108.

In one embodiment, the FAR is encoded by a nucleic acid having at least 60% identity to the nucleic acid encoding a Plodia interpunctella FAR, as set forth in SEQ ID NO: 44 or SEQ ID NO: 34, SEQ ID NO: 98 or SEQ ID NO: 100.

In one embodiment, the FAR is encoded by a nucleic acid having at least 60% identity to the nucleic acid encoding a Spodoptera exigua FAR, as set forth in SEQ ID NO: 36, SEQ ID NO: 40, or SEQ ID NO: 116. In one embodiment, the FAR is encoded by a nucleic acid having at least 60% identity to the nucleic acid encoding a Spodoptera littoralis FAR, as set forth in SEQ ID NO: 88. In one embodiment, the FAR is encoded by a nucleic acid having at least 60% identity to the nucleic acid encoding a Spodoptera frugiperda FAR, as set forth in SEQ ID NO: 92 or SEQ ID NO: 94.

In one embodiment, the FAR is encoded by a nucleic acid having at least 60% identity to the nucleic acid encoding a Spodoptera litura FAR, as set forth in SEQ ID NO: 38.

In one embodiment, the FAR is encoded by a nucleic acid having at least 60% identity to the nucleic acid encoding a Trichoplusia ni FAR, as set forth in SEQ ID NO: 42, SEQ ID NO: 118, SEQ ID NO: 120 or SEQ ID NO: 122.

In one embodiment, the FAR is encoded by a nucleic acid having at least 60% identity to the nucleic acid encoding a Heliothis virescens FAR, as set forth in SEQ ID NO: 72.

In one embodiment, the FAR is encoded by a nucleic acid having at least 60% identity to the nucleic acid encoding an Yponomeuta rorellus FAR, as set forth in SEQ ID NO: 74.

In one embodiment, the FAR is encoded by a nucleic acid having at least 60% identity to the nucleic acid encoding an Ostrinia nubilalis FAR, as set forth in SEQ ID NO: 76.

In one embodiment, the FAR is encoded by a nucleic acid having at least 60% identity to the nucleic acid encoding an Ostrinia furnacalis FAR, as set forth in SEQ ID NO: 110. In one embodiment, the FAR is encoded by a nucleic acid having at least 60% identity to the nucleic acid encoding an Ostrinia zag FAR, as set forth in SEQ ID NO:

112. In one embodiment, the FAR is encoded by a nucleic acid having at least 60% identity to the nucleic acid encoding an Ostrinia zea FAR, as set forth in SEQ ID NO: 114.

In one embodiment, the FAR is encoded by a nucleic acid having at least 60% identity to the nucleic acid encoding an Agrotis segetum FAR, as set forth in SEQ ID NO: 78. In one embodiment, the FAR is encoded by a nucleic acid having at least 60% identity to the nucleic acid encoding an Agrotis ipsilon FAR, as set forth in SEQ ID NO: 90.

In one embodiment, the FAR is encoded by a nucleic acid having at least 60% identity to the nucleic acid encoding a Marinobacter algicola FAR, as set forth in SEQ ID NO: 80.

In one embodiment, the FAR is encoded by a nucleic acid having at least 60% identity to the nucleic acid encoding a Tyta alba FAR, as set forth in SEQ ID NO: 82.

In one embodiment, the FAR is encoded by a nucleic acid having at least 60% identity to the nucleic acid encoding a Chilo suppressalis FAR, as set forth in SEQ ID NO: 84.

In one embodiment, the FAR is encoded by a nucleic acid having at least 60% identity to the nucleic acid encoding a Bombus lapidarius FAR, as set forth in SEQ ID NO: 86.

In one embodiment, the FAR is encoded by a nucleic acid having at least 60% identity to the nucleic acid encoding a Plutella xylostella FAR, as set forth in SEQ ID NO: 96.

In one embodiment, the FAR is encoded by a nucleic acid having at least 60% identity to the nucleic acid encoding a Manducta sexta FAR, as set forth in SEQ ID NO: 102.

In one embodiment, the FAR is encoded by a nucleic acid having at least 60% identity to the nucleic acid encoding a Chrysodeixis includens FAR, as set forth in SEQ ID NO: 124 or SEQ ID NO: 126.

In one embodiment, the FAR is encoded by a nucleic acid having at least 60% identity to the nucleic acid encoding a Cydia pomonella FAR, as set forth in SEQ ID NO: 128.

In one embodiment, the FAR is encoded by a nucleic acid having at least 60% identity to the nucleic acid encoding a Grapholita molesta FAR, as set forth in SEQ ID NO: 130.

Herein, a nucleic acid having at least 60% identity to a given nucleic acid may have at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to the given nucleic acid, or more.

The system of nucleic acids can thus comprise a plurality of polynucleotides encoding the enzymes that are to be expressed in the yeast cell, in particular a heterologous Δ13 desaturase as described herein, and optionally a further heterologous desaturase as described herein, and further optionally a FAR and/or additional enzymes such as an Ncb5or.

### Recovery

It may be desirable to recover the products obtained by the methods disclosed herein. Thus, the present methods may comprise a further step of recovering the desaturated fatty alcohol, the desaturated fatty alcohol acetate, and/or the desaturated fatty aldehyde produced according to the methods presented herein.

In some embodiments, the method comprises a step of recovering the desaturated fatty alcohols. In other embodiments, the method comprises a step of recovering the desaturated fatty alcohol acetates.

Methods for recovering the products obtained by the present invention are known in the art and may comprise an extraction with a hydrophobic solvent such as decane, hexane or a vegetable oil.

The recovered products may be modified further, for example may the desaturated fatty alcohols be converted to the corresponding desaturated fatty aldehydes as described herein above. In embodiments where desaturated fatty aldehydes are directly produced in the culture medium, e.g. in vivo or by contacting the cells with the relevant enzymes, the desaturated fatty aldehydes may also be recovered.

As described in application WO 2021/078452 filed by same applicant on 22 Sep. 2020 and entitled “Improved methods for production, recovery and secretion of hydrophobic compounds in a fermentation”, when a fermentation system is used to cultivate the cell, in particular a yeast cell, which is capable of producing desaturated fatty alcohols, desaturated fatty alcohol acetates and/or desaturated fatty aldehydes, the addition of an extractant to the culture medium may further increase titers and extracellular secretion. In some embodiments, the medium comprises an extractant in an amount equal to or greater than its cloud concentration in an aqueous solution, wherein the extractant a non-ionic surfactant such as an antifoaming agent, preferably a polyethoxylated surfactant selected from: a polyethylene polypropylene glycol, a mixture of polyether dispersions, an antifoaming agent comprising polyethylene glycol monostearate such as simethicone and ethoxylated and propoxylated C16-C18 alcohol-based antifoaming agents and combinations thereof. In some embodiments:
the non-ionic surfactant is an ethoxylated and propoxylated C16-C18 alcohol-based antifoaming agent, such as C16-C18 alkyl alcohol ethoxylate propoxylate (CAS number 68002-96-0), and wherein the culture medium comprises at least 1% vol/vol of C16-C18 alkyl alcohol ethoxylate propoxylate, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol C16-C18 alkyl alcohol ethoxylate propoxylate, or more,
the non-ionic surfactant is a polyethylene polypropylene glycol, for example Kollliphor® P407 (CAS number 9003-11-6), and wherein the culture medium comprises at least 10% vol/vol of polyethylene polypropylene glycol such as Kolliphor® P407, such as at least 11% vol/vol, such as at least 12% vol/vol, such as at least 13% vol/vol, such as at least 14% vol/vol, such as at least 15% vol/vol, such as at least 16% vol/vol, such as at least 17% vol/vol, such as at least 18% vol/vol, such as at least 19% vol/vol, such as at least 20% vol/vol, such as at least 25% vol/vol, such as at least 30% vol/vol, such as at least 35% vol/vol of polyethylene polypropylene glycol such as Kolliphor® P407, or more,
the non-ionic surfactant is a mixture of polyether dispersions, such as antifoam 204, and wherein the culture medium comprises at least 1% vol/vol of a mixture of polyether dispersions such as antifoam 204, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol of a mixture of polyether dispersions such as antifoam 204, or more; and/or
the non-ionic surfactant is a non-ionic surfactant comprising polyethylene glycol monostearate such as simethicone, and wherein the culture medium comprises at least 1% vol/vol of polyethylene glycol monostearate or simethicone, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol polyethylene glycol monostearate or simethicone, or more.

In other embodiments, the culture medium comprises the extractant in an amount greater than its cloud concentration by at least 50%, such as at least 100%, such as at least 150%, such as at least 200%, such as at least 250%, such as at least 300%, such as at least 350%, such as at least 400%, such as at least 500%, such as at least 750%, such as at least 1000%, or more, and/or wherein the culture medium comprises the extractant in an amount at least 2-fold its cloud concentration, such as at least 3-fold its cloud concentration, such as at least 4-fold its cloud concentration, such as at least 5-fold its cloud concentration, such as at least 6-fold its cloud concentration, such as at least 7-fold its cloud concentration, such as at least 8-fold its cloud concentration, such as at least 9-fold its cloud concentration, such as at least 10-fold its cloud concentration, such as at least 12.5-fold its cloud concentration, such as at least 15-fold its cloud concentration, such as at least 17.5-fold its cloud concentration, such as at least 20-fold its cloud concentration, such as at least 25-fold its cloud concentration, such as at least 30-fold its cloud concentration.

The recovered products, i.e. the desaturated fatty alcohols, the desaturated fatty alcohol acetates, and/or the desaturated fatty aldehydes, may also be formulated into a pheromone composition, such as described in the section “Pheromone composition”. In one embodiment, said recovered products formulated into a pheromone composition are (Z11, Z13)-hexadecadien-1-ol, (Z11, Z13)-hexadecadien-1-ol acetate, and/or (Z11, Z13)-hexadecadienal. The composition may further comprise one or more additional compounds such as a liquid or solid carrier or substrate. Fatty aldehydes obtained from said fatty alcohols may also be comprised in such compositions.

Fatty acids can be recovered by methods known in the art. The recovered lipids are hydrolysed into free fatty acids and esterified to fatty acid alkyl ester, followed by a reduction to either fatty alcohols or fatty aldehydes.

### Kit

Provided herein is a kit of parts for performing the present methods. The kit of parts may comprise a yeast cell “ready to use” as described herein. In one embodiments, the yeast cell is a Yarrowia cell, such as a Yarrowia lipolytica cell.

In one embodiment, the yeast cell is a Saccharomyces cell, such as a Saccharomyces cerevisiae cell.

In one embodiment, the kit of parts comprises a nucleic acid or system of nucleic acids encoding the activities of interest to be introduced in the organism, such as the system of nucleic acids described in the section “Nucleic acid” herein. The nucleic acid or system of nucleic acids may be provided as a plurality of nucleic acid constructs, such as a plurality of vectors, wherein each vector encodes one or several of the desired activities.

The kit of parts may optionally comprise the yeast cell to be modified.

The kit of parts may also comprise instructions for use.

In some embodiments, the kit of parts comprises all or a combination of the above.

### Pheromone Composition

The present invention provides compounds, in particular desaturated fatty alcohols, and desaturated fatty alcohol acetates as well as derivatives thereof such as desaturated fatty aldehydes, and their use. In particular, the desaturated compounds obtainable using the present cells and methods are useful as components of pheromone compositions. Such pheromone compositions may be useful for integrated pest management. They can be used as is known in the art for e.g. mating disruption.

Thus, the desaturated fatty alcohols, the desaturated fatty alcohol acetates, and the desaturated fatty aldehydes obtainable by the present methods or using the present yeast cells may be formulated in a pheromone composition. In one embodiment, (Z13)-fatty alcohol, (Z11, Z13)-hexadecadien-1-ol, (Z11, Z13)-hexadecadienal and/or (Z11, Z13)-hexadecadien-1-ol acetate are formulated in a pheromone composition.

Such pheromone compositions may be used as integrated pest management products, which can be used in a method of monitoring the presence of pest or in a method of disrupting the mating of pest.

Thus, provided herein is a method of monitoring the presence of pest or disrupting the mating of pest, said method comprising the steps of:
i) producing (Z11, Z13)-hexadecadien-1-ol and/or a Z13-fatty alcohol by the methods described herein;
ii) converting the Z13-fatty alcohol and/or (Z11, Z13)-hexadecadien-1-ol into a Z13-fatty alcohol acetate and/or (Z11, Z13)-hexadecadien-1-ol acetate, respectively;
iii) converting the Z13-fatty alcohol and/or (Z11, Z13)-hexadecadien-1-ol into a Z13-fatty aldehyde and/or (Z11, Z13)-hexadecadienal, respectively;
iv) recovering said Z13-fatty alcohol, (Z11, Z13)-hexadecadien-1-ol, Z13-fatty alcohol acetate, (Z11, Z13)-hexadecadien-1-ol acetate, Z13-fatty aldehyde, and/or (Z11, Z13)-hexadecadienal
v) formulating said Z13-fatty alcohol, (Z11, Z13)-hexadecadien-1-ol, Z13-fatty alcohol acetate, (Z11, Z13)-hexadecadien-1-ol acetate, Z13-fatty aldehyde, and/or (Z11, Z13)-hexadecadienal as a pheromone composition; and
vi) employing said pheromone composition as an integrated pest management composition.

Also provided herein is a pheromone composition obtainable by a method comprising the following steps:
i) producing (Z11, Z13)-hexadecadien-1-ol and/or a (Z13)-fatty alcohol by the methods described herein;
ii) converting the (Z13)-fatty alcohol and/or (Z11, Z13)-hexadecadien-1-ol into a (Z13)-fatty alcohol acetate and/or (Z11, Z13)-hexadecadien-1-ol acetate, respectively;
iii) converting the (Z13)-fatty alcohol and/or (Z11, Z13)-hexadecadien-1-ol into a (Z13)-fatty aldehyde and/or (Z11, Z13)-hexadecadienal, respectively;
iv) recovering said Z13-fatty alcohol, (Z11, Z13)-hexadecadien-1-ol, Z13-fatty alcohol acetate, (Z11, Z13)-hexadecadien-1-ol acetate, Z13-fatty aldehyde, and/or (Z11, Z13)-hexadecadienal;
v) formulating said (Z13)-fatty alcohol, (Z11, Z13)-hexadecadien-1-ol, Z13-fatty alcohol acetate, (Z11, Z13)-hexadecadien-1-ol acetate, (Z13)-fatty aldehyde, and/or (Z11, Z13)-hexadecadienal as a pheromone composition.

Further provided herein is a pheromone composition comprising a Z13-fatty alcohol, (Z11, Z13)-hexadecadien-1-ol, a Z13-fatty alcohol acetate, (Z11, Z13)-hexadecadien-1-ol acetate, a Z13-fatty aldehyde and/or (Z11, Z13)-hexadecadienal, wherein said pheromone composition comprises at least 20% biobased carbon.

The pheromone composition may thus comprise between 1 and 100% Z13-fatty alcohol, between 1 and 100% (Z11, Z13)-hexadecadien-1-ol, between 1 and 100% Z13-fatty alcohol acetate, between 1 and 100% (Z11, Z13)-hexadecadien-1-ol acetate, between 1 and 100% Z13-fatty aldehyde and/or between 1 and 100% (Z11, Z13)-hexadecadienal.

In some embodiments, the ratio of (Z11, Z13)-hexadecadien-1-ol to the Z13-fatty alcohol, (Z11, Z13)-hexadecadien-1-ol acetate to the Z13-fatty alcohol acetate, and/or (Z11, Z13)-hexadecadienal to the Z13-fatty aldehyde in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z11, Z13)-hexadecadien-1-ol to the Z13-fatty alcohol in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z11, Z13)-hexadecadien-1-ol acetate to the Z13-fatty alcohol acetate in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z11, Z13)-hexadecadienal to the Z13-fatty aldehyde in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z11, Z13)-hexadecadien-1-ol to a Z11-fatty alcohol, (Z11, Z13)-hexadecadien-1-ol acetate to a Z11-fatty alcohol acetate, and/or (Z11, Z13)-hexadecadienal to a Z11-fatty aldehyde in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z11, Z13)-hexadecadien-1-ol to a Z11-fatty alcohol in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z11, Z13)-hexadecadien-1-ol acetate to a Z11-fatty alcohol acetate in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z11, Z13)-hexadecadienal to a Z11-fatty aldehyde in the pheromone composition is of at least 0.1, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z11, Z13)-hexadecadien-1-ol to Z11,Z13-hexadecadienal and/or (Z11, Z13)-hexadecadien-1-ol to (Z11, Z13)-hexadecadien-1-ol acetate in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z11, Z13)-hexadecadien-1-ol to Z11,Z13-hexadecadienal in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z11, Z13)-hexadecadien-1-ol to (Z11, Z13)-hexadecadien-1-ol acetate in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the pheromone composition comprises at least 30% biobased carbon, alternatively 40% biobased carbon, alternatively 50% biobased carbon, alternatively 60% biobased carbon, alternatively 70% biobased carbon, alternatively 75% biobased carbon, alternatively 80% biobased carbon, alternatively 85% biobased carbon, alternatively 90% biobased carbon, alternatively 95% biobased carbon.

In some embodiments, the pheromone composition comprises at least 80% biobased carbon, alternatively 85% biobased carbon, alternatively 90% biobased carbon, alternatively 95% biobased carbon, alternatively 96% biobased carbon, alternatively 97% biobased carbon, alternatively 98% biobased carbon, alternatively 99% biobased carbon.

In some embodiments, the pheromone composition comprises 30-100% biobased carbon, alternatively 40-100% biobased carbon, alternatively 50-100% biobased carbon, alternatively 60-100% biobased carbon, alternatively 70-100% biobased carbon, alternatively 75-100% biobased carbon, alternatively 80-100% biobased carbon, alternatively 85-100% biobased carbon, alternatively 90-100% biobased carbon, alternatively 95-100% biobased carbon.

In some embodiments, the pheromone composition comprises 80-100% biobased carbon, alternatively 85-100% biobased carbon, alternatively 90-100% biobased carbon, alternatively 95-100% biobased carbon, alternatively 96-100% biobased carbon, alternatively 97-100% biobased carbon, alternatively 98-100% biobased carbon, alternatively 99-100% biobased carbon.

In some embodiments, the pheromone composition comprises (Z11, Z13)-hexadecadien-1-ol, and comprises 80-100% biobased carbon, alternatively 85-100% biobased carbon, alternatively 90-100% biobased carbon, alternatively 95-100% biobased carbon, alternatively 96-100% biobased carbon, alternatively 97-100% biobased carbon, alternatively 98-100% biobased carbon, alternatively 99-100% biobased carbon. In some embodiments, the pheromone composition comprises (Z11, Z13)-hexadecadien-1-ol, and comprises 100% biobased carbon.

In further some embodiments, the pheromone composition comprises a mix of fatty alcohols including (Z11, Z13)-hexadecadien-1-ol, and comprises 80-100% biobased carbon, alternatively 85-100% biobased carbon, alternatively 90-100% biobased carbon, alternatively 95-100% biobased carbon, alternatively 96-100% biobased carbon, alternatively 97-100% biobased carbon, alternatively 98-100% biobased carbon, alternatively 99-100% biobased carbon. In further some embodiments, the pheromone composition comprises a mix of fatty alcohols including (Z11, Z13)-hexadecadien-1-ol and comprises 100% biobased carbon.

Further provided herein is a pheromone composition comprising Z13-fatty alcohol, (Z11, Z13)-hexadecadien-1-ol, Z13-fatty alcohol acetate, (Z11, Z13)-hexadecadien-1-ol acetate, Z13-fatty aldehyde and/or (Z11, Z13)-hexadecadienal, wherein said pheromone composition has a radioactive ¹⁴C level of at least 20%.

In some embodiments, the pheromone composition has a radioactive 14° C. level of at least 30%, alternatively 40%, alternatively 50%, alternatively 60%, alternatively 70%, alternatively 75%, alternatively 80%, alternatively 85%, alternatively 90%, alternatively 95%.

In some embodiments, the pheromone composition has a radioactive ¹⁴C level of at least 80%, alternatively 85%, alternatively 90%, alternatively 95%, alternatively 96%, alternatively 97%, alternatively 98%, alternatively 99%.

In some embodiments, the pheromone composition has a radioactive ¹⁴C level between 30-100%, alternatively 40-100%, alternatively 50-100%, alternatively 60-100%, alternatively 70-100%, alternatively 75-100%, alternatively 80-100%, alternatively 85-100%, alternatively 90-100%, alternatively 95-100%.

In some embodiments, the pheromone composition has a radioactive 14° C. level between 80-100%, alternatively 85-100%, alternatively 90-100%, alternatively 95-100%, alternatively 96-100%, alternatively 97-100%, alternatively 98-100% carbon, alternatively 99-100%.

Also provided herein is a method for producing a pheromone composition according to the invention comprising the following steps:
i) producing (Z11, Z13)-hexadecadien-1-ol and/or a Z13-fatty alcohol by the method as disclosed herein;
ii) converting the Z13-fatty alcohol and/or (Z11, Z13)-hexadecadien-1-ol into a Z13-fatty alcohol acetate and/or (Z11, Z13)-hexadecadien-1-ol acetate, respectively;
iii) converting the Z13-fatty alcohol and/or (Z11, Z13)-hexadecadien-1-ol into a Z13-fatty aldehyde and/or (Z11, Z13)-hexadecadienal, respectively;
iv) recovering said Z13-fatty alcohol, (Z11, Z13)-hexadecadien-1-ol, Z13-fatty alcohol acetate, (Z11, Z13)-hexadecadien-1-ol acetate, Z13-fatty aldehyde, and/or (Z11, Z13)-hexadecadienal;
v) formulating said Z13-fatty alcohol, (Z11, Z13)-hexadecadien-1-ol, Z13-fatty alcohol acetate, (Z11, Z13)-hexadecadien-1-ol acetate, Z13-fatty aldehyde and/or (Z11, Z13)-hexadecadienal as a pheromone composition, optionally comprising one or more additional compounds such as a liquid or solid carrier or substrate;
vi) optionally determining the biobased carbon content or radioactive ¹⁴C level of the pheromone composition.

Pheromone compositions as disclosed herein may be used as biopesticides. Such compositions can be sprayed or dispensed on a culture, in a field or in an orchard. They can also, as is known in the art, be soaked e.g. onto a rubber septa, or mixed with other components. In one embodiment, said compositions are placed in a device, such as a pheromone dispenser, which diffuses the pheromone composition. The dispenser may for example release pheromones at a constant, pre-adjustable, rate. This can result in mating disruption, thereby preventing pest reproduction, or it can be used in combination with a trapping device to entrap the pests. Non-limiting examples of pests against which the present pheromone compositions can be used are: the navel orangeworm Amyelois transitella, the grape leaffolder Desmia funeralis, Cedar processionary moth Thaumetopoea bonjeani, the iron prominent Notodonta dromedarius. Accordingly, use of the present compositions on a culture can lead to increased crop yield, with substantially no environmental impact.

The relative amounts of fatty alcohols and fatty alcohol acetates in the present pheromone compositions may vary depending on the nature of the crop and/or of the pest to be controlled; geographical variations may also exist. Determining the optimal relative amounts may thus require routine optimisation. The pheromone compositions may also comprise fatty aldehydes.

Examples of compositions used in pest control can be found in Kehat & Dunkelblum (1993) for H. armigera; in Alfaro et al. (2009) for C. suppressalis; in Eizaguirre et al. (2002) for S. nonagrioides; in Wu et al. (2012) for P. xylostella; in Bari et al. (2003) for P. carduidactyla; in Zhu et al. (1999) for P. interpunctella; in Wakamura (1987) for S. exigua; and in Brady et al. (1971) for C. cautella.

In some embodiments, the pheromone composition may further comprise one or more additional compounds such as a liquid or solid carrier or substrate. For example, suitable carriers or substrate include vegetable oils, refined mineral oils or fractions thereof, rubbers, plastics, silica, diatomaceous earth, wax matrix and cellulose powder.

The pheromone composition may be formulated as is known in the art. For example, it may be in the form of a solution, a gel, a powder. The pheromone composition may be formulated so that it can be easily dispensed, as is known in the art.

The present mating disruption methods may be employed in fields of transgenic crops.

Also provided herein is a method for reducing or delaying the emergence of resistance to a pesticidal trait; the method may be an integrated resistance management method.

Thus are disclosed pre-emptive and responsive methods to delay the development of resistance in a pest such as an insect, for example any of the insects listed herein, to a transgenic insecticidal crop and/or to chemical insecticide, i.e. a pre-emptive strategy. Also disclosed are methods to rescue one or more pest's susceptibility to transgenic insecticidal crops and/or chemical insecticides once resistance has developed, i.e. a responsive strategy. In some embodiments, the method comprises the application of a pheromone composition, such as obtained by the methods disclosed herein, to an agricultural area comprising a field population, wherein transgenic crops comprising one or more insecticidal traits such as transgenic insecticidal traits active against one of the insects listed herein, and optionally a refuge comprising crops devoid of insecticidal traits, to disrupt mating of the pest, thereby delaying the emergence of resistance to the insecticidal trait. The present compositions may thus be used in combination with any of the methods described in WO 2017/112887.

Also provided herein is a method for preventing or reducing crop damage from a pest such as an insect as listed herein. Such methods comprise applying mating disruption to a field by applying a pheromone composition as disclosed herein, and disrupting the expression of one or more target genes in one or more pests, thereby reducing or preventing crop damage in the field. Disruption of expression of one or more target genes can be achieved using RNAi, for example as described in WO 2017/205751.

The present compositions may thus be used in combination with any of the methods described in WO 2017/205751.

### Pheromone Compounds

Also provided herein is a pheromone compound selected from the group consisting of a Z13-fatty alcohol, (Z11, Z13)-hexadecadien-1-ol, a Z13-fatty alcohol acetate, (Z11, Z13)-hexadecadien-1-ol acetate, a Z13-fatty aldehyde and/or (Z11, Z13)-hexadecadienal, wherein said pheromone compound comprises at least 20% biobased carbon.

In some embodiments, the pheromone compound comprises at least 30% biobased carbon, alternatively 40% biobased carbon, alternatively 50% biobased carbon, alternatively 60% biobased carbon, alternatively 70% biobased carbon, alternatively 75% biobased carbon, alternatively 80% biobased carbon, alternatively 85% biobased carbon, alternatively 90% biobased carbon, alternatively 95% biobased carbon.

In some embodiments, the pheromone compound comprises at least 80% biobased carbon, alternatively 85% biobased carbon, alternatively 90% biobased carbon, alternatively 95% biobased carbon, alternatively 96% biobased carbon, alternatively 97% biobased carbon, alternatively 98% biobased carbon, alternatively 99% biobased carbon. In some embodiments, the pheromone compound comprises 100% biobased carbon.

In some embodiments, the pheromone compound comprises 30-100% biobased carbon, alternatively 40-100% biobased carbon, alternatively 50-100% biobased carbon, alternatively 60-100% biobased carbon, alternatively 70-100% biobased carbon, alternatively 75-100% biobased carbon, alternatively 80-100% biobased carbon, alternatively 85-100% biobased carbon, alternatively 90-100% biobased carbon, alternatively 95-100% biobased carbon.

In some embodiments, the pheromone compound comprises 80-100% biobased carbon, alternatively 85-100% biobased carbon, alternatively 90-100% biobased carbon, alternatively 95-100% biobased carbon, alternatively 96-100% biobased carbon, alternatively 97-100% biobased carbon, alternatively 98-100% biobased carbon, alternatively 99-100% biobased carbon.

In some embodiments, the pheromone compound is (Z11, Z13)-hexadecadien-1-ol, and comprises 80-100% biobased carbon, alternatively 85-100% biobased carbon, alternatively 90-100% biobased carbon, alternatively 95-100% biobased carbon, alternatively 96-100% biobased carbon, alternatively 97-100% biobased carbon, alternatively 98-100% biobased carbon, alternatively 99-100% biobased carbon. In some embodiments, the pheromone compound is (Z11, Z13)-hexadecadien-1-ol, and comprises 100% biobased carbon.

Also provided herein is a pheromone compound selected from the group consisting of a Z13-fatty alcohol, (Z11, Z13)-hexadecadien-1-ol, a Z13-fatty alcohol acetate, (Z11, Z13)-hexadecadien-1-ol acetate, a Z13-fatty aldehyde and/or (Z11, Z13)-hexadecadienal, wherein said pheromone compound has a radioactive ¹⁴C level of at least 20%.

In some embodiments, the pheromone compound has a radioactive ¹⁴C level of at least 30%, alternatively 40%, alternatively 50%, alternatively 60%, alternatively 70%, alternatively 75%, alternatively 80%, alternatively 85%, alternatively 90%, alternatively 95%.

In some embodiments, the pheromone compound has a radioactive ¹⁴C level of at least 80%, alternatively 85%, alternatively 90%, alternatively 95%, alternatively 96%, alternatively 97%, alternatively 98%, alternatively 99%.

In some embodiments, the pheromone compound has a radioactive ¹⁴C level between 30-100%, alternatively 40-100%, alternatively 50-100%, alternatively 60-100%, alternatively 70-100%, alternatively 75-100%, alternatively 80-100%, alternatively 85-100%, alternatively 90-100%, alternatively 95-100%.

In some embodiments, the pheromone compound has a radioactive ¹⁴C level between 80-100%, alternatively 85-100%, alternatively 90-100%, alternatively 95-100%, alternatively 96-100%, alternatively 97-100%, alternatively 98-100% carbon, alternatively 99-100%.

Further provided herein is a method for producing a da pheromone compound according to the invention, in a yeast cell, said method comprising the steps of:
i) providing a yeast cell expressing a heterologous Δ13 fatty acyl-CoA desaturase,
ii) incubating said yeast cell in a medium under conditions allowing expression of said heterologous Δ13 fatty acyl-CoA desaturase, thereby producing said pheromone compound.

In some embodiments, the yeast cell expresses a further heterologous desaturase.

In some embodiments, the yeast cell further expresses at least one alcohol-forming fatty acyl-CoA reductase (FAR).

In some embodiments, the method further comprises a step of determining the biobased carbon content or radioactive ¹⁴C level of the pheromone compound.

### Biobased Products

“Biobased” products may be defined as products wherein:
1. the total carbon content of the product is at least 30%, and
2. the carbon content of a renewable raw material (biobased) is at least 20%.

Both fossil and renewable raw materials consist mainly of carbon (C). Carbon occurs in several isotopes. Isotope ¹⁴C is radioactive and occurs naturally in all living organisms (plants, animals, etc) in a fixed relative concentration which is nearly identical to the relative ¹⁴C concentration in the atmosphere. At this concentration, the radioactivity level of ¹⁴C is 100%. Once an organism is no longer living, this concentration, and thus the radioactivity rate, decays with a half-life of approximately 5700 years. The radioactive ¹⁴C level of an unknown substance can therefore help determine how old the carbon contained in the substance is.

“Young” carbon (0 to 10 years) derived from renewable raw materials, such as plants or animals, has a relative isotope ¹⁴C concentration which is nearly identical to the relative ¹⁴C concentration in the atmosphere and the radioactive ¹⁴C level of such young carbon is thus about 100%.

“Old” carbon (millions of years) derived from synthetic or fossil (petrochemical) sources is greatly depleted from isotope ¹⁴C as the age of such synthetic and fossil sources far exceeds the half-life of isotope ¹⁴C which is approximately 5700 years. Hence, carbon derived from synthetic or fossil sources has a relative isotope ¹⁴C concentration around 0% and the radioactive ¹⁴C level of such old carbon is thus about 0%.

In one embodiment the term “radioactive ¹⁴C level” refer to the total radioactive ¹⁴C level of a given substance, product or composition, as defined above.

The isotope ¹⁴C method may be used to determine the concentration of young (renewable) materials in comparison with the concentration of old (fossil) resources. The carbon content of a renewable raw material is referred to as the “biobased carbon content”. The carbon content of a renewable raw material or the “biobased carbon content” may be determined as described below.

When measuring the biobased carbon content, the result may be reported as “% biobased carbon”. This indicates the percentage carbon from “natural” (plant or animal by-product) sources versus “synthetic” or “fossil” (petrochemical) sources. For reference, 100% biobased carbon indicates that a material is entirely sourced from plants or animal by-products and 0% biobased carbon indicates that a material did not contain any carbon from plants or animal by-products. A value in between represents a mixture of natural and fossil sources.

Example: If a product has a radioactive ¹⁴C level of 80%, it means that the product consists of 80% renewable and 20% fossil carbon (C). In other words, the product is 80% biobased.

The analytical measurement may be cited as “percent modern carbon (pMC)”. This is the percentage of ¹⁴C measured in the sample relative to a modern reference standard (NIST 4990C). The % Biobased Carbon content is calculated from pMC by applying a small adjustment factor for ¹⁴C in carbon dioxide in air today. It is important to note that all internationally recognized standards using ¹⁴C assume that the plant or biomass feedstocks were obtained from natural environments. pMC may be analysed by a standard test method, such as “ASTM D6866”.

### EXAMPLES

### Example 1: Construction of BioBricks and Plasmids

For expression in Saccharomyces cerevisiae genes were synthetized by GeneArt (ThermoFisher), and directly cloned by Gateway cloning into donor vector pDONR221 followed by subcloning into the S. cerevisiae expression vector pYEX-CHT-DEST (encoding an uracil and leucine selection marker cassette), resulting in recombinant expression vector pYEX-CHT-Desat16, pYEX-CHT-Desat16-AtrAATQ and pYEX-CHT-Desat16-AtrAATQ-FAR1-AtrAATQ.

For expression in Yarrowia lipolytica, genes were synthesized by GeneArt (Life Technologies) in codon-optimized versions for Yarrowia lipolytica. All the genes were amplified by PCR using Phusion U Hot Start DNA Polymerase (ThermoFisher) to obtain the fragments for cloning into yeast expression vectors. The primers are listed in Table 1 and the resulting DNA fragments (BioBricks) are listed in Table 2. The PCR products were separated on a 1%-agarose gel containing Midori Green Advance (Nippon Genetics Europe GmbH). PCR products of the correct size were excised from the gel and purified using the Nucleospin Gel and PCR Clean-up kit (Macherey-Nagel).

Yeast vectors with USER cassette were linearized with FastDigest SfaAl (ThermoFisher) for 2 hours at 37° C. and then nicked with Nb.Bsml (New England Biolabs) for 1 hour at 65° C. The resulting vectors containing sticky ends were separated by gel electrophoresis, excised from the gel, and gel-purified using the Nucleospin Gel and PCR Clean-up kit (Macherey-Nagel). The DNA fragments were cloned into vectors by USER-cloning as described in (Holkenbrink, et al., 2018). The reaction was transformed into chemically competent E. coli DHα cells and the cells were plated on Lysogeny Broth (LB) agar plates with 100 mg/L ampicillin. The plates were incubated overnight at 37° C. and the resulting colonies were screened by colony PCR. The plasmids were purified from overnight E. coli liquid cultures and the correct cloning was confirmed by sequencing. The constructed vectors are listed in Table 3.

Strains marked with “***” were constructed as follows. The indicated genes were amplified with primers gene-specific containing a 5′-overhang of “ACTTTTTGCAGTACUAACCGCAG” in the forward primer and a 3′-overhang of “CACGCGAU” in the reverse primer. The first “ATG” of the target gene sequence was omitted. These PCR products were cloned together with BB9454 either into integrative vectors or episomal vectors as described in (Holkenbrink, et al., 2018)

TABLE 1
Primers

| | | NCBI | |
|---|---|---|---|
| | | accession | Hybridises at positions |
| Primer ID | Template | number | (or SEQ ID NO) |
| PR10595 | Yali0C | NC_006069 | 1244252 . . . 1244265 |
| PR10604 | pCfB6630* | — | 3458 . . . 3498 |
| PR10607 | Yali0A | NC_006067 | 483924 . . . 483945 |
| PR10852 | Yali0E | NC_006071 | 784691 . . . 784713 |
| PR11110 | pCfB6681 | — | 2306 . . . 2336 |
| PR11111 | pCfB6681* | — | 5129 . . . 5152 |
| PR14148 | Yali0E | NC_006071 | 784691 . . . 784708 |
| PR14279 | Yali0C | NC_006069 | 1244254 . . . 1244265 |
| PR14620 | Yali0A | NC_006067 | 2188565 . . . 2188546 |
| PR15781 | Yali0B | NC_006068 | 1803359 . . . 1803341 |
| PR15788 | Yali0A | NC_006067 | 456893 . . . 456922 |
| PR15789 | Yali0F | NC_006072 | 1948664 . . . 1948678 |
| PR15930 | Yali0C | NC_006069 | 1243743 . . . 1243762 |
| PR16594 | SEQ ID NO: 14 | — | 6 . . . 21 |
| PR16595 | SEQ ID NO: 14 | — | 1348 . . . 1368 |
| PR18214 | Yali0C | NC_006069 | 1243743 . . . 1243761 |
| PR18255 | Yali0E | NC_006071 | 2882052 . . . 2882071 |
| PR18256 | Yali0E | NC_006071 | 2882071 . . . 2882052 |
| PR18928 | Yali0C | NC_006069 | 1244239 . . . 1244226 |
| PR21764 | SEQ ID NO: 8 | — | 963 . . . 981 |
| PR21806 | SEQ ID NO: 4 | — | 4 . . . 21 |
| PR21807 | SEQ ID NO: 4 | — | 999 . . . 1017 |
| PR22074 | SEQ ID NO: 8 | — | 4 . . . 23 |
| PR22297 | SEQ ID NO: 4 | — | 999 . . . 1017 |
| PR23166 | Yali0E | NC_006071 | 2086416 . . . 2086402 |
| PR23167 | Yali0E | NC_006071 | 2085907 . . . 2085919 |
| PR23168 | Yali0E | NC_006071 | 2086480 . . . 2086497 |
| PR23169 | Yali0E | NC_006071 | 2087001 . . . 2086986 |
| PR25094 | | | SEQ ID NO: 17 |
| PR25309 | Yali0C | NC_006069 | 1244237 . . . 1244223 |
| PR25381 | | | SEQ ID NO: 18 |
| PR25423 | SEQ ID NO: 2 | — | 4 . . . 20 |
| PR25424 | SEQ ID NO: 2 | — | 1030 . . . 1047 |
| PR25520 | SEQ ID NO: 16 | — | 4 . . . 20 |
| PR25521 | SEQ ID NO: 16 | — | 1613 . . . 1632 |

TABLE 2
DNA fragments (BioBricks) obtained by PCR
using the indicated template and primers

| BioBrick | | | Forward | Reverse |
|---|---|---|---|---|
| ID | Description | Template | primer | primer |
| BB1135 | Vector backbone | pCfB6681* | PR11110 | PR11111 |
| BB1631 | Regions downstream of | pCfB4586* | PR14148 | PR15781 |
| | Y. lipolytica pex20 and | | | |
| | lip2 | | | |
| BB1635 | Promoter for gRNA | pCfB4589* | PR10607 | PR15788 |
| | expression | | | |
| BB1636 | Terminator region for | pCfB4589* | PR15789 | PR10604 |
| | gRNA expression | | | |
| B1688 | region upstream of | ST4840** | PR14279 | PR15930 |
| | Y. lipolytica tef1 | | | |
| BB1740 | Fatty acyl-CoA reductase | SEQ ID NO: 14 | PR16594 | PR16595 |
| | from Helicoverpa | | | |
| | armigera | | | |
| BB2093 | region upstream of | ST4840** | PR10595 | PR18214 |
| | Y. lipolytica tef1 | | | |
| BB2719 | region upstream of | ST4840** | PR18928 | PR18214 |
| | Y. lipolytica tef1 | | | |
| BB7935 | Spodoptera litura | SEQ ID NO: 4 | PR21806 | PR21807 |
| | desaturase Desat38 | | | |
| BB8047 | Amyelois transitella | SEQ ID NO: 8 | PR22074 | PR21764 |
| | desaturase Desat16 | | | |
| BB8250 | Region upstream of tef1 | pBP7948 | PR10595 | PR22297 |
| | fused to | | | |
| | Spodoptera litura | | | |
| | desaturase Desat38 | | | |
| BB8679 | Genomic region of | ST4840** | PR23167 | PR23166 |
| | Yarrowia lipolytica | | | |
| BB8680 | Genomic region of | ST4840** | PR23168 | PR23169 |
| | Yarrowia lipolytica | | | |
| BB8955 | Regions downstream of | pBP8662 | PR10852 | PR14620 |
| | Y. lipolytica pex20 and | | | |
| | lip2 | | | |
| BB9454 | region upstream of | ST4840** | PR25309 | PR18214 |
| | Y. lipolytica tef1 | | | |
| BB9506 | Regions downstream of | pBP9002 | PR25381 | PR25094 |
| | Y. lipolytica pex20 and | | | |
| | lip2 | | | |
| BB9529 | Amyelois transitella | SEQ ID NO: 2 | PR25423 | PR25424 |
| | desaturase AtrAATQ | | | |
| BB9608 | Fatty acyl-CoA reductase | SEQ ID NO: 16 | PR25520 | PR25521 |
| | from Amyelois transitella | | | |

*Holkenbrink et al., 2018
**Holkenbrink et al., 2020

TABLE 3
Vectors

| | | Parent | |
|---|---|---|---|
| Plasmid ID | Resistance | plasmid | BioBricks |
| pYEX-CHT-Desat16 | — | pYEX- | SEQ ID NO: 47 |
| | | CHT*** | |
| pYEX-CHT- | — | | SEQ ID NO: 47, SEQ ID |
| Desat16-AtrAATQ | | | NO: 46 |
| pYEX-CHT- | — | pYEX- | SEQ ID NO: 47, SEQ ID |
| Desat16-AtrAATQ- | | CHT*** | NO: 46, SEQ ID NO: 48 |
| FAR1-AtrAATQ | | | |
| pYEX-CHT- | — | pYEX- | SEQ ID NO: 47, SEQ ID |
| Desat16-AtrAATQ- | | CHT*** | NO: 46, SEQ ID NO: 49 |
| FAR33-AtrAATQ | | | |
| pYEX-CHT- | — | pYEX- | SEQ ID NO: 52, SEQ ID |
| Desat45-AtrAATQ- | | CHT*** | NO: 46, SEQ ID NO: 49 |
| FAR33-AtrAATQ | | | |
| pYEX-CHT- | — | pYEX- | SEQ ID NO: 50, SEQ ID |
| Desat44-AtrAATQ- | | CHT*** | NO: 46, SEQ ID NO: 49 |
| FAR33-AtrAATQ | | | |
| pYEX-CHT-Tpi- | — | pYEX- | SEQ ID NO: 54 |
| PGFAD | | CHT*** | |
| pBP7948 | — | pCfB6685* | BB7935, BB2719 |
| pBP8131 | — | pCfB6679* | BB2093, BB8047 |
| pBP8138 | — | pCfB6679* | BB8250 |
| pBP8662 | — | | BB1135, BB8679, BB8680, |
| | | | BB1631 |
| pBP9002 | Nourseothricin | pCfB3405* | BB8955 |
| pBP9004 | Hygromycin | pCfB3431** | BB8955 |
| pBP9560 | — | pBP9002 | BB1688, BB1740 |
| pBP9725 | Hygromycin | pBP9699 | BB9454, BB9529 |
| pBP9827 | Nourseothricin | pBP9698 | BB9454, BB9608 |
| pBP9698 | Nourseothricin | pCfB3405* | BB9506 |
| pBP9699 | Hygromycin | pCfB3431** | BB9506 |
| pCfB6638 | Nourseothricin | pCfB3405* | BB1635, BB1636, PR18255, |
| | | | PR18256 |

*Holkenbrink et al., 2018
**Holkenbrink et al., 2020
***Patel et al., 2003

### Example 2: Construction of Yeast Strains

Saccharomyces cerevisiae strains were constructed by transformation of DNA vectors as described in Ding and Löfstedt 2015. The recombinant expression vector pYEX-CHT-Desat16, pYEX-CHT-Desat16-AtrAATQ, PYEX-CHT-Desat16-AtrAATQ-FAR33-AtrAATQ, pYEX-CHT-Desat45-AtrAATQ-FAR33-AtrAATQ, pYEX-CHT-Desat44-AtrAATQ-FAR33-AtrAATQ and pYEX-CHT-Tpi-PGFAD were transformed into S. cerevisiae strain Aole1 Aelo1 (MATa elo1::HIS3 ole1::LEU2 ade2 his3 leu2 ura3) resulting in strain ST201104_BJD and ST201105_BJD, while vector pYEX-CHT-Desat16-AtrAATQ-FAR1-AtrAATQ was introduced into the S. cerevisiae strain INVSc (MATa HIS3 LEU2 trp1-289 ura3-52) resulting in strain ST210327_BJD (Table 4). The transformations were performed using the S. cerevisiae easy yeast transformation kit (Thermofisher). For selection of uracil (and leucine) prototrophs, cells were grown on synthetic complete medium containing 0.7% YNB (w/o amino acids, with ammonium sulfate), agar, drop-out medium lacking uracil (Formedium™ LTD, Norwich, England), 2% glucose, 0.5% tergitol (type Nonidet NP-40, Merck), 0.01% adenine (Merck) and 0.5 mM oleic acid (Merck) as extra fatty acid source. Four days after transformation at 30° C., individual colonies were picked and directly cultivated as described in Example 3.

Yarrowia lipolytica strains were constructed by transformation of DNA vectors as described in Holkenbrink et al., 2018. Integrative vectors were linearized with FastDigest Notl prior to transformation. When needed, helper vectors to promote the integration into specific genomic regions were co-transformed with the integrative plasmid or DNA repair fragments listed in Table 3. Strains were selected on yeast peptone dextrose (YPD) agar with appropriate antibiotics selection. Correct genotype was confirmed by colony PCR and when needed by sequencing. The resulting strains are listed in Table 4.

TABLE 4
Yeast strains

| | Yeast | Overexpressed | Parent | |
|---|---|---|---|---|
| Name | species | gene | Strain | DNA |
| Δole1 Δelo1 | S. cerevisiae | — | | |
| ST201104_BJD | S. cerevisiae | Desat16 | Δole1 | pYEX-CHT- |
| | | | Δelo1 | Desat16 |
| ST201105_BJD | S. cerevisiae | Desat16 AtrAATQ | Δole1 | pYEX-CHT- |
| | | | Δelo1 | Desat16- |
| | | | | AtrAATQ |
| INVSc | S. cerevisiae | — | | |
| ST210327_BJD | S. cerevisiae | Desat16 AtrAATQ | INVSc | pYEX-CHT- |
| | | FAR1 | | Desat16- |
| | | | | AtrAATQ-FAR1- |
| | | | | AtrAATQ |
| Sc_Desat16_{—} | S. cerevisiae | Desat16 AtrAATQ | Δole1 | pYEX-CHT- |
| AtrAATQ_FAR33 | | FAR33 | Δelo1 | Desat16- |
| | | | | AtrAATQ- |
| | | | | FAR33-AtrAATQ |
| Sc_Desat45_{—} | S. cerevisiae | Desat45 AtrAATQ | Δole1 | pYEX-CHT- |
| AtrAATQ_FAR33 | | FAR33 | Δelo1 | Desat45- |
| | | | | AtrAATQ- |
| | | | | FAR33-AtrAATQ |
| Sc_Desat44_{—} | S. cerevisiae | Desat44 AtrAATQ | Δole1 | pYEX-CHT- |
| AtrAATQ_FAR33 | | FAR33 | Δelo1 | Desat44- |
| | | | | AtrAATQ- |
| | | | | FAR33-AtrAATQ |
| Sc_Tpi_PGFAD | S. cerevisiae | Tpi-PGFAD | Δole1 | pYEX-CHT-Tpi- |
| | | | Δelo1 | PGFAD |
| ST6629* | Y. lipolytica | — | | |
| ST8403 | Y. lipolytica | Desat16 | ST6629 | pBP8131, |
| | | | | pCfB6638 |
| ST8407 | Y. lipolytica | Desat38 | ST6629 | pBP8138, |
| | | | | pCfB6638 |
| ST10615 | Y. lipolytica | Desat38 | ST8407 | pBP9004 |
| ST10750 | Y. lipolytica | Desat38 | ST8407 | pBP9725 |
| | | AtrAATQ | | |
| ST10857 | Y. lipolytica | Desat16 | ST8403 | pBP9725 |
| | | AtrAATQ | | |
| ST10858 | Y. lipolytica | Desat16 | ST8403 | pBP9004 |
| ST11304 | Y. lipolytica | Desat38 | ST10750 | pBP9002 |
| | | AtrAATQ | | |
| ST11305 | Y. lipolytica | Desat38 | ST10750 | pBP9560 |
| | | AtrAATQ FAR1 | | |
| ST11306 | Y. lipolytica | Desat38 | ST10750 | pBP9827 |
| | | AtrAATQ FAR33 | | |
| ST12040** | Y. lipolytica | AtrAATQ | ST6629 | SEQ ID NO: 2 |
| ST12323** | Y. lipolytica | Desat16 | ST12040 | |
| ST12326** | Y. lipolytica | Desat21 | ST12040 | |
| ST12327** | Y. lipolytica | Desat37 | ST12040 | |
| ST12328** | Y. lipolytica | Desat38 | ST12040 | |
| ST12329** | Y. lipolytica | Desat44 | ST12040 | SEQ ID NO: 138 |
| ST12330** | Y. lipolytica | Desat45 | ST12040 | |
| ST12332** | Y. lipolytica | Desat51 | ST12040 | |
| ST12333** | Y. lipolytica | Desat63 | ST12040 | |
| ST12334** | Y. lipolytica | Desat65 | ST12040 | |
| ST12335** | Y. lipolytica | Desat71 | ST12040 | |
| ST12336** | Y. lipolytica | Desat79 | ST12040 | |
| ST12337** | Y. lipolytica | Desat72 | ST12040 | |
| ST12338** | Y. lipolytica | Desat70 | ST12040 | |
| ST12339** | Y. lipolytica | Desat73 | ST12040 | |
| ST12323** | Y. lipolytica | Desat16 | ST12040 | |
| ST12326** | Y. lipolytica | Desat21 | ST12040 | |
| ST12327** | Y. lipolytica | Desat37 | ST12040 | |
| ST12328** | Y. lipolytica | Desat38 | ST12040 | |
| ST12329** | Y. lipolytica | Desat44 | ST12040 | SEQ ID NO: 138 |
| ST12330** | Y. lipolytica | Desat45 | ST12040 | |
| ST12332** | Y. lipolytica | Desat51 | ST12040 | |
| ST12333** | Y. lipolytica | Desat63 | ST12040 | |
| ST12334** | Y. lipolytica | Desat65 | ST12040 | |
| ST12335** | Y. lipolytica | Desat71 | ST12040 | |
| ST12336** | Y. lipolytica | Desat79 | ST12040 | |
| ST12337** | Y. lipolytica | Desat72 | ST12040 | |
| ST12338** | Y. lipolytica | Desat70 | ST12040 | |
| ST12339** | Y. lipolytica | Desat73 | ST12040 | |
| ST12575** | Y. lipolytica | Desat37 | ST6629 | SEQ ID NO: 30, |
| | | AtrAATQ | | SEQ ID NO: 2 |
| ST12493** | Y. lipolytica | pCfB3405* | ST12575 | |
| ST12583** | Y. lipolytica | FAR1 | ST12575 | |
| ST12456** | Y. lipolytica | FAR4 | ST12575 | |
| ST12457** | Y. lipolytica | FAR5 | ST12575 | |
| ST12458** | Y. lipolytica | FAR6 | ST12575 | |
| ST12459** | Y. lipolytica | FAR8 | ST12575 | |
| ST12460** | Y. lipolytica | FAR9 | ST12575 | |
| ST12462** | Y. lipolytica | FAR12 | ST12575 | |
| ST12463** | Y. lipolytica | FAR42 | ST12575 | |
| ST12465** | Y. lipolytica | FAR25 | ST12575 | |
| ST12466** | Y. lipolytica | FAR13 | ST12575 | |
| ST12467** | Y. lipolytica | FAR14 | ST12575 | |
| ST12468** | Y. lipolytica | FAR15 | ST12575 | |
| ST12469** | Y. lipolytica | FAR16 | ST12575 | |
| ST12470** | Y. lipolytica | FAR17 | ST12575 | |
| ST12471** | Y. lipolytica | FAR18 | ST12575 | |
| ST12472** | Y. lipolytica | FAR19 | ST12575 | |
| ST12473** | Y. lipolytica | FAR20 | ST12575 | |
| ST12475** | Y. lipolytica | FAR22 | ST12575 | |
| ST12477** | Y. lipolytica | FAR27 | ST12575 | |
| ST12478** | Y. lipolytica | FAR28 | ST12575 | |
| ST12479** | Y. lipolytica | FAR29 | ST12575 | |
| ST12480** | Y. lipolytica | FAR30 | ST12575 | |
| ST12482** | Y. lipolytica | FAR43 | ST12575 | |
| ST12483** | Y. lipolytica | FAR33 | ST12575 | |
| ST12484** | Y. lipolytica | FAR34 | ST12575 | |
| ST12485** | Y. lipolytica | FAR35 | ST12575 | |
| ST12486** | Y. lipolytica | FAR44 | ST12575 | |
| ST12487** | Y. lipolytica | FAR48 | ST12575 | |
| ST12488** | Y. lipolytica | FAR49 | ST12575 | |
| ST12489** | Y. lipolytica | FAR45 | ST12575 | |
| ST12491** | Y. lipolytica | FAR38 | ST12575 | |
| ST12492** | Y. lipolytica | FAR40 | ST12575 | |
| ST12564** | Y. lipolytica | FAR41 | ST12575 | |
| ST12565** | Y. lipolytica | FAR51 | ST12575 | |
| ST12566** | Y. lipolytica | FAR52 | ST12575 | |
| ST12567** | Y. lipolytica | FAR47 | ST12575 | |
| ST12568** | Y. lipolytica | FAR46 | ST12575 | |
| ST12569** | Y. lipolytica | FAR50 | ST12575 | |
| ST12648** | Y. lipolytica | Desat38 | ST6629 | |
| | | Desat83 | | |
| ST12649** | Y. lipolytica | Desat38 | ST6629 | |
| | | Desat84 | | |
| ST12650** | Y. lipolytica | Desat38 | ST6629 | |
| | | Desat85 | | |
| ST12611** | Y. lipolytica | Desat38 | ST6629 | |
| | | NdDes1 | | |
| ST12613** | Y. lipolytica | Desat38 | ST6629 | |
| STNOW | Y. lipolytica | AtrAATQ | | SEQ ID NO: 2, |
| | | FAR1 | | SEQ ID NO: 14, |
| | | Desat16 | | SEQ ID NO: 8, |
| | | Desat38 | | SED ID NO: 4 |

*Holkenbrink et al., 2020
**For description of strain constructions see text

### Example 3: Cultivation of Strains and Analysis of Fatty Alcohols and Fatty Acid Methyl Esters (FAME)

S. cerevisiae strains were cultivated as follows. Four days after transformation and incubation at 30° C., individual colonies were picked up and inoculated into 2 mL synthetic complete medium. The cultures were incubated at 30° C. at 300 rpm for 48 hours. Yeast cultures were diluted to an OD600 of 0.4 in 4 mL fresh selective medium containing 2% galactose (1 mM CuSO₄) and cultured in 30° C. in a shaking incubator at 30° C. for additional 48 hours.

For the analysis of fatty acids of Saccharomyces cerevisiae yeast cells were harvested by centrifugation at 3,000 rpm and the supernatant was discarded. Total lipids were extracted from the yeast cell pellet by addition of 3.75 mL of methanol/chloroform (2:1, v/v), in a glass tube. 10 μL of 19:Me (1 μM/μL in heptane) were added as internal standard. One mL of acetic acid (0.15 M) and 1.25 ml of water were added to the tube to wash the chloroform phase. Tubes were vortexed vigorously and centrifuged at 2000 rpm for 2 min. The bottom chloroform phase, about 1 mL, containing the total lipids, was transferred to a new glass tube. Fatty acid methyl esters (FAMEs) were made from this total lipid extract. The solution of total lipids was evaporated to dryness under gentle nitrogen flow. One mL of sulfuric acid (2% in methanol) was added to the tube, which was then vortexed vigorously and incubated at 90° C. for an hour. After incubation, 1 ml of water was added, mixed well, and then 1 mL of hexane was used to extract the FAMEs. Fatty alcohols were extracted from S. cerevisiae cells using 800 μL of heptane plus sonication. 10 μL of 17:OH (1 μM/μL in heptane) were added as internal standard. After brief centrifugation, the supernatant was transferred to a new tube and subjected to GC-MS analysis. The methyl esters and fatty alcohols were subjected to GC-MS analyses on a Hewlett Packard 6890 GC coupled to a mass spectrometer HP 5973. The GC was equipped with an INNOWax column (30 m×0.25 mm i.d.×0.25 μm film thickness, Agilent Technologies), and helium was used as carrier gas (average velocity: 33 cm/s). The MS was operated in electron impact mode (70 eV), scanning between m/z 30 and m/z 400, and the injector was configured in splitless mode at 220° C. The oven temperature was set to 80° C. for 1 min, then increased at a rate of 10° C./min up to 210° C., followed by a hold at 210° C. for 15 min, and then increased at a rate of 10° C./min up to 230° C. followed by a hold at 230° C. for 20 min. Data were analyzed using the ChemStation software (Agilent, Technologies, USA).

Yarrowia lipolytica strains were inoculated from a YPD agar plate (10 g/L yeast extract, 10 g/L peptone, 20 g/L glucose, 15 g/L agar agar) to an initial OD600 of 0.1-0.2 into 2.5 mL YPG medium (10 g/L yeast extract, 10 g/L peptone, 40 g/L glycerol) in 24 well-plates (EnzyScreen). The plates were incubated at 28° C., shaken at 300 rpm. After 22 h, the plates were centrifuged for 5 min at 4° C. and 3,000×g. The supernatant was discarded and the cells were resuspended in 1.25 mL production medium per well (50 g/L glycerol, 5 g/L yeast extract, 4 g/L KH₂PO₄, 1.5 g/L MgSO₄, 0.2 g/L NaCl, 0.265 g/L CaCl₂)·2H₂O, 2 mL/L trace elements solution: 4.5 g/L CaCl₂)·2H₂O, 4.5 g/L ZnSO₄·7H₂O, 3 g/L FeSO₄·7H₂O, 1 g/L H₃BO₃, 1 g/L MnCl₂·4H₂O, 0.4 g/L N Na₂MoO₄·2H₂O, 0.3 g/L CoCl₂·6H₂O, 0.1 g/L CuSO₄·5H₂O, 0.1 g/L KI, 15 g/L EDTA). The media was supplemented with antibiotics if necessary. The plate was incubated for 28 hours at 28° C., shaken at 300 rpm.

For analysis of fatty alcohols of Yarrowia lipolytica, 1 mL of each vial was harvested by centrifugation for 5 min at 4° C. and 3,000×g. Each cell pellet was extracted with 1 ml of ethyl acetate:ethanol (84:15) and 10 μL of Z10-17:Me (2 mg/mL) as internal standard was added to the cell pellet. The samples were vortexed for 20 sec and incubated for 1 hour at room temperature, followed by 5 min of vortexing. 300 μL of H2O was added to each sample. The samples were vortexed and centrifuged for 5 min at 21° C. and 3,000×g. The upper organic phase was analyzed via gas chromatography-mass spectrometry (GC-MS). GC-MS analyses were performed on an Agilent 7820A GC coupled to a mass selective detector Agilent 5977B. The GC was equipped with an DB Fatwax column (30 m×0.25 mm×0.25 μm), and helium was used as carrier gas. The MS was operated in electron impact mode (70 eV), scanning between m/z 30 and 400, and the injector was configured in split mode 20:1 at 220° C. Oven temperature was set to 80° C. for 1 min, then increased at a rate of 20° C./min to 210° C., followed by a hold at 210° C. for 7 min, and then increased at a rate of 20 C/min to 230° C. Compounds were identified by comparison of retention times and mass spectra of the reference compounds. Compounds were quantified by the ion 55.1 m/z. Data were analyzed by the Agilent Masshunter software. The concentrations of fatty alcohols were calculated based on standard calibration curves prepared with reference standards. For analysis of the fatty acids of Yarrowia lipolytica, 1 mL of each vial was harvested by centrifugation for 5 min at 4° C. and 3,000×g. Each pellet was extracted with 1000 μL 1M HCl in methanol (anhydrous). The samples were vortexed for 20 sec and placed in the 80° C. water bath for 2 h. The samples were vortexed every 30 min for 10 sec. After the samples were cooled down to room temperature, 1000 μL of 1M NaOH in methanol (anhydrous), 500 μL of NaCl saturated H2O, 990 μL of hexane and 10 μL of Z10-17:Me (2 mg/mL) as internal standard were added. The samples were vortexed and centrifuged for 5 min at 21° C. and 3,000×g. The upper organic phase was analyzed via GC-MS as described above.

The samples described in Example 9 were analyzed as follows. The upper organic phase was analyzed via gas chromatography-Flame Ion Detection (GC-FID). GC-FID analyses were performed on an Agilent 7890B GC system. The GC was equipped with an HP-5 column (30 m×0.25 mm×0.25 μm), and hydrogen was used as carrier gas. The FID was operated at 300° C. with air flow of 400 mL/min and hydrogen flow of 30 mL/min and makeup gas flow (N2) at 25 mL/min. The injector was configured in split mode 40:1 at 220° C. Oven temperature was set to 150° C. for 3 min, then increased at a rate of 10° C./min to 210° C., and then increased at a rate of 20 C/min to 300° C. Compounds were identified by comparison of retention times of the reference compounds. Data were analyzed by the Agilent Masshunter software. The concentrations of fatty alcohols were calculated based on standard calibration curves prepared with reference standards.

### Example 4: Production of Z11,Z13-16 Acid in Yeast

The ΔZ11-desaturase Desat16 from Amyelois transitella was either expressed alone (ST201104_BJD) or in combination with ΔZ13-desaturase AtrAATQ from Amyelois transitella (ST201105_BJD), respectively, in Saccharomyces cerevisiae Aole1 Aelo1 in which both the yeast native fatty acyl-CoA desaturase-encoding gene OLE1 and the fatty acid elongase-encoding gene ELO1 were deleted (Table 3). The strains were cultivated as described in Example 3 and lipids present in the cell pellets were extracted, converted into methyl ester and subjected to GC-MS analyses. The derivatized extracts of both strains, ST201104_BJD and ST201105_BJD, were confirmed to contain Z11-16:Me, while the extract of strain ST201105 additionally contained the double-unsaturated Z11,Z13-16:Me (Table 5).

TABLE 5
Production of Z11,Z13-16:Acid in Saccharomyces cerevisiae

| | Desaturases | Z11-16:Me | Z11,Z13-16:Me |
|---|---|---|---|
| Strain ID | expressed | (mg/L) | (mg/L) |
| Δole1 Δelo1 | — | 0 | 0 |
| ST201104_BJD | Desat16 | 1.9 ± 0.2 | 0 |
| ST201105_BJD | Desat16 AtrAATQ | 1.4 ± 0.1 | 0.5 ± 0.1 |

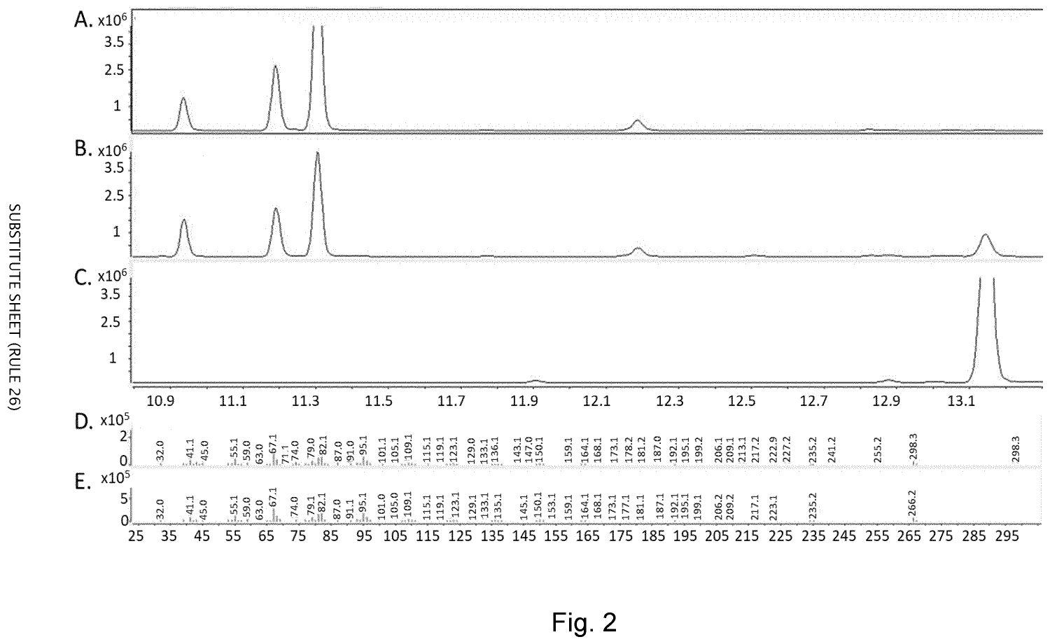

Two Δ11 desaturases, Desat16 and Desat38, from Amyelois transitella and Spodoptera litura, respectively, were either expressed individually (ST10858 and ST10615) or in combination with Δ13 desaturase AtrAATQ from Amyelois transitella (ST10857 and ST10750) in the yeast Yarrowia lipolytica. The strains were cultivated as described in Example 3 and lipids present in the cell pellets were extracted, converted into methyl ester and subjected to GC-MS chromatography. The derivatized extracts of strains ST10858 and ST10615 were confirmed to contain Z11-16:Me, while extracts of strains ST10857 and ST10750 additionally contained the double-unsaturated Z11,Z13-16:Me (Table 6; FIG. 2). The concentration of Z11-16:Me in extracts of strains ST10867 and ST10750 was lower, as expected, because part of Z11-16:CoA was unsaturated by AtrAATQ into Z11,Z13-16:CoA.

TABLE 6
Production of Z11,Z13-16:Acid in Yarrowia lipolytica

| | Desaturases | Z11-16:Me | Z11,Z13- |
|---|---|---|---|
| Strain ID | expressed | (mg/L) | 16:Me (mg/L) |
| ST10858 | Desat16 | 88 ± 5 | 0 ± 0 |
| ST10857 | Desat16 AtrAATQ | 55 ± 8 | 11 ± 1 |
| ST10615 | Desat38 | 130 ± 11 | 0 ± 0 |
| ST10750 | Desat38 AtrAATQ | 79 ± 3 | 15 ± 1 |

### Example 5: Production of Z11,Z13-16:OH in Yeast

S. cerevisiae strain ST210327_BJD expressing Δ11 desaturase Desat16, Δ13 desaturase AtrAATQ from Amyelois transitella and FAR1 from Helicoverpa armigera was cultivated as described in Example 3. Strain ST201105_BJD, only expressing Desat16 and AtrAATQ, served as a negative control. After GC-MS analysis, Z11,Z13-16:OH was detected in extracts of strain ST210327_BJD, but not in extracts of strain ST201105_BJD (Table 7).

TABLE 7
Production of Z11,Z13-16:OH in Saccharomyces cerevisiae

| | Desaturases and | Z11-16:OH | Z11,Z13-16:OH |
|---|---|---|---|
| Strain ID | reductases expressed | (mg/L) | (mg/L) |
| ST201105_BJD | Desat16 AtrAATQ | 0 ± 0 | 0 ± 0 |
| ST210327_BJD | Desat16 AtrAATQ FAR1 | 3.9 ± 0.2 | 16.3 ± 1.3 |

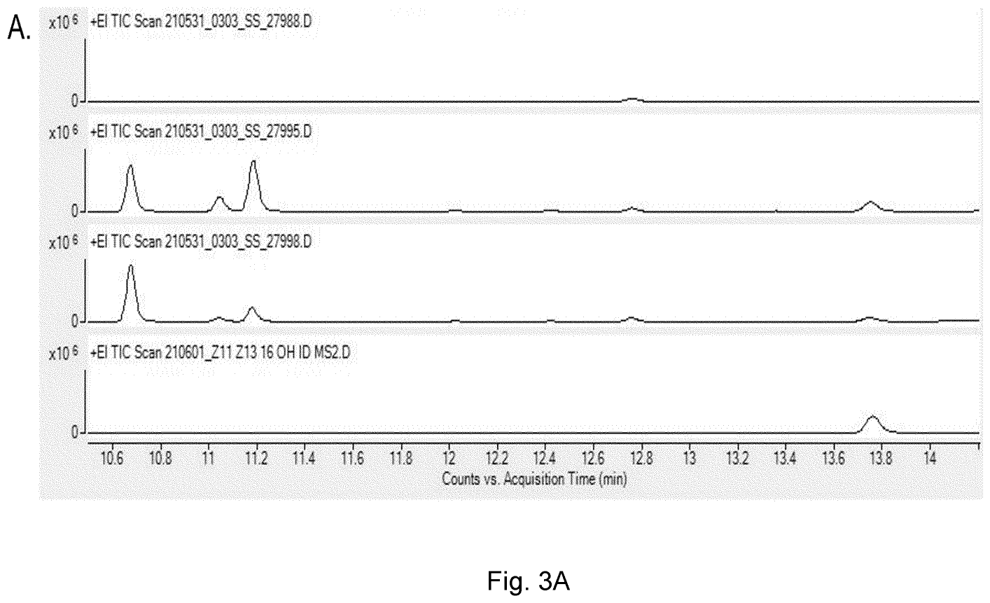

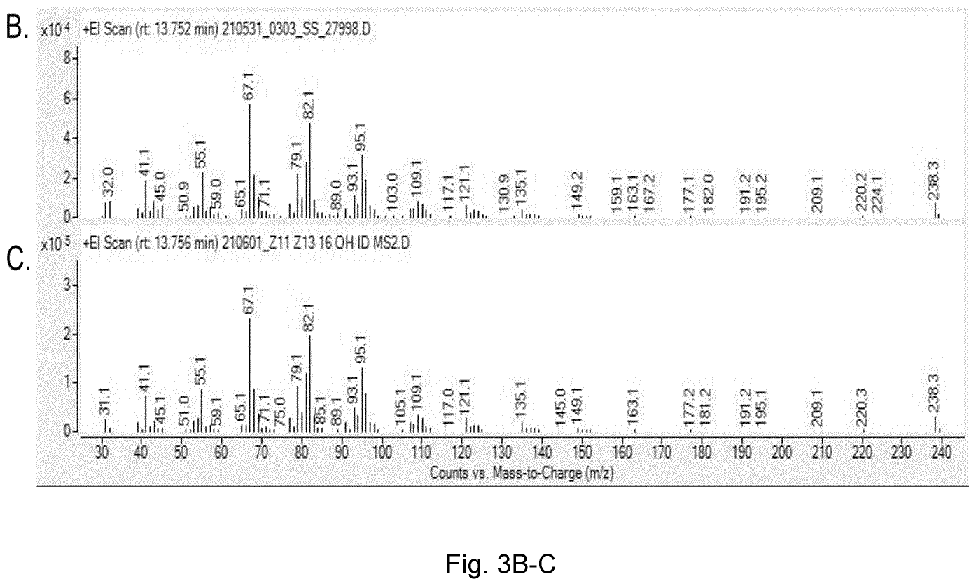

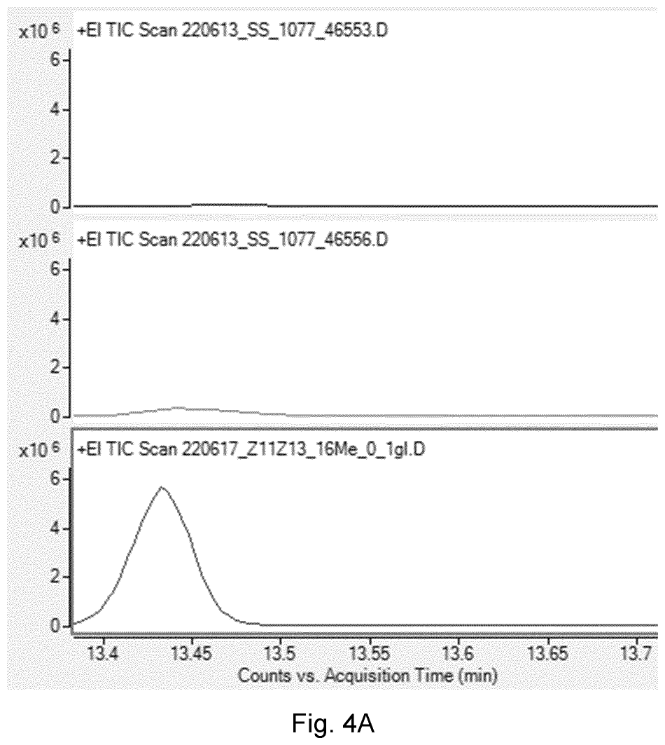

The strains of Y. lipolytica ST11305 and ST11306 express Δ11 desaturase Desat38 from Spodoptera litura and Δ13 desaturase AtrAATQ from Amyelois transitella in combination with fatty acyl-CoA reductases FAR1 from Helicoverpa armigera or FAR33 from Amyelois transitella, respectively. Strain ST11304 serves as control strain expressing 411 desaturase Desat38 and Δ13 desaturase AtrAATQ, but no fatty acyl-CoA reductase. The strains were cultivated and fatty alcohols were extracted as described in Example 3. The control strain ST11304 did not produce any fatty alcohols as expected (FIG. 3). Strain ST11305, expressing FAR1, produced 270 mg/L of Z11-16:OH and 30 mg/L Z11,Z13-16:OH (FIG. 3). Strain ST11306 produced 70 mg/L Z11-16:OH and 15 mg/L Z11,Z13-16:OH. It is worth mentioning that strains expressing either FAR1 (ST11305) or FAR33 (ST11306) produced similar amounts of 16:OH and Z11,Z13-16:OH while ST11306 produced lower amounts of Z11-16:OH than ST11305. This indicates that FAR33 has a lower activity towards Z11-16:CoA than FAR1. Such a property is advantageous for obtaining Z11,Z13-16:OH with higher purity.

Z11, Z13-16:OH can be recovered from the fermentation broth and chemically oxidized to give Z11,Z13-16:Ald (WO2021/078452) or acetylated to give Z11,Z13-16:OAc. Z11,13-16:OH,Z11,Z13-16:Ald, and Z11,Z13-16:OAc can be formulated and used for monitoring or mating disruption of insects that use this compound as their sex pheromone component, for example, the navel orangeworm Amyelois transitella.

### Example 6: Measurement of Biobased Carbon Content

The biobased carbon content of pheromone compounds according to the invention is measured by using the analytical measurement that may be cited as “percent modern carbon (pMC)”. This is the percentage of isotope ¹⁴C measured in the sample relative to a modern reference standard (NIST 4990C). The % biobased carbon content is calculated from pMC by applying a small adjustment factor for isotope 14° C. in carbon dioxide in air today.

The pheromone compounds according to the invention are expected to have a biobased carbon content above 80%.

### Example 7: Oxidation of Z11,Z13-16:OH to Corresponding Aldehyde Z11,Z13-16:Ald

A mixture of primary fatty alcohols containing 73 wt % Z11,Z13-16:OH ((Z11,Z13)-hexadecadien-1-ol) mixture was used as a representative sample for conversion to aldehydes.

Z11,Z13-16:OH mixture (8 g), 2,2′-bipyridine (0.25 g), 2,2,6,6-tetramethyl piperidinyl oxyl (0.14 g) and 1-methylimidazole (0.13 g) was added to acetonitrile (20 ml). To aforementioned solution, tetrakisacetonitrilecopper(I) trifluoromethanesulfonate in 10 ml acetonitrile was added.

The temperature of the reaction mixture was controlled to 30° C. while air was sparged through the solution at a rate of 1 l/min. The air sparging was halted after 164 min and the reaction mixture was diluted in 1-heptane (40 ml) and the mixture extracted with water (20 ml). The top phase was evaporated to 10 mbar at 60° C. giving a product containing 65.5 wt % Z11,Z13-16:Ald ((Z11,Z13)-hexadecadienal) with a residual amount of 3.4 wt % Z11,Z13-16:OH (Table 8).

TABLE 8
Oxidation of Z11,Z13-16:OH to Z11,Z13-16:Ald

| Start product: | End product |
|---|---|
| 73 wt % Z11,Z13-16:OH | 3.4 wt % Z11,Z13-16:OH |
| | 65.5 wt % Z11,Z13-16:Ald |

These data shows that the fatty alcohol Z11, Z13-16:OH was chemically oxidized to the corresponding aldehyde, Z11,Z13-16:Ald.

### Example 8: Production of Z11,Z13-16:Me in Yarrowia lipolytica with Various ΔZ11-Desaturases

The Δ13 desaturase AtrAATQ was co-expressed with individual Δ11 desaturases originating from various organisms in the yeast Yarrowia lipolytica. The strains were cultivated and the FAMEs analyzed as described in Example 3 with the exception that the cultivation in YPG medium was extended from 22 to 47 hours.

Derivatized extracts of all strains, ST12323, ST12326-12330, and ST12332-12339 contained Z11,Z13-16:Me independent of the Δ11 desaturase variant expressed (Table 9).

TABLE 9
Production of Z11,Z13-16:Me in Yarrowia lipolytica

| Strain ID | Δ11 desaturase | Z11,Z13-16:Me |
|---|---|---|
| ST12323 | Desat16 | + |
| ST12326 | Desat21 | + |
| ST12327 | Desat37 | + |
| ST12328 | Desat38 | + |
| ST12329 | Desat44 | + |
| ST12330 | Desat45 | + |
| ST12332 | Desat51 | + |
| ST12333 | Desat63 | + |
| ST12334 | Desat65 | + |
| ST12335 | Desat71 | + |
| ST12336 | Desat79 | + |
| ST12337 | Desat72 | + |
| ST12338 | Desat70 | + |
| ST12339 | Desat73 | + |

### Example 9: Production of Z11,Z13-16:OH in Yarrowia lipolytica with Alternative Fatty Acyl-CoA Reductases

The Δ11 desaturase Desat37 from Spodoptera exigua and Δ13 desaturase AtrAATQ were co-expressed with individual fatty acyl-CoA reductases (FARs) originating from various organisms (Table 4). The strains were cultivated and the fatty alcohols analyzed on the GC-FID as described in Example 3 with the exception that the cultivation in YPG medium was extended from 22 to 47 hours.

Extracts of strains ST12456-12460, ST12462-12463, ST12465-12473, ST12475, ST12477-12480, ST12482-12492, ST12564-12569, and ST12583 contained Z11,Z13-16:OH, while the extract of the control strain ST12493, not expressing any fatty acyl-CoA reductase, did not contain any Z11,Z13-16:OH (Table 10).

TABLE 10
Production of Z11,Z13-16:OH in Yarrowia lipolytica

| Strain ID | FAR | Z11,Z13-16:OH |
|---|---|---|
| ST12456 | FAR4 | + |
| ST12457 | FAR5 | + |
| ST12458 | FAR6 | + |
| ST12459 | FAR8 | + |
| ST12460 | FAR9 | + |
| ST12462 | FAR12 | + |
| ST12463 | FAR42 | + |
| ST12465 | FAR25 | + |
| ST12466 | FAR13 | + |
| ST12467 | FAR14 | + |
| ST12468 | FAR15 | + |
| ST12469 | FAR16 | + |
| ST12470 | FAR17 | + |
| ST12471 | FAR18 | + |
| ST12472 | FAR19 | + |
| ST12473 | FAR20 | + |
| ST12475 | FAR22 | + |
| ST12477 | FAR27 | + |
| ST12478 | FAR28 | + |
| ST12479 | FAR29 | + |
| ST12480 | FAR30 | + |
| ST12482 | FAR43 | + |
| ST12483 | FAR33 | + |
| ST12484 | FAR34 | + |
| ST12485 | FAR35 | + |
| ST12486 | FAR44 | + |
| ST12487 | FAR48 | + |
| ST12488 | FAR49 | + |
| ST12489 | FAR45 | + |
| ST12491 | FAR38 | + |
| ST12492 | FAR40 | + |
| ST12493 | − | − |
| ST12564 | FAR41 | + |
| ST12565 | FAR51 | + |
| ST12566 | FAR52 | + |
| ST12567 | FAR47 | + |
| ST12568 | FAR46 | + |
| ST12569 | FAR50 | + |
| ST12583 | FAR1 | + |

### Example 10: Production of Z11,Z13-16:OH in Saccharomyces cerevisiae with Alternative Fatty Acyl Reductase

The Δ11 desaturases Desat16 from Amyelois transitella, Desat45 from Plutella xylostella and Desat44 from Chilo suppressalis were co-expressed with Δ13 desaturase AtrAATQ and fatty acyl-CoA reductase FAR33 from Amyelois transitella in the yeast S. cerevisiae, respectively (Table 11). The strains were constructed and cultivated as described in Example 2 and Example 3, respectively.

The three strains produced Z11,Z13-16:OH. Production of Z11-16:OH could not be detected in these samples.

TABLE 11
Production of Z11,Z13-16:OH in Saccharomyces cerevisiae

| | | Z11,Z13-16:OH |
|---|---|---|
| Strain ID | Genes expressed | (mg/L) |
| Sc_Desat16_{—} | Desat16, AtrAATQ, FAR33 | + |
| AtrAATQ_FAR33 | | |
| Sc_Desat45_{—} | Desat45, AtrAATQ, FAR33 | + |
| AtrAATQ_FAR33 | | |
| Sc_Desat44_{—} | Desat44, AtrAATQ, FAR33 | + |
| AtrAATQ_FAR33 | | |

### Example 11: Expression of NdDes1 in Yarrowia lipolytica

The Δ11 desaturase Desat38 from Spodoptera litura was either expressed alone or in combination with the desaturase NdDes1 from Notodonta dromedarius. The strains were cultivated and the FAMEs analyzed as described in Example 3.

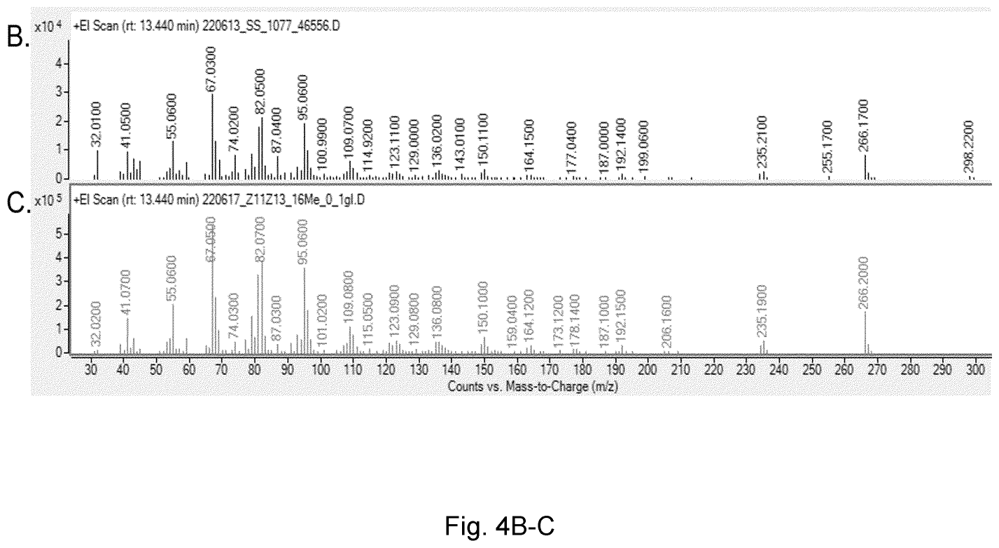

Derivatized extracts of strain ST12611 co-expressing Desat38 and NdDes1 contained Z11,Z13-16:Me while extracts of control strain ST12613 only expressing Desat38 did not (Table 12). The Z11,Z13-16:Me peak eluted at a retention time of 13.44 min both in the pure Z11,Z13-16:Me-standard als well as extract of ST12611 (FIG. 4A). The mass spectra of the peak eluting at 13.44 min in the ST12613-extract matched the mass spectra of the pure Z11,Z13-16:Me-standard (FIG. 4B-C).

TABLE 12
Production of Z11,Z13-16:Acid in Yarrowia lipolytica

| Strain ID | Genes expressed | Z11,Z13-16:Me |
|---|---|---|
| ST12611 | Desat38, NdDes1 | + |
| ST12613 | Desat38 | − |

### Example 12: Expression of Tpi-PGFAD in Saccharomyces cerevisiae

Saccharomyces cerevisiae Sc_Tpi_PGFAD was cultivated and FAME were analyzed as described in Example 3.

Strain Sc_Tpi_PGFAD produced (Z)-11-hexadecenoic acid and enyne precursor 11-hexadecynoic acid as described in Serra et al., 2007. However, no Z11,Z13-16:Acid was detected, meaning Tpi_PGFAD can not produce any Z11,Z13-16:Acid. Serra et al. detected (Z)-13-hexadecen-11-ynoic acid when a Tpi-PGFAD expressing yeast was contacted with the enyne precursor 11-hexadecynoic acid (Table 13).

TABLE 13
Detection of fatty acids produced by Saccharomyces cerevisiae Sc_Tpi_PGFAD

| | | | | (Z)-13- |
|---|---|---|---|---|
| | (Z)-11- | 11- | | hexadecen- |
| | hexadecenoic | hexadecynoic | Z11,Z13- | 11-ynoic |
| Strain ID | acid | acid | 16:Acid | acid |
| Sc_Tpi_PGFAD | + | + | − | − |

### Example 13: Production of Z11,Z13-16:Acid in Yarrowia lipolytica Using Alternative Δ13 Desaturases

Fusion-protein variants of AtrAATQ were designed and co-expressed with Δ11 desaturase Desat38. Strain ST12648 expressed a fusion protein, Desat83, in which the N-terminus (amino acid residues 1-20) and C-terminus (amino acid residues 302-348) of AtrAATQ were replaced with those of the Amyelois transitella desaturase Desat16 (N-terminus, amino acid residues 1-20; C-terminus, amino acid residues 302-326), ST12649 expressed a fusion protein, Desat84, in which the N- and C-terminus of AtrAATQ were replaced with the N-terminus of Cadra cautella desaturase Desat70 (amino acid residues 1-19) and C-terminus Plutella xylostella desaturase Desat45 (amino acid residues 305-349), respectively, and ST12650 expresses a fusion protein, Desat85, in which the N- and C-terminus of AtrAATQ were replaced with the N-terminus of Plutella xylostella desaturase Desat45 (amino acid residues 1-23) and C-terminus Cadra cautella desaturase Desat70 (amino acid residues 301-342). The sequence identities of the fusion proteins can be seen in the table below (Table 13).

TABLE 13
Sequence identity percentage between desaturases

| | | 1 | 2 | 3 | 4 | 5 |
|---|---|---|---|---|---|---|
| AtrAATQ | 1 | | 89.66 | 87.93 | 85.19 | 58.69 |
| Desat83 | 2 | 89.66 | | 87.86 | 87.28 | 57.76 |
| Desat84 | 3 | 87.93 | 87.86 | | 85.19 | 58.97 |
| Desat85 | 4 | 85.19 | 87.28 | 85.19 | | 58.86 |
| NdDes1 | 5 | 58.69 | 57.76 | 58.97 | 58.86 | |

The strains ST12648, ST12649 and ST12650 were cultivated and FAME were extracted as described in Example 3, with the exception that the cells were incubated for 47 hours in YPG medium instead of 22 hours. Derivatized extracts of all three strains ST12648, ST12649 and ST12650 contained Z11,Z13-16:Me (Table 14).

TABLE 14
Production of Z11,Z13-16:Acid in Yarrowia lipolytica

| Strain ID | Desaturases | Z11,Z13-16:Me |
|---|---|---|
| ST12648 | Desat38 Desat83 | + |
| ST12649 | Desat38 Desat84 | + |
| ST12650 | Desat38 Desat85 | + |

### Example 14: Measurement of Biobased Carbon Content

The biobased carbon content of a pheromone compound of the invention was measured by using the analytical measurement that may be cited as “percent modern carbon (pMC)”. This is the percentage of isotope ¹⁴C measured in the sample relative to a modern reference standard (NIST 4990C). The % biobased carbon content is calculated from pMC by applying a small adjustment factor for isotope ¹⁴C in carbon dioxide in air today.

Strain STNOW, engineered for production of Z11,Z13-16:OH, was fermented and the product was recovered. The product sample was composed of a mix of fatty alcohols including Z11,Z13-16:OH. The “percent modern carbon (pMC)” of the product sample was analyzed by the standard test method “ASTM D6866”. The percent of modern carbon was determined to be 100.71±0.31 pMC, corresponding to 100% of biobased carbon content (Table 15). This implies that the pMC of Z11,Z13-16:OH contained in the product sample also was 100%.

TABLE 15
Determination of percent of modern carbon in product samle

| Strain ID | Product sample | pMC |
|---|---|---|
| STNOW | Mix of fatty alcohols, | 100.71 ± 0.31 |
| | including Z11,Z13-16:OH | |

### Sequence Overview

All DNA sequences were codon-optimized for Yarrowia lipolytica with the exceptions of SEQ ID NO: 10, 12, 20, 22, 46, 47, 48, 49, 50, 52, 72, and 74, which were codon-optimized for Saccharomyces cerevisiae, and SEQ ID NO: 54 and SEQ ID NO: 62 which were the native sequences.

| SEQ ID NO: | Name and accesion number | Organism |
|---|---|---|
| 1 | AtrAATQ (amino acid), | Amyelois transitella |
| | XP_013190816.1 | |
| 2 | AtrAATQ (DNA), codon-optimised | Amyelois transitella |
| | for Y. lipolytica | |
| 3 | Desat38 (amino acid) | Spodoptera litura |
| 4 | Desat38 (DNA), codon-optimised | Spodoptera litura |
| | for Y. lipolytica | |
| 5 | Desat20 (amino acid) | Spodoptera littoralis |
| 6 | Desat20 (DNA), codon-optimised | Spodoptera littoralis |
| | for Y. lipolytica | |
| 7 | Desat16 (amino acid) | Amyelois transitella |
| 8 | Desat16 (DNA), codon-optimised | Amyelois transitella |
| | for Y. lipolytica | |
| 9 | Desat19 (amino acid) | Agrotis segetum |
| 10 | Desat19 (DNA), codon-optimised | Agrotis segetum |
| | for S. cerevisiae | |
| 11 | Desat21 (amino acid) | Trichoplusia ni |
| 12 | Desat21 (DNA), codon-optimised | Trichoplusia ni |
| | for S. cerevisiae | |
| 13 | FAR1 (amino acid) | Helicoverpa armigera |
| 14 | FAR1 (DNA), codon-optimised for | Helicoverpa armigera |
| | Y. lipolytica | |
| 15 | FAR33 (amino acid) | Amyelois transitella |
| 16 | FAR33 (DNA), codon-optimised | Amyelois transitella |
| | for Y. lipolytica | |
| 17 | PR25094 (DNA) | synthetic construct |
| 18 | PR25381 (DNA) | synthetic construct |
| 19 | FAR4 (amino acid) | Heliothis subflexa |
| 20 | FAR4 (DNA), codon-optimised for | Heliothis subflexa |
| | S. cerevisiae | |
| 21 | FAR6 (amino acid) | Helicoverpa assulta |
| 22 | FAR6 (DNA), codon-optimised for | Helicoverpa assulta |
| | S. cerevisiae | |
| 23 | DgNcb5or (amino acid) | Drosophila grimshawi |
| 24 | DmNcb5or (amino acid) | Drosophila melanogaster |
| 25 | HsNcb5or (amino acid) | Homo sapiens |
| 26 | SlitNcb5or (amino acid) | Spodoptera litura |
| 27 | CpoNcb5or1 (amino acid) | Cydia pomonella |
| 28 | Desat51 (DNA), codon-optimised | Helicoverpa zea |
| | for Y. lipolytica | |
| 29 | Desat51 (amino acid) | Helicoverpa zea |
| 30 | Desat37 (DNA), codon-optimised | Spodoptera exigua |
| | for Y. lipolytica | |
| 31 | Desat37 (amino acid) | Spodoptera exigua |
| 32 | Desat45 (DNA), codon-optimised | Plutella xylostella |
| | for Y. lipolytica | |
| 33 | Desat45 (amino acid) | Plutella xylostella |
| 34 | FAR32 (DNA), codon-optimised | Plodia interpunctella |
| | for Y. lipolytica | |
| 35 | FAR32 (amino acid) | Plodia interpunctella |
| 36 | FAR16 (DNA), codon-optimised | Spodoptera exigua |
| | for Y. lipolytica | |
| 37 | FAR16 (amino acid) | Spodoptera exigua |
| 38 | FAR19 (DNA), codon-optimised | Spodoptera litura |
| | for Y. lipolytica | |
| 39 | FAR19 (amino acid) | Spodoptera litura |
| 40 | FAR17 (DNA), codon-optimised | Spodoptera exigua |
| | for Y. lipolytica | |
| 41 | FAR17 (amino acid) | Spodoptera exigua |
| 42 | FAR38 (DNA), codon-optimised | Trichoplusia ni |
| | for Y. lipolytica | |
| 43 | FAR38 (amino acid) | Trichoplusia ni |
| 44 | FAR28 (DNA), codon-optimised | Plodia interpunctella |
| | for Y. lipolytica | |
| 45 | FAR28 (amino acid) | Plodia interpunctella |
| 46 | AtrAATQ (DNA), codon-optimised | Amyelois transitella |
| | to S. cerevisiae | |
| 47 | Desat16 (DNA), codon-optimised | Amyelois transitella |
| | for S. cerevisiae | |
| 48 | FAR1 (DNA), codon-optimised for | Helicoverpa armigera |
| | S. cerevisiae | |
| 49 | FAR33 (DNA), codon-optimised | Amyelois transitella |
| | to S. cerevisiae | |
| 50 | Desat44 (DNA), codon-optimised | Chilo suppressalis |
| | for S. cerevisiae | |
| 51 | Desat44 (amino acid) | Chilo suppressalis |
| 52 | Desat45 (DNA), codon-optimised | Plutella xylostella |
| | to S. cerevisiae | |
| 53 | AseNcb5or (amino acid) | Agrotis segetum |
| 54 | Tpi_PGFAD (DNA), native | Thaumetopoea |
| | | pityocampa |
| 55 | Tpi_PGFAD (amino acid) | Thaumetopoea |
| | | pityocampa |
| 56 | NdDes1 (amino acid) | Notodonta dromedarius |
| 57 | NdDes1 (DNA), codon-optimised | Notodonta dromedarius |
| | for Y. lipolytica | |
| 58 | Desat63 (DNA), codon-optimised | Diatraea saccharalis |
| | for Y. lipolytica | |
| 59 | Desat63 (amino acid) | Diatraea saccharalis |
| 60 | Desat65 (DNA), codon-optimised | Plodia interpunctella |
| | for Y. lipolytica | |
| 61 | Desat65 (amino acid) | Plodia interpunctella |
| 62 | Desat71 (DNA), native | Lobesia botrana |
| 63 | Desat71 (amino acid) | Lobesia botrana |
| 64 | Desat79 (DNA), codon-optimised | Lobesia botrana |
| | for Y. lipolytica | |
| 65 | Desat79 (amino acid) | Lobesia botrana |
| 66 | Desat72 (DNA), codon-optimised | Antheraea pernyi |
| | for Y. lipolytica | |
| 67 | Desat72 (amino acid) | Antheraea pernyi |
| 68 | Desat70 (DNA), codon-optimised | Cadra cautella |
| | for Y. lipolytica | |
| 69 | Desat70 (amino acid) | Cadra cautella |
| 70 | Desat73 (DNA), codon-optimised | Yponomeuta padella |
| | for Y. lipolytica | |
| 71 | Desat73 (amino acid) | Yponomeuta padella |
| 72 | FAR5 (DNA), codon-optimised for | Heliothis virescens |
| | S. cerevisiae | |
| 73 | FAR5 (amino acid) | Heliothis virescens |
| 74 | FAR8 (DNA), codon-optimised for | Yponomeuta rorellus |
| | S. cerevisiae | |
| 75 | FAR8 (amino acid) | Yponomeuta rorellus |
| 76 | FAR9 (DNA), codon-optimised for | Ostrinia nubilalis |
| | Y. lipolytica | |
| 77 | FAR9 (amino acid) | Ostrinia nubilalis |
| 78 | FAR12 (DNA), codon-optimised | Agrotis segetum |
| | for Y. lipolytica | |
| 79 | FAR12 (amino acid) | Agrotis segetum |
| 80 | FAR42 (DNA), codon-optimised | Marinobacter algicola |
| | for Y. lipolytica | |
| 81 | FAR42 (amino acid) | Marinobacter algicola |
| 82 | FAR25 (DNA), codon-optimised | Tyta alba |
| | for Y. lipolytica | |
| 83 | FAR25 (amino acid) | Tyta alba |
| 84 | FAR13 (DNA), codon-optimised | Chilo suppressalis |
| | for Y. lipolytica | |
| 85 | FAR13 (amino acid) | Chilo suppressalis |
| 86 | FAR14 (DNA), codon-optimised | Bombus lapidarius |
| | for Y. lipolytica | |
| 87 | FAR14 (amino acid) | Bombus lapidarius |
| 88 | FAR15 (DNA), codon-optimised | Spodoptera littoralis |
| | for Y. lipolytica | |
| 89 | FAR15 (amino acid) | Spodoptera littoralis |
| 90 | FAR18 (DNA), codon-optimised | Agrotis ipsilon |
| | for Y. lipolytica | |
| 91 | FAR18 (amino acid) | Agrotis ipsilon |
| 92 | FAR20 (DNA), codon-optimised | Spodoptera frugiperda |
| | for Y. lipolytica | |
| 93 | FAR20 (amino acid) | Spodoptera frugiperda |
| 94 | FAR22 (DNA), codon-optimised | Spodoptera frugiperda |
| | for Y. lipolytica | |
| 95 | FAR22 (amino acid) | Spodoptera frugiperda |
| 96 | FAR27 (DNA), codon-optimised | Plutella xylostella |
| | for Y. lipolytica | |
| 97 | FAR27 (amino acid) | Plutella xylostella |
| 98 | FAR29 (DNA), codon-optimised | Plodia interpunctella |
| | for Y. lipolytica | |
| 99 | FAR29 (amino acid) | Plodia interpunctella |
| 100 | FAR30 (DNA), codon-optimised | Plodia interpunctella |
| | for Y. lipolytica | |
| 101 | FAR30 (amino acid) | Plodia interpunctella |
| 102 | FAR43 (DNA), codon-optimised | Manducta sexta |
| | for Y. lipolytica | |
| 103 | FAR43 (amino acid) | Manducta sexta |
| 104 | BterNcb5or (amino acid) | Bombus terrestris |
| 105 | LboNcb5or (amino acid) | Lobesia botrana |
| 106 | FAR34 (DNA), codon-optimised | Amyelois transitella |
| | for Y. lipolytica | |
| 107 | FAR34 (amino acid) | Amyelois transitella |
| 108 | FAR35 (DNA), codon-optimised | Amyelois transitella |
| | for Y. lipolytica | |
| 109 | FAR35 (amino acid) | Amyelois transitella |
| 110 | FAR44 (DNA), codon-optimised | Ostrinia furnacalis |
| | for Y. lipolytica | |
| 111 | FAR44 (amino acid) | Ostrinia furnacalis |
| 112 | FAR48 (DNA), codon-optimised | Ostrinia zag |
| | for Y. lipolytica | |
| 113 | FAR48 (amino acid) | Ostrinia zag |
| 114 | FAR49 (DNA), codon-optimised | Ostrinia zea |
| | for Y. lipolytica | |
| 115 | FAR49 (amino acid) | Ostrinia zea |
| 116 | FAR45 (DNA), codon-optimised | Spodoptera exigua |
| | for Y. lipolytica | |
| 117 | FAR45 (amino acid) | Spodoptera exigua |
| 118 | FAR40 (DNA), codon-optimised | Trichoplusia ni |
| | for Y. lipolytica | |
| 119 | FAR40 (amino acid) | Trichoplusia ni |
| 120 | FAR41 (DNA), codon-optimised | Trichoplusia ni |
| | for Y. lipolytica | |
| 121 | FAR41 (amino acid) | Trichoplusia ni |
| 122 | FAR51 (DNA), codon-optimised | Trichoplusia ni |
| | for Y. lipolytica | |
| 123 | FAR51 (amino acid) | Trichoplusia ni |
| 124 | FAR52 (DNA), codon-optimised | Chrysodeixis includens |
| | for Y. lipolytica | |
| 125 | FAR52 (amino acid) | Chrysodeixis includens |
| 126 | FAR47 (DNA), codon-optimised | Chrysodeixis includens |
| | for Y. lipolytica | |
| 127 | FAR47 (amino acid) | Chrysodeixis includens |
| 128 | FAR46 (DNA), codon-optimised | Cydia pomonella |
| | for Y. lipolytica | |
| 129 | FAR46 (amino acid) | Cydia pomonella |
| 130 | FAR50 (DNA), codon-optimised | Grapholita molesta |
| | for Y. lipolytica | |
| 131 | FAR50 (amino acid) | Grapholita molesta |
| 132 | Desat83 (amino acid) | synthetic construct |
| 133 | Desat84 (amino acid) | synthetic construct |
| 134 | Desat85 (amino acid) | synthetic construct |
| 135 | Desat83 (DNA), codon-optimised | synthetic construct |
| | for Y. lipolytica | |
| 136 | Desat84 (DNA), codon-optimised | synthetic construct |
| | for Y. lipolytica | |
| 137 | Desat85 (DNA), codon-optimised | synthetic construct |
| | for Y. lipolytica | |
| 138 | Desat44 (DNA), codon-optimised | Chilo suppressalis |
| | for Y. lipolytica | |

### REFERENCES

Alfaro et al. 2009. Optimization of pheromone dispenser density for managing the rice striped stem borer, Chilo suppressalis (Walker), by mating disruption. Crop Protection. 28:567-572.
Bari, 2003. Development of pheromone mating disruption strategies for the suppression of the artichoke plume moth in artichokes grown on the central coast of California. ISHS Acta Horticulturae 660: V International Congress on Artichoke. doi: 10.17660/ActaHortic.2004.660.80
Borodina, I., & Lorántfy, B. (2021). Patentnr. WO 2021/078452 Δ1.
Deng, B et al. (2010). Study of the individual cytochrome b5 and cytochrome b5 reductase domains of Ncb5or reveals a unique heme pocket and a possible role the CS domain. The Journal of Biological Chemistry, 30181-30191.
Ding, B., & Löfstedt, C. (2015). Ding B J, Löfstedt C. Analysis of the Agrotis segetum pheromone gland transcriptome in the light of sex pheromone biosynthesis. BMC Genomics.
Eizaguirre et al. 2002. Effects of mating disruption against the Mediterranean corn borer, Sesamia nonagrioides, on the European corn borer Ostrinia nubilalis. Use of pheromones and other semiochemicals in integrated production IOBC wprs Bulletin.
Holkenbrink et al., 2018. EasyCloneYALI: CRISPR/Cas9-Based Synthetic Toolbox for Engineering of the Yeast Yarrowia lipolytica. Biotechnol J., September; 13(9)
Holkenbrink et al., 2020. Production of moth sex pheromones for pest control by yeast fermentation. Metab Eng. 2020 November; 62:312-321.
Jensen et al., 2014. EasyClone: method for iterative chromosomal integration of multiple genes in Saccharomyces cerevisiae. FEMS Yeast Research, 238-248.
Kehat & Dunkelblum, 1993. Sex Pheromones: achievements in monitoring and mating disruption of cotton pests in Israel, Achieves of Insect Biochemistry and Physiology. 22:425-431.
Patel, O., Fernley, R., & MacReadie, I. (2003). Saccharomyces cerevisiae expression vectors with thrombin-cleavable N- and C-terminal 6×(His) tags. Biotechnology Letters.
Petkevicius et al., 2021. Biotechnological production of the European corn borer sex pheromone in the yeast Yarrowia lipolytica. Biotechnology J., June; 16(6)
Tsakraklides et al. (2018). High-oleate yeast oil without polyunsaturated fatty acids. Biotechnology for biofuels, 11, 131. https://doi.org/10.1186/s13068-018-1131-y
Wakamura et al. (1987). Sex pheromone of the beet armyworm Spodoptera exigua (Hubner) (Lepidoptera: Noctuidae): Field attraction of male moths in Japan to (Z,E)-9 12-tetradecadienylacetate and (Z)-9 tetradecen-1-ol. Applied Entomology and Zoology, 22(3), 348-351.
Wu et al., 2012. Management of diamondback moth, Plutella xylostella (Lepidoptera: Plutellidae) by mating disruption. Insect Science 19 (6), 643-648.
Xia et al., 2019. Multi-Functional Desaturases in Two Spodoptera Moths with Δ11 and Δ12 Desaturation Activities. J Chem Ecol 45, 378-387.
Zhu et al., 1999. Reidentification of the female sex pheromone of the Indian meal moth, Plodia interpunctella: Evidence for a four-component pheromone blend. Entomologia Experimentalis Et Applicata, 92(2), p. 137-146.

### Items

1. A yeast cell capable of producing a desaturated fatty acyl-coenzyme A (fatty acyl-CoA) having n′ double bond(s), wherein at least one of said double bond(s) is at position 13,
wherein said yeast cell expresses a heterologous Δ13 desaturase, said desaturase being capable of introducing a double bond at position 13 in a saturated or desaturated fatty acyl-CoA, preferably a desaturated fatty acyl-CoA, having a carbon chain length of at least 14 and having n double bond(s),
wherein n and n′ are integers,
wherein 0≤n≤3 and wherein 1≤n′≤4.
2. A yeast cell capable of producing a desaturated fatty acyl-coenzyme A (fatty acyl-CoA) having n′ double bond(s), wherein at least one of said double bond(s) is at position 13,
wherein said yeast cell expresses a heterologous Δ13 fatty acyl-CoA desaturase, said desaturase being capable of introducing a double bond at position 13 in a saturated fatty acyl-CoA or a desaturated fatty acyl-CoA having a carbon chain consisting solely of single and double bonds, preferably a desaturated fatty acyl-CoA, wherein said fatty acyl-CoA has a carbon chain length of at least 14 and has n double bond(s),
wherein n and n′ are integers,
wherein 0≤n≤3 and wherein 1≤n′≤4.
3. The yeast cell according to any one of items 1 to 2, wherein said heterologous Δ13 desaturase is native to an organism of a genus selected from the group consisting of Amyelois, Desmia, Thaumetopoea and Notodonta.
4. The yeast cell according to any one of the preceding items, wherein said heterologous Δ13 desaturase is native to an organism of a species selected from the group consisting of Amyelois transitella, Desmia funeralis, Thaumetopoea bonjeani and Notodonta dromedarius.
5. The yeast cell according to any one of the preceding items, wherein said heterologous Δ13 desaturase is an Amyelois desaturase, such as an Amyelois transitella desaturase, or a Notodonta desaturase, such as a Notodonta dromedarius desaturase.
6. The yeast cell according to any one of the preceding items, wherein said heterologous Δ13 desaturase is AtrAATQ as set forth in SEQ ID NO: 1 or a functional variant thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
7. The yeast cell according to any one of the preceding items, wherein said heterologous Δ13 desaturase is NdDes1 as set forth in SEQ ID NO: 56 or a functional variant thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
8. The yeast cell according to any one of the preceding items, wherein said heterologous Δ13 desaturase is selected from the desaturases set forth in SEQ ID NO: 132, SEQ ID NO: 133, and SEQ ID NO: 134.
9. The yeast cell according to any one of the preceding items, wherein the saturated fatty acyl-CoA or desaturated fatty acyl-CoA used as a substrate and the desaturated fatty acyl-CoA having n′ double bonds have the same carbon chain length.
10. The yeast cell according to any one of the preceding items, wherein said saturated or desaturated fatty acyl-CoA having a carbon chain length of at least 14 and having n double bonds, has a carbon length of at least 15, such as at least 16.
11. The yeast cell according to any one of the preceding items, wherein said saturated or desaturated fatty acyl-CoA having a carbon chain length of at least 14 and having n double bonds is hexadecanoyl-CoA or hexadecenoyl-CoA.
12. The yeast cell according to any one of the preceding items, wherein n=1.
13. The yeast cell according to any one of the preceding items, wherein said saturated or desaturated fatty acyl-CoA having a carbon chain length of at least 14 and having n double bonds is a desaturated (Z11)-hexadecenoyl-CoA.
14. The yeast cell according to any one of the preceding items, wherein said desaturated fatty acyl-CoA having n′ double bond(s) has a carbon chain length of at least 14, such as at least 15, such as at least 16, such as at least 17, such as at least 18, such as at least 19, such as at least 20, such as at least 21, such as at least 22.
15. The yeast cell according to any one of the preceding items, wherein said desaturated fatty acyl-CoA having n′ double bond(s) has a carbon chain length of at the most 20, such as at the most 19, such as at the most 18, such as at the most 17, such as at the most 16, such as at least 17, such as at least 18, such as at least 19, such as at least 20, such as at least 21, such as at least 22.
16. The yeast cell according to any one of the preceding items, wherein n′=1.
17. The yeast cell according to any one of the preceding items, wherein said desaturated fatty acyl-CoA having n′ double bond(s) is (Z13)-hexadecenoyl-CoA.
18. The yeast cell according to any one of the preceding items, wherein n′=2.
19. The yeast cell according to any one of the preceding items, wherein said desaturated fatty acyl-CoA having n′ double bond(s) is (Z11, Z13)-hexadecadienoyl-CoA.
20. The yeast cell according to any one of the preceding items, wherein said yeast cell expresses a further heterologous desaturase.
21. The yeast cell according to any one of the preceding items, wherein said further heterologous desaturase is capable of introducing a double bond at any position which is not position 13 in a saturated or desaturated fatty acyl-CoA having a carbon chain length of at least 14 and having n double bonds.
22. The yeast cell according to any one of the preceding items, wherein said further heterologous desaturase is capable of introducing a double bond at least in position 11.
23. The yeast cell according to any one of the preceding items, wherein said further heterologous desaturase is a Δ11 desaturase.
24. The yeast cell according to any one of the preceding items, wherein said further heterologous desaturase has a higher specificity towards hexadecanoyl-CoA or hexadecenoyl-CoA than towards tetradecanoyl-CoA or tetradecenoyl-CoA, respectively.
25. The yeast cell according to any one of the preceding items, wherein said further heterologous desaturase is a Spodoptera desaturase, or an Amyelois desaturase, an Agrotis desaturase, a Trichoplusia desaturase, a Plutella desaturase, or a Helicoverpa desaturase, a Chilo desaturase, a Diatraea desaturase, a Plodia desaturase, a Lobesia desaturase, a Antheraea desaturase, a Cadra desaturase, or an Yponomeuta desaturase, such as a Spodoptera litura desaturase, a Spodoptera littoralis desaturase, a Spodoptera exigua desaturase, an Amyelois transitella desaturase, an Agrotis segetum desaturase, a Trichoplusia ni desaturase, a Plutella xylostella desaturase, a Helicoverpa zea desaturase, a Chilo suppressalis desaturase, a Diatraea saccharalis desaturase, a Plodia interpunctella desaturase, a Lobesia botrana desaturase, an Antheraea pernyi desaturase, a Cadra cautella desaturase, or a Yponomeuta padella desaturase.
26. The yeast cell according any one of the preceding items, wherein said further heterologous desaturase is selected from the group consisting Desat38 (SEQ ID NO: 3), Desat20 (SEQ ID NO: 5), Desat16 (SEQ ID NO: 7), Desat19 (SEQ ID NO: 9), Desat21 (SEQ ID NO: 11), Desat37 (SEQ ID NO: 31), Desat45 (SEQ ID NO: 33), Desat51 (SEQ ID NO: 29), Desat44 (SEQ ID NO: 51), Desat63 (SEQ ID NO: 59), Desat65 (SEQ ID NO: 61), Desat71 (SEQ ID NO: 63), Desat79 (SEQ ID NO: 65), Desat72 (SEQ ID NO: 67), Desat70 (SEQ ID NO: 69), and Desat73 (SEQ ID NO: 71), or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
27. The yeast cell according to any one of the preceding items, wherein the yeast cell is capable of producing (Z11,Z13)-hexadecadien-1-ol, said yeast cell further expressing at least one alcohol-forming fatty acyl-CoA reductase (FAR) capable of converting at least a part of (Z11, Z13)-hexadecadienoyl-CoA into (Z11,Z13)-hexadecadien-1-ol.
28. The yeast cell according to any one of the preceding items, wherein the FAR is native to an organism of a genus selected from the group consisting of Agrotis, Amyelois, Bicyclus, Bombus, Chilo, Cydia, Helicoverpa, Heliothis, Lobesia, Ostrinia, Plodia, Plutella, Spodoptera, Trichoplusia, Tyta, Yponomeuta, Ostrinia, Marinobacter, Manducta, Chrysodeixis, and Grapholita, such as Agrotis ipsilon, Agrotis segetum, Amyelois transitella, Bicyclus anynana, Bombus lapidarius, Chilo suppressalis, Cydia pomonella, Helicoverpa armigera, Helicoverpa assulta, Heliothis subflexa, Heliothis virescens, Lobesia botrana, Ostrinia nubilalis, Plodia interpunctella, Plutella xylostella, Spodoptera exigua, Spodoptera frugiperda, Spodoptera littoralis, Spodoptera litura, Trichoplusia ni, Tyta alba, Yponomeuta rorellus, Marinobacter algicola, Manducta sexta, Ostrinia nubilalis, Ostrinia furnacalis, Ostrinia zag, Ostrinia zea, Chrysodeixis includens, and Grapholita molesta, especially Spodoptera exigua, Helicoverpa armigera, Spodoptera litura, Amyelois transitella and Agrotis ipsilon.
29. The yeast cell according to any one of the preceding items, wherein the FAR is selected from the group consisting of FAR1 (SEQ ID NO: 13), FAR33 (SEQ ID NO: 15), FAR4 (SEQ ID NO: 19), FAR6 (SEQ ID NO: 21), FAR28 (SEQ ID NO: 45), FAR32 (SEQ ID NO: 35), FAR16 (SEQ ID NO: 37), FAR17 (SEQ ID NO: 41), FAR19 (SEQ ID NO: 39), FAR38 (SEQ ID NO: 43), FAR5 (SEQ ID NO: 73), FAR8 (SEQ ID NO: 75), FAR9 (SEQ ID NO: 77), FAR12 (SEQ ID NO: 79), FAR42 (SEQ ID NO: 81), FAR25 (SEQ ID NO: 83), FAR13 (SEQ ID NO: 85), FAR14 (SEQ ID NO: 87), FAR15 (SEQ ID NO: 89), FAR18 (SEQ ID NO: 91), FAR20 (SEQ ID NO: 93), FAR22 (SEQ ID NO: 95), FAR27 (SEQ ID NO: 97), FAR29 (SEQ ID NO: 99), FAR30 (SEQ ID NO: 101), FAR43 (SEQ ID NO: 103), FAR34 (SEQ ID NO: 107), FAR35 (SEQ ID NO: 109), FAR44 (SEQ ID NO: 111), FAR48 (SEQ ID NO: 113), FAR49 (SEQ ID NO: 115), FAR45 (SEQ ID NO: 117), FAR40 (SEQ ID NO: 119), FAR41 (SEQ ID NO: 121), FAR51 (SEQ ID NO: 123), FAR52 (SEQ ID NO: 125), FAR47 (SEQ ID NO: 127), FAR46 (SEQ ID NO: 129), and FAR50 (SEQ ID NO: 131), or functional variants thereof having at least 70% similarity, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
30. The yeast cell according to any one of the preceding items, wherein the FAR has a lower specificity towards fatty acyl-CoA having n double bond(s), than fatty acyl-CoA having n′ double bond(s).
31. The yeast cell according to any one of the preceding items, wherein the FAR has a lower specificity towards (Z11)-hexadecenoyl-CoA and/or hexadecanoyl-CoA than (Z11, Z13)-hexadecenoyl-CoA.
32. The yeast cell according to any one of the preceding items, wherein the FAR has a higher specificity towards fatty acyl-CoA having n′ double bond(s), than fatty acyl-CoA having n double bond(s).
33. The yeast according to any one of the preceding items, wherein the FAR has a higher specificity towards (Z11,Z13)-hexadecenoyl-CoA than (Z11)-hexadecenoyl-CoA and/or hexadecanoyl-CoA.
34. The yeast cell according to any one of the preceding items, wherein the yeast cell belongs to a genus selected from Saccharomyces, Pichia, Yarrowia, Kluyveromyces, Candida, Rhodotorula, Rhodosporidium, Cryptococcus, Trichosporon and Lipomyces, optionally wherein the yeast cell belongs to a species selected from Saccharomyces cerevisiae, Saccharomyces boulardi, Pichia pastoris, Kluyveromyces marxianus, Candida tropicalis, Cryptococcus albidus, Lipomyces lipofera, Lipomyces starkeyi, Rhodosporidium toruloides, Rhodotorula glutinis, Trichosporon pullulan and Yarrowia lipolytica, preferably the yeast cell is a Yarrowia lipolytica cell or a Saccharomyces cerevisiae cell.
35. The yeast cell according to any one of the preceding items, further expressing a heterologous NAD(P)H cytochrome b5 oxidoreductase (Ncb5or).
36. The yeast cell according to any one of the preceding items, wherein the Ncb5or is native to a plant, an insect or a mammal, such as Homo sapiens.
37. The yeast cell according to any one of the preceding items, wherein the Ncb5or is native to an insect, such as an insect of the genus Agrotis, Amyelois, Aphantopus, Arctia, Bicyclus, Bombus, Bombyx, Chilo, Cydia, Danaus, Drosophila, Eumeta, Galleria, Helicoverpa, Heliothis, Hyposmocoma, Leptidea, Lobesia, Manduca, Operophtera, Ostrinia, Papilio, Papilio, Papilio, Pieris, Plutella, Spodoptera, Trichoplusia, and Vanessa.
38. The yeast cell according to any one of the preceding items, wherein the Ncb5or is native to an insect selected from Agrotis segetum, Amyelois transitella, Aphantopus hyperantus, Arctia plantaginis, Bicyclus anynana, Bombus terrestris, Bombyx mandarina, Bombyx mori, Chilo suppressalis, Cydia pomonella, Danaus plexippus, Drosophila grimshawi, Drosophila melanogaster, Eumeta japonica, Galleria mellonella, Helicoverpa armigera, Heliothis virescens, Hyposmocoma kahamanoa, Leptidea sinapis, Lobesia botrana, Manduca sexta, Operophtera brumata, Ostrinia furnacalis, Papilio machaon, Papilio polytes, Papilio xuthus, Pieris rapae, Plutella xylostella, Spodoptera frugiperda, Spodoptera litura, Trichoplusia ni, and Vanessa tameamea.
39. The yeast according to any one of the preceding items, wherein the Ncb5or is selected from the group of Ncb5ors set forth in SEQ ID NOs: 23 to 27, SEQ ID NO: 53, SEQ ID NO: 104 and SEQ ID NO: 105, or functional variants having at least 70% identity or similarity thereto, such as at least 75% identity, such as at least 80% identity, such as at least 85% identity, such as at least 90% identity, such as at least 95% identity or similarity thereto.
40. The yeast cell according to any one of the preceding items, having a mutation leading to partial or total loss of activity of one or more of HFD1, HFD4, GPAT, FAO1, PEX10, such as having at least a mutation leading to partial or total loss of activity of FAO1 and one or more of HFD1, HFD4, PEX10 or GPAT.
41. The yeast cell according to any one of the preceding items, having a mutation in PEX10 and at least one of HFD1, HFD4, FAO1 and/or GPAT or a homologue thereof.
42. The yeast cell according to any one of the preceding items, having a mutation in FAO1 and at least one of HFD1, HFD4, PEX10 and/or GPAT or a homologue thereof.
43. The yeast cell according to any one of the preceding items, wherein HFD1, HFD4, PEX10 and/or FAO1 or a functional variant thereof having at least 60% similarity or identify thereto is deleted or mutated, resulting in partial loss of total loss of activity of Hfd1, Hfd4, Pex10 and/or Fao1, and/or wherein GPAT or a functional variant thereof having at least 60% similarity or identity thereto is mutated, resulting in reduced activity of GPAT.
44. The yeast cell according to any one of the preceding items, comprising a mutation in at least one POX gene, such as a POX gene selected from the group consisting of POX1, POX2, POX3, POX4, POX5, and POX6.
45. The yeast cell according to any one of the preceding items, wherein POX1, POX2, POX3), POX4, POX5, and/or POX6 or a functional variant thereof having at least 60% similarity or identify thereto, is deleted or mutated, resulting in partial loss of total loss of activity of Pox1, Pox2, Pox3, Pox4, Pox5 and/or Pox6.
46. The yeast cell according to any one of the preceding items, wherein said yeast cell further expresses:
   a heterologous cytochrome b5;
   a heterologous cytochrome b5 reductase;
   a hemoglobin;
   a heterologous thioesterase gene; and/or
   a fusion protein of a fatty acyl synthase and of a thioesterase.
47. The yeast cell according to any one of the preceding items, having an inactivation or modification of:
   native elongase(s) resulting in total or partial loss of activity;
   thioesterases(s) resulting in total or partial loss of activity; and/or
   activity of native fatty aldehyde dehydrogenase(s), fatty alcohol oxidase(s), peroxisome biogenesis factor and/or fatty acyl synthase(s).
48. The yeast cell according to any one of the preceding items, wherein the yeast cell is further modified to increase the availability of hexadecanoyl-CoA and/or hexadecenoyl-CoA.
49. The yeast cell according to any one of the preceding items, wherein at least one of the genes encoding the heterologous Δ13 desaturase, the further heterologous desaturase or the FAR is present in a high copy number.
50. The yeast cell according to any one of the preceding items, wherein at least one of the genes encoding the heterologous Δ13 desaturase, the further heterologous desaturase or the FAR is under the control of an inducible promoter.
51. The yeast cell according to any one of the preceding items, wherein at least one of the genes encoding the heterologous Δ13 desaturase, the further heterologous desaturase or the FAR are each independently comprised within the genome of the cell or within a vector comprised within the yeast cell.
52. The yeast cell according to any one of the preceding items, wherein the yeast cell is capable of producing (Z11, Z13)-hexadecenoic acid with a titer of at least 1 mg/L, such as at least 1.5 mg/L, such as at least 1.7 mg/L or more.
53. The yeast cell according to any one of the preceding items, wherein the yeast cell is capable of producing a Z13-fatty acid and/or (Z11, Z13)-hexadecadienoic acid with a titer of at least 20 mg/L, such as at least 50 mg/L, such as at least 60 mg/L, such as at least 70 mg/L, such as at least 80 mg/L, such as at least 90 mg/L, such as at least 100 mg/L, such as at least 150 mg/L, such as at least 200 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more.
54. The yeast cell according to any one of the preceding items, wherein the yeast cell is capable of producing a Z13-fatty alcohol and/or (Z11, Z13)-hexadecadien-1-ol with a titer of at least 0.005 mg/L, such as at least 0.01 mg/L, such as at least 0.02 mg/L, such as at least 0.03 mg/L, such as at least 0.05 mg/L, such as at least 0.06 mg/L, such as at least 0.075 mg/L, such as at least 0.1 mg/L, such as at least 0.2 mg/L, such as at least 0.3 mg/L, such as at least 0.4 mg/L, such as at least 0.5 mg/L, such as at least 0.6 mg/L, such as at least 0.7 mg/L, such as at least 0.8 mg/L, such as at least 0.9 mg/L, such as at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L or more.
55. A method for producing a desaturated fatty acyl-CoA having n′ double bond(s), wherein at least one of said double bond(s) is at position 13 in a yeast cell, said method comprising the steps of:
   i) providing a yeast cell according to any one of items 1 to 54,
   ii) incubating said yeast cell in a medium under conditions allowing expression of a heterologous Δ13 desaturase, said desaturase being capable of introducing a double bond at position 13 in a saturated or desaturated fatty acyl-CoA, preferably a desaturated fatty-acyl-CoA, having a carbon chain length of at least 14 and having n double bonds,
      wherein n and n′ are integers,
      wherein 0≤n≤3 and wherein 1≤n′≤4,
      thereby producing a desaturated fatty acyl-CoA having n′ double bond(s),
      wherein at least one of said double bond(s) is at position 13.
56. A method for producing a desaturated fatty acyl-CoA having n′ double bond(s), wherein at least one of said double bond(s) is at position 13, in a yeast cell, said method comprising the steps of:
   i) providing a yeast cell as disclosed herein,
   ii) incubating said yeast cell in a medium under conditions allowing expression of a heterologous Δ13 desaturase, said desaturase being capable of introducing a double bond at position 13 in a saturated fatty-acyl-CoA or a desaturated fatty acyl-CoA having a carbon chain consisting solely of single and double bonds, preferably a desaturated fatty-acyl-CoA, wherein said fatty acyl-CoA has a carbon chain length of at least 14 and has n double bonds,
      wherein n and n′ are integers,
      wherein 0≤n≤3 and wherein 1≤n′≤4,
      thereby producing a desaturated fatty acyl-CoA having n′ double bond(s),
      wherein at least one of said double bond(s) is at position 13.
57. The method according to any one of items 55 to 56, wherein said heterologous Δ13 desaturase is as defined in any one of the preceding items.
58. The method according to any one of items 55 to 57, wherein said further heterologous desaturase is as defined in any one of the preceding items.
59. The method according to any one of items 55 to 58, wherein said desaturated fatty acyl-CoA having n′ double bond(s) is as defined in any one of the preceding items.
60. The method according to any one of items 55 to 59, wherein said saturated or desaturated fatty acyl-CoA having a carbon chain length of at least 14 and having n double bonds is as defined in any one of the preceding items.
61. The method according to any one of items 55 to 60, wherein said yeast cell further expresses at least one alcohol-forming fatty acyl-CoA reductase (FAR) capable of converting at least part (Z11, Z13)-hexadecadienoyl-CoA into (Z11, Z13)-hexadecadien-1-ol.
62. The method according to any one of items 55 to 61, wherein said FAR is as defined in any one of the preceding items.
63. The method according to any one of items 55 to 62, further comprising the step of recovering and formulating the Z13-fatty alcohol and/or the (Z11, Z13)-hexadecadien-1-ol in a pheromone composition.
64. The method according to any one of items 55 to 63, further comprising the step of recovering the Z13-fatty alcohol and/or (Z11, Z13)-hexadecadien-1-ol.
65. The method according to any one of items 55 to 64, wherein the Z13-fatty alcohol and/or the (Z11, Z13)-hexadecadien-1-ol is further converted into a Z13-fatty alcohol acetate and/or (Z11, Z13)-hexadecadien-1-ol acetate, respectively.
66. The method according to any one of items 55 to 64, wherein the Z13-fatty alcohol and/or the (Z11, Z13)-hexadecadien-1-ol is further converted into a Z13-fatty aldehyde and/or (Z11, Z13)-hexadecadienal, respectively.
67. The method according to any one of items 57 to 66, wherein the conversion of the Z13-fatty alcohol and/or the (Z11, Z13)-hexadecadien-1-ol to a Z13-fatty alcohol acetate and/or (Z11, Z13)-hexadecadien-1-ol acetate is:
   a. performed in vivo by further expressing in the yeast cell an acetyltransferase capable of converting the Z13-fatty alcohol and/or the (Z11, Z13)-hexadecadien-1-ol to a Z13-fatty alcohol acetate and/or (Z11, Z13)-hexadecadien-1-ol acetate, respectively; or
   b. performed in vitro, e.g. by chemical conversion.
68. The method according to any one of items 57 to 67, wherein the Z13-fatty alcohol and/or the (Z11, Z13)-hexadecadien-1-ol is chemically oxidized into a Z13-fatty aldehyde and/or (Z11, Z13)-hexadecadienal.
69. The method according to any one of items 57 to 67, wherein the Z13-fatty alcohol and/or the (Z11, Z13)-hexadecadien-1-ol is acetylated into a Z13-fatty alcohol acetate and/or (Z11, Z13)-hexadecadien-1-ol acetate, optionally wherein the acetylation is performed chemically.
70. A nucleic acid or a system of nucleic acids for modifying a yeast cell, said nucleic acid or system of nucleic acids comprising at least one polynucleotide encoding a heterologous Δ13 desaturase said desaturase being capable of introducing a double bond at position 13 in a saturated or desaturated fatty acyl-CoA having a carbon chain length of at least 14 and having n double bonds thereby producing a desaturated fatty acyl-CoA having n′ double bond(s),
   wherein at least one of said double bond(s) is at position 13,
   wherein n and n′ are integers,
   wherein 0≤n≤3 and wherein 1≤n′≤4.
71. A nucleic acid or a system of nucleic acids for modifying a yeast cell, said nucleic acid or system of nucleic acids comprising at least one polynucleotide encoding a heterologous Δ13 desaturase said desaturase being capable of introducing a double bond at position 13 in a saturated fatty acyl-CoA or a desaturated fatty acyl-CoA having a carbon chain consisting solely of single and double bonds, preferably a desaturated fatty-acyl-CoA, wherein said fatty acyl-CoA has a carbon chain length of at least 14 and has n double bonds, thereby producing a desaturated fatty acyl-CoA having n′ double bond(s), wherein at least one of said double bond(s) is at position 13,
   wherein n and n′ are integers,
   wherein 0≤n≤3 and wherein 1≤n′≤4.
72. The nucleic acid or system of nucleic acids according to any one of items 70 to 71, wherein the heterologous Δ13 desaturase is encoded by the sequence set forth in SEQ ID NO: 2, SEQ ID NO: 46 and/or SEQ ID NO: 57, or functional variants thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.
73. The nucleic acid or system of nucleic acids according to any one of items 70 to 72, wherein said nucleic acid or system of nucleic acids further comprise a polynucleotide encoding a further heterologous desaturase capable of introducing a double bond at any position which is not position 13 in a saturated or desaturated fatty acyl-CoA having a carbon chain length of at least 14 and having n double bonds.
74. The nucleic acid or the system according to any one of items 70 to 73, wherein said further heterologous desaturase is encoded by a nucleic acid having at least 60% identity to a nucleic acid selected from the group of nucleic acids as set forth in SEQ ID NO: 4, SEQ ID NO: 6, SEQ ID NO: 8, SEQ ID NO: 10, SEQ ID NO: 12, SEQ ID NO: 28, SEQ ID NO: 30, SEQ ID NO: 32, SEQ ID NO: 47, SEQ ID NO: 52, SEQ ID NO: 50, SEQ ID NO: 138, SEQ ID NO: 58, SEQ ID NO: 60, SEQ ID NO: 62, SEQ ID NO: 64, SEQ ID NO: 66, SEQ ID NO: 68, and SEQ ID NO: 70, or homologues thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.
75. The nucleic acid or the system according to any one of items 70 to 74, wherein the nucleic acid or system of nucleic acids further comprise a polynucleotide encoding a fatty acyl-CoA reductase (FAR) capable of converting at least a part of (Z11, Z13)-hexadecadienoyl-CoA into (Z11, Z13)-hexadecadien-1-ol.
76. The nucleic acid or the system according to item 75, wherein the FAR is encoded by a nucleic acid having at least 60% identity to a nucleic acid selected from the group of FARs set forth in SEQ ID NO: 14, SEQ ID NO: 16, SEQ ID NO: 20, SEQ ID NO: 22, SEQ ID NO: 36, SEQ ID NO: 38, SEQ ID NO: 40, SEQ ID NO: 42, SEQ ID NO: 44, SEQ ID NO: 48, SEQ ID NO: 49, SEQ ID NO: 72, SEQ ID NO: 74, SEQ ID NO: 76, SEQ ID NO: 110, SEQ ID NO: 112, SEQ ID NO: 114, SEQ ID NO: 78, SEQ ID NO: 90, SEQ ID NO: 80, SEQ ID NO: 82, SEQ ID NO: 84, SEQ ID NO: 86, SEQ ID NO: 88, SEQ ID NO: 92, SEQ ID NO: 94, SEQ ID NO: 116, SEQ ID NO: 96, SEQ ID NO: 98, SEQ ID NO: 100, SEQ ID NO: 102, SEQ ID NO: 106, SEQ ID NO: 108, SEQ ID NO: 118, SEQ ID NO: 120, SEQ ID NO: 122, SEQ ID NO: 124, SEQ ID NO: 126, SEQ ID NO: 128 and SEQ ID NO: 130, or homologues thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.
77. A method of monitoring the presence of pest or disrupting the mating of pest, said method comprising the steps of:
   i) producing (Z11, Z13)-hexadecadien-1-ol and/or a Z13-fatty alcohol by the method according to any one of items 55 to 64;
   ii) converting the Z13-fatty alcohol and/or (Z11, Z13)-hexadecadien-1-ol into a Z13-fatty alcohol acetate and/or (Z11, Z13)-hexadecadien-1-ol acetate, respectively;
   iii) converting the Z13-fatty alcohol and/or (Z11, Z13)hexadecadien-1-ol into a Z13-fatty aldehyde and/or (Z11, Z13)-hexadecadienal, respectively;
   iv) recovering said Z13-fatty alcohol, (Z11, Z13)-hexadecadien-1-ol, Z13-fatty alcohol acetate, (Z11, Z13)-hexadecadien-1-ol acetate, Z13-fatty aldehyde, and/or (Z11, Z13)-hexadecadienal;
   v) formulating said Z13-fatty alcohol, (Z11, Z13)-hexadecadien-1-ol, Z13-fatty alcohol acetate, (Z11, Z13)-hexadecadien-1-ol acetate, Z13-fatty aldehyde and/or (Z11, Z13)-hexadecadienal as a pheromone composition; and
   vi) employing said pheromone composition as an integrated pest management composition.
78. A pheromone composition obtainable by the method comprising the following steps:
   i. producing (Z11, Z13)-hexadecadien-1-ol and/or a Z13-fatty alcohol by the method according to any one of items 55 to 64;
   ii. converting the Z13-fatty alcohol and/or (Z11, Z13)-hexadecadien-1-ol into a Z13-fatty alcohol acetate and/or (Z11, Z13)-hexadecadien-1-ol acetate, respectively;
   iii. converting the Z13-fatty alcohol and/or (Z11, Z13)-hexadecadien-1-ol into a Z13-fatty aldehyde and/or (Z11, Z13)-hexadecadienal, respectively;
   iv. recovering said Z13-fatty alcohol, (Z11, Z13)-hexadecadien-1-ol, Z13-fatty alcohol acetate, (Z11, Z13)-hexadecadien-1-ol acetate, Z13-fatty aldehyde, and/or (Z11, Z13)-hexadecadienal;
   v. formulating said Z13-fatty alcohol, (Z11, Z13)-hexadecadien-1-ol, Z13-fatty alcohol acetate, (Z11, Z13)-hexadecadien-1-ol acetate, Z13-fatty aldehyde and/or (Z11, Z13)-hexadecadienal as a pheromone composition.
79. A pheromone composition comprising a Z13-fatty alcohol, (Z11, Z13)-hexadecadien-1-ol, a Z13-fatty alcohol acetate, (Z11, Z13)-hexadecadien-1-ol acetate, a Z13-fatty aldehyde and/or (Z11, Z13)-hexadecadienal, wherein said pheromone composition comprises at least 20% biobased carbon, such as at least 30% biobased carbon, such as at least 40% biobased carbon, such as at least 50% biobased carbon, such as at least 60% biobased carbon, such as at least 70% biobased carbon, such as at least 75% biobased carbon, such as at least 80% biobased carbon, such as at least 85% biobased carbon, such as at least 90% biobased carbon, such as at least 95% biobased carbon, such as 100% biobased carbon.
80. A pheromone compound selected from the group consisting of a Z13-fatty alcohol, (Z11, Z13)-hexadecadien-1-ol, a Z13-fatty alcohol acetate, (Z11, Z13)-hexadecadien-1-ol acetate, a Z13-fatty aldehyde and/or (Z11, Z13)-hexadecadienal, wherein said pheromone compound comprises at least 20% biobased carbon, such as at least 30% biobased carbon, such as at least 40% biobased carbon, such as at least 50% biobased carbon, such as at least 60% biobased carbon, such as at least 70% biobased carbon, such as at least 75% biobased carbon, such as at least 80% biobased carbon, such as at least 85% biobased carbon, such as at least 90% biobased carbon, such as at least 95% biobased carbon, such as 100% biobased carbon.
81. The pheromone composition according to item 79, further comprising one or more additional compounds selected from the group consisting of liquid carriers, solid carriers and substrates.
82. The pheromone composition according to any of items 79 or 81, further comprising one or more compounds selected from the group consisting of vegetable oils, refined mineral oils or fractions thereof, rubbers, plastics, silica, diatomaceous earth, wax matrix and cellulose powder.
83. The pheromone composition according to any of items 79 or 81-82, wherein said pheromone composition is in the form of a solution, a gel, or a powder.
84. The pheromone composition according to any of items 79 or 81-83, wherein said pheromone composition is formulated so that it can be dispensed.
85. The pheromone composition according to any of items 79 or 81-84, wherein the ratio of (Z11, Z13)-hexadecadien-1-ol to the Z11-fatty alcohol, (Z11, Z13)-hexadecadien-1-ol acetate to the Z11-fatty alcohol acetate, and/or (Z11, Z13)-hexadecadienal to the Z11-fatty aldehyde in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.
86. The pheromone composition according to any of items 79 or 81-85, wherein the ratio of (Z11, Z13)-hexadecadien-1-ol to Z11,Z13-hexadecadienal and/or (Z11, Z13)-hexadecadien-1-ol to (Z11, Z13)-hexadecadien-1-ol acetate in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.
87. The pheromone composition according to any of items 79 or 81-86, wherein the ratio of (Z11, Z13)-hexadecadien-1-ol to a Z13-fatty alcohol, (Z11, Z13)-hexadecadien-1-ol acetate to a Z13-fatty alcohol acetate, and/or (Z11, Z13)-hexadecadienal to a Z13-fatty aldehyde in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.
88. The pheromone composition or the pheromone compound according to any of items 79-87, wherein said pheromone composition or said pheromone compound comprises at least 30% biobased carbon, alternatively 40% biobased carbon, alternatively 50% biobased carbon, alternatively 60% biobased carbon, alternatively 70% biobased carbon, alternatively 75% biobased carbon, alternatively 80% biobased carbon, alternatively 85% biobased carbon, alternatively 90% biobased carbon, alternatively 95% biobased carbon.
89. The pheromone composition or the pheromone compound according to any of items 79-88, wherein said pheromone composition or said pheromone compound comprises at least 80% biobased carbon, alternatively 85% biobased carbon, alternatively 90% biobased carbon, alternatively 95% biobased carbon, alternatively 96% biobased carbon, alternatively 97% biobased carbon, alternatively 98% biobased carbon, alternatively 99% biobased carbon.
90. The pheromone composition or the pheromone compound according to any of items 79-89, wherein said pheromone composition or said pheromone compound comprises 30-100% biobased carbon, alternatively 40-100% biobased carbon, alternatively 50-100% biobased carbon, alternatively 60-100% biobased carbon, alternatively 70-100% biobased carbon, alternatively 75-100% biobased carbon, alternatively 80-100% biobased carbon, alternatively 85-100% biobased carbon, alternatively 90-100% biobased carbon, alternatively 95-100% biobased carbon.
91. The pheromone composition or the pheromone compound according to any of items 79-90, wherein said pheromone composition or said pheromone compound comprises 80-100% biobased carbon, alternatively 85-100% biobased carbon, alternatively 90-100% biobased carbon, alternatively 95-100% biobased carbon, alternatively 96-100% biobased carbon, alternatively 97-100% biobased carbon, alternatively 98-100% biobased carbon, alternatively 99-100% biobased carbon.
92. A method for producing a pheromone composition according to any of items 79 or 81-91 comprising the following steps:
   i. producing (Z11, Z13)-hexadecadien-1-ol and/or a Z13-fatty alcohol by the method according to any one of items 55 to 64;
   ii. converting the Z13-fatty alcohol and/or (Z11, Z13)-hexadecadien-1-ol into a Z13-fatty alcohol acetate and/or (Z11, Z13)-hexadecadien-1-ol acetate, respectively;
   iii. converting the Z13-fatty alcohol and/or (Z11, Z13)-hexadecadien-1-ol into a Z13-fatty aldehyde and/or (Z11, Z13)-hexadecadienal, respectively;
   iv. recovering said Z13-fatty alcohol, (Z11, Z13)-hexadecadien-1-ol, Z13-fatty alcohol acetate, (Z11, Z13)-hexadecadien-1-ol acetate, Z13-fatty aldehyde, and/or (Z11, Z13)-hexadecadienal;
   v. formulating said Z13-fatty alcohol, (Z11, Z13)-hexadecadien-1-ol, Z13-fatty alcohol acetate, (Z11, Z13)-hexadecadien-1-ol acetate, Z13-fatty aldehyde and/or (Z11, Z13)-hexadecadienal as a pheromone composition, optionally comprising one or more additional compounds such as a liquid or solid carrier or substrate;
   vi. optionally determining the biobased carbon content of the pheromone composition.
93. A method for producing a pheromone compound according to any of items 80-91, in a yeast cell, said method comprising the steps of:
   i) providing a yeast cell expressing a heterologous Δ13 fatty acyl-CoA desaturase,
   ii) incubating said yeast cell in a medium under conditions allowing expression of said heterologous Δ13 fatty acyl-CoA desaturase,
thereby producing said pheromone compound.
94. A yeast cell capable of producing a pheromone compound according to any of items 80-91, wherein said yeast cell expresses a heterologous Δ13 fatty acyl-CoA desaturase.
95. The method or the yeast cell according to any of items 93-94, wherein said yeast cell expresses a further heterologous desaturase.
96. The method or the yeast cell according to any of items 93-95, wherein said yeast cell further expresses at least one alcohol-forming fatty acyl-CoA reductase (FAR).
97. The method according to any of items 93-96 further comprising a step of determining the biobased carbon content of the pheromone compound.
98. Use of an Amyelois transitella desaturase in a method for introducing a double bond at position 13 in a saturated or desaturated fatty acyl-CoA.
99. The use according to item 98, wherein the desaturated fatty acyl-CoA has a carbon length of 14 or more, such as 15, such as 16, such as 17 or more and/or the saturated fatty acid methyl ester has a carbon length of 14 or more, such as 15, such as 16, such as 17 or more.
100. The use according to any one of items 98 to 99, wherein the desaturated fatty acyl-CoA has a carbon chain consisting solely of single and double bonds.
101. The use according to any one of items 98 to100, wherein the Amyelois transitella desaturase is AtrAATQ (SEQ ID NO: 1) or functional variants thereof having at least 70% similarity, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
102. Use of a Notodonta dromedarius desaturase in a method for introducing a double bond at position 13 in a saturated or a desaturated fatty acyl-CoA.
103. The use according to item 102, wherein the desaturated fatty acyl-CoA has a carbon length of 14 or more, such as 15, such as 16, such as 17 or more and/or the saturated fatty acid methyl ester has a carbon length of 14 or more, such as 15, such as 16, such as 17 or more.
104. The use according to any one of items 102 to 103, wherein the desaturated fatty acyl-CoA has a carbon chain consisting solely of single and double bonds.
105. The use according to any one of items 102 to 104, wherein the Notodonta dromedarius desaturase is NdDes1 (SEQ ID NO: 56) or functional variants thereof having at least 70% similarity, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
106. The use according to any one of items 98 to 105, wherein the method is performed in vitro.
107. The use according to any one of items 98 to 106, wherein the method is performed in vivo, preferably wherein the method is performed in a yeast cell according to any one of items 1 to 54.
108. A (Z11, Z13)-hexadecadienoyl-CoA obtainable by the method according to any one of items 55 to 66.
109. A (Z11, Z13)-hexadecadien-1-ol obtainable by the method according to any one of items 61 to 66.
110. A fermentation broth comprising the yeast cell according to any one of items 1 to 54.
111. A yeast cell capable of producing (Z11, Z13)-hexadecadienoyl-CoA, said yeast cell expressing
   i) at least one first heterologous desaturase capable of introducing a first double bond at position 11 in a hexadecanoyl-CoA, thereby converting said hexadecanoyl-CoA to (Z11)-hexadecenoyl-CoA; and
   ii) at least one second heterologous desaturase capable of introducing a second double bond at position 13 in (Z11)-hexadecenoyl-CoA, thereby converting (Z11)-hexadecenoyl-CoA into (Z11, Z13)-hexadecadienoyl-CoA.
112. A yeast cell capable of producing a desaturated fatty acyl-CoA, said yeast cell expressing an AtrAATQ (SEQ ID NO: 1) desaturase capable of introducing a cis-double bond at position 13 in a fatty acyl-CoA, thereby converting the fatty acyl-CoA into a Z13-fatty acyl-CoA.
113. A yeast cell capable of producing a desaturated fatty acyl-CoA, said yeast cell expressing a NdDes1 (SEQ ID NO: 56) desaturase capable of introducing a cis-double bond at position 13 in a fatty acyl-CoA, thereby converting the fatty acyl-CoA into a Z13-fatty acyl-CoA.
114. A method for producing (Z11, Z13)-hexadecadien-1-ol, said method comprising the steps of:
   i) Providing a yeast cell according to any one of items 1 to 54;
   ii) Incubating said yeast cell in a medium under conditions allowing expression of:
      a. a further desaturase, said desaturase being capable of introducing a double bond at position 11 in hexadecanoyl-CoA, thereby converting at least part of hexadecanoyl-CoA into (Z11)-hexadecenoyl-CoA;
      b. a heterologous Δ13 desaturase, said desaturase being capable of introducing a double bond at position 13 in (Z11)-hexadecenoyl-CoA, thereby converting at least part of (Z11)-hexadecenoyl-CoA into (Z11, Z13)-hexadecadienoyl-CoA;
      c. a FAR, said FAR being capable of converting at least part of (Z11, Z13)-hexadecadienoyl-CoA into (Z11, Z13)-hexadecadien-1-ol;
   iii) optionally, recovering the (Z11, Z13)-hexadecadien-1-ol;
   iv) optionally, converting the (Z11, Z13)-hexadecadien-1-ol into (Z11, Z13)-hexadecadien-1-ol acetate;
   v) optionally, converting the (Z11, Z13)-hexadecadien-1-ol into (Z11, Z13)-hexadecadienal;
   vi) optionally, recovering said Z13-fatty alcohol, (Z11, Z13)-hexadecadien-1-ol, Z13-fatty alcohol acetate, (Z11, Z13)-hexadecadien-1-ol acetate, Z13-fatty aldehyde, and/or (Z11, Z13)-hexadecadienal;
   vii) optionally, formulating the (Z11, Z13)-hexadecadien-1-ol and/or the (Z11, Z13)-hexadecadienal into a pheromone composition.

1. A pheromone composition comprising a Z13-fatty alcohol, (Z11, Z13)-hexadecadien-1-ol, a Z13-fatty alcohol acetate, (Z11, Z13)-hexadecadien-1-ol acetate, a Z13-fatty aldehyde and/or (Z11, Z13)-hexadecadienal, wherein said pheromone composition comprises at least 70% biobased carbon, such as at least 75% biobased carbon, such as at least 80% biobased carbon, such as at least 85% biobased carbon, such as at least 90% biobased carbon, such as at least 95% biobased carbon, such as 100% biobased carbon.

2. A pheromone compound selected from the group consisting of a Z13-fatty alcohol, (Z11, Z13)-hexadecadien-1-ol, a Z13-fatty alcohol acetate, (Z11, Z13)-hexadecadien-1-ol acetate, a Z13-fatty aldehyde and/or (Z11, Z13)-hexadecadienal, wherein said pheromone compound comprises at least 70% biobased carbon, such as at least 75% biobased carbon, such as at least 80% biobased carbon, such as at least 85% biobased carbon, such as at least 90% biobased carbon, such as at least 95% biobased carbon, such as 100% biobased carbon.

3. A yeast cell capable of producing a desaturated fatty acyl-coenzyme A (fatty acyl-CoA) having n′ double bond(s), wherein at least one of said double bond(s) is at position 13,
wherein said yeast cell expresses a heterologous Δ13 fatty acyl-CoA desaturase, said desaturase being capable of introducing a double bond at position 13 in a saturated fatty acyl-CoA or a desaturated fatty acyl-CoA having a carbon chain consisting solely of single and double bonds, preferably a desaturated fatty acyl-CoA, wherein said fatty acyl-CoA has a carbon chain length of at least 14 and has n double bond(s),
wherein n and n′ are integers,
wherein 0≤n≤3 and wherein 1≤n′≤4.

4. The yeast cell according to claim 3, wherein said heterologous Δ13 desaturase is native to an organism of a genus selected from the group consisting of Amyelois, Desmia and Notodonta, such as to a species selected from the group consisting of Amyelois transitella, Desmia funeralis, Thaumetopoea bonjeani and Notodonta dromedarius, optionally wherein said heterologous Δ13 desaturase is an Amyelois desaturase, such as an Amyelois transitella desaturase or a Notodonta desaturase, such as a Notodonta dromedarius desaturase, further optionally wherein said heterologous Δ13 desaturase is AtrAATQ as set forth in SEQ ID NO: 1 or NdDes1 as set forth in SEQ ID NO:
56, or a functional homologue thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

5. The yeast cell according to any one of claims 3 to 4, wherein said heterologous Δ13 desaturase is AtrAATQ as set forth in SEQ ID NO: 1 or NdDes1 as set forth in SEQ ID NO: 56, or a functional homologue thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

6. The yeast cell according to any one of claims 3 to 5, wherein said saturated or desaturated fatty acyl-CoA having a carbon chain length of at least 14 and having n double bonds:
i) has a carbon length of at least 15, such as at least 16;
ii) is hexadecanoyl-CoA or hexadecenoyl-CoA;
iii) has one double bond (i.e. n=1); and/or
iv) is (Z11)-hexadecenoyl-CoA.

7. The yeast cell according to any one of claims 3 to 6, wherein said desaturated fatty acyl-CoA having n′ double bond(s):
i) has a carbon chain length of at the most 20, such as at the most 19, such as at the most 18, such as at the most 17, such as at the most 16, such as at least 17, such as at least 18, such as at least 19, such as at least 20, such as at least 21, such as at least 22;
ii) has one double bond (i.e. n′=1);
iii) has two double bonds (i.e. n′=2);
iv) is (Z13)-hexadecenoyl-CoA; and/or
v) is (Z11, Z13)-hexadecadienoyl-CoA.

8. The yeast cell according to any one of the preceding claims, wherein n′=2.

9. The yeast cell according to any one of claims 3 to 8, wherein said yeast cell expresses a further heterologous desaturase; optionally wherein said further heterologous desaturase is capable of introducing a double bond at any position which is not position 13 in a saturated or desaturated fatty acyl-CoA having a carbon chain length of at least 14 and having n double bonds; optionally wherein said further heterologous desaturase is capable of introducing a double bond at least in position 11; further optionally wherein said further heterologous desaturase is a Δ11 desaturase.

10. The yeast cell according to any one of claims 3 to 9, wherein said further heterologous desaturase is a Spodoptera desaturase, an Amyelois desaturase, an Agrotis desaturase, a Trichoplusia desaturase, a Plutella desaturase, a Helicoverpa desaturase, a Chilo desaturase, a Diatraea desaturase, a Plodia desaturase, a Lobesia desaturase, a Antheraea desaturase, a Cadra desaturase, or an Yponomeuta desaturase, such as a Spodoptera litura desaturase, a Spodoptera littoralis desaturase, a Spodoptera exigua desaturase, an Amyelois transitella desaturase, an Agrotis segetum desaturase, a Trichoplusia ni desaturase, a Plutella xylostella desaturase, a Helicoverpa zea desaturase, a Chilo suppressalis desaturase, a Diatraea saccharalis desaturase, a Plodia interpunctella desaturase, a Lobesia botrana desaturase, an Antheraea pernyi desaturase, a Cadra cautella desaturase, or a Yponomeuta padella desaturase, optionally wherein said further heterologous desaturase is a desaturase selected from the group consisting of Desat38 (SEQ ID NO: 3), Desat20 (SEQ ID NO: 5), Desat16 (SEQ ID NO: 7), Desat19 (SEQ ID NO: 9), Desat21 (SEQ ID NO: 11), Desat37 (SEQ ID NO: 31), Desat45 (SEQ ID NO: 33), Desat51 (SEQ ID NO: 29), Desat44 (SEQ ID NO: 51), Desat63 (SEQ ID NO: 59), Desat65 (SEQ ID NO: 61), Desat71 (SEQ ID NO: 63), Desat79 (SEQ ID NO: 65), Desat72 (SEQ ID NO: 67), Desat70 (SEQ ID NO: 69, and Desat73 (SEQ ID NO: 71), or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

11. The yeast cell according to any one of claims 3 to 10, wherein the yeast cell is capable of producing (Z11, Z13)-hexadecadien-1-ol, said yeast cell further expressing at least one alcohol-forming fatty acyl-CoA reductase (FAR) capable of converting at least a part of (Z11, Z13)-hexadecadienoyl-CoA into (Z11, Z13)-hexadecadien-1-ol; optionally wherein the FAR is native to an organism of a genus selected from the group consisting of Agrotis, Amyelois, Bicyclus, Bombus, Chilo, Cydia, Helicoverpa, Heliothis, Lobesia, Ostrinia, Plodia, Plutella, Spodoptera, Trichoplusia, Tyta, Yponomeuta, Ostrinia, Marinobacter, Manducta, Chrysodeixis, and Grapholita, such as Agrotis ipsilon, Agrotis segetum, Amyelois transitella, Bicyclus anynana, Bombus lapidarius, Chilo suppressalis, Cydia pomonella, Helicoverpa armigera, Helicoverpa assulta, Heliothis subflexa, Heliothis virescens, Lobesia botrana, Ostrinia nubilalis, Plodia interpunctella, Plutella xylostella, Spodoptera exigua, Spodoptera frugiperda, Spodoptera littoralis, Spodoptera litura, Trichoplusia ni, Tyta alba, Yponomeuta rorellus, Marinobacter algicola, Manducta sexta, Ostrinia nubilalis, Ostrinia furnacalis, Ostrinia zag, Ostrinia zea, Chrysodeixis includens, and Grapholita molesta, especially Spodoptera exigua, Helicoverpa armigera, Spodoptera litura, Amyelois transitella and Agrotis ipsilon; further optionally wherein the FAR is selected from the group consisting of FAR1 (SEQ ID NO: 13, FAR33 (SEQ ID NO: 15), FAR4 (SEQ ID NO: 19), FAR6 (SEQ ID NO: 21), FAR28 (SEQ ID NO: 45), FAR32 (SEQ ID NO: 35), FAR16 (SEQ ID NO: 37), FAR17 (SEQ ID NO: 41), FAR19 (SEQ ID NO: 39), FAR38 (SEQ ID NO: 43), FAR5 (SEQ ID NO: 73), FAR8 (SEQ ID NO: 75), FAR9 (SEQ ID NO: 77), FAR12 (SEQ ID NO: 79), FAR42 (SEQ ID NO: 81), FAR25 (SEQ ID NO: 83), FAR13 (SEQ ID NO: 85), FAR14 (SEQ ID NO: 87), FAR15 (SEQ ID NO: 89), FAR18 (SEQ ID NO: 91), FAR20 (SEQ ID NO: 93), FAR22 (SEQ ID NO: 95), FAR27 (SEQ ID NO: 97), FAR29 (SEQ ID NO: 99), FAR30 (SEQ ID NO: 101), FAR43 (SEQ ID NO: 103), FAR34 (SEQ ID NO: 107), FAR35 (SEQ ID NO: 109, FAR44 (SEQ ID NO: 111), FAR48 (SEQ ID NO: 113), FAR49 (SEQ ID NO: 115), FAR45 (SEQ ID NO: 117), FAR40 (SEQ ID NO: 119), FAR41 (SEQ ID NO: 121), FAR51 (SEQ ID NO: 123), FAR52 (SEQ ID NO: 125), FAR47 (SEQ ID NO: 127), FAR46 (SEQ ID NO: 129), and FAR50 (SEQ ID NO: 131), or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

12. The yeast cell according to any one of claims 3 to 11, wherein the yeast cell belongs to a genus selected from Saccharomyces, Pichia, Yarrowia, Kluyveromyces, Candida, Rhodotorula, Rhodosporidium, Cryptococcus, Trichosporon and Lipomyces, optionally wherein the yeast cell belongs to a species selected from Saccharomyces cerevisiae, Saccharomyces boulardi, Pichia pastoris, Kluyveromyces marxianus, Candida tropicalis, Cryptococcus albidus, Lipomyces lipofera, Lipomyces starkeyi, Rhodosporidium toruloides, Rhodotorula glutinis, Trichosporon pullulan and Yarrowia lipolytica, preferably the yeast cell is a Yarrowia lipolytica cell or a Saccharomyces cerevisiae cell.

13. The yeast cell according to any one of claims 3 to 12, wherein the yeast cell is capable of producing:
i) (Z11, Z13)-hexadecadienoic acid with a titer of at least 1 mg/L, such as at least 1.5 mg/L, such as at least 1.7 mg/L or more;
ii) a Z13-fatty acid and/or (Z11, Z13)-hexadecadienoic acid with a titer of at least 20 mg/L, such as at least 50 mg/L, such as at least 60 mg/L, such as at least 70 mg/L, such as at least 80 mg/L, such as at least 90 mg/L, such as at least 100 mg/L, such as at least 150 mg/L, such as at least 200 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more; and/or
iii) a Z13-fatty alcohol and/or (Z11, Z13)-hexadecadien-1-ol with a titer of at least 0.005 mg/L, such as at least 0.01 mg/L, such as at least 0.02 mg/L, such as at least 0.03 mg/L, such as at least 0.05 mg/L, such as at least 0.06 mg/L, such as at least 0.075 mg/L, such as at least 0.1 mg/L, such as at least 0.2 mg/L, such as at least 0.3 mg/L, such as at least 0.4 mg/L, such as at least 0.5 mg/L, such as at least 0.6 mg/L, such as at least 0.7 mg/L, such as at least 0.8 mg/L, such as at least 0.9 mg/L, such as at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L or more.

14. A method for producing a desaturated fatty acyl-CoA having n′ double bond(s), wherein at least one of said double bond(s) is at position 13, in a yeast cell, said method comprising the steps of:
i) providing a yeast cell according to any one of claims 3 and 13, ii) incubating said yeast cell in a medium under conditions allowing expression of a heterologous Δ13 desaturase, said desaturase being capable of introducing a double bond at position 13 in a saturated fatty acyl-CoA or a desaturated fatty acyl-CoA having a carbon chain consisting solely of single and double bonds, preferably a desaturated fatty-acyl-CoA, having a carbon chain length of at least 14 and having n double bonds,
wherein n and n′ are integers,
wherein 0≤n≤3 and wherein 1≤n′≤4,
thereby producing a desaturated fatty acyl-CoA having n′ double bond(s),
wherein at least one of said double bond(s) is at position 13.

15. The method according to claim 14, wherein:
i) said heterologous Δ13 desaturase is as defined in any one of claims 4 to 5;
ii) said further heterologous desaturase is as defined in any one of claims 8 and 10;
iii) said yeast cell is as defined in any one of claims 3 and 13;
iv) said desaturated fatty acyl-CoA having n′ double bond(s) is as defined in claim 7; and/or
v) said saturated or desaturated fatty acyl-CoA having a carbon chain length of at least 14 and having n double bonds is as defined in claim 6.

16. The method according to any one of claims 14 to 15, wherein said yeast cell further expresses at least one alcohol-forming fatty acyl-CoA reductase (FAR) capable of converting at least part of (Z13)-hexadecenoyl-CoA and/or (Z11, Z13)-hexadecadienoyl-CoA into a Z13-fatty alcohol and/or (Z11, Z13)-hexadecadien-1-ol, respectively, optionally wherein said FAR is as defined in claim 11.

17. The method according to any one of claims 14 to 16, further comprising the steps of:
i) recovering the Z13-fatty alcohol and/or (Z11, Z13)-hexadecadien-1-ol;
ii) converting the Z13-fatty alcohol and/or (Z11,Z13)-hexadecadien-1-ol into a Z13-fatty alcohol acetate and/or (Z11,Z13)-hexadecadien-1-yl acetate, respectively;
iii) converting the Z13-fatty alcohol and/or (Z11, Z13)-hexadecadien-1-ol into a Z13-fatty aldehyde and/or (Z11, Z13)-hexadecadienal,
respectively,
iv) recovering said Z13-fatty alcohol, (Z11, Z13)-hexadecadien-1-ol, Z13-fatty alcohol acetate, (Z11, Z13)-hexadecadien-1-ol acetate, Z13-fatty aldehyde, and/or (Z11, Z13)-hexadecadienal; and/or
v) formulating said Z13-fatty alcohol, (Z11, Z13)-hexadecadien-1-ol, Z13-fatty alcohol acetate, (Z11,Z13)-hexadecadien-1-yl acetate, Z13-fatty aldehyde, and/or (Z11, Z13)-hexadecadienal in a pheromone composition.

18. A pheromone composition obtainable by a method comprising the following steps:
i) producing (Z11, Z13)-hexadecadien-1-ol and/or a Z13-fatty alcohol by the method according to any one of claims 14 to 17;
ii) recovering the (Z11, Z13)-hexadecadien-1-ol and/or Z13-fatty alcohol;
iii) converting the Z13-fatty alcohol and/or (Z11, Z13)-hexadecadien-1-ol into a Z13-fatty alcohol acetate and/or (Z11, Z13)-hexadecadien-1-ol acetate, respectively;
iv) converting the Z13-fatty alcohol and/or (Z11, Z13)-hexadecadien-1-ol into a Z13-fatty aldehyde and/or (Z11, Z13)-hexadecadienal, respectively;
v) recovering said Z13-fatty alcohol, (Z11, Z13)-hexadecadien-1-ol, Z13-fatty alcohol acetate, (Z11, Z13)-hexadecadien-1-ol acetate, Z13-fatty aldehyde, and/or (Z11, Z13)-hexadecadienal;
vi) formulating said Z13-fatty alcohol, (Z11, Z13)-hexadecadien-1-ol, Z13-fatty alcohol acetate, (Z11, Z13)-hexadecadien-1-ol acetate, Z13-fatty aldehyde, and/or (Z11, Z13)-hexadecadienal as a pheromone composition.

19. Use of an Amyelois transitella desaturase in a method for introducing a double bond at position 13 in a saturated or desaturated fatty acyl-CoA, optionally wherein the Amyelois transitella desaturase is AtrAATQ (SEQ ID NO: 1) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

20. Use of an Notodonta dromedarius desaturase in a method for introducing a double bond at position 13 in a saturated or desaturated fatty acyl-CoA, optionally wherein the Notodonta dromedarius desaturase is NdDes1 (SEQ ID NO: 56) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

21. Use of an Amyelois transitella desaturase in a method for introducing a double bond at position 13 in a saturated fatty acyl-CoA or a desaturated fatty acyl-CoA having a carbon chain consisting solely of single and double bonds, optionally wherein the Amyelois transitella desaturase is AtrAATQ (SEQ ID NO: 1) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

22. Use of an Notodonta dromedarius desaturase in a method for introducing a double bond at position 13 in a saturated fatty acyl-CoA or a desaturated fatty acyl-CoA having a carbon chain consisting solely of single and double bonds, optionally wherein the Notodonta dromedarius desaturase is NdDes1 (SEQ ID NO: 56) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
